(19) United States
(12) Patent Application Publication
(10) Pub. No.: US 2022/0184217 A1
(43) Pub. Date: Jun. 16, 2022

(54) HSP90-BINDING CONJUGATES AND FORMULATIONS THEREOF

(71) Applicant: TARVEDA THERAPEUTICS, INC., Watertown, MA (US)

(72) Inventors: Brian H. White, Malden, MA (US); Mark T. Bilodeau, Waltham, MA (US); Ashis K. Saha, Arlington, MA (US)

(21) Appl. No.: 17/600,692

(22) PCT Filed: Apr. 1, 2020
(86) PCT No.: PCT/US2020/026137

§ 371 Date: Oct. 1, 2021

Related U.S. Application Data
(60) Provisional application No. 62/986,245, filed on Mar. 6, 2020.
(60) Provisional application No. 62/828,645, filed on Apr. 3, 2019.

(51) Int. Cl. A61K 47/54; A61K 51/04

(52) U.S. Cl. CPC A61K 47/545; A61K 51/0459; A61K 51/0482; A61K 51/0497

(57) ABSTRACT

Conjugates of an active agent attached to a targeting moiety, such as at least one HSP90 binding moiety, via a linker, have been designed. Such conjugates can provide improved temporospatial delivery of the active agent, improved biodistribution and penetration in tumor, and/or decreased toxicity. Methods of making the conjugates and the formulations thereof are provided. Methods of administering the formulations to a subject in need thereof are provided, for example, to treat or prevent cancer.

### REFERENCED TO RELATED APPLICATIONS

The present application claims priority to U.S. Provisional Patent Application No. 62/986,245, filed Mar. 6, 2020, entitled HSP90-TARGETING CONJUGATES AND FORMULATIONS THEREOF, and U.S. Provisional Patent Application No. 62/828,645, filed Apr. 3, 2019, entitled HSP90-TARGETING CONJUGATES AND FORMULATIONS THEREOF, the contents of each of which are herein incorporated by reference in their entirety.

### FIELD OF THE DISCLOSURE

The invention relates to the use of molecules targeting heat shock proteins including heat shock protein 90 (HSP90), e.g., for treating cancer.

### BACKGROUND

Heat shock protein 90 (HSP90) is a molecular chaperone that is important for maintaining stability and function of numerous client proteins. It is considered a major therapeutic target for anticancer drug development.

### SUMMARY OF THE DISCLOSURE

The present application provides a conjugate comprising an active agent coupled to an HSP90 targeting moiety by a linker and a pharmaceutical composition comprising such a conjugate. Methods of making and using such conjugates are also provided.

### BRIEF DESCRIPTION OF DRAWINGS

FIG. 1 shows the biodistribution data of the radioactive analog of CMP51 (comprising Lu177) in the NCI-H460 tumor model. CMP51 demonstrates selective tumor exposure with the exception of kidney.

FIG. 2 shows the biodistribution data of the non-radioactive analog of CMP6 (comprising Lu175) in the NCI-H460 tumor model. CMP6 demonstrates selective tumor retention over kidney and liver.

### DETAILED DESCRIPTION

Applicants have designed HSP90 targeting conjugates comprising an active agent. Such targeting can, for example, improve the amount of active agent at a site and decrease active agent toxicity to the subject. HSP90 targeting conjugates of the present invention have deep and rapid tumor penetration. High accumulation and long retention time of HSP90 targeting conjugates enable the use of cytotoxic and non-cytotoxic payloads, such as radionuclides, chemotherapeutic agents, kinase inhibitors, or immuno-oncology modulators.

As used herein, “toxicity” refers to the capacity of a substance or composition to be harmful or poisonous to a cell, tissue organism or cellular environment. Low toxicity refers to a reduced capacity of a substance or composition to be harmful or poisonous to a cell, tissue organism or cellular environment. Such reduced or low toxicity may be relative to a standard measure, relative to a treatment or relative to the absence of a treatment.

Toxicity may further be measured relative to a subject's weight loss where weight loss over 15%, over 20% or over 30% of the body weight is indicative of toxicity. Other metrics of toxicity may also be measured such as patient presentation metrics including lethargy and general malaiase. Neutropenia or thrombopenia may also be metrics of toxicity.

Pharmacologic indicators of toxicity include elevated AST/ALT levels, neurotoxicity, kidney damage, GI damage and the like.

In addition, the toxicity of a conjugate containing an HSP90 targeting moiety linked to an active agent for cells that do not overexpress HSP90 is predicted to be decreased compared to the toxicity of the active agent alone. Without committing to any particular theory, applicants believe that this feature is because the ability of the conjugated active agent to be retained in a normal cell is decreased relative to a tumor cell.

In some embodiments, the active agent and the targeting moiety, when connected by a linker into a conjugate, have synergistic effects. The efficacy of the conjugate is better than the active agent and/or the targeting moiety alone.

In some embodiments, the potency of the active agent is reduced when it is connected to a targeting moiety by a cleavable linker. Upon cleavage of the linker at a target site, such as a tumor site, the active agent is released and full potency is recovered.

It is an object of the invention to provide improved compounds, compositions, and formulations for temporospatial drug delivery.

It is further an object of the invention to provide methods of making improved compounds, compositions, and formulations for temporospatial drug delivery.

It is also an object of the invention to provide methods of administering the improved compounds, compositions, and formulations to individuals in need thereof.

### I. Conjugates

Conjugates include an active agent or prodrug thereof attached to a targeting moiety, e.g., a molecule that can bind to HSP90, by a linker. The conjugates can be a conjugate between a single active agent and a single targeting moiety, e.g., a conjugate having the structure X-Y-Z where X is the targeting moiety, Y is the linker, and Z is the active agent.

In some embodiments the conjugate contains more than one targeting moiety, more than one linker, more than one active agent, or any combination thereof. The conjugate can have any number of targeting moieties, linkers, and active agents. The conjugate can have the structure X-Y-Z-Y-X, (X-Y)ₙ-Z, X-(Y-Z)ₙ, Xₙ-Y-Z, X-Y-Zₙ, (X-Y-Z)ₙ, (X-Y-Z-Y)ₙ-Z, where X is a targeting moiety, Y is a linker, Z is an active agent, and n is an integer between 1 and 50, between 2 and 20, for example, between 1 and 5. Each occurrence of X, Y, and Z can be the same or different, e.g., the conjugate can contain more than one type of targeting moiety, more than one type of linker, and/or more than one type of active agent.

The conjugate can contain more than one targeting moiety attached to a single active agent. For example, the conjugate can include an active agent with multiple targeting moieties each attached via a different linker. The conjugate can have the structure X-Y-Z-Y-X where each X is a targeting moiety that may be the same or different, each Y is a linker that may be the same or different, and Z is the active agent.

The conjugate can contain more than one active agent attached to a single targeting moiety. For example, the conjugate can include a targeting moiety with multiple active agents each attached via a different linker. The conjugate can have the structure Z-Y-X-Y-Z where X is the targeting moiety, each Y is a linker that may be the same or different, and each Z is an active agent that may be the same or different.

### A. Active Agents

A conjugate as described herein contains at least one active agent (a first active agent). The conjugate can contain more than one active agent, that can be the same or different from the first active agent. The active agent can be a therapeutic, prophylactic, diagnostic, or nutritional agent. A variety of active agents are known in the art and they or analogs and derivatives thereof may be used in the conjugates described herein. The active agent can be a protein or peptide, small molecule, nucleic acid or nucleic acid molecule, lipid, sugar, glycolipid, glycoprotein, lipoprotein, or combination thereof. In some embodiments, the active agent is an antigen, an adjuvant, radioactive, an imaging agent (e.g., a fluorescent moiety) or a polynucleotide. In some embodiments the active agent is an organometallic compound or a radioactive element. The active agent has chemical functionality for covalent attachment to a linker or is modified to an analog or derivative for the purpose of covalent attachment to a linker.

In certain embodiments, the active agent of the conjugate comprises a predetermined molar weight percentage from about 1% to about 10%, or about 10% to about 20%, or about 20% to about 30%, or about 30% to about 40%, or about 40% to about 50%, or about 50% to about 60%, or about 60% to about 70%, or about 70% to about 80%, or about 80% to about 90%, or about 90% to about 99% such that the sum of the molar weight percentages of the components of the conjugate is 100%. The amount of active agent(s) of the conjugate may also be expressed in terms of proportion to the targeting ligand(s). For example, the present teachings provide a ratio of active agent to ligand of about 10:1, 9:1, 8:1, 7:1, 6:1, 5:1, 4:1, 3:1, 2:1, 1:1, 1:2, 1:3, 1:4; 1:5, 1:6, 1:7, 1:8, 1:9, or 1:10.

### Radioactive Agents

In some embodiments, the active agent Z is a radioactive agent or a chemical moiety that binds to a radionuclide (such as a radioisotope), such as a metal chelating group. A variety of radionuclides have emission properties, including a, (3, y, and Auger emissions, that may be used for therapeutic and/or diagnostic purposes. For example, the active agent Z may comprise a radioisotope, such as Y-90, Y-86, I-131, Re-186, Re-188, Y-90, Bi-212, At-211, Zr-89, Sr-89, Ho-166, Sm-153, Cu-67, Cu-64, Lu-177, Ac-225, Pb-203, Bi-213, Th-227, Pb-212, Ra-223, P-32, Sc-47, Br-77, Rh-105, Pd-103, Ag-111, Pr-142, Pm-149, Gd-159, Ir-194 and Pt-199.

In some embodiments, the active agent comprises an imaging probe, such as a radiolabel (e.g., a radioisotope). Non-limiting examples of radioisotopes for imaging include 1-124, 1-131, In-111, Re-186, Re-188, Y-90, Bi-212, At-211, Sr-89, Ho-166, Sm-153, Cu-60, Cu-67, Cu-64, Lu-177, Ac-225, Bi-213, Th-227, Pb-212, Ra-223, P-32, Sc-47, Br-76, Br-77, Rh-105, Pd-103, Ag-111, Pr-142, Pm-149, Gd-159, In-111, Ir-194, Pt-199, Tc-99m, Co-57, Ga-66, Ga-67, Ga-68, Kr-81m, Rb-82, Sr-92, Tl-201, Y-86, Zr-89, C-11, N-13, O-15 and F-18.

In some embodiments, the active agent Z comprises a radioactive agent, a chelating agent, or a radioactive agent attached to a chelating agent. A conjugate comprising a radioactive agent (e.g., a radioisotope) attached to a chelating agent is a radioactive analog of a conjugate with a chelating agent alone or with a chelating agent attached to a non-radioactive isotope.

The chelating agent may be a metal chelating agent that binds to a metal including a metallic nuclide. The chelating agent may also be a moiety that is attached to a non-metal active agent. The chelating agent may be acyclic or macrocyclic. Non-limiting examples of chelating agents include 1,4,7,10-tetraazacyclododecane-1,4,7,10-tetraacetic acid (DOTA); DOTA derivative: DO3A; diethylenetriamine-N,N,N′,N″,N″-pentaacetic acid (DTPA); DTPA derivatives: 2-(p-SCN-Bz)-6-methyl-DTPA, CHX-A″-DTPA, and the cyclic anhydride of DTPA (CA-DTPA); 1,4,7-triazacyclononane-1,4-7-triacetic acid (NOTA); NOTA derivatives (e.g., BCNOTA, p-NCS-Bz-NOTA, BCNOT); 6-hydrazinonicotinamide (HYNIC); ethylenediamine tetraacetic acid (EDTA); N,N-ethylene-di-L-cysteine; N,N-bis(2,2-dimethyl-2-mercaptoethyl)ethylenediamine-N,N-diacetic acid (6SS); 1-(4-carboxymethoxybenzyl)-N—N′-bis[(2-mercapto-2,2-dimethyl)ethyl]-1,2-ethylenediamine-N,N′-diacetic acid (B6SS); Deferoxamine (DFO); 1,1,1-tris(aminomethyl)ethane (TAME); tris(aminomethyl)ethane-N,N,N′,N′,N″,N″-hexaacetic acid (TAME Hex); O-hydroxybenzyl iminodiacetic acid; 1,4,7-triazacyclononane (TACN); 1,4,7,10-tretraazacyclododecane (cyclen); 1,4,7-triazacyclononane-1-succinic acid-4,7-diacetic acid (NODASA); 1-(1-carboxy-3-carboxypropyl)-4,7-bis-(carboxymethyl)-1,4,7-triazacyclononane (NODAGA); 1,4,7-tris(2-mercaptoethyl)-1,4,7-triazacylclonane (triazacyclononane-TM); 1,4,7-triazacyclononane-N,N′,N″-tris(methylenephosphonic)acid (NOTP); 1, 4, 8, 11-tetraazacyclotetradecane-N,N′,N″,N′″-tetraacetic acid (TETA); 1,4,7,10,13-pentaazacyclopentadecane-N,N′,N″,N′″,N″″-pentaacetic acid (PEPA), 1,4,7,10,13,16-hexaazacyclohexadecane-N,N′,N″,N′″,N″″,N′″″-hexaacetic acid (HEHA); 1,4,7,10-tetrakis(carbamoylmethyl)-1,4,7,10-tetraazacyclododecane (TCMC); and derivatives or analogs thereof.

In some embodiments, the chelating agents are polyaminocarboxylate agents, such as ethylenediamine tetraacetic acid (EDTA), diethylenetriamine pentaacetic acid (DTPA), 1,4,7,10-tetra-azacylcododecane-N,N′,N″,N′″-tetraacetic acid (DOTA), or derivatives thereof. They can coordinate with metals such as Fe, In, Ga, Zr, Y, Bi, Pb, or Ac.

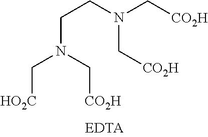 [SMILES: O=C(O)CN(CCN(CC(=O)O)CC(=O)O)CC(=O)O]

In some embodiments, the cheating agents are macrocyclic agents: 1,4,7-Triazacyclononane-N,N′,N″-triacetic acid (NOTA), 1,4,7,10-tetraazacyclododecane-N,N′,N″,N′″-tetraacetic acid (TETA), 1,4,7,10,13-pentaazacyclopentadecane-N,N′,N″,N′″,N″″-pentaacetic acid (PEPA), 1,4,7,10,13,16-hexaazacyclohexadecane-N,N′,N″,N′″,N″″,N′″″-hexaacetic acid (HEHA), or derivatives thereof.

Non-limiting examples of DTPA and derivatives thereof are:

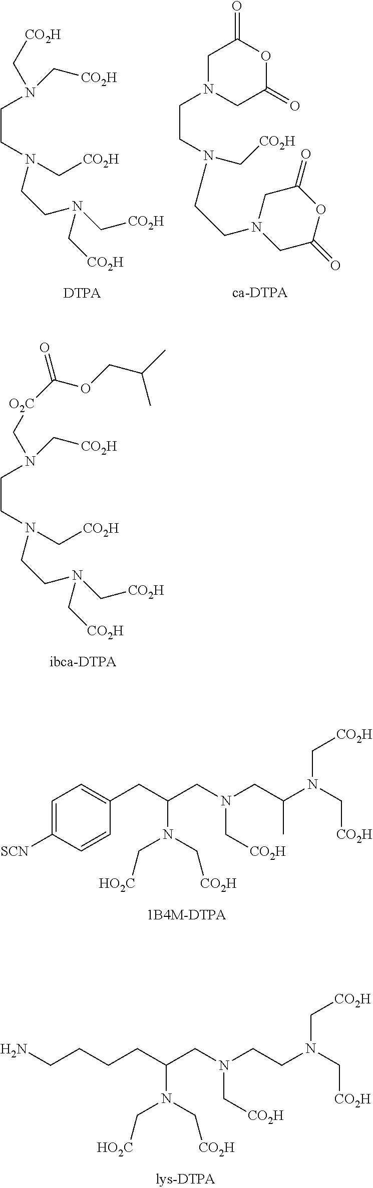

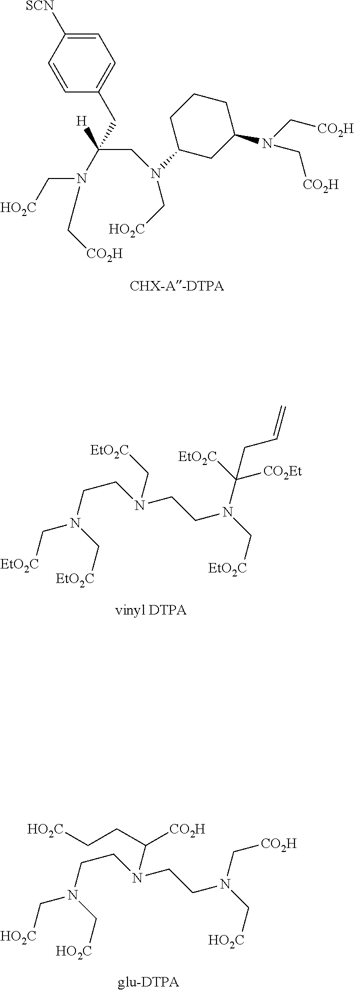

Non-limiting examples of DOTA and derivatives thereof are:

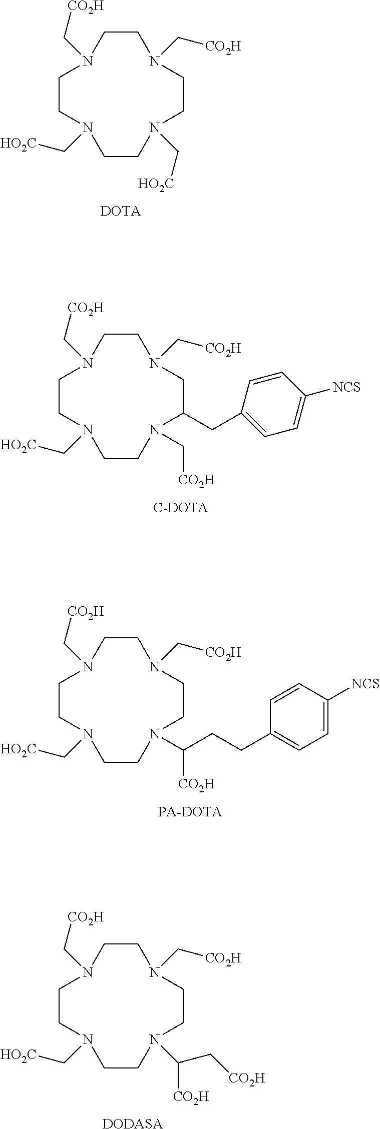

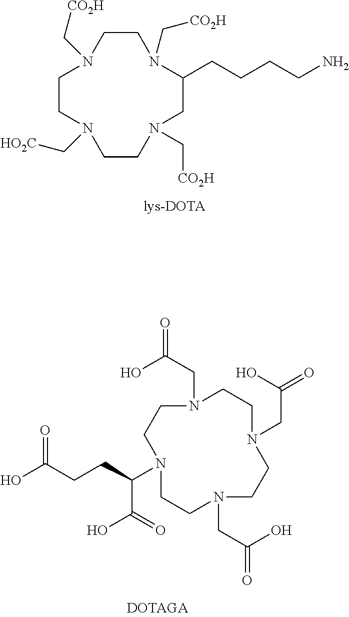

In some embodiments, the conjugates of the present disclosure comprise DOTA, DOTAGA, or any derivative/analog thereof as a chelating agent. Any chelating agent disclosed in Eisenwiener et al., Bioorg Med Chem Lett., vol. 10(18):2133 (2000), the contents of which are incorporated herein by reference in their entirety, may be used as a chelating agent, such as 1,4,7,10-Tetraazacyclododecane-1,4,7,10-tetraacetic acid, α-(2-carboxyethyl) (DOTAGA) or 1,4,7,10-Tetraazacyclododecane-1,4,7-triacetic acid, 10-(1,2-dicarboxyethyl) (DOTASA).

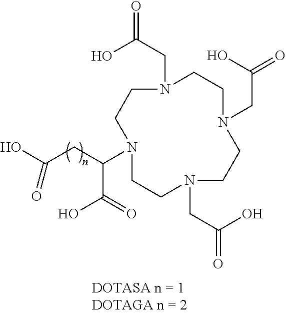

Other non-limiting examples of chelating agents are:

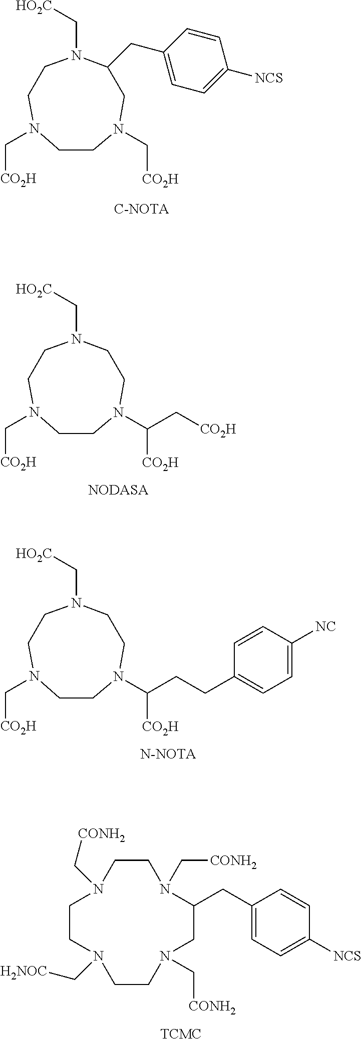

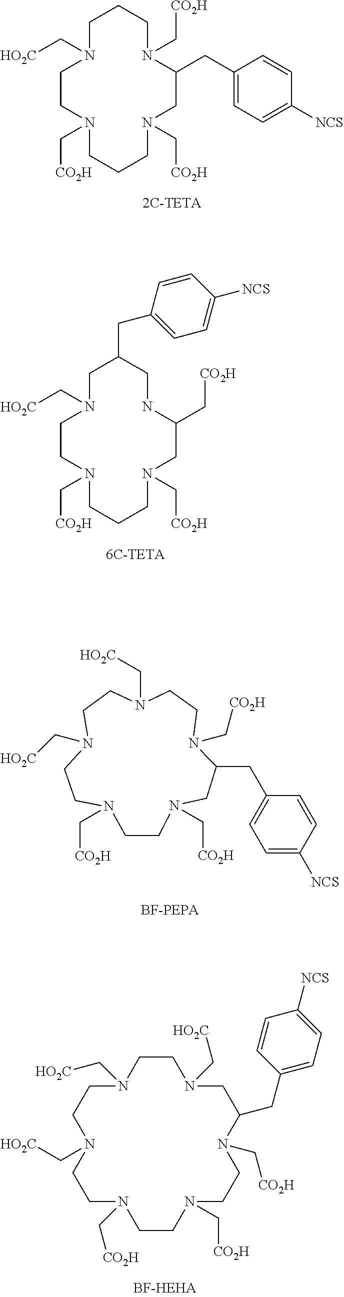

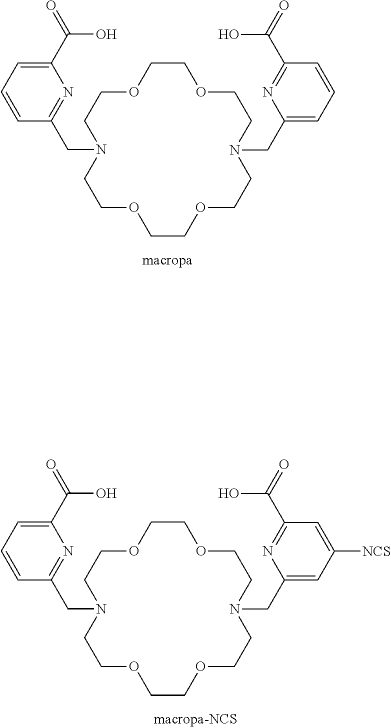

### B. Linkers

The conjugates contain one or more linkers attaching the active agents and targeting moieties. The linker, Y, is bound to one or more active agents and one or more targeting ligands to form a conjugate. The linker Y is attached to the targeting moiety X and the active agent Z by functional groups independently selected from an ester bond, disulfide, amide, acylhydrazone, ether, carbamate, carbonate, sulfonamide, alkyl, aryl, heteroaryl, thioether, and urea. Alternatively the linker can be attached to either the targeting ligand or the active drug by a group such as provided by the conjugation between a thiol and a maleimide, an azide and an alkyne. In some embodiments, the linker is a small molecule. In some embodiments, the linker is independently selected from the group consisting alkyl, cycloalkyl, heterocyclyl, aryl, and heteroaryl, wherein each of the alkyl, alkenyl, cycloalkyl, heterocyclyl, aryl, and heteroaryl groups optionally is substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl, wherein each of the carboxyl, carbamoyl, ether, alkoxy, aryloxy, amino, amide, carbamate, alkyl, alkenyl, alkynyl, aryl, arylalkyl, cycloalkyl, heteroaryl, or heterocyclyl is optionally substituted with one or more groups, each independently selected from halogen, cyano, nitro, hydroxyl, carboxyl, carbamoyl, ether, alkoxy, aryloxy, amino, amide, carbamate, alkyl, alkenyl, alkynyl, aryl, arylalkyl, cycloalkyl, heteroaryl, heterocyclyl.

In some embodiments, the linker comprises a cleavable functionality that is cleavable. The cleavable functionality may be hydrolyzed in vivo or may be designed to be hydrolyzed enzymatically, for example by Cathepsin B. A “cleavable” linker, as used herein, refers to any linker which can be cleaved physically or chemically. Examples for physical cleavage may be cleavage by light, radioactive emission or heat, while examples for chemical cleavage include cleavage by re-dox-reactions, hydrolysis, pH-dependent cleavage or cleavage by enzymes. For example, the cleavable functionality may be a disulfide bond or a carbamate bond.

In some embodiments the alkyl chain of the linker may optionally be interrupted by one or more atoms or groups selected from —O—, —C(═O)—, —NR, —O—C(═O)—NR—, —S—, —S—S—. The linker may be selected from dicarboxylate derivatives of succinic acid, glutaric acid or diglycolic acid. In some embodiments, the linker Y may be X′—R¹—Y′—R²—Z′ and the conjugate can be a compound according to Formula Ia:

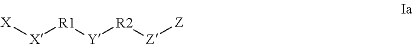

wherein X is a targeting moiety defined above; Z is an active agent; X′, R¹, Y′, R² and Z′ are as defined herein.

X′ is either absent or independently selected from carbonyl, amide, urea, amino, ester, aryl, arylcarbonyl, aryloxy, arylamino, one or more natural or unnatural amino acids, thio or succinimido; R¹ and R² are either absent or comprised of alkyl, substituted alkyl, aryl, substituted aryl, polyethylene glycol (2-30 units); Y′ is absent, substituted or unsubstituted 1,2-diaminoethane, polyethylene glycol (2-30 units) or an amide; Z′ is either absent or independently selected from carbonyl, amide, urea, amino, ester, aryl, arylcarbonyl, aryloxy, arylamino, thio or succinimido. In some embodiments, the linker can allow one active agent molecule to be linked to two or more ligands, or one ligand to be linked to two or more active agent molecule.

In some embodiments, the linker Y may be Am and the conjugate can be a compound according to Formula Ib:

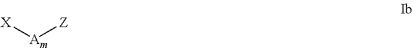

wherein A is defined herein, m=0-20.

A in Formula Ia is a spacer unit, either absent or independently selected from the following substituents. For each substituent, the dashed lines represent substitution sites with X, Z or another independently selected unit of A wherein the X, Z, or A can be attached on either side of the substituent:

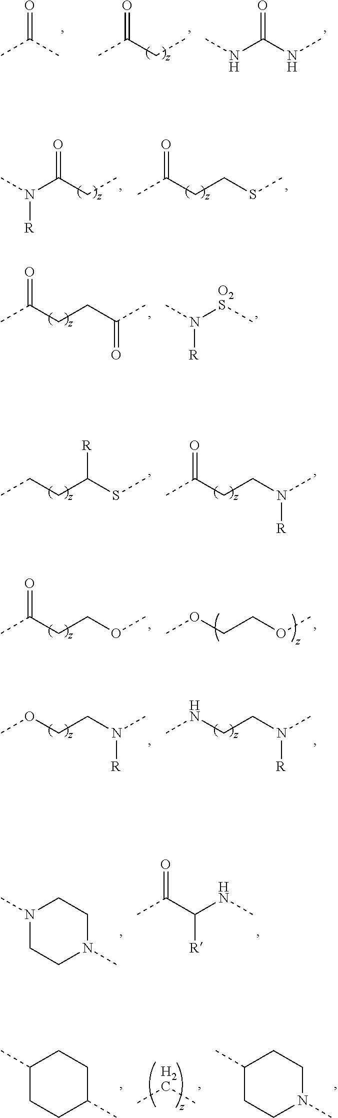

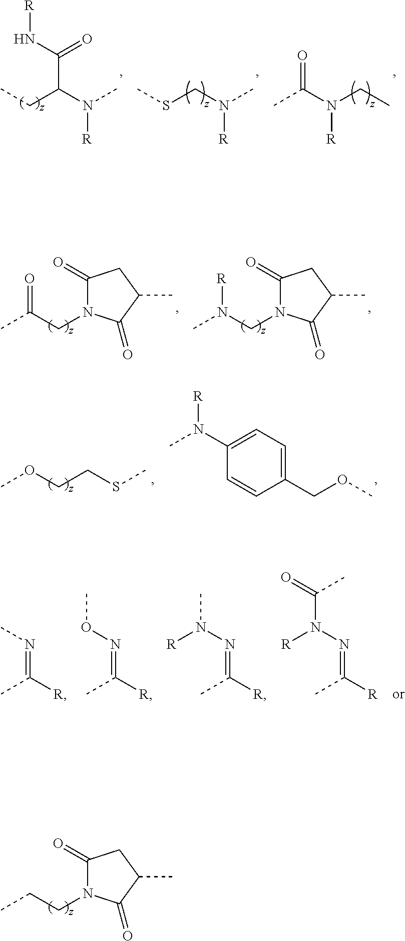

wherein z=0-40, R is H or an optionally substituted alkyl group, and R′ is any side chain found in either natural or unnatural amino acids.

In some embodiments, the conjugate may be a compound according to Formula Ic:

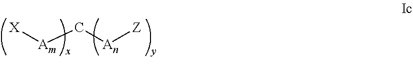

wherein A is defined above, m=0-40, n=0-40, x=1-5, y=1-5, and C is a branching element defined herein.

C in Formula Ic is a branched unit containing three to six functionalities for covalently attaching spacer units, ligands, or active drugs, selected from amines, carboxylic acids, thiols, or succinimides, including amino acids such as lysine, 2,3-diaminopropanoic acid, 2,4-diaminobutyric acid, glutamic acid, aspartic acid, and cysteine.

### C. HSP90 Targeting Moieties

Targeting ligands (also referred to as targeting moieties) as described herein include any molecule that can bind one or more HSP90 proteins. Such targeting ligands can be peptides, antibody mimetics, nucleic acids (e.g., aptamers), polypeptides (e.g., antibodies), glycoproteins, small molecules, carbohydrates, or lipids.

The targeting moiety, X, can be any HSP90 binding moiety such as, but not limited to, natural compounds (e.g., geldanamycin and radicicol), and synthetic compounds such as geldanamycin analogue 17-AAG (i.e., 17-allylaminogeldanamycin), a purine-scaffold HSP90 inhibitor series including PU24FC1 (He H., et al, J. Med. Chem., vol. 49:381 (2006), the contents of which are incorporated herein by reference in their entirety), BIIB021 (Lundgren K., et al, Mol. Cancer Ther., vol. 8(4):921 (2009), the contents of which are incorporated herein by reference in their entirety), 4,5-diarylpyrazoles (Cheung K. M., et al, Bioorg. Med. Chem. Lett., vol. 15:3338 (2005), the contents of which are incorporated herein by reference in their entirety), 3-aryl,4-carboxamide pyrazoles (Brough P. A., et al, Bioorg. Med. Chem. Lett., vol. 15: 5197 (2005), the contents of which are incorporated herein by reference in their entirety), 4,5-diarylisoxazoles (Brough P. A., et al, J. Med. Chem., vol. 51:196 (2008), the contents of which are incorporated herein by reference in their entirety), 3,4-diaryl pyrazole resorcinol derivative (Dymock B. W., et al, J. Med. Chem., vol. 48:4212 (2005), the contents of which are incorporated herein by reference in their entirety), thieno[2,3-d]pyrimidine (WO2005034950 to VERNALIS et al., the contents of which are incorporated herein by reference in their entirety), aryl triazole derivatives of Formula I in EP2655345 to Giannini et al., the contents of which are incorporated herein by reference in their entirety, or any other example of HSP90 binding ligands or their derivatives/analogs.

In some embodiments, the HSP90 binding moiety may be heterocyclic derivatives containing three heteroatoms. WO2009134110 to MATULIS et al., the contents of which are incorporated herein by reference in their entirety, discloses 4,5-diaryl thiadiazoles which demonstrate good HSP90 binding affinity. Even though they have rather modest cell growth inhibition, they may be used as HSP90 binding moiety in conjugates of the present invention. Another class of aza-heterocyclic adducts, namely triazole derivatives or their analogs, may be used as HSP90 binding moiety in conjugates of the present invention. For example, the 1,2,4-triazole scaffold has been profusely documented as possessing HSP90 inhibiting properties. WO2009139916 to BURLISON et al. (Synta Pharmaceuticals Corp.), the contents of which are incorporated herein by reference in their entirety, discloses tricyclic 1,2,4-triazole derivatives inhibiting HSP90 at high micromolar concentrations. Any tricyclic 1,2,4-triazole derivatives disclosed in WO2009139916 or their derivatives/analogs may be used as HSP90 binding moiety in conjugates of the present invention. Any trisubstituted 1,2,4-triazole derivatives disclosed in WO 2010017479 and WO 2010017545 (Synta Pharmaceuticals Corp.) or their derivatives/analogs, the contents of which are incorporated herein by reference in their entirety, may be used as HSP90 binding moiety in conjugates of the present invention. In another example, a triazolone-containing HSP90 inhibitor named ganetespib (previously referred as to STA-9090, or as its highly soluble phosphate prodrug STA-1474) disclosed in WO2006055760 (Synta Pharmaceuticals Corp.), the contents of which are incorporated herein by reference in their entirety, or its derivatives/analogs may be used as HSP90 binding moiety in conjugates of the present invention.

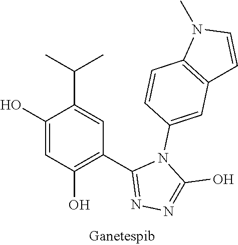 [SMILES: CC(C)c1cc(-c2nnc(O)n2-c2ccc3c(ccn3C)c2)c(O)cc1O]

In some embodiments, ganetespib or its derivatives/analogs may be used a targeting moiety. Non-limiting examples of ganetespib derivatives/analogs are shown below.

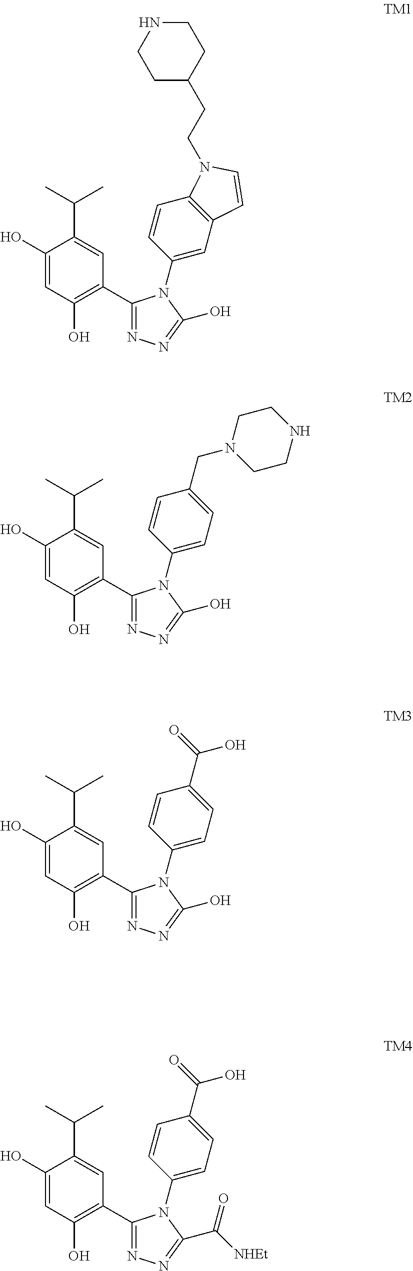

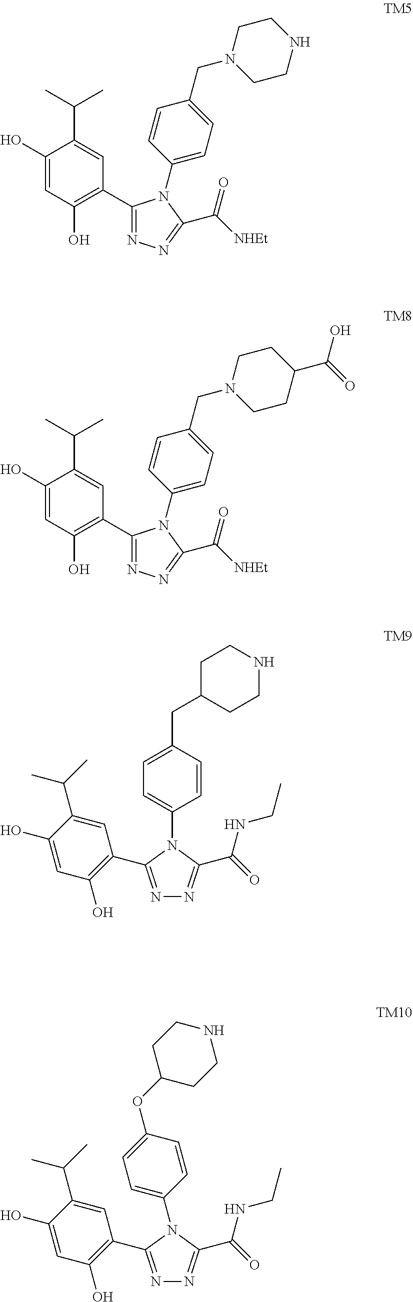

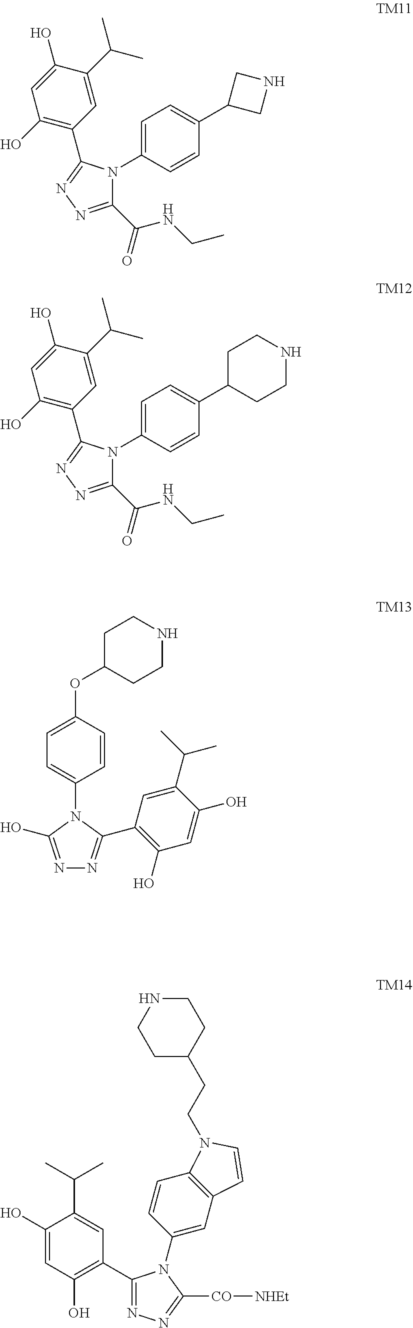

In some embodiments, Onalespib (AT13387) or its derivatives/analogs may be used as a targeting moiety in the conjugates of the present invention. Onalespib and non-limiting examples of Onalespib derivatives/analogs are shown below.

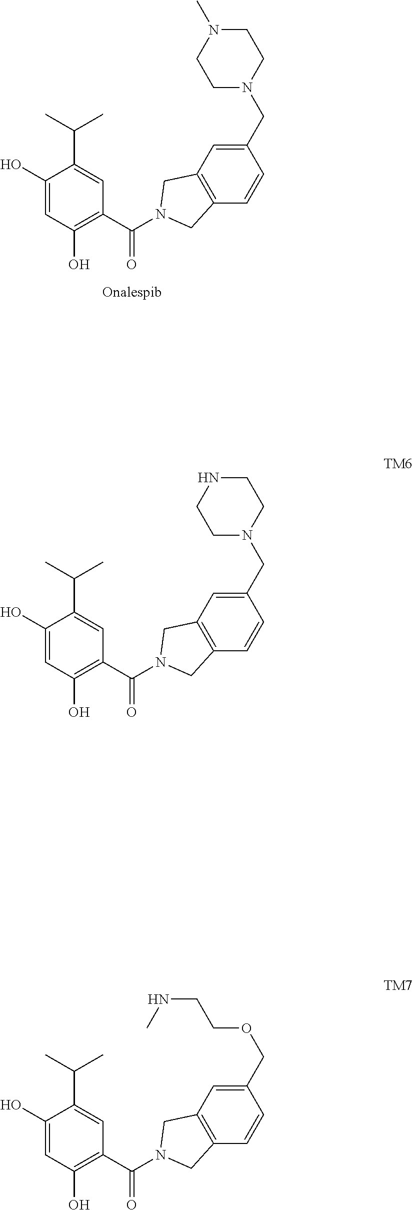 [SMILES: CC(C)c1cc(C(=O)N2Cc3ccc(CN4CCN(C)CC4)cc3C2)c(O)cc1O ; CC(C)c1cc(C(=O)N2Cc3ccc(CN4CCNCC4)cc3C2)c(O)cc1O ; CNCCOCc1ccc2c(c1)CN(C(=O)c1cc(C(C)C)c(O)cc1O)C2]

In some embodiments, the targeting moiety comprises AUY-922, or an analog/derivative/fragment thereof. In one embodiments, the targeting moiety has a structure of

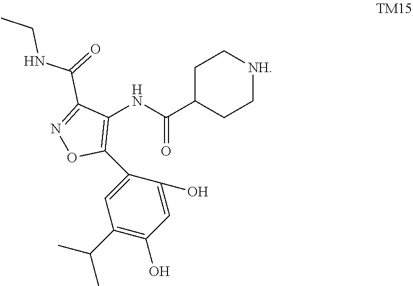 [SMILES: CCNC(=O)c1noc(-c2cc(C(C)C)c(O)cc2O)c1NC(=O)C1CCNCC1]

Any HSP90 ligand or HSP90 inhibitor disclosed in WO2013158644, WO2015038649, WO2015066053, WO2015116774, WO2015134464, WO2015143004, WO2015184246, the contents of which are incorporated herein by reference in their entirety, or their derivatives/analogs may be used as HSP90 binding moiety in the conjugates of the present invention, such as:

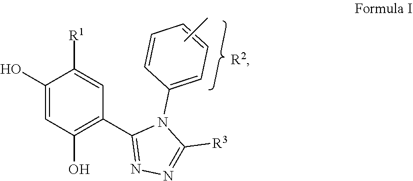

wherein R1 may be alkyl, aryl, halide, carboxamide or sulfonamide; R2 may be alkyl, cycloalkyl, aryl or heteroaryl, wherein when R2 is a 6 membered aryl or heteroaryl, R2 is substituted at the 3- and 4-positions relative to the connection point on the triazole ring, through which a linker L is attached; and R3 may be SH, OH, —CONHR4, aryl or heteroaryl, wherein when R3 is a 6 membered aryl or heteroaryl, R3 is substituted at the 3 or 4 position;

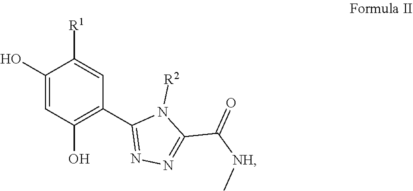

wherein R1 may be alkyl, aryl, halo, carboxamido, sulfonamido; and R2 may be optionally substituted alkyl, cycloalkyl, aryl or heteroaryl. Examples of such compounds include 5-(2,4-dihydroxy-5-isopropylphenyl)-N-(2-morpholinoethyl)-4-(4-(morpholinomethyl)phenyl)-4H-1,2,4-triazole-3-carboxamide and 5-(2,4-dihydroxy-5-isopropylphenyl)-4-(4-(4-methylpiperazin-1-yl)phenyl)-N-(2,2,2-trifluoroethyl)-4H-1,2,4-triazole-3-carboxamide;

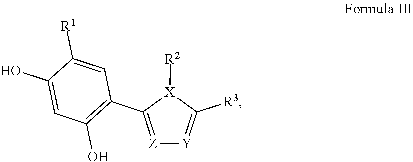

wherein X, Y, and Z may independently be CH, N, O or S (with appropriate substitutions and satisfying the valency of the corresponding atoms and aromaticity of the ring); R1 may be alkyl, aryl, halide, carboxamido or sulfonamido; R2 may be substituted alkyl, cycloalkyl, aryl or heteroaryl, where a linker L is connected directly or to the extended substitutions on these rings; R3 may be SH, OH, NR4R5 AND —CONHR6, to which an effector moiety may be connected; R4 and R5 may independently be H, alkyl, aryl, or heteroaryl; and R6 may be alkyl, aryl, or heteroaryl, having a minimum of one functional group to which an effector moiety may be connected; or

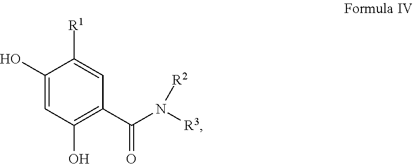

wherein R1 may be alkyl, aryl, halo, carboxamido or sulfonamido; R2 and R3 are independently C1-C5 hydrocarbyl groups optionally substituted with one or more of hydroxy, halogen, C1-C2 alkoxy, amino, mono- and di-C1-C2 alkylamino; 5- to 12-membered aryl or heteroaryl groups; or, R2 and R3, taken together with the nitrogen atom to which they are attached, form a 4- to 8-membered monocyclic heterocyclic group, of which up to 5 ring members are selected from O, N and S. Examples of such compounds include AT-13387.

The HSP90 targeting moiety may be Ganetespib, Luminespib (AUY-922, NVP-AUY922), Debio-0932, MPC-3100, Onalespib (AT-13387), SNX-2112, 17-amino-geldanamycin hydroquinone, PU-H71, or derivatives/analogs thereof.

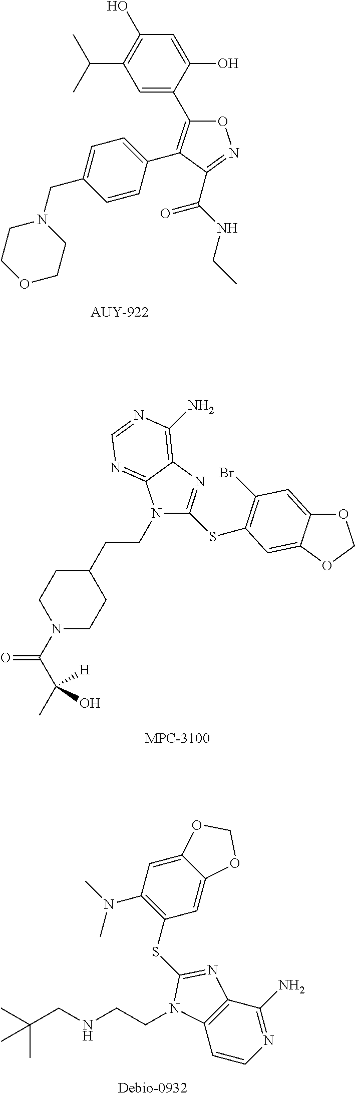

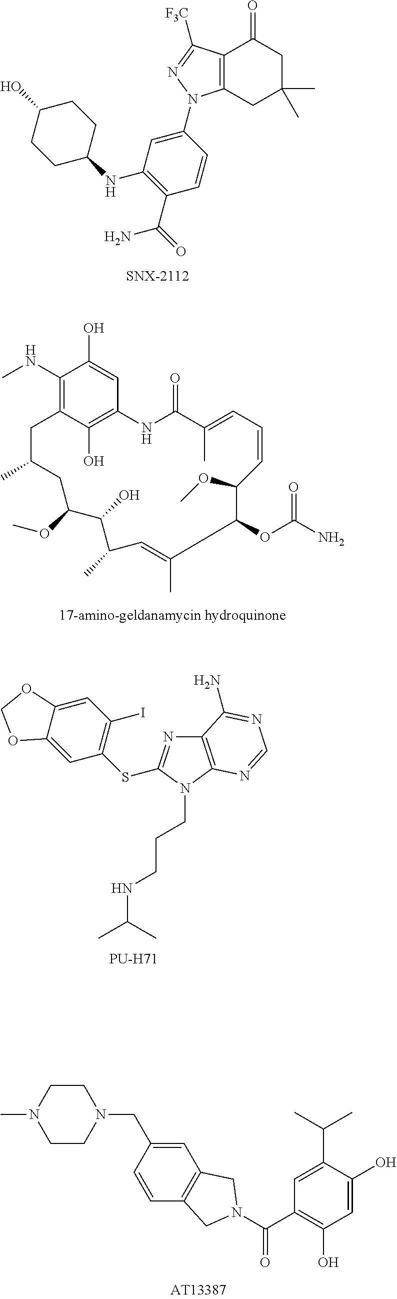

The HSP90 targeting moiety may be SNX5422 (PF-04929113), or any other HSP90 inhibitors disclosed in U.S. Pat. No. 8,080,556 (Pfizer), WO2008096218 (Pfizer), WO2006117669 (Pfizer), WO2008059368 (Pfizer), WO2008053319 (Pfizer), WO2006117669 (Pfizer), EP1885701 (Novartis), EP1776110 (Novartis), EP2572709 (Novartis), WO2012131413 (Debiopharm), or WO2012131468 (Debiopharm), the contents of each of which are incorporated herein by reference in their entirety.

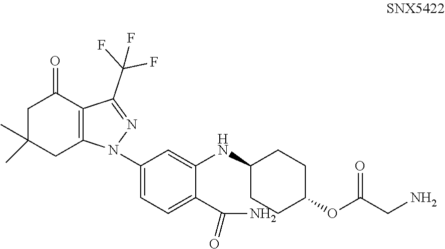 [SMILES: CC1(C)CC(=O)c2c(C(F)(F)F)nn(-c3ccc(C(N)=O)c(N[C@H]4CC[C@H](OC(=O)CN)CC4)c3)c2C1]

The HSP90 targeting moiety may also be PU-H71, an HSP90 inhibitor that is ¹²⁴I radiolabeled for PET imaging or its derivatives/analogs.

Conjugates comprising SNX-2112, 17-amino-geldanamycin hydroquinone, PU-H71, or AT13387 may have a structure of:

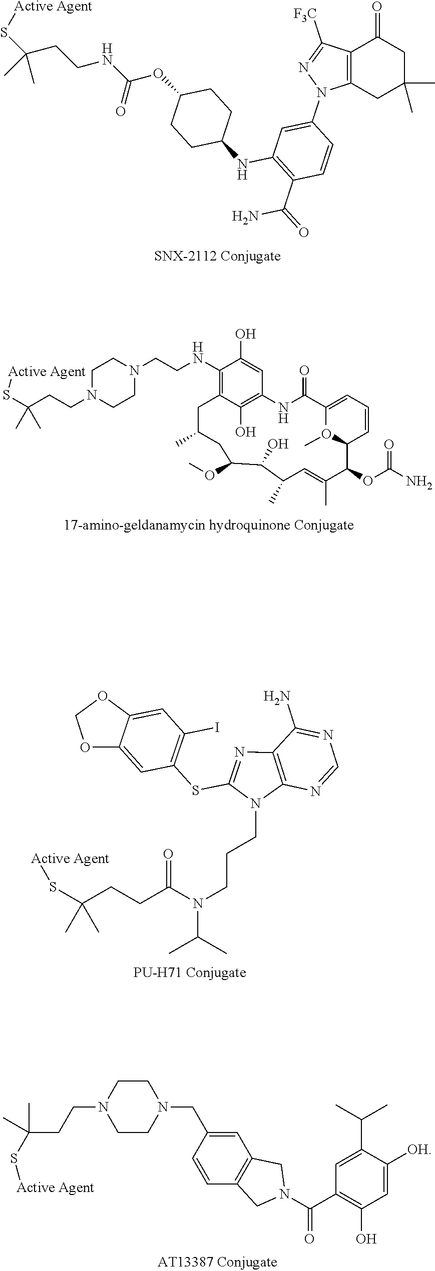

In some embodiments, the targeting moiety comprises an imaging probe, such as a radiolabel (e.g., a radioisotope). Non-limiting examples of radioisotopes include I-131, Re-186, Re-188, Y-90, Bi-212, At-211, Sr-89, Ho-166, Sm-153, Cu-67, Cu-64, Lu-177, Ac-225, Bi-213, Th-227, Pb-212, Ra-223, P-32, Sc-47, Br-77, Rh-105, Pd-103, Ag-111, Pr-142, Pm-149, Gd-159, Ir-194, Pt-199, Tc-99m, Co-57, Ga-67, Kr-81m, Rb-82, Sr-92, Tl-201, C-11, N-13, O-15 and F-18.

In some embodiments, the conjugates of the present disclosure comprise more than one targeting moiety. For example, the conjugate may comprise 2, 3, 4, or 5 HSP90 targeting moieties.

Extracellular HSP90 (eHSP90)

In normal cells, secretion of HSP90 occurs when cells are under environmental stress such as heat, drugs, cytokines, UV, and/or gamma rays. The main function of the extracellular HSP90 (eHSP90) is to help tissue repair by promoting the cells at the edge of damaged tissue to migrate into the damaged area. However, in tumors, constitutively activated oncogenes trigger HSP90 secretion even without any environmental stress. Secreted Hsp90 by tumors eHSP90α promotes both tumor and tumor stroma cell migration during invasion and metastasis. The extracellular promotility function of HSP90α depends on a 115-amino acid fragment (F-5) on the surface of HSP90 (Li et al., Int Rev Cell Mol Biol., vol. 303:203-235 (2013), the contents of which are incorporated herein by reference in their entirety). eHSP90 has been shown to be present on the surface of tumor cells and to also be capable of being internalized (Crowe et al., ACS Chem. Biol., vol. 12:1047-1055 (2017)). The surface expression of eHSP90 in tumor cells thus represents a target for directing therapies selectively to tumors over healthy cells. Therefore, eHSP90 (eHSP90α in particular) may be a good target for treating tumors.

In some embodiments, the targeting moiety selectively binds to eHSP90. In some embodiments, the targeting moiety binds to F-5 region of eHSP90.

In some embodiments, the targeting moiety has low cell-permeability and prefers to bind to cell surface eHSP90. In some embodiments, the targeting moiety is cell-impermeable and binds exclusive to eHSP90. In some embodiments, the conjugates comprising the targeting moieties have a low cell permeability or is cell-impermeable.

In some embodiments, the targeting moieties comprise HS-23, HS-131, (disclosed in Crowe et al., ACS Chem. Biol., vol. 12:1047-1055 (2017), the contents of which are incorporated herein by reference in their entirety) or DMAG-N-oxide (a cell-impermeable for of 17-AAG disclosed in Tsutsumi et al., Oncogene, vol. 27(17):2478-2487 (2008), the contents of which are incorporated herein by reference in their entirety), or analog/derivative thereof, the structures shown below.

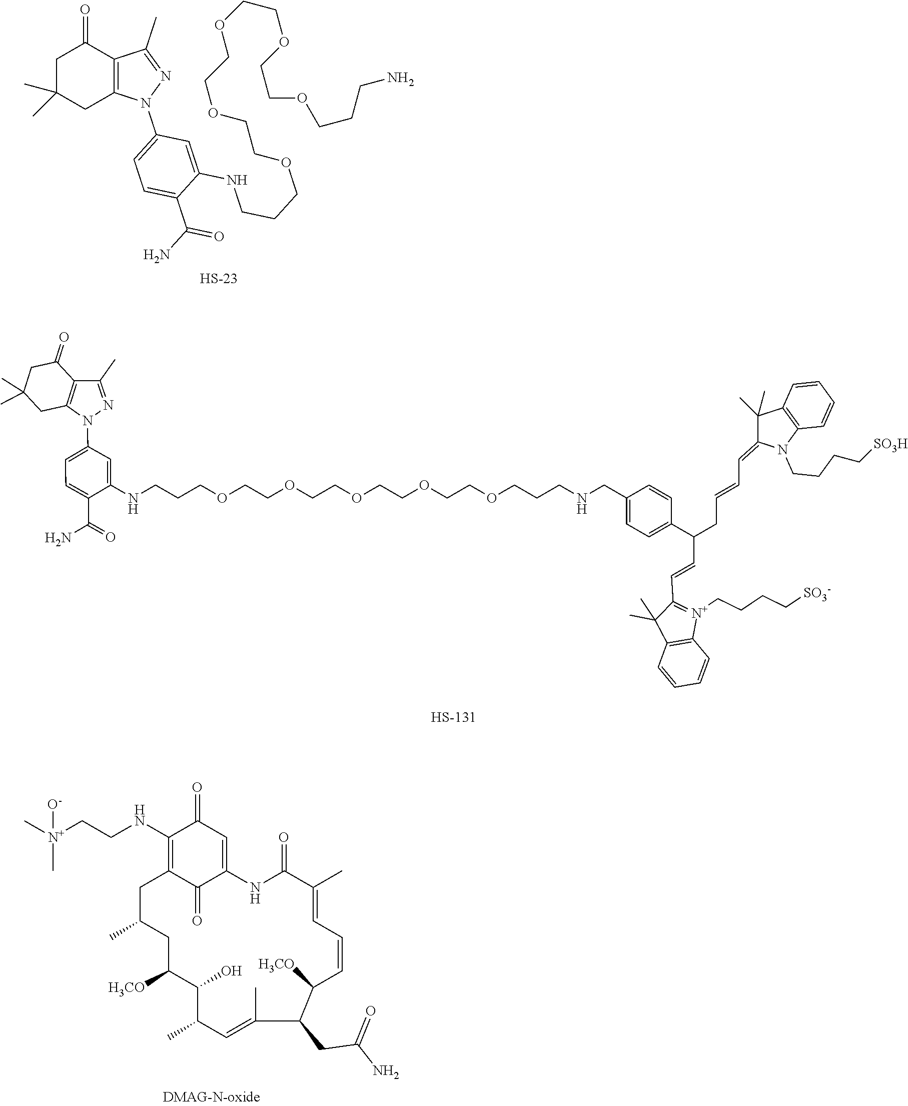

In certain embodiments, the targeting moiety or moieties of the conjugate are present at a predetermined molar weight percentage from about 0.1% to about 10%, or about 1% to about 10%, or about 10% to about 20%, or about 20% to about 30%, or about 30% to about 40%, or about 40% to about 50%, or about 50% to about 60%, or about 60% to about 70%, or about 70% to about 80%, or about 80% to about 90%, or about 90% to about 99% such that the sum of the molar weight percentages of the components of the conjugate is 100%. The amount of targeting moieties of the conjugate may also be expressed in terms of proportion to the active agent(s), for example, in a ratio of ligand to active agent of about 10:1, 9:1, 8:1, 7:1, 6:1, 5:1, 4:1, 3:1, 2:1, 1:1, 1:2, 1:3, 1:4; 1:5, 1:6, 1:7, 1:8, 1:9, or 1:10.

### D. Pharmacokinetic Modulating Unit

The conjugates of the present invention may further comprise at least one external linker connected to a reacting group that reacts with a functional group on a protein or an engineered protein or derivatives/analogs/mimics thereof, or comprise at least one external linker connected to a pharmacokinetic modulating unit (PMU). The external linkers connecting the conjugates and the reacting group or the pharmacokinetic modulating units may be cleavable linkers that allow release of the conjugates. Hence, the conjugates may be separated from the protein or pharmacokinetic modulating units as needed.

Any reacting group or PMU (such as PMUs comprising polymers) disclosed in WO2017/197241, the contents of which are incorporated herein by reference in their entirety, may be attached to the conjugates of the present disclosure.

In some embodiments, the conjugate comprises a protein-binding reacting group attached to its active agent. In some embodiments, the conjugate comprises a protein-binding reacting group attached to its targeting moiety. In some embodiments, the conjugate comprises a protein-binding reacting group attached to its linker. The reacting group binds to a protein reversibly or irreversibly. The protein may be a naturally occurring protein, such as a serum or plasma protein, or a fragment thereof. Particular examples include Fc neonatal receptor (FcRn), thyroxine-binding protein, transthyretin, α1-acid glycoprotein (AAG), transferrin, fibrinogen, albumin, an immunoglobulin, α-2-macroglobulin, a lipoprotein, or fragments thereof. The reacting group may bind to such a protein via covalent bonds or non-covalent interactions, such as hydrogen bonds, ionic bonds, van der Waals interactions, and hydrophobic bonds.

In some embodiments, the protein-binding reacting group may bind to a serum protein via non-covalent interactions. For example, the reacting group may be saturated fatty acids that bind to albumin with weak affinities (10⁻⁴ to 10⁻⁵ M). Non-limiting examples of such fatty acids may include myristic acid (a fatty acid with 14 carbon atoms) and palmitic acid (a fatty acid with 16 carbon atoms). Other non-limiting examples of the reacting groups include a naphthalene acylsulfonamide group, a diphenylcyclohexanol phosphate ester group, a 6-(4-(4-iodophenyl) butanamido)hexanoate group (‘Albu’-tag), a series of peptides having the core sequence of DICLPRWGCLW including SA21 (a cyclic peptide with 18 amino acids Ac-RLIEDICLPRWGCLWEDD-NH₂) disclosed by Dennis et al. in J. Biol. Chem., vol. 277:35035 (2002), the contents of which are incorporated herein by reference in their entirety.

A protein-binding reacting group may comprise a structure of:

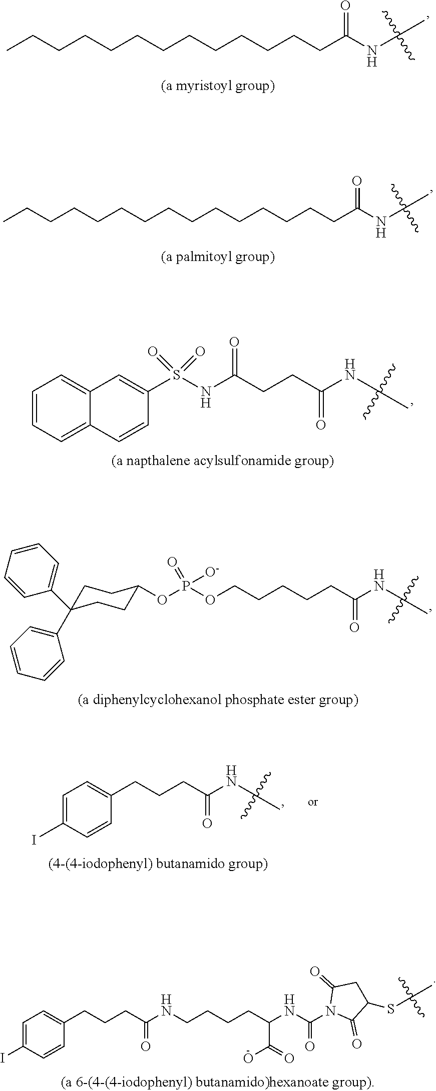 [SMILES: *NC(=O)CCCCCCCCCCCCC ; *NC(=O)CCCCCCCCCCCCCCC ; *NC(=O)CCC(=O)NS(=O)(=O)c1ccc2ccccc2c1 ; *NC(=O)CCCCCOP(=O)([O-])OC1CCC(c2ccccc2)(c2ccccc2)CC1 ; *NC(=O)CCCc1ccc(I)cc1 ; *SC1CC(=O)N(C(=O)NC(CCCCNC(=O)CCCc2ccc(I)cc2)C(=O)O)C1=O]

In some embodiments, the protein-binding reacting group may comprise any peptide-fatty acid albumin-binding ligand disclosed in Zorzi et al., Nature Communications, vol. 8:16092, (2017), the contents of which are incorporated herein by reference in their entirety. These peptide-fatty acid albumin-binding ligands comprise a fatty acid connected to a short peptide, e.g., a heptapeptide, via an amino acid side chain. The fatty acid may be linked to the short peptide via its carboxylic group to the side chain of lysine. The fatty acid binds to albumin with an affinity in the micromolar range and the short peptide enhances the affinity by forming additional contacts to albumin. The peptide-fatty acid ligands may have a general structure of:

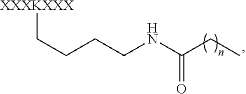

wherein X=any amino acid (such as Gly or Ser), K=Lys, n=12 (myristic acid), 14 (palmitic acid), or 16 (stearic acid).

In some embodiments, any albumin-binding functional group disclosed in U.S. Pat. No. 9,670,482 (Bicycle Therapeutics), the contents of which are incorporated herein in their entirety, may be used as a protein-binding reacting group in the present application. In some embodiments, the protein-binding reacting group comprises a fluorene ring and binds to albumin non-covalently and/or reversibly. As a non-limiting example, the protein-binding reacting group comprises a fluorenylmethyloxycarbonyl (FMOC) group. Optionally, the protein-binding reacting group comprises at least one amino acid attached to FMOC, such as Lys, Trp, Gly, or Phe. For example, the small molecule may comprise Fmoc-Lys-, Fmoc-Gly-, Fmoc-Phe-, Fmoc-GGSGD-, Fmoc-FGGGD-, Fmoc-FGSGD-, Fmoc-WGSGD-, Fmoc-WGGGA, or Fmoc-Trp-GGG.

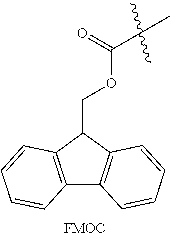 [SMILES: *C(=O)OCC1c2ccccc2-c2ccccc21]

Non-Limiting Examples of Conjugates

In some embodiments, the conjugate comprises at least one HSP90 targeting moiety attached to a chelating agent for a radioactive agent with a linker. The HSP90 targeting moiety may be a ganetespib analog or derivative (such as TM1, TM2, TM3, TM4, TM5, TM8, TM9, TM10, TM11, TM12, TM13, or TM14), an onalespib analog or derivative (such as TM6 or TM7), or TM15. The radioactive agent may comprise any radioisotope, such as lutetium 177 (Lu177 or ¹⁷⁷Lu). The lutetium isotope 177 (¹⁷⁷Lu) in a conjugate renders radioactivity to the conjugate. The chelating agent may be any suitable chelating agent, such as DOTA or DOTAGA. The molecular weight of the conjugates may be less than 5000 Da, such as between about 1000 Da and about 3000 Da, or between about 1500 Da and 2500 Da.

In some embodiments, the conjugate has a structure of Formula X:

wherein TM(s) refers to at least one targeting moiety that binds to HSP90. TM is selected from TM1, TM2, TM3, TM4, TM5, TM8, TM9, TM10, TM11, TM12, TM13, TM14, TM6, TM7, or TM15. In some embodiments, the conjugate has 1 targeting moiety. In some embodiments, the conjugate has 2 targeting moieties. In some embodiments, the conjugate has 3 targeting moieties. In some embodiments, the conjugate has 4 targeting moieties.

In some embodiments, the conjugate has a structure of Formula X10:

TM is selected from TM1, TM2, TM3, TM4, TM5, TM8, TM9, TM10, TM11, TM12, TM13, TM14, TM6, TM7, or TM15.

In some embodiments, the conjugate has a structure of Formula X1:

TM is selected from TM1, TM2, TM3, TM4, TM5, TM8, TM9, TM10, TM11, TM12, TM13, TM14, TM6, TM7, or TM15. Non-limiting examples of conjugates encompassed by Formula X1 include CMP24, CMP26, and CMP27.

In some embodiments, the conjugate has a structure of Formula X20:

TM is selected from TM1, TM2, TM3, TM4, TM5, TM8, TM9, TM10, TM11, TM12, TM13, TM14, TM6, TM7, or TM15.

In some embodiments, the conjugate has a structure of Formula X2:

TM is selected from TM1, TM2, TM3, TM4, TM5, TM8, TM9, TM10, TM11, TM12, TM13, TM14, TM6, TM7, or TM15. Non-limiting examples of conjugates encompassed by Formula X2 include CMP22, CMP8, CMP9, CMP10, CMP11, CMP12, CMP 17, CMP18, CMP19, CMP20, CMP21, CMP25, CMP 28, T25, T26, T30, T31, T40, and T43.

Non-limiting examples of conjugates of the current disclosure that are encompassed by Formula X include CMP7, CMP8, CMP9, CMP10, CMP11, CMP12, CMP17, CMP18, CMP19, CMP20, CMP21, CMP22, CMP24, CMP25, CMP26, CMP27, CMP28, T18, T19, T20, T21, T25, T26, T30, T31, T43, or T40:

TABLE 1
Non-limiting Examples of Conjugates
Structures

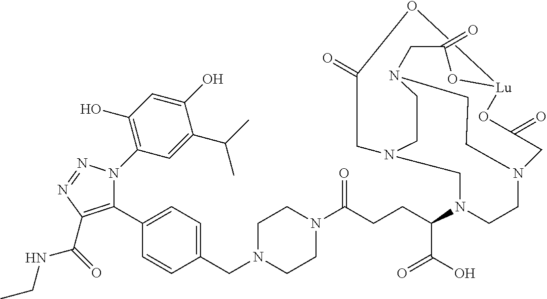

CMP22: comprising TM5; encompassed by Formula X2

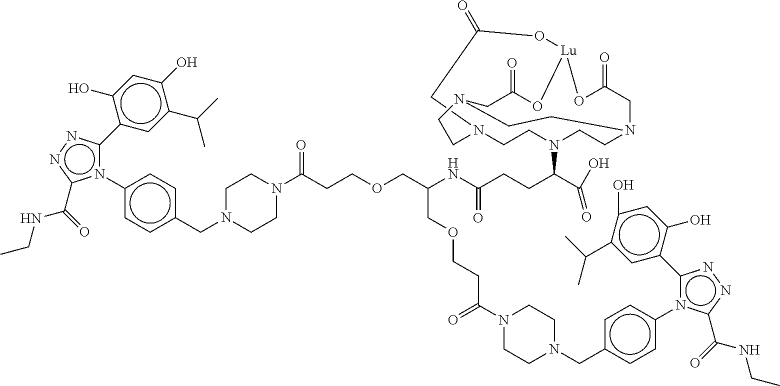

CMP7: comprising 2 TM9s; encompassed by Formula X2

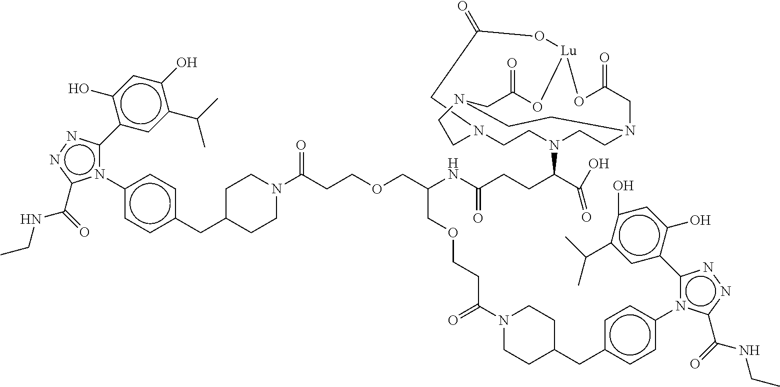

CMP8: comprising 2 TM5s; encompassed by Formula X2

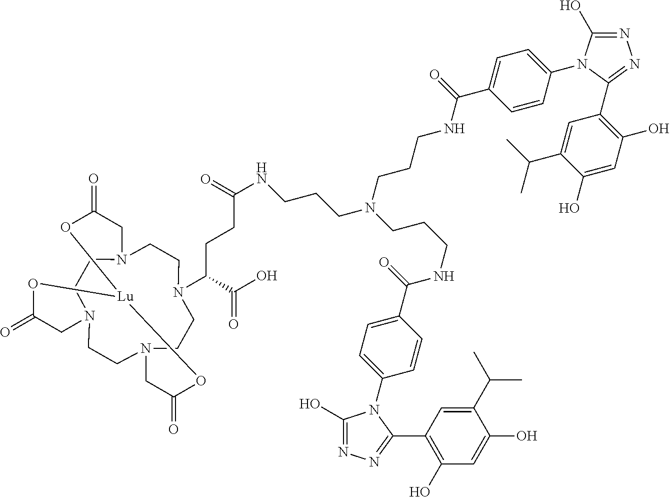

CMP10: comprising 2 TM3s; encompassed by Formula X2

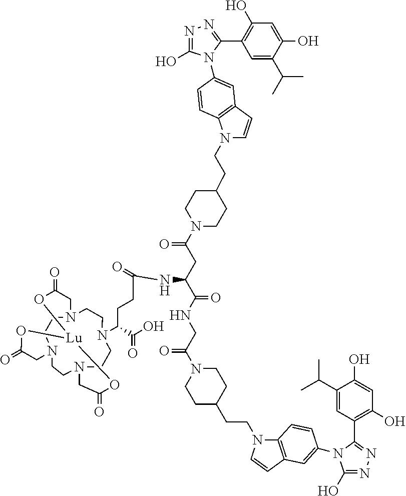

CMP 11: comprising 2 TM1s; encompassed by Formula X2

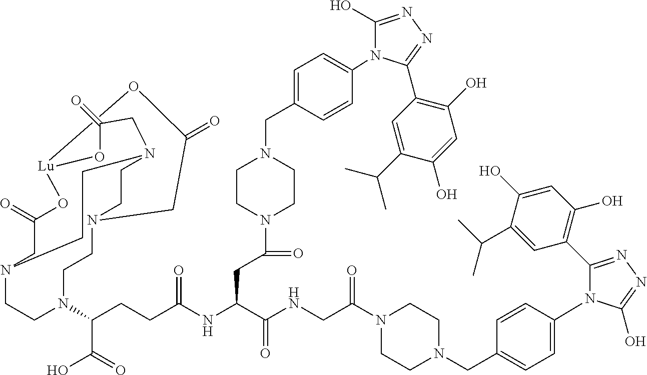

CMP12: comprising 2 TM2s; encompassed by Formula X2

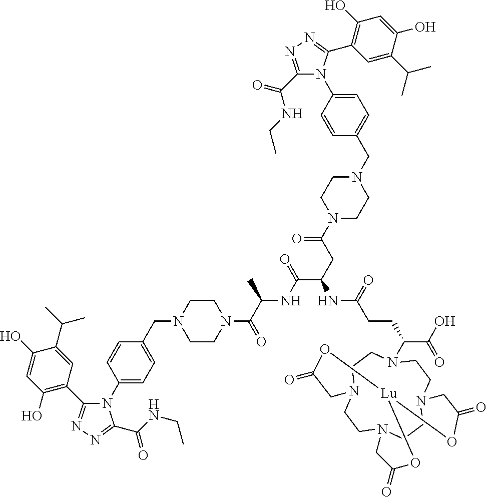

CMP17: comprising 2 TM5s; encompassed by Formula X2

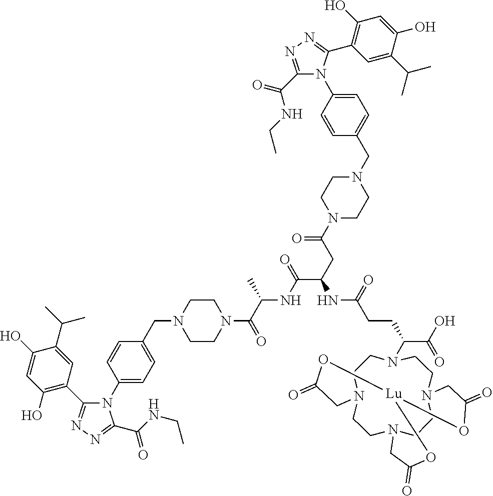

CMP18: comprising 2 TM5s; encompassed by Formula X2

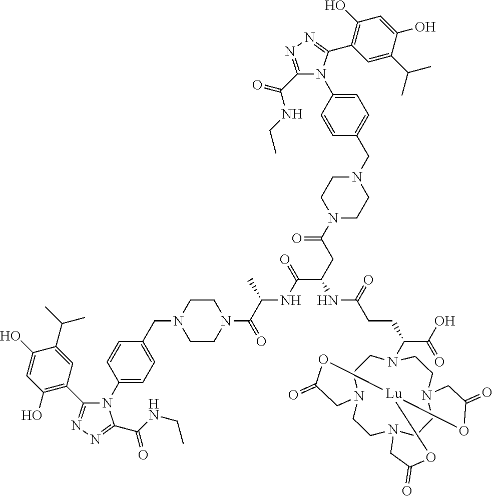

CMP19: comprising 2 TM5s; encompassed by Formula X2

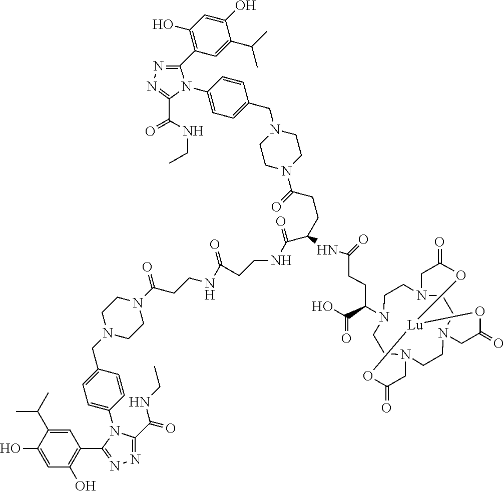

CMP20: comprising 2 TM5s; encompassed by Formula X2

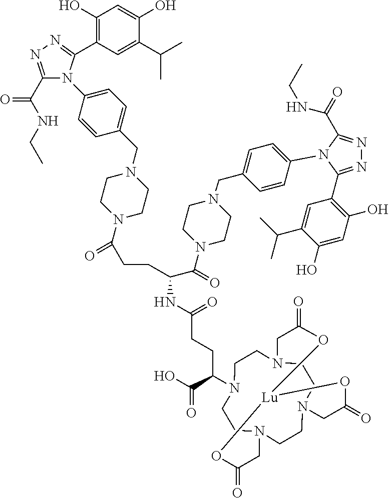

CMP21: comprising 2 TM5s; encompassed by Formula X2

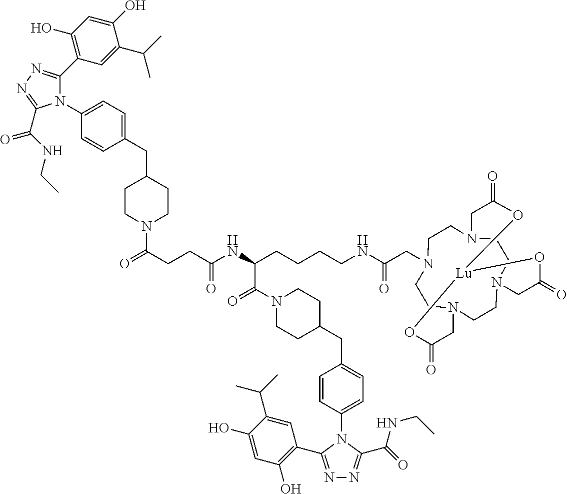

CMP24: comprising 2 TM9s; encompassed by Formula X1

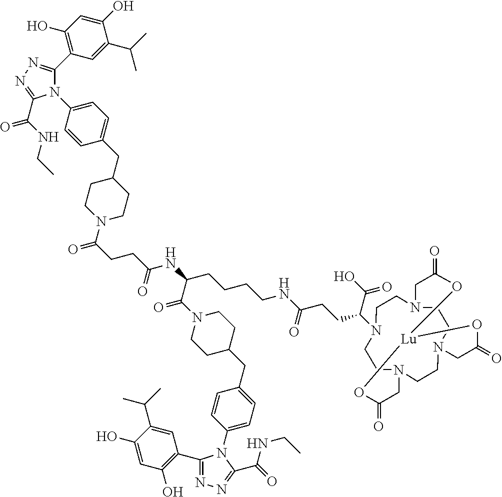

CMP25: comprising 2 TM9s; encompassed by Formula X2

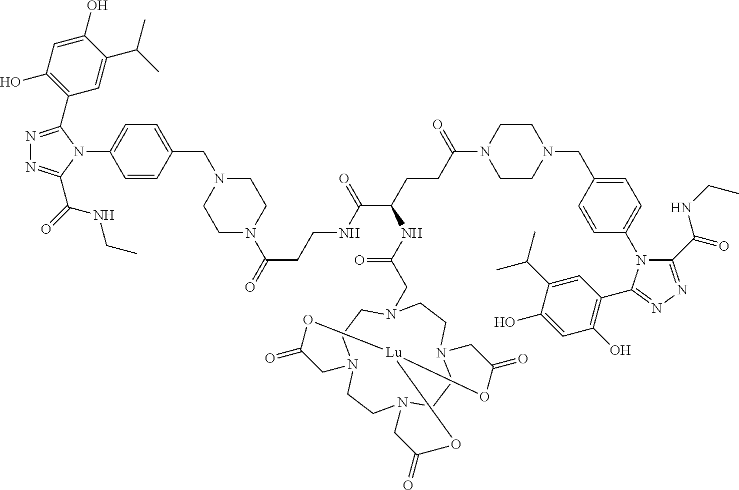

CMP26: comprising 2 TM5s; encompassed by Formula X1

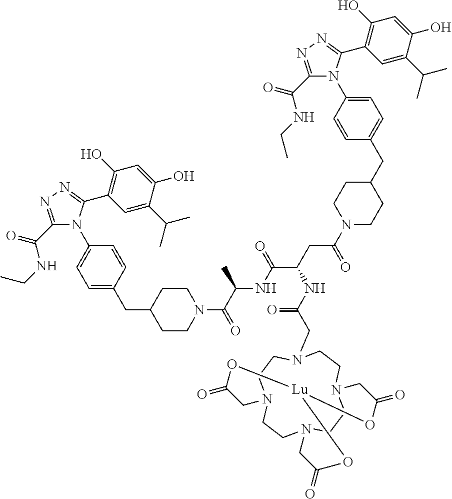

CMP27: comprising 2 TM9s; encompassed by Formula X1

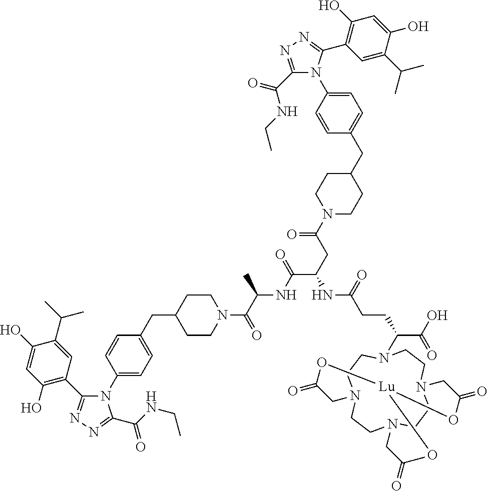

CMP28: comprising 2 TM9s; encompassed by Formula X2

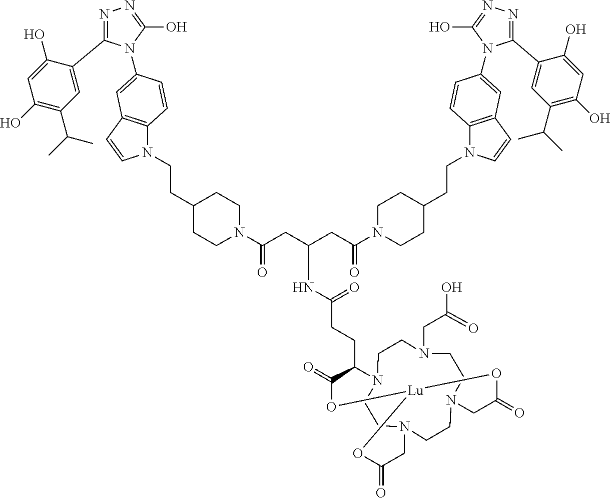

T18: comprising 2 TM1s; encompassed by X2

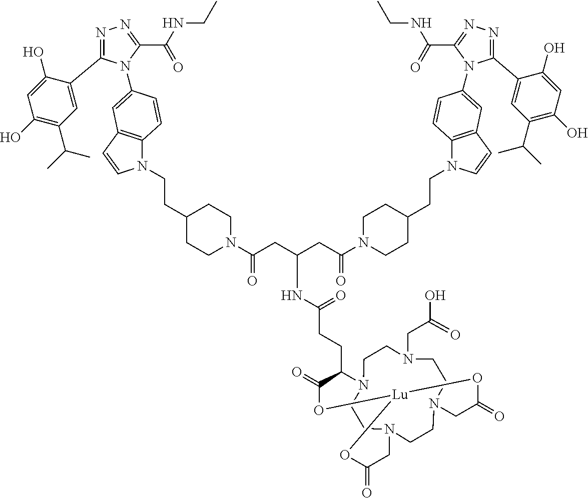

T19: comprising 2 TM14s; encompassed by X2

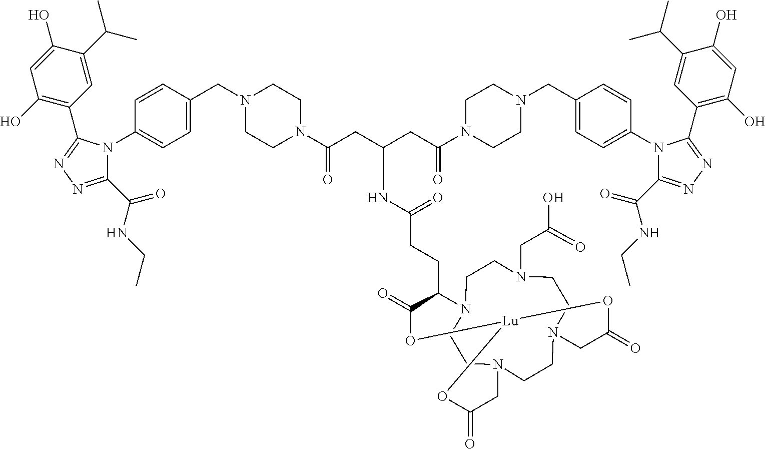

T20: comprising 2 TM5s; encompassed by X2

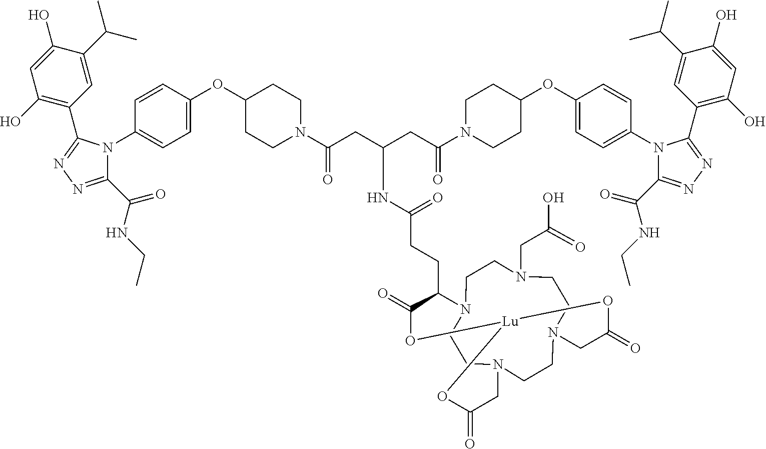

T21: comprising 2 TM10s; encompassed by X2

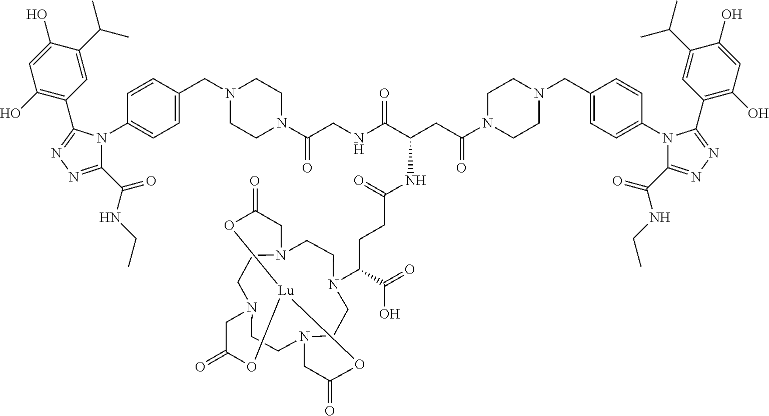

T25: comprising 2 TM5s; encompassed by X2

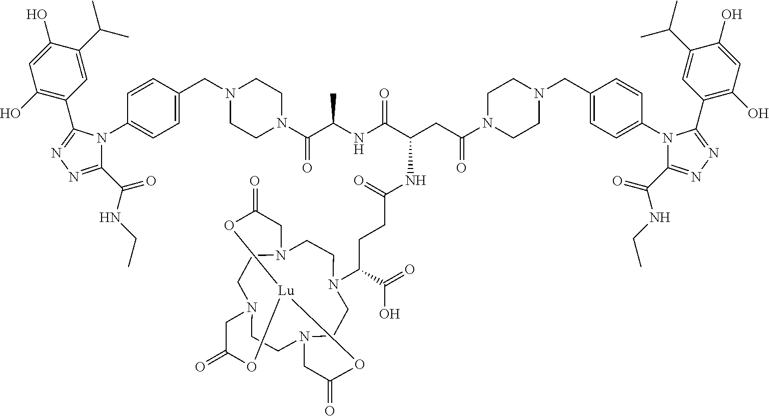

T26: comprising 2 TM5s; encompassed by X2

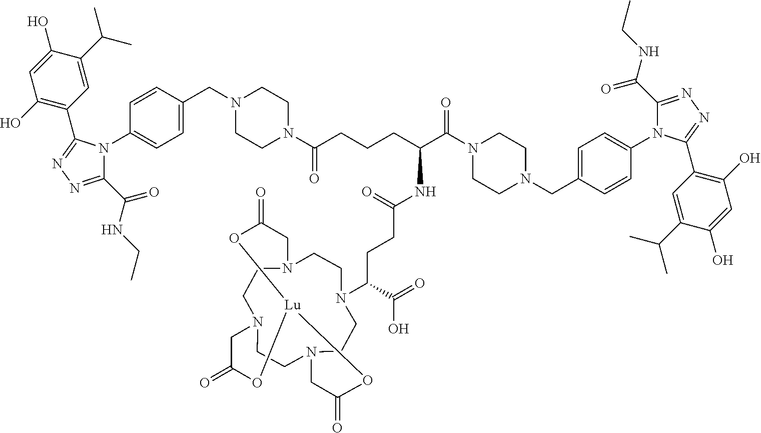

T30: comprising 2 TM5s; encompassed by X2

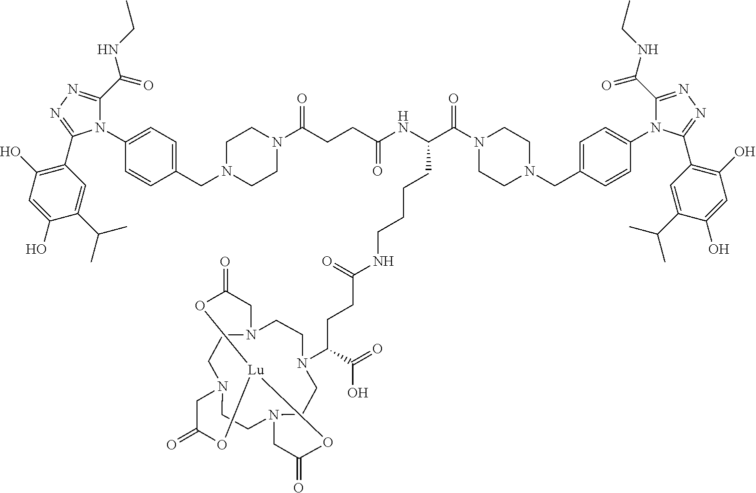

T31: comprising 2 TM5s; encompassed by X2

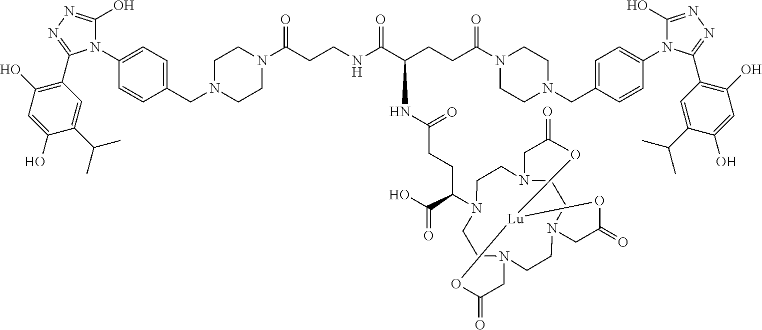

T43: comprising 2 TM5s; encompassed by X2

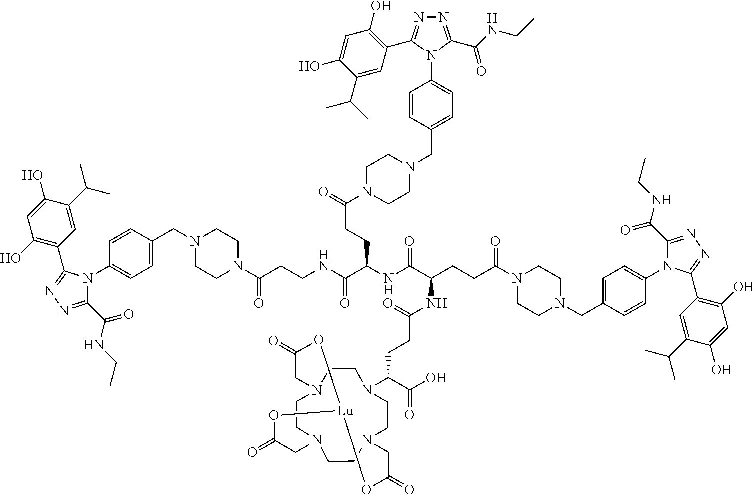

T40: comprising 3 TM5s; encompassed by X2

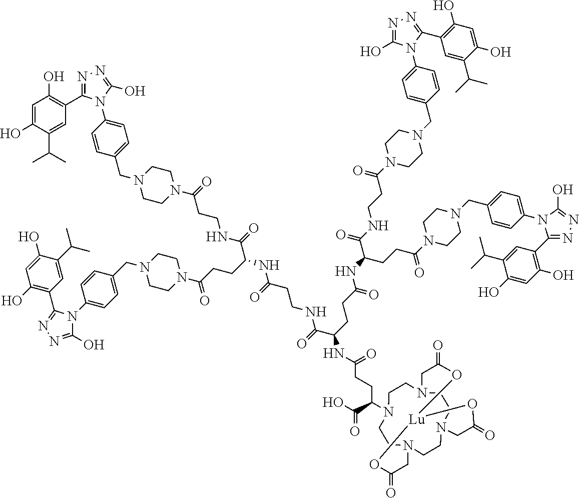

CMP9: comprising 4 TM5s; encompassed by Formula X2

The conjugate analog structures without the radioactive agent are included in Table 1′. The conjugates may comprise any radioisotope, such as Y-90, Y-86, I-131, Re-186, Re-188, Y-90, Bi-212, At-211, Zr-89, Sr-89, Ho-166, Sm-153, Cu-67, Cu-64, Lu-177, Ac-225, Pb-203, Bi-213, Th-227, Pb-212, Ra-223, P-32, Sc-47, Br-77, Rh-105, Pd-103, Ag-111, Pr-142, Pm-149, Gd-159, Ir-194 and Pt-199.

TABLE 1′
Non-limiting Examples of Conjugates (without radioactive agent)
Structures

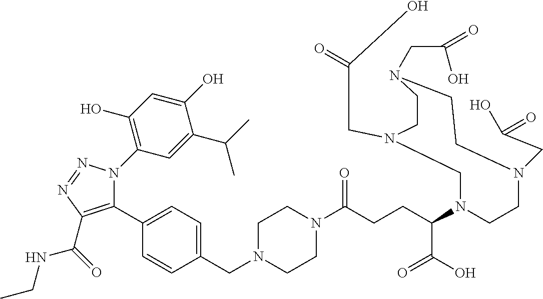 [SMILES: CCNC(=O)c1nnn(-c2cc(C(C)C)c(O)cc2O)c1-c1ccc(CN2CCN(C(=O)CC[C@H](C(=O)O)N3CCN(CC(=O)O)CCN(CC(=O)O)CCN(CC(=O)O)C3)CC2)cc1]

CMP22′: comprising TM5; encompassed by Formula X2

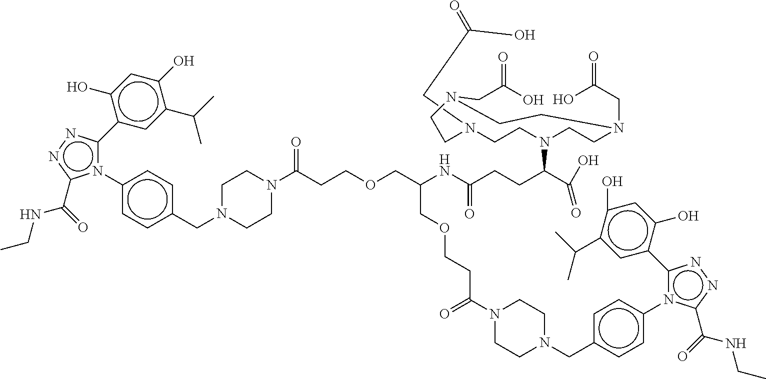

CMP7′: comprising 2 TM9s; encompassed by Formula X2

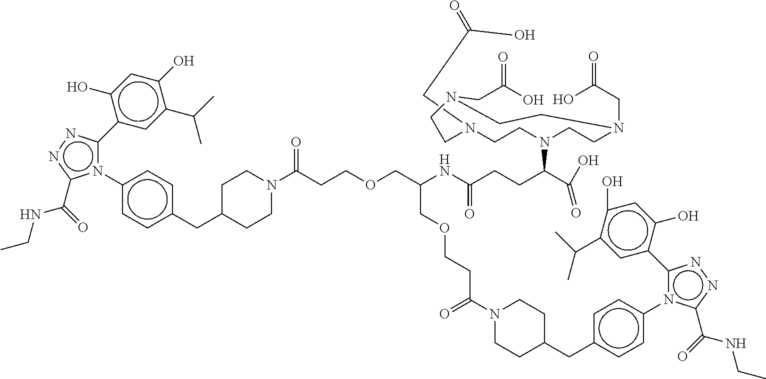

CMP8′: comprising 2 TM5s; encompassed by Formula X2

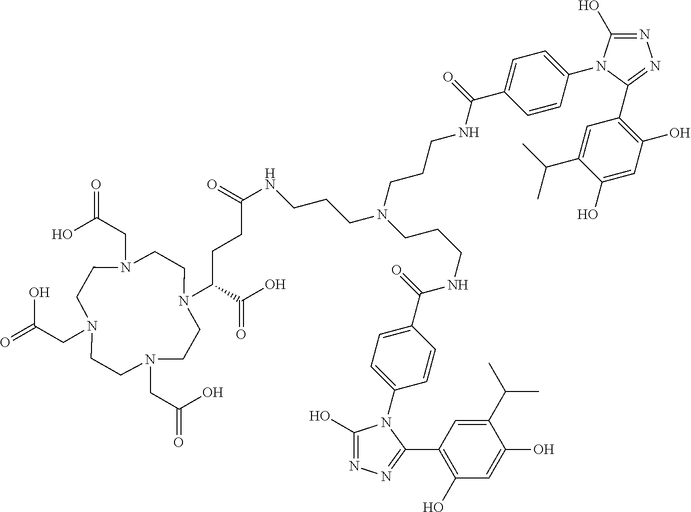 [SMILES: CC(C)c1cc(-c2nnc(O)n2-c2ccc(C(=O)NCCCN(CCCNC(=O)CC[C@H](C(=O)O)N3CCN(CC(=O)O)CCN(CC(=O)O)CCN(CC(=O)O)CC3)CCCNC(=O)c3ccc(-n4c(O)nnc4-c4cc(C(C)C)c(O)cc4O)cc3)cc2)c(O)cc1O]

CMP10′: comprising 2 TM3s; encompassed by Formula X2

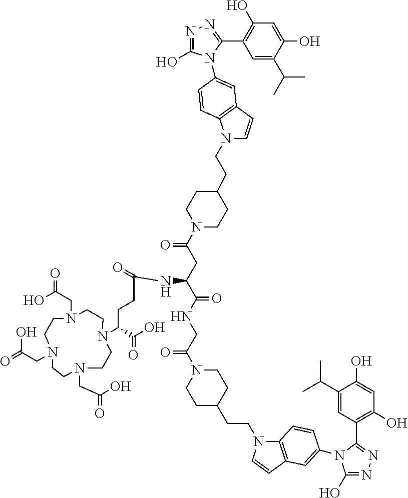 [SMILES: CC(C)c1cc(-c2nnc(O)n2-c2ccc3c(ccn3CCC3CCN(C(=O)CNC(=O)[C@H](CC(=O)N4CCC(CCn5ccc6cc(-n7c(O)nnc7-c7cc(C(C)C)c(O)cc7O)ccc65)CC4)NC(=O)CC[C@H](C(=O)O)N4CCN(CC(=O)O)CCN(CC(=O)O)CCN(CC(=O)O)CC4)CC3)c2)c(O)cc1O]

CMP 11′: comprising 2 TM1s; encompassed by Formula X2

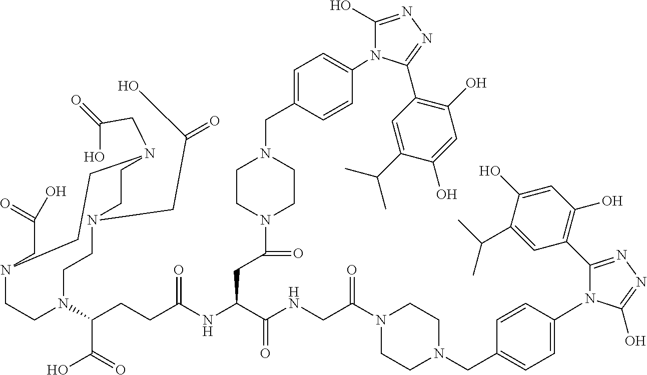 [SMILES: CC(C)c1cc(-c2nnc(O)n2-c2ccc(CN3CCN(C(=O)CNC(=O)[C@H](CC(=O)N4CCN(Cc5ccc(-n6c(O)nnc6-c6cc(C(C)C)c(O)cc6O)cc5)CC4)NC(=O)CC[C@H](C(=O)O)N4CCN(CC(=O)O)CCN(CC(=O)O)CCN(CC(=O)O)CC4)CC3)cc2)c(O)cc1O]

CMP12′: comprising 2 TM2s; encompassed by Formula X2

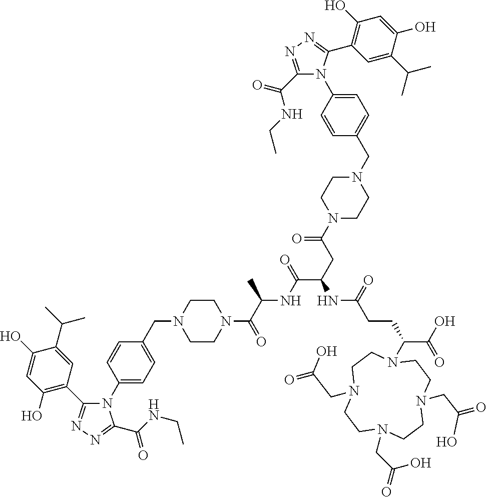 [SMILES: CCNC(=O)c1nnc(-c2cc(C(C)C)c(O)cc2O)n1-c1ccc(CN2CCN(C(=O)C[C@@H](NC(=O)CC[C@H](C(=O)O)N3CCN(CC(=O)O)CCN(CC(=O)O)CCN(CC(=O)O)CC3)C(=O)N[C@H](C)C(=O)N3CCN(Cc4ccc(-n5c(C(=O)NCC)nnc5-c5cc(C(C)C)c(O)cc5O)cc4)CC3)CC2)cc1]

CMP17′: comprising 2 TM5s; encompassed by Formula X2

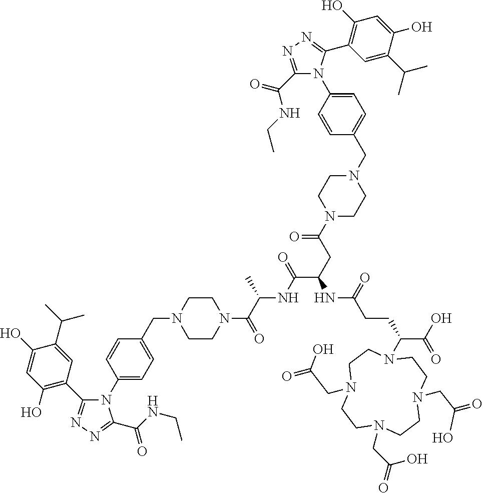 [SMILES: CCNC(=O)c1nnc(-c2cc(C(C)C)c(O)cc2O)n1-c1ccc(CN2CCN(C(=O)C[C@@H](NC(=O)CC[C@H](C(=O)O)N3CCN(CC(=O)O)CCN(CC(=O)O)CCN(CC(=O)O)CC3)C(=O)N[C@@H](C)C(=O)N3CCN(Cc4ccc(-n5c(C(=O)NCC)nnc5-c5cc(C(C)C)c(O)cc5O)cc4)CC3)CC2)cc1]

CMP18′: comprising 2 TM5s; encompassed by Formula X2

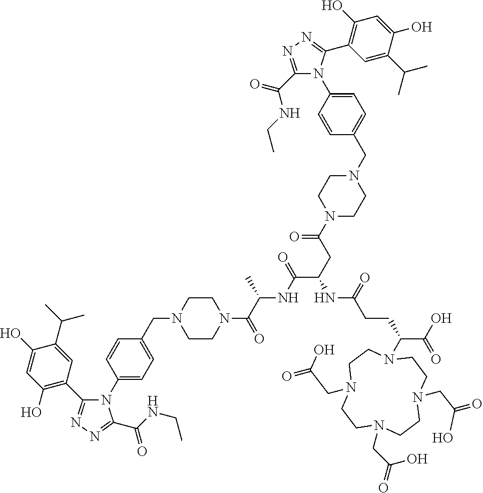 [SMILES: CCNC(=O)c1nnc(-c2cc(C(C)C)c(O)cc2O)n1-c1ccc(CN2CCN(C(=O)C[C@H](NC(=O)CC[C@H](C(=O)O)N3CCN(CC(=O)O)CCN(CC(=O)O)CCN(CC(=O)O)CC3)C(=O)N[C@@H](C)C(=O)N3CCN(Cc4ccc(-n5c(C(=O)NCC)nnc5-c5cc(C(C)C)c(O)cc5O)cc4)CC3)CC2)cc1]

CMP19′: comprising 2 TM5s; encompassed by Formula X2

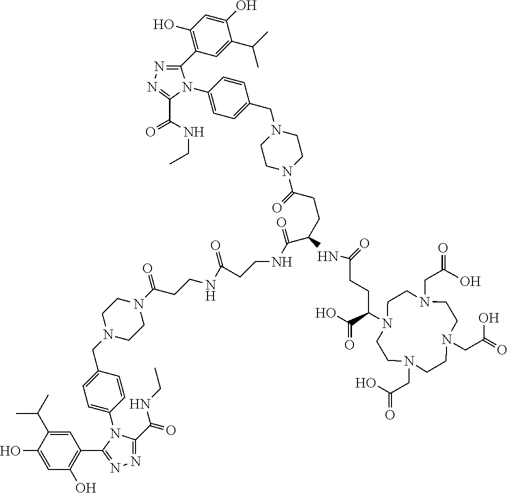 [SMILES: CCNC(=O)c1nnc(-c2cc(C(C)C)c(O)cc2O)n1-c1ccc(CN2CCN(C(=O)CCNC(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CC[C@H](C(=O)O)N3CCN(CC(=O)O)CCN(CC(=O)O)CCN(CC(=O)O)CC3)CC2)cc1]

CMP20′: comprising 2 TM5s; encompassed by Formula X2

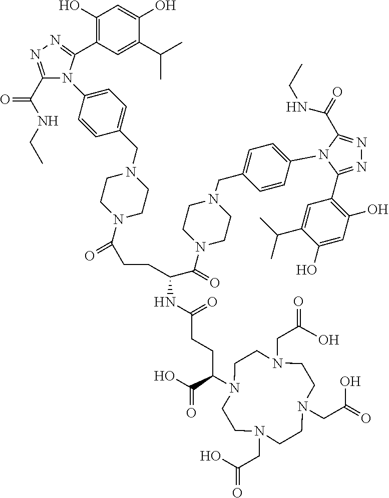

CMP21′: comprising 2 TM5s; encompassed by Formula X2

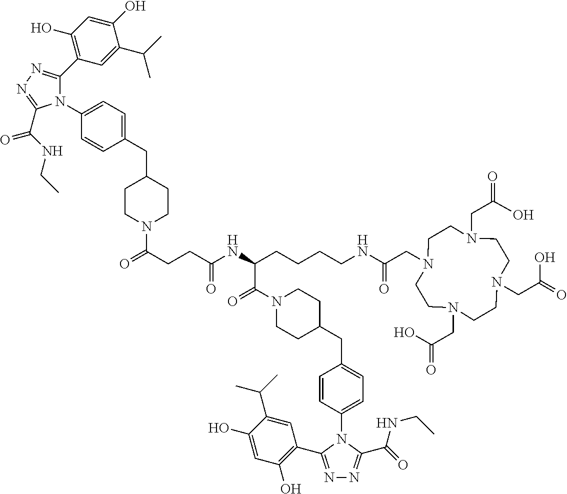

CMP24′: comprising 2 TM9s; encompassed by Formula X1

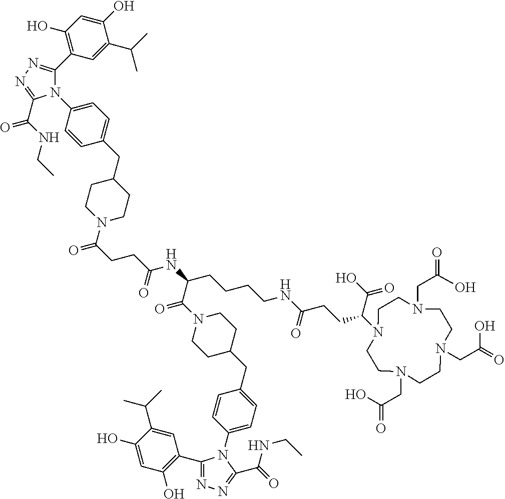 [SMILES: CCNC(=O)c1nnc(-c2cc(C(C)C)c(O)cc2O)n1-c1ccc(CC2CCN(C(=O)CCC(=O)N[C@@H](CCCCNC(=O)CC[C@H](C(=O)O)N3CCN(CC(=O)O)CCN(CC(=O)O)CCN(CC(=O)O)CC3)C(=O)N3CCC(Cc4ccc(-n5c(C(=O)NCC)nnc5-c5cc(C(C)C)c(O)cc5O)cc4)CC3)CC2)cc1]

CMP25′: comprising 2 TM9s; encompassed by Formula X2

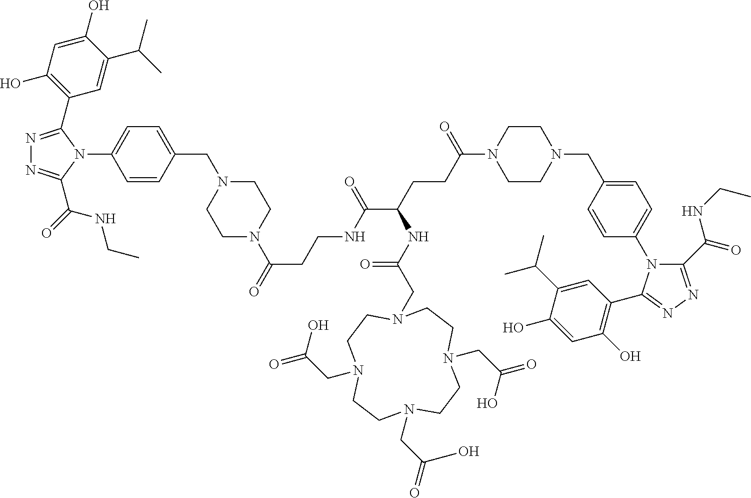 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CN3CCN(CC(=O)O)CCN(CC(=O)O)CCN(CC(=O)O)CC3)CC2)cc1]

CMP26′: comprising 2 TM5s; encompassed by Formula X1

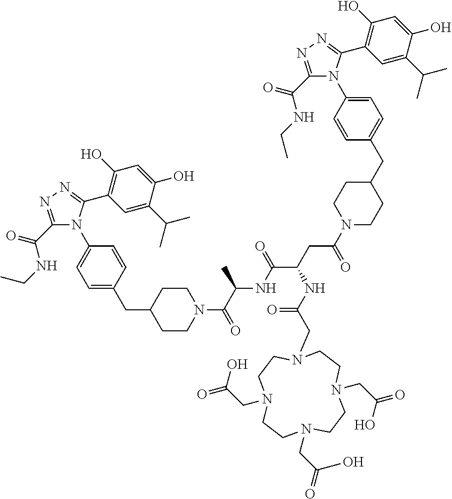 [SMILES: CCNC(=O)c1nnc(-c2cc(C(C)C)c(O)cc2O)n1-c1ccc(CC2CCN(C(=O)C[C@H](NC(=O)CN3CCN(CC(=O)O)CCN(CC(=O)O)CCN(CC(=O)O)CC3)C(=O)N[C@H](C)C(=O)N3CCC(Cc4ccc(-n5c(C(=O)NCC)nnc5-c5cc(C(C)C)c(O)cc5O)cc4)CC3)CC2)cc1]

CMP27′: comprising 2 TM9s; encompassed by Formula X1

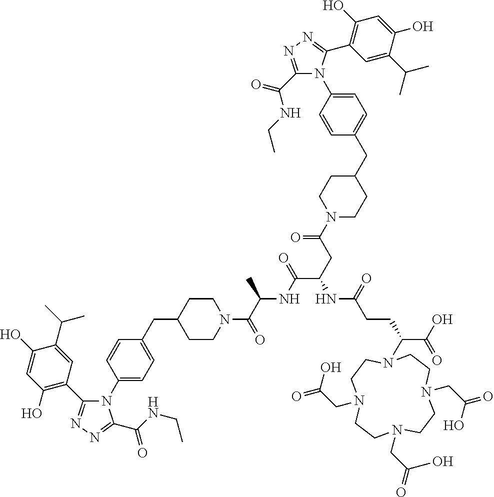 [SMILES: CCNC(=O)c1nnc(-c2cc(C(C)C)c(O)cc2O)n1-c1ccc(CC2CCN(C(=O)C[C@H](NC(=O)CC[C@H](C(=O)O)N3CCN(CC(=O)O)CCN(CC(=O)O)CCN(CC(=O)O)CC3)C(=O)N[C@H](C)C(=O)N3CCC(Cc4ccc(-n5c(C(=O)NCC)nnc5-c5cc(C(C)C)c(O)cc5O)cc4)CC3)CC2)cc1]

CMP28′: comprising 2 TM9s; encompassed by Formula X2

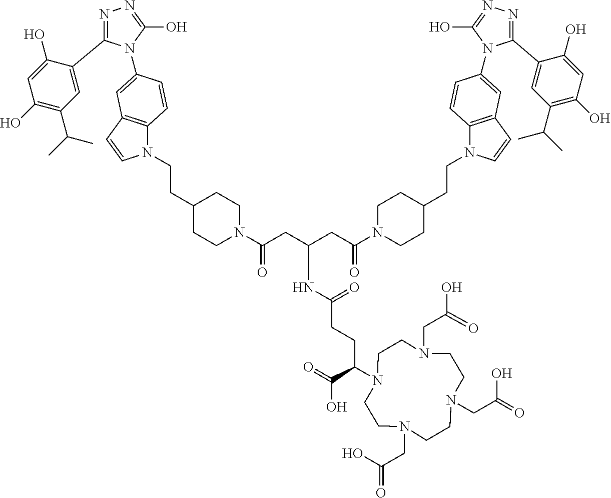 [SMILES: CC(C)c1cc(-c2nnc(O)n2-c2ccc3c(ccn3CCC3CCN(C(=O)CC(CC(=O)N4CCC(CCn5ccc6cc(-n7c(O)nnc7-c7cc(C(C)C)c(O)cc7O)ccc65)CC4)NC(=O)CC[C@H](C(=O)O)N4CCN(CC(=O)O)CCN(CC(=O)O)CCN(CC(=O)O)CC4)CC3)c2)c(O)cc1O]

T18′: comprising 2 TM1s; encompassed by X2

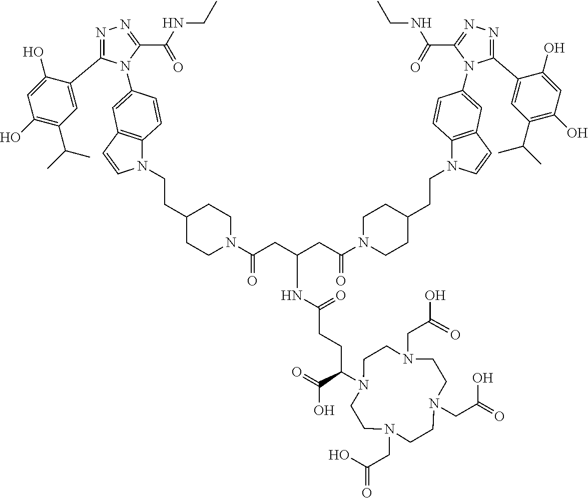 [SMILES: CCNC(=O)c1nnc(-c2cc(C(C)C)c(O)cc2O)n1-c1ccc2c(ccn2CCC2CCN(C(=O)CC(CC(=O)N3CCC(CCn4ccc5cc(-n6c(C(=O)NCC)nnc6-c6cc(C(C)C)c(O)cc6O)ccc54)CC3)NC(=O)CC[C@H](C(=O)O)N3CCN(CC(=O)O)CCN(CC(=O)O)CCN(CC(=O)O)CC3)CC2)c1]

T19′: comprising 2 TM14s; encompassed by X2

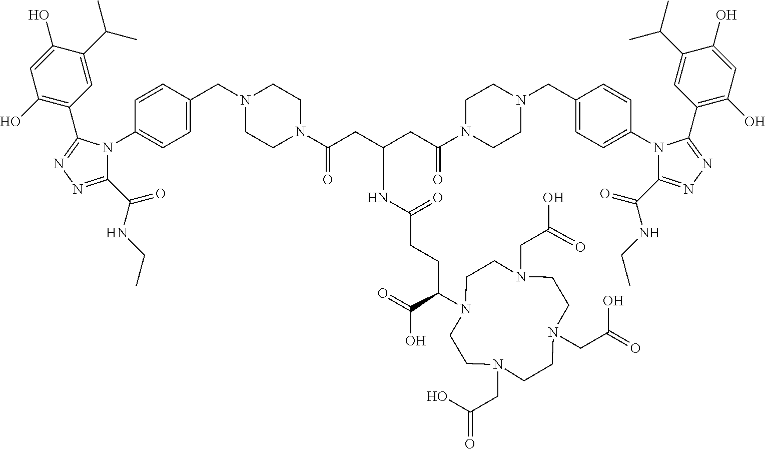 [SMILES: CCNC(=O)c1nnc(-c2cc(C(C)C)c(O)cc2O)n1-c1ccc(CN2CCN(C(=O)CC(CC(=O)N3CCN(Cc4ccc(-n5c(C(=O)NCC)nnc5-c5cc(C(C)C)c(O)cc5O)cc4)CC3)NC(=O)CC[C@H](C(=O)O)N3CCN(CC(=O)O)CCN(CC(=O)O)CCN(CC(=O)O)CC3)CC2)cc1]

T20′: comprising 2 TM5s; encompassed by X2

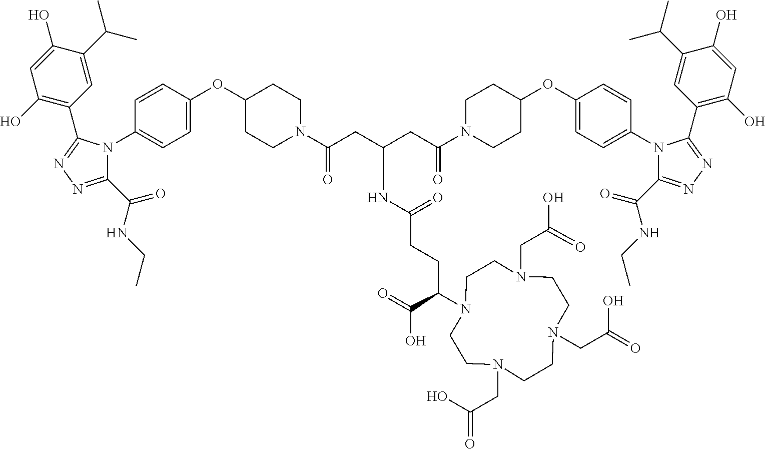 [SMILES: CCNC(=O)c1nnc(-c2cc(C(C)C)c(O)cc2O)n1-c1ccc(OC2CCN(C(=O)CC(CC(=O)N3CCC(Oc4ccc(-n5c(C(=O)NCC)nnc5-c5cc(C(C)C)c(O)cc5O)cc4)CC3)NC(=O)CC[C@H](C(=O)O)N3CCN(CC(=O)O)CCN(CC(=O)O)CCN(CC(=O)O)CC3)CC2)cc1]

T21′: comprising 2 TM10s; encompassed by X2

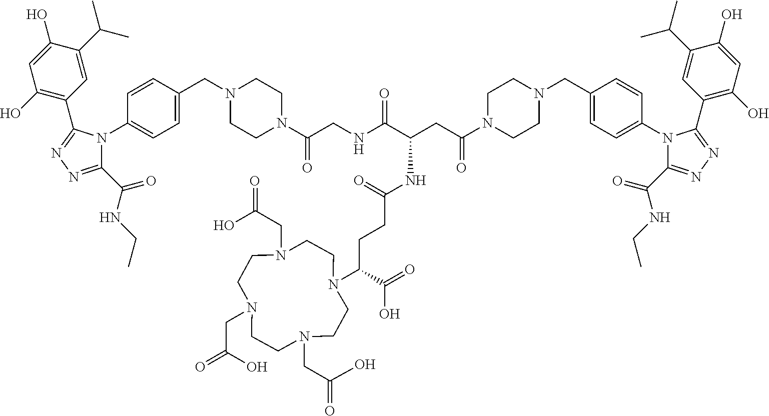 [SMILES: CCNC(=O)c1nnc(-c2cc(C(C)C)c(O)cc2O)n1-c1ccc(CN2CCN(C(=O)CNC(=O)[C@H](CC(=O)N3CCN(Cc4ccc(-n5c(C(=O)NCC)nnc5-c5cc(C(C)C)c(O)cc5O)cc4)CC3)NC(=O)CC[C@H](C(=O)O)N3CCN(CC(=O)O)CCN(CC(=O)O)CCN(CC(=O)O)CC3)CC2)cc1]

T25′: comprising 2 TM5s; encompassed by X2

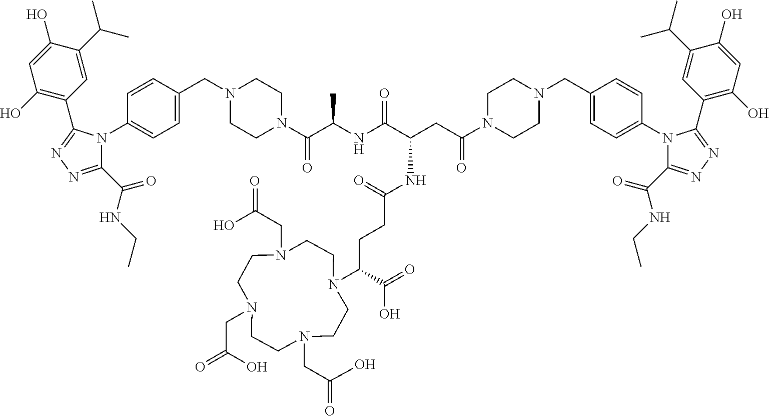 [SMILES: CCNC(=O)c1nnc(-c2cc(C(C)C)c(O)cc2O)n1-c1ccc(CN2CCN(C(=O)C[C@H](NC(=O)CC[C@H](C(=O)O)N3CCN(CC(=O)O)CCN(CC(=O)O)CCN(CC(=O)O)CC3)C(=O)N[C@H](C)C(=O)N3CCN(Cc4ccc(-n5c(C(=O)NCC)nnc5-c5cc(C(C)C)c(O)cc5O)cc4)CC3)CC2)cc1]

T26′: comprising 2 TM5s; encompassed by X2

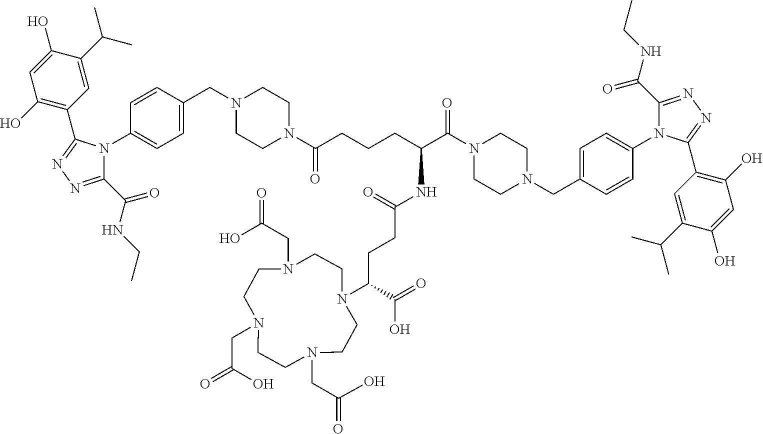 [SMILES: CCNC(=O)c1nnc(-c2cc(C(C)C)c(O)cc2O)n1-c1ccc(CN2CCN(C(=O)CCC[C@H](NC(=O)CC[C@H](C(=O)O)N3CCN(CC(=O)O)CCN(CC(=O)O)CCN(CC(=O)O)CC3)C(=O)N3CCN(Cc4ccc(-n5c(C(=O)NCC)nnc5-c5cc(C(C)C)c(O)cc5O)cc4)CC3)CC2)cc1]

T30′: comprising 2 TM5s; encompassed by X2

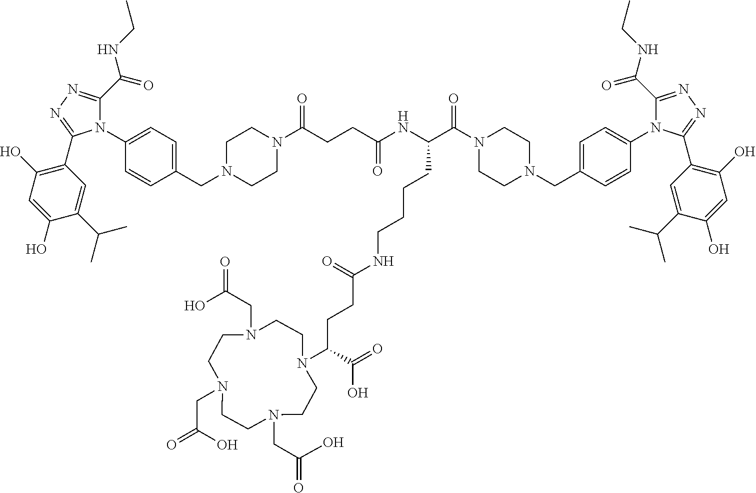 [SMILES: CCNC(=O)c1nnc(-c2cc(C(C)C)c(O)cc2O)n1-c1ccc(CN2CCN(C(=O)CCC(=O)N[C@@H](CCCCNC(=O)CC[C@H](C(=O)O)N3CCN(CC(=O)O)CCN(CC(=O)O)CCN(CC(=O)O)CC3)C(=O)N3CCN(Cc4ccc(-n5c(C(=O)NCC)nnc5-c5cc(C(C)C)c(O)cc5O)cc4)CC3)CC2)cc1]

T31′: comprising 2 TM5s; encompassed by X2

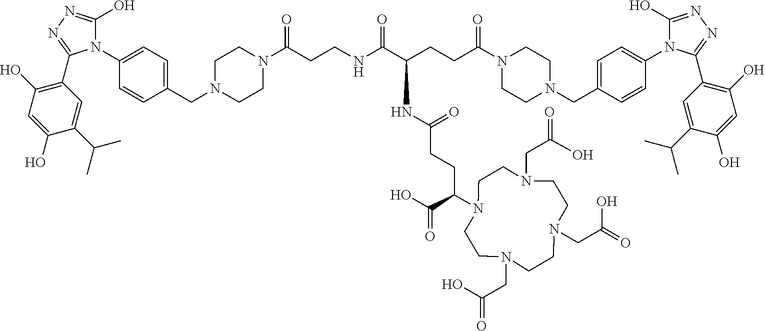 [SMILES: CC(C)c1cc(-c2nnc(O)n2-c2ccc(CN3CCN(C(=O)CCNC(=O)[C@@H](CCC(=O)N4CCN(Cc5ccc(-n6c(O)nnc6-c6cc(C(C)C)c(O)cc6O)cc5)CC4)NC(=O)CC[C@H](C(=O)O)N4CCN(CC(=O)O)CCN(CC(=O)O)CCN(CC(=O)O)CC4)CC3)cc2)c(O)cc1O]

T43′: comprising 2 TM5s; encompassed by X2

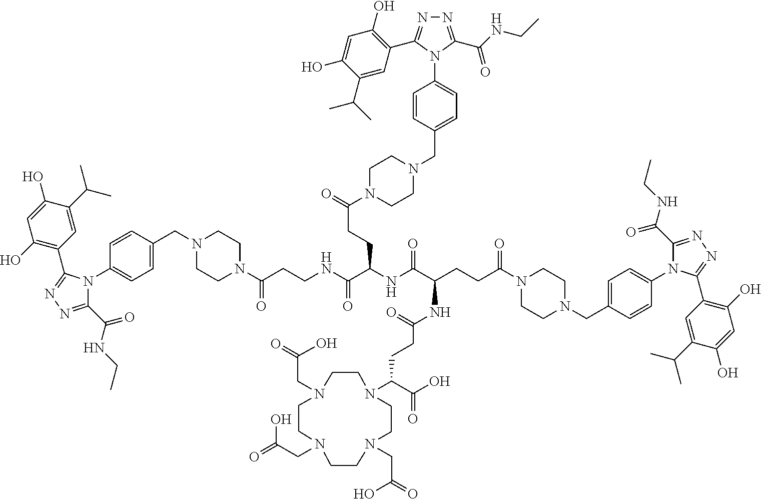 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)[C@@H](CCC(=O)N3CCN(Cc4ccc(-n5c(C(=O)NCC)nnc5-c5cc(C(C)C)c(O)cc5O)cc4)CC3)NC(=O)CC[C@H](C(=O)O)N3CCN(CC(=O)O)CCN(CC(=O)O)CCN(CC(=O)O)CC3)CC2)cc1]

T40′: comprising 3 TM5s; encompassed by X2

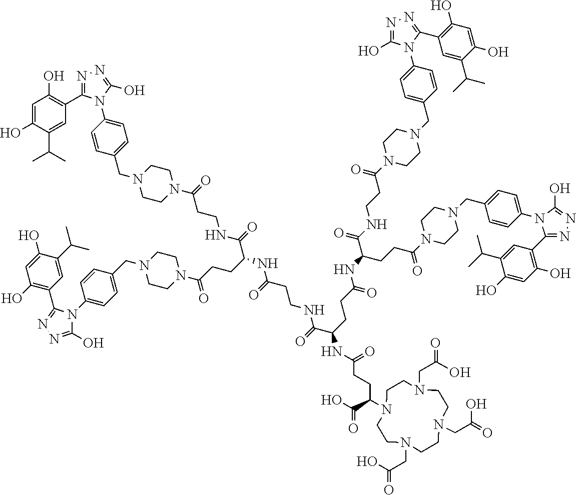 [SMILES: CC(C)c1cc(-c2nnc(O)n2-c2ccc(CN3CCN(C(=O)CCNC(=O)[C@@H](CCC(=O)N4CCN(Cc5ccc(-n6c(O)nnc6-c6cc(C(C)C)c(O)cc6O)cc5)CC4)NC(=O)CCNC(=O)[C@@H](CCC(=O)N[C@H](CCC(=O)N4CCN(Cc5ccc(-n6c(O)nnc6-c6cc(C(C)C)c(O)cc6O)cc5)CC4)C(=O)NCCC(=O)N4CCN(Cc5ccc(-n6c(O)nnc6-c6cc(C(C)C)c(O)cc6O)cc5)CC4)NC(=O)CC[C@H](C(=O)O)N4CCN(CC(=O)O)CCN(CC(=O)O)CCN(CC(=O)O)CC4)CC3)cc2)c(O)cc1O]

CMP9′: comprising 4 TM5s; encompassed by Formula X2

In some embodiments, the conjugate comprises at least at least one TM1 as a ligand. In some embodiments, the conjugate comprises at least one amino acid spacer and comprises 1 TM1 as a ligand. In some embodiments, the conjugate comprises at least one amino acid spacer and comprises two TM1s as ligands. Non-limiting examples of the conjugates include CMP11 and T18.

In some embodiments, the conjugate comprises at least at least one TM2 as a ligand. In some embodiments, the conjugate comprises at least one amino acid spacer and comprises 1 TM2 as a ligand. In some embodiments, the conjugate comprises at least one amino acid spacer and comprises two TM2s as ligands. Non-limiting examples of the conjugates include CMP12.

In some embodiments, the conjugate comprises at least at least one TM3 as a ligand. In some embodiments, the conjugate comprises at least one amino acid spacer and comprises 1 TM3 as a ligand. In some embodiments, the conjugate comprises at least one amino acid spacer and comprises two TM3s as ligands. Non-limiting examples of the conjugates include CMP10.

In some embodiments, the conjugate comprises at least at least one TM5 as a ligand. In some embodiments, the conjugate comprises at least one amino acid spacer and comprises 1 TM5 as a ligand, such as CMP22. In some embodiments, the conjugate comprises at least one amino acid spacer and comprises two TM5s as ligands. Non-limiting examples of the conjugates include CMP8, CMP17, CMP18, CIMP19, CMP 20, CMP21, CMP26, T20, T25, T26, T30, T31, and T43. In some embodiments, the conjugate comprises at least one amino acid spacer and comprises 3 TM5s as ligands. Non-limiting examples of the conjugates include T40. In some embodiments, the conjugate comprises at least one amino acid spacer and comprises 4 TM5s as ligands. Non-limiting examples of the conjugates include CMP9.

In some embodiments, the conjugate comprises at least at least one TM9 as a ligand. In some embodiments, the conjugate comprises at least one amino acid spacer and comprises 1 TM9 as a ligand. In some embodiments, the conjugate comprises at least one amino acid spacer and comprises two TM9s as ligands. Non-limiting examples of the conjugates include CMP24, CMP25, CMP27, and CMP28.

In some embodiments, the conjugate comprises at least at least one TM10 as a ligand. In some embodiments, the conjugate comprises at least one amino acid spacer and comprises 1 TM10 as a ligand. In some embodiments, the conjugate comprises at least one amino acid spacer and comprises two TM10s as ligands. Non-limiting examples of conjugates include CMP7, and T21.

In some embodiments, the conjugate comprises at least at least one TM14 as a ligand. In some embodiments, the conjugate comprises at least one amino acid spacer and comprises 1 TM14 as a ligand. In some embodiments, the conjugate comprises at least one amino acid spacer and comprises two TM14s as ligands. Non-limiting examples of conjugates include T19.

### Amino Acid Spacers

In some embodiments, the conjugate comprises at least one HSP90 targeting moiety attached to a chelating agent for a radioactive agent with a linker. The HSP90 targeting moiety may be a ganetespib analog or derivative (such as TM1, TM2, TM3, TM4, TM5, TM8, TM9, TM10, TM11, TM12, TM13, or TM14), an onalespib analog or derivative (such as TM6 or TM7), or TM15. The linker may comprise a spacer made of at least one amino acid or analog(s) thereof, such as 2 amino acids or analogs thereof, 3 amino acids or analogs thereof, 4 amino acids or analogs thereof, or 5 amino acids or analogs thereof. The amino acids or analogs thereof may be D amino acids. The amino acids or analogs thereof may be anionic (e.g., DGlu), cationic (e.g., DLys), or uncharged (e.g., Sar, where Sar=N-methyl glycine). The spacer may be DGlu-DGlu-DLys, DLys-DLys-DGlu, DGlu-DGlu-DGlu, DLys-DLys-DLys, Sar-DLys-Sar, Sar-Sar-Sar, Sar-DGlu-Sar, Ala-Asp-D-Ser, Ala-Asp-L-Ser, or Glu. Not willing to be bound by any theory, the spacer affects biodistribution of the conjugates and may reduce liver uptake of the conjugates. HSP90 binding affinity is maintained regardless of what charges are present on the spacer.

In some embodiments, the conjugate has a structure of Formula A:

wherein TM(s) refers to at least one targeting moiety that binds to HSP90. In some embodiments, the conjugate has 2 targeting moieties. In some embodiments, the conjugate has 3 targeting moieties. In some embodiments, the conjugate has 4 targeting moieties. Non-limiting examples of the conjugates encompassed by Formula A1 include T1, T10, T27, T28, CMP13, CMP16, T2, T4, T5, T6, T8, T9, T29, T39, CMP14, CMP15, CMP23, CMP37, CMP38, CMP43, CMP44, CMP45, CMP46, CMP47, CMP48, CMP49, and CMP50.

In some embodiments, the conjugate has a structure of Formula A10:

TM is selected from TM1, TM2, TM3, TM4, TM5, TM8, TM9, TM10, TM11, TM12, TM13, TM14, TM6, TM7, or TM15.

In some embodiments, the conjugate has a structure of Formula A1:

TM is selected from TM1, TM2, TM3, TM4, TM5, TM8, TM9, TM10, TM11, TM12, TM13, TM14, TM6, TM7, or TM15. Non-limiting examples of the conjugates encompassed by Formula A1 include CMP23, CMP37, and CMP38.

In some embodiments, the conjugate has a structure of Formula A20:

TM is selected from TM1, TM2, TM3, TM4, TM5, TM8, TM9, TM10, TM11, TM12, TM13, TM14, TM6, TM7, or TM15.

In some embodiments, the conjugate has a structure of Formula A2:

TM is selected from TM1, TM2, TM3, TM4, TM5, TM8, TM9, TM10, TM11, TM12, TM13, TM14, TM6, TM7, or TM15. Non-limiting examples of the conjugates encompassed by Formula A2 include T10, T27, T28, CMP13, CMP16, T2, T4, T5, T6, T8, T9, T29, T39, CMP14, CMP15, CMP43, CMP44, CMP45, CMP46, CMP47, CMP48, CMP49, and CMP50.

Non-limiting examples of conjugates of the current disclosure that are encompassed by Formula A include T1, T2, T4, T5, T6, T8, T9, T10, T27, T28, T29, T39, CMP13, CMP14, CMP15, CMP16, CMP23, CMP37, CMP38, CMP43, CMP44, CMP45, CMP46, CMP47, CMP48, CMP49, or CMP50:

TABLE 2
Non-Limiting Examples of Conjugates Comprising Amino Acid Spacers
Structures

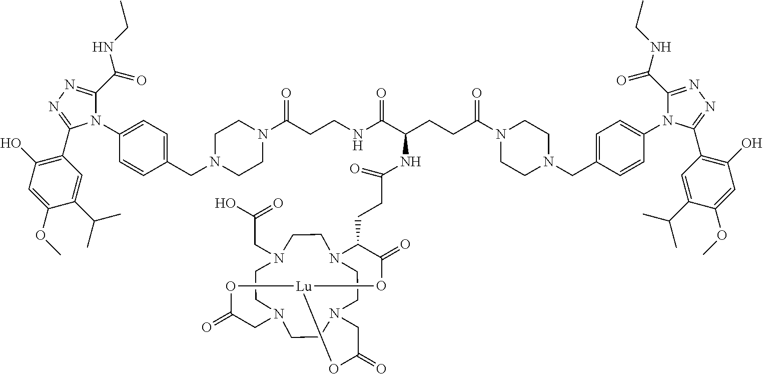

T1: comprising 2 TM5s; encompassed by Formula A2

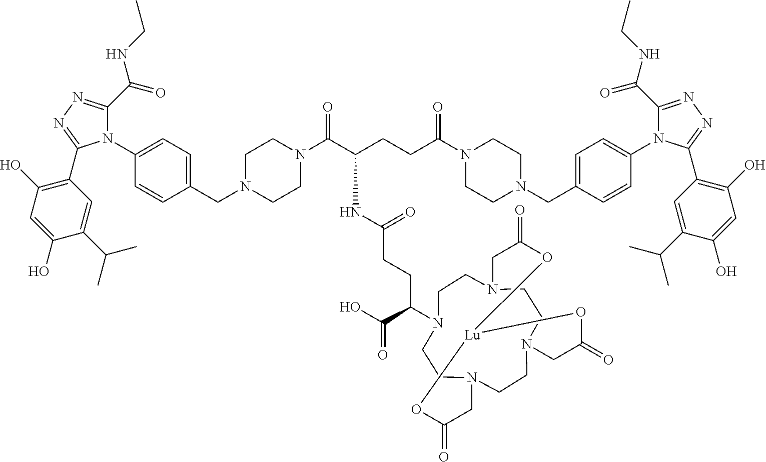

T2: comprising 2 TM5s; encompassed by A2

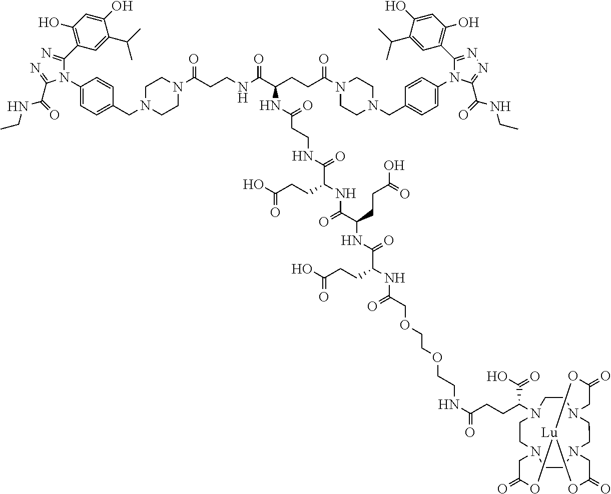

T4: comprising 2 TM5s; encompassed by A2

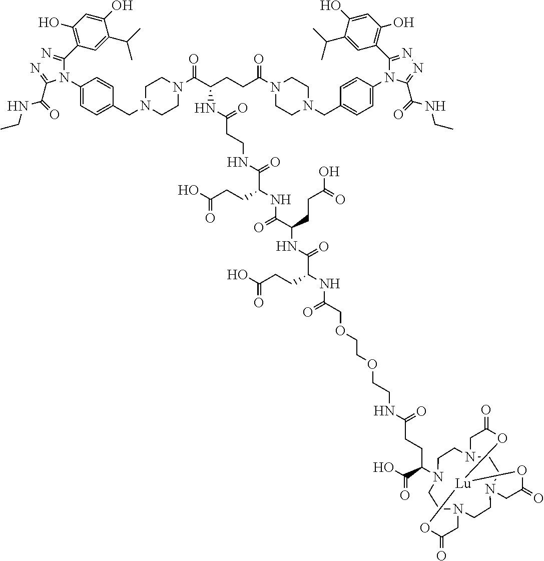

T5: comprising 2 TM5s; encompassed by A2

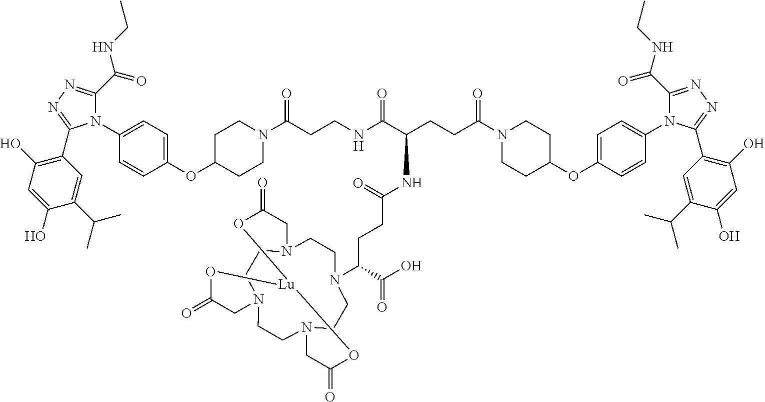

T6: comprising 2 TM10s; encompassed by A2

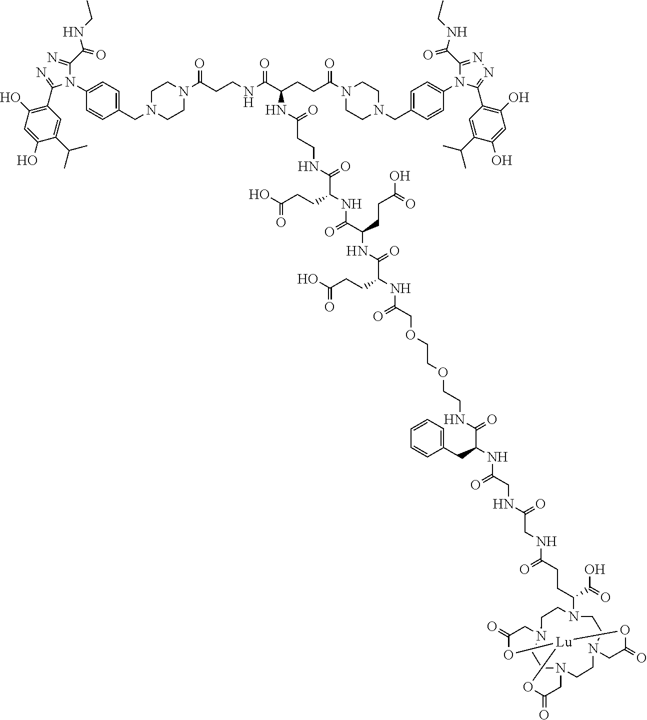

T8: comprising 2 TM5s; encompassed by A2

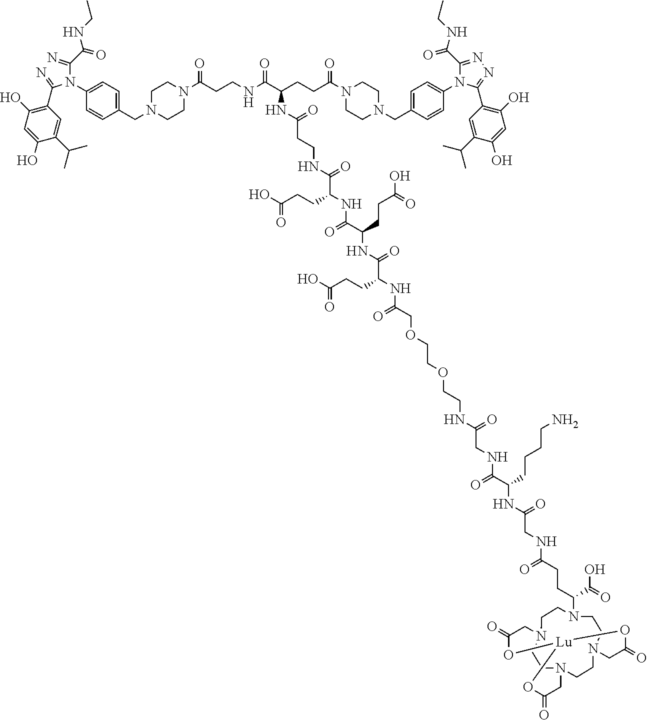

T9: comprising 2 TM5s; encompassed by A2

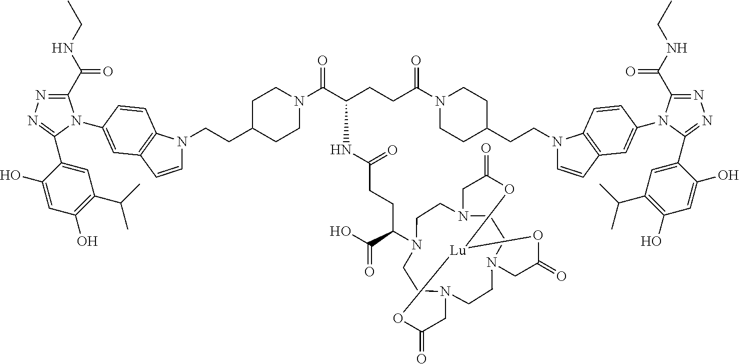

T10: comprising 2 TM1s; encompassed by A2

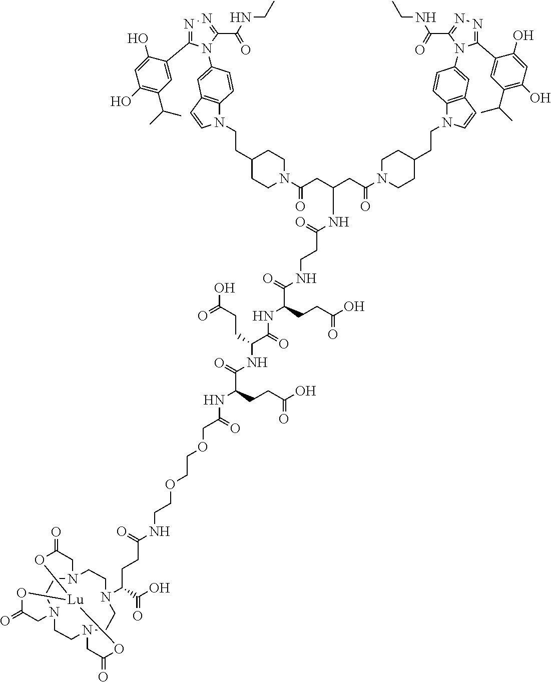

T27: comprising 2 TM1s; encompassed by A2

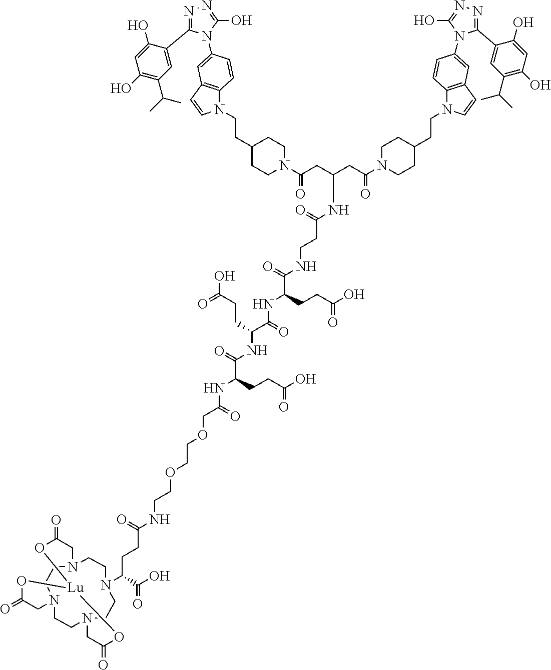

T28: comprising 2 TM1s; encompassed by A2

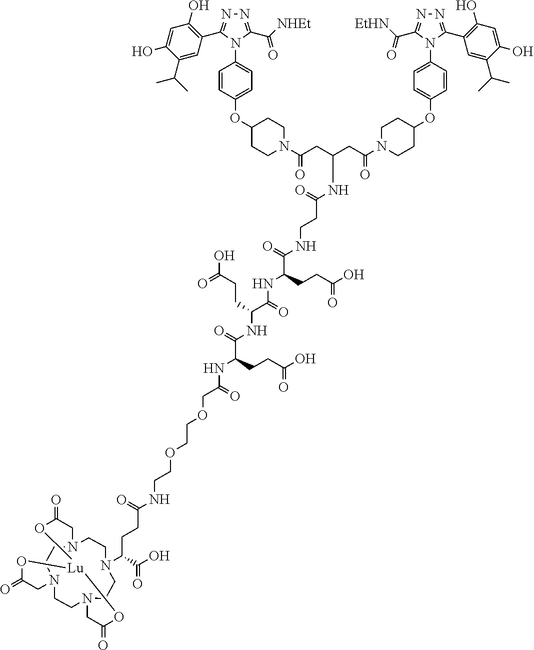

T29: comprising 2 TM10s; encompassed by A2

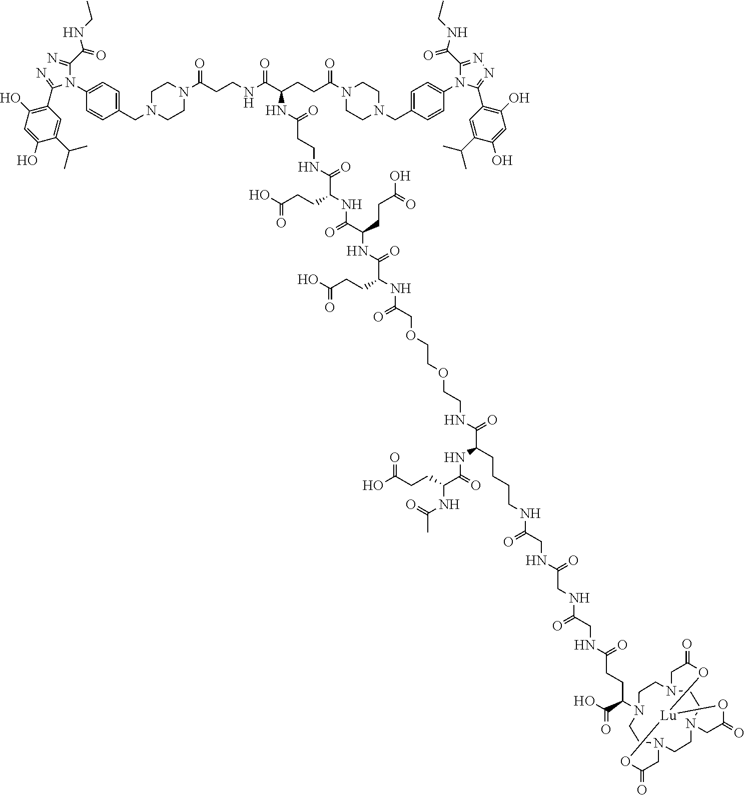

T39: comprising 2 TM5s; encompassed by A2

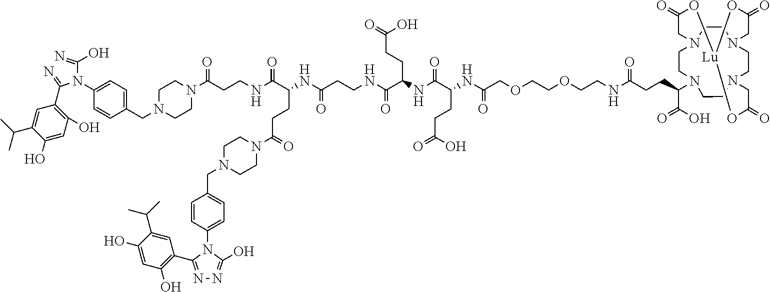

CMP13: comprising 2 TM2s; encompassed by A2

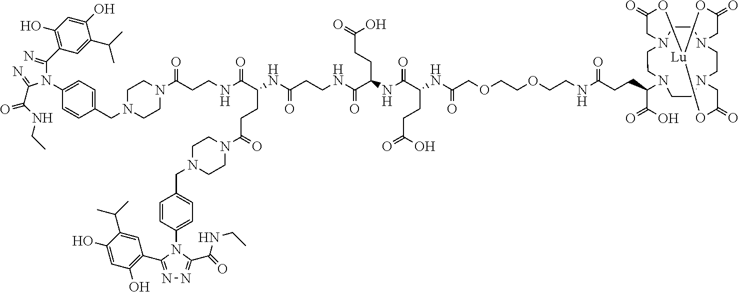

CMP14: comprising 2 TM5s; encompassed by A2

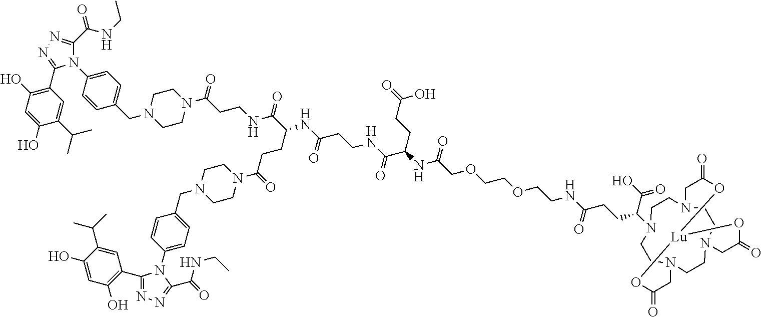

CMP15: comprising 2 TM5s; encompassed by A2

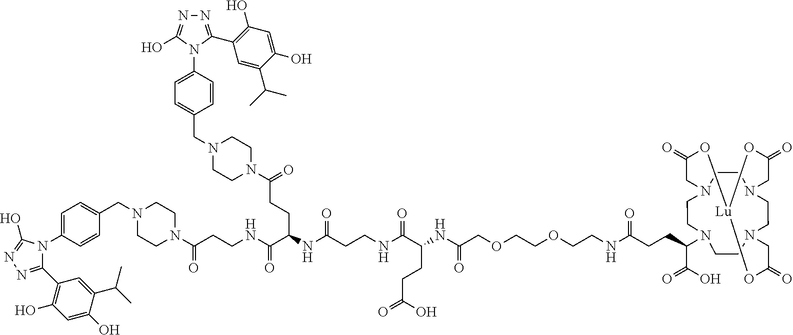

CMP16: comprising 2 TM2s; encompassed by A2

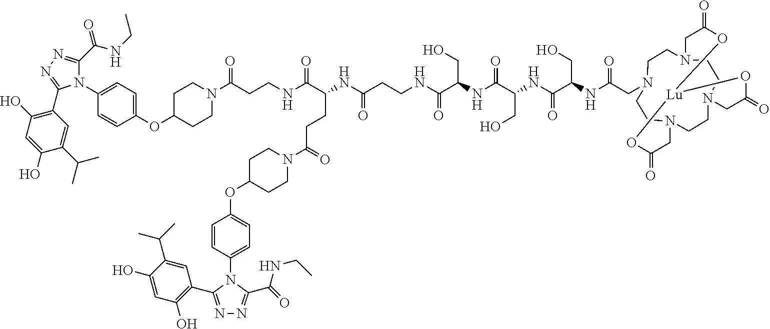

CMP37: comprising 2 TM10s; encompassed by Formula A1

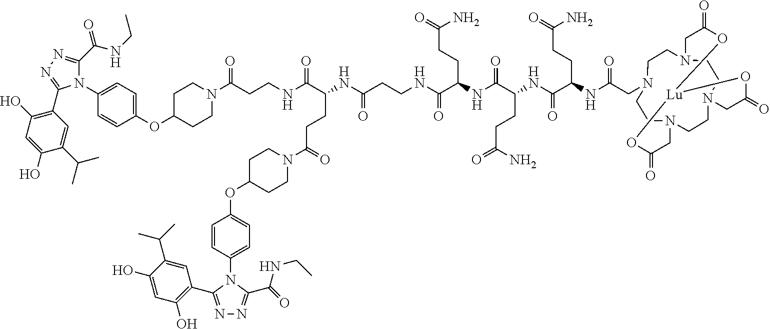

CMP38: comprising 2 TM10s; encompassed by Formula A1

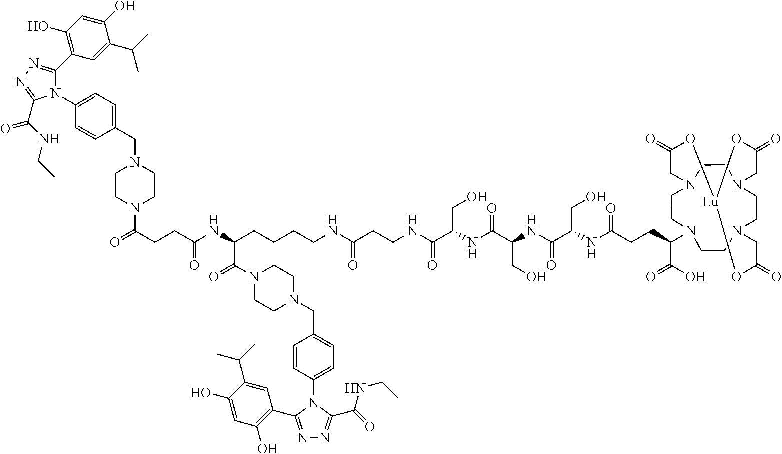

CMP43: comprising 2 TM5s; encompassed by Formula A2

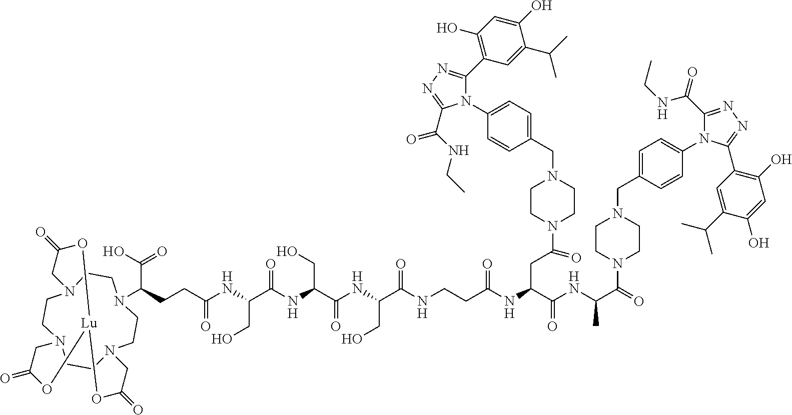

CMP44: comprising 2 TM5s; encompassed by Formula A2

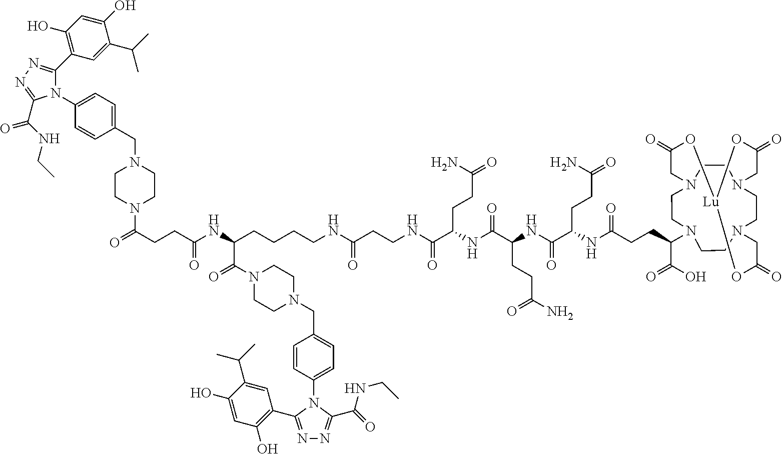

CMP45: comprising 2 TM5s; encompassed by Formula A2

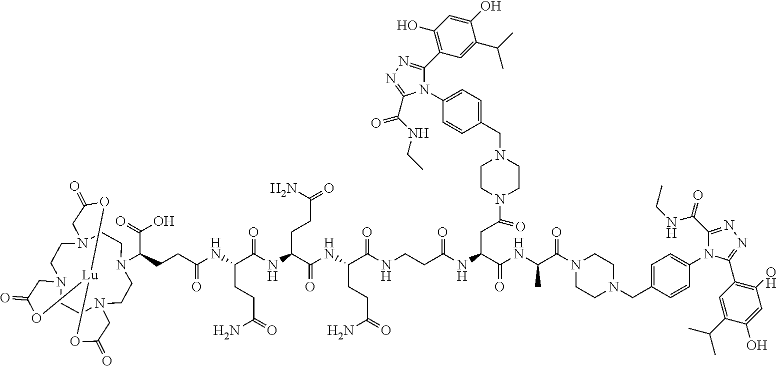

CMP46: comprising 2 TM5s; encompassed by Formula A2

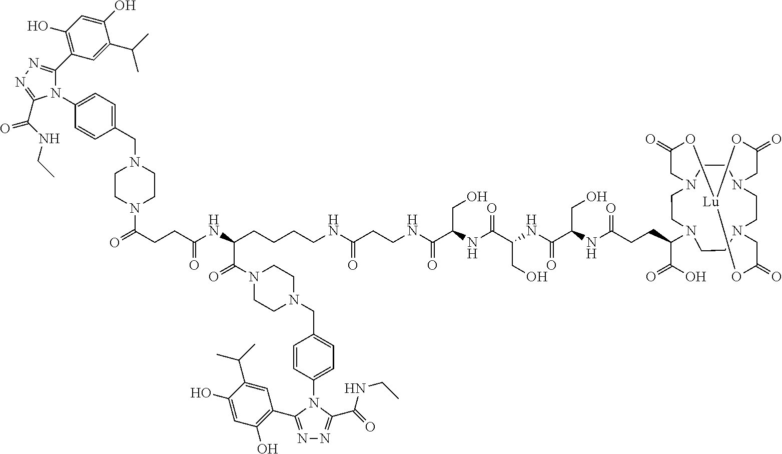

CMP47: comprising 2 TM5s; encompassed by Formula A2

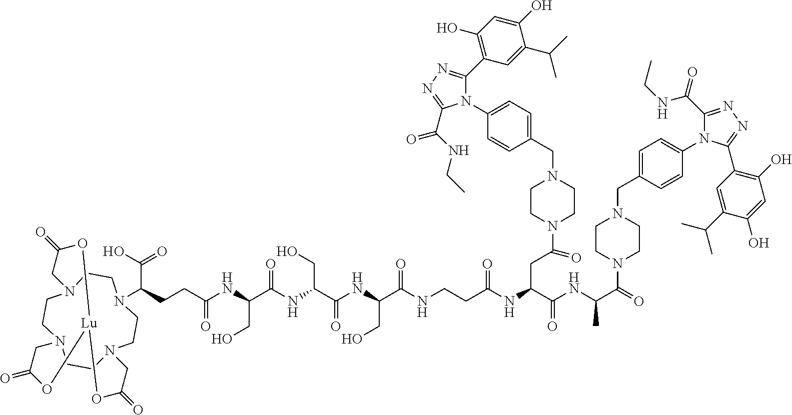

CMP48: comprising 2 TM5s; encompassed by Formula A2

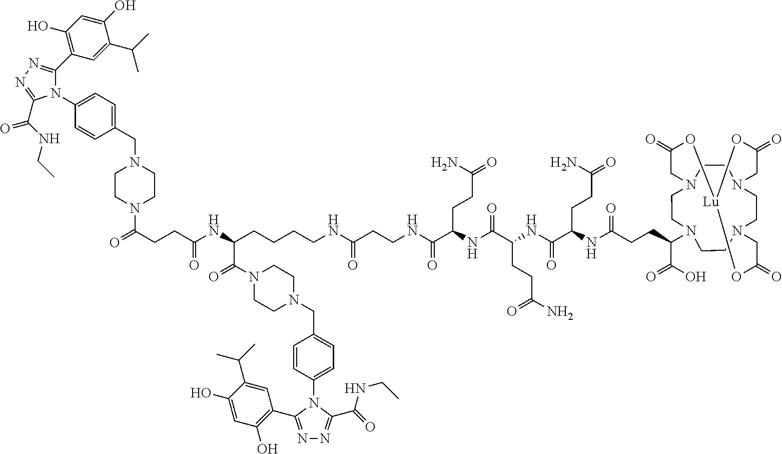

CMP49: comprising 2 TM5s; encompassed by Formula A2

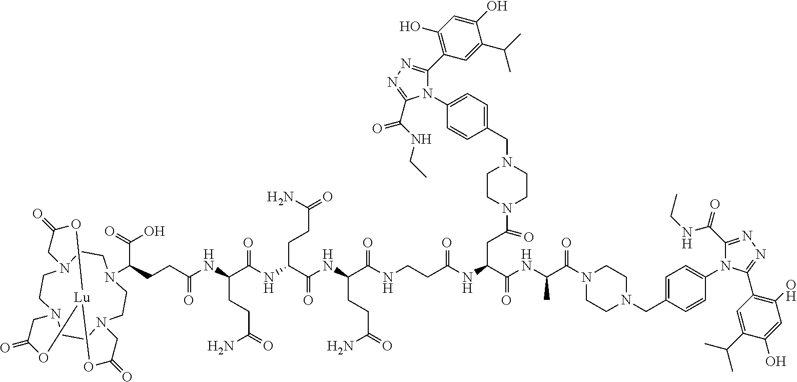

CMP50: comprising 2 TM5s; encompassed by Formula A2

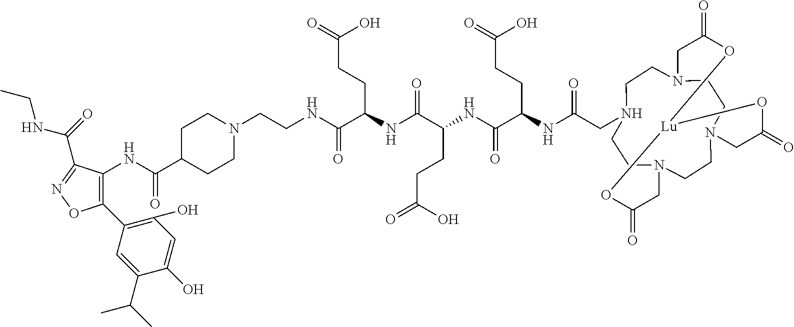

CMP23: comprising TM15; encompassed by A1

The conjugate analog structures without the radioactive agent are included in Table 2′. The conjugates may comprise any radioisotope, such as Y-90, Y-86, I-131, Re-186, Re-188, Y-90, Bi-212, At-211, Zr-89, Sr-89, Ho-166, Sm-153, Cu-67, Cu-64, Lu-177, Ac-225, Pb-203, Bi-213, Th-227, Pb-212, Ra-223, P-32, Sc-47, Br-77, Rh-105, Pd-103, Ag-111, Pr-142, Pm-149, Gd-159, Ir-194 and Pt-199.

TABLE 2′
Non-Limiting Examples of Conjugates Comprising Amino Acid
Spacers (without radioactive agent)
Structures

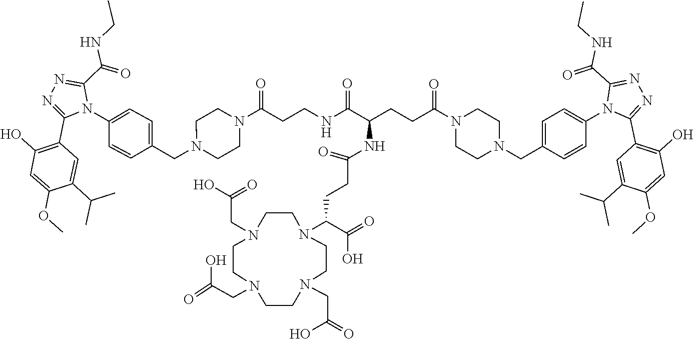 [SMILES: CCNC(=O)c1nnc(-c2cc(C(C)C)c(OC)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(OC)cc5O)cc4)CC3)NC(=O)CC[C@H](C(=O)O)N3CCN(CC(=O)O)CCN(CC(=O)O)CCN(CC(=O)O)CC3)CC2)cc1]

T1′: comprising 2 TM5s; encompassed by Formula A2

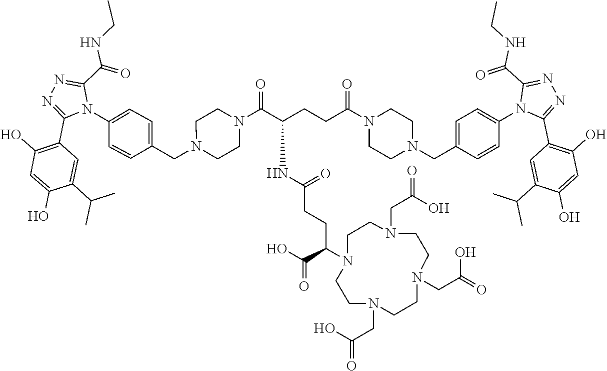 [SMILES: CCNC(=O)c1nnc(-c2cc(C(C)C)c(O)cc2O)n1-c1ccc(CN2CCN(C(=O)CC[C@H](NC(=O)CC[C@H](C(=O)O)N3CCN(CC(=O)O)CCN(CC(=O)O)CCN(CC(=O)O)CC3)C(=O)N3CCN(Cc4ccc(-n5c(C(=O)NCC)nnc5-c5cc(C(C)C)c(O)cc5O)cc4)CC3)CC2)cc1]

T2′: comprising 2 TM5s; encompassed by A2

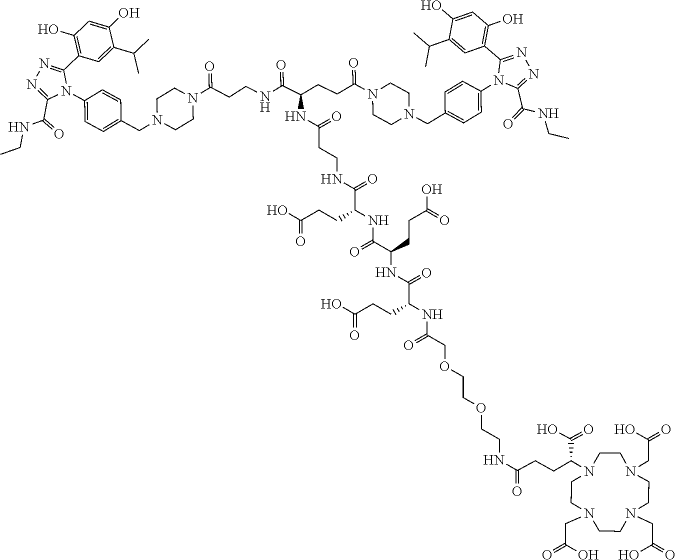 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)[C@@H](CCC(=O)O)NC(=O)[C@@H](CCC(=O)O)NC(=O)[C@@H](CCC(=O)O)NC(=O)COCCOCCNC(=O)CC[C@H](C(=O)O)N3CCN(CC(=O)O)CCN(CC(=O)O)CCN(CC(=O)O)CC3)CC2)cc1]

T4′: comprising 2 TM5s; encompassed by A2

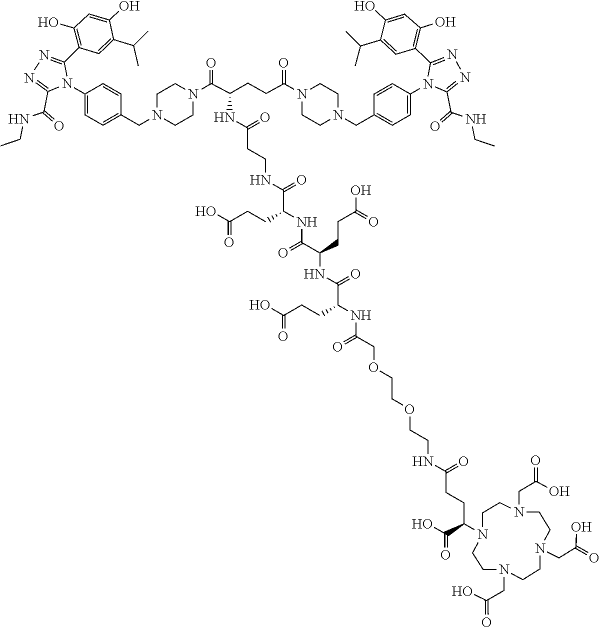 [SMILES: CCNC(=O)c1nnc(-c2cc(C(C)C)c(O)cc2O)n1-c1ccc(CN2CCN(C(=O)CC[C@H](NC(=O)CCNC(=O)[C@@H](CCC(=O)O)NC(=O)[C@@H](CCC(=O)O)NC(=O)[C@@H](CCC(=O)O)NC(=O)COCCOCCNC(=O)CC[C@H](C(=O)O)N3CCN(CC(=O)O)CCN(CC(=O)O)CCN(CC(=O)O)CC3)C(=O)N3CCN(Cc4ccc(-n5c(C(=O)NCC)nnc5-c5cc(C(C)C)c(O)cc5O)cc4)CC3)CC2)cc1]

T5′: comprising 2 TM5s; encompassed by A2

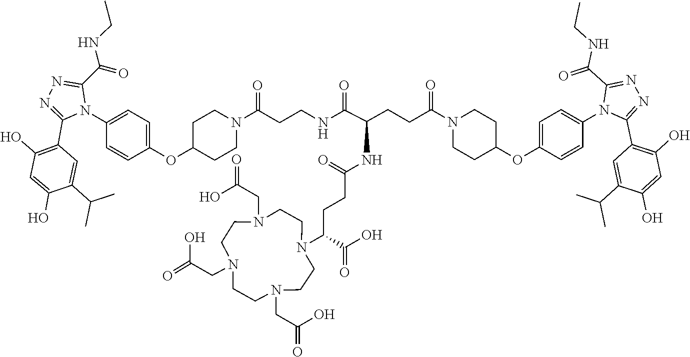 [SMILES: CCNC(=O)c1nnc(-c2cc(C(C)C)c(O)cc2O)n1-c1ccc(OC2CCN(C(=O)CCNC(=O)[C@@H](CCC(=O)N3CCC(Oc4ccc(-n5c(C(=O)NCC)nnc5-c5cc(C(C)C)c(O)cc5O)cc4)CC3)NC(=O)CC[C@H](C(=O)O)N3CCN(CC(=O)O)CCN(CC(=O)O)CCN(CC(=O)O)CC3)CC2)cc1]

T6′: comprising 2 TM10s; encompassed by A2

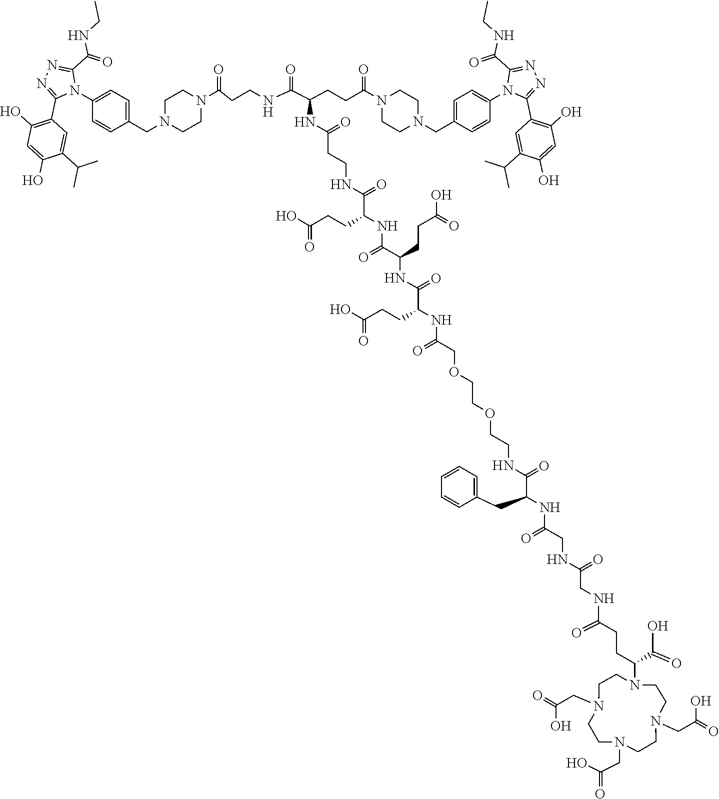 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)[C@@H](CCC(=O)O)NC(=O)[C@@H](CCC(=O)O)NC(=O)[C@@H](CCC(=O)O)NC(=O)COCCOCCNC(=O)[C@H](Cc3ccccc3)NC(=O)CNC(=O)CNC(=O)CC[C@H](C(=O)O)N3CCN(CC(=O)O)CCN(CC(=O)O)CCN(CC(=O)O)CC3)CC2)cc1]

T8′: comprising 2 TM5s; encompassed by A2

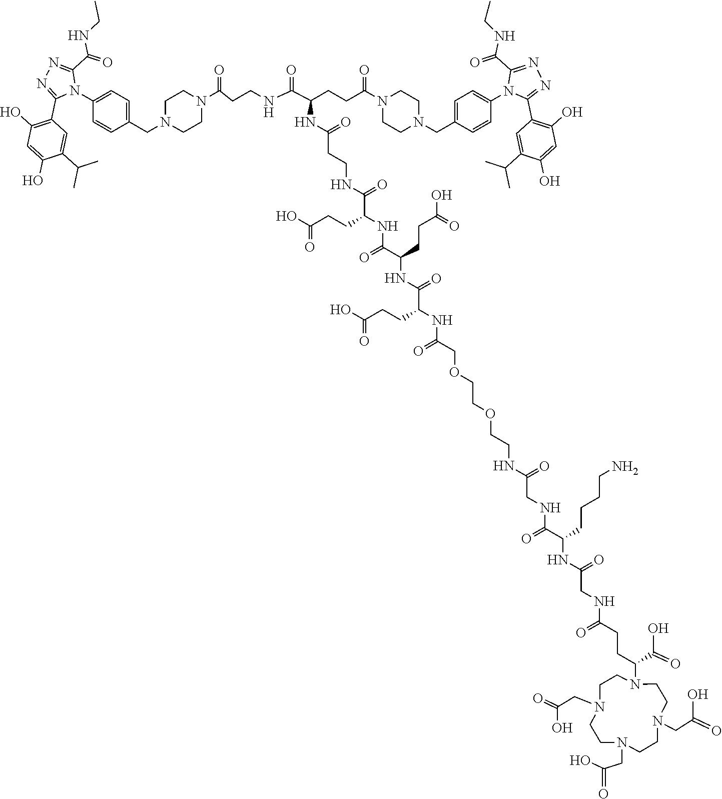 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)[C@@H](CCC(=O)O)NC(=O)[C@@H](CCC(=O)O)NC(=O)[C@@H](CCC(=O)O)NC(=O)COCCOCCNC(=O)CNC(=O)[C@H](CCCCN)NC(=O)CNC(=O)CC[C@H](C(=O)O)N3CCN(CC(=O)O)CCN(CC(=O)O)CCN(CC(=O)O)CC3)CC2)cc1]

T9′: comprising 2 TM5s; encompassed by A2

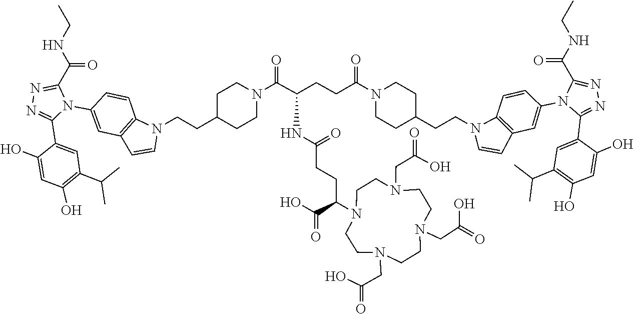 [SMILES: CCNC(=O)c1nnc(-c2cc(C(C)C)c(O)cc2O)n1-c1ccc2c(ccn2CCC2CCN(C(=O)CC[C@H](NC(=O)CC[C@H](C(=O)O)N3CCN(CC(=O)O)CCN(CC(=O)O)CCN(CC(=O)O)CC3)C(=O)N3CCC(CCn4ccc5cc(-n6c(C(=O)NCC)nnc6-c6cc(C(C)C)c(O)cc6O)ccc54)CC3)CC2)c1]

T10′: comprising 2 TM1s; encompassed by A2

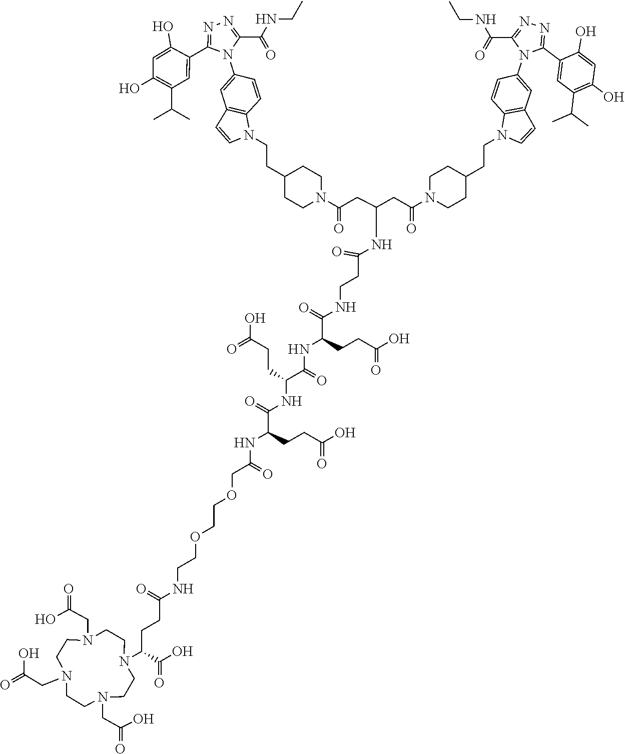 [SMILES: CCNC(=O)c1nnc(-c2cc(C(C)C)c(O)cc2O)n1-c1ccc2c(ccn2CCC2CCN(C(=O)CC(CC(=O)N3CCC(CCn4ccc5cc(-n6c(C(=O)NCC)nnc6-c6cc(C(C)C)c(O)cc6O)ccc54)CC3)NC(=O)CCNC(=O)[C@@H](CCC(=O)O)NC(=O)[C@@H](CCC(=O)O)NC(=O)[C@@H](CCC(=O)O)NC(=O)COCCOCCNC(=O)CC[C@H](C(=O)O)N3CCN(CC(=O)O)CCN(CC(=O)O)CCN(CC(=O)O)CC3)CC2)c1]

T27′: comprising 2 TM1s; encompassed by A2

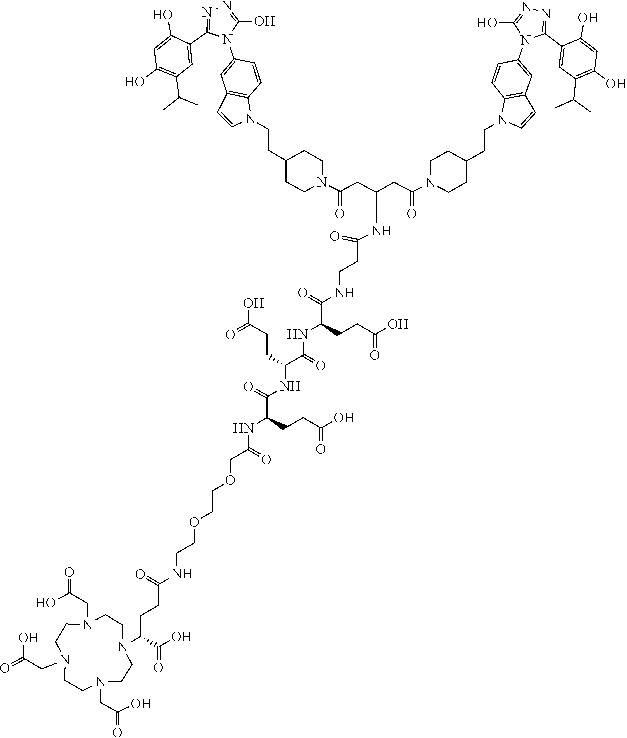 [SMILES: CC(C)c1cc(-c2nnc(O)n2-c2ccc3c(ccn3CCC3CCN(C(=O)CC(CC(=O)N4CCC(CCn5ccc6cc(-n7c(O)nnc7-c7cc(C(C)C)c(O)cc7O)ccc65)CC4)NC(=O)CCNC(=O)[C@@H](CCC(=O)O)NC(=O)[C@@H](CCC(=O)O)NC(=O)[C@@H](CCC(=O)O)NC(=O)COCCOCCNC(=O)CC[C@H](C(=O)O)N4CCN(CC(=O)O)CCN(CC(=O)O)CCN(CC(=O)O)CC4)CC3)c2)c(O)cc1O]

T28′: comprising 2 TM1s; encompassed by A2

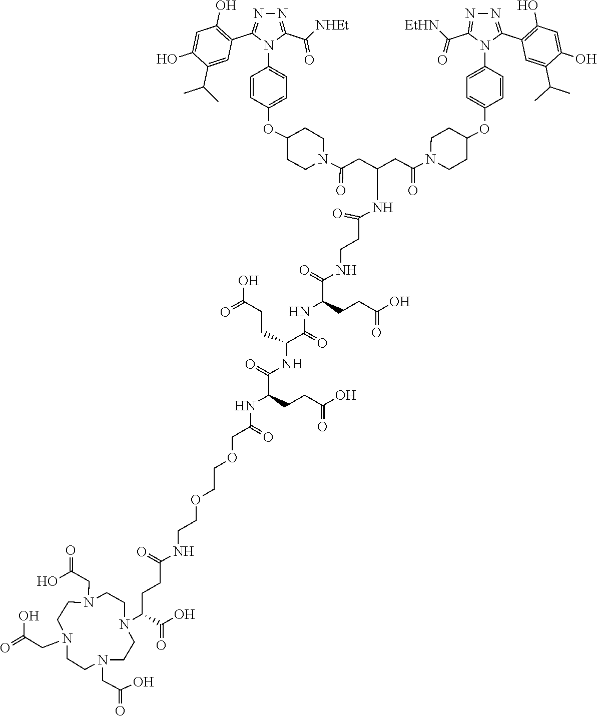

T29′: comprising 2 TM10s; encompassed by A2

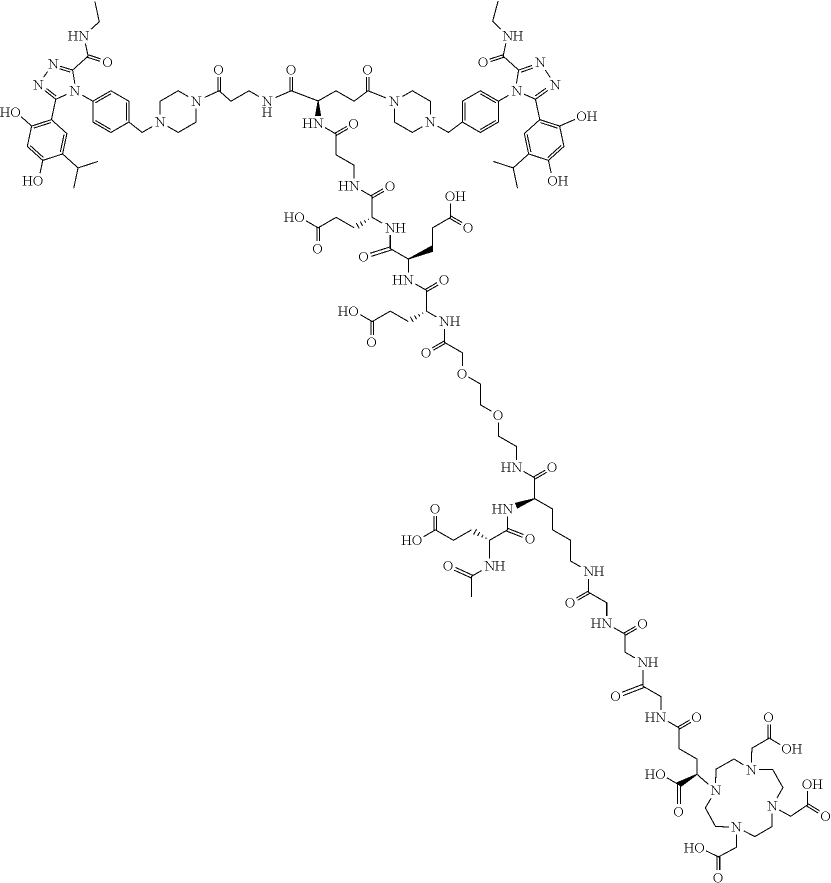 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)[C@@H](CCC(=O)O)NC(=O)[C@@H](CCC(=O)O)NC(=O)[C@@H](CCC(=O)O)NC(=O)COCCOCCNC(=O)[C@@H](CCCCNC(=O)CNC(=O)CNC(=O)CNC(=O)CC[C@H](C(=O)O)N3CCN(CC(=O)O)CCN(CC(=O)O)CCN(CC(=O)O)CC3)NC(=O)[C@@H](CCC(=O)O)NC(C)=O)CC2)cc1]

T39′: comprising 2 TM5s; encompassed by A2

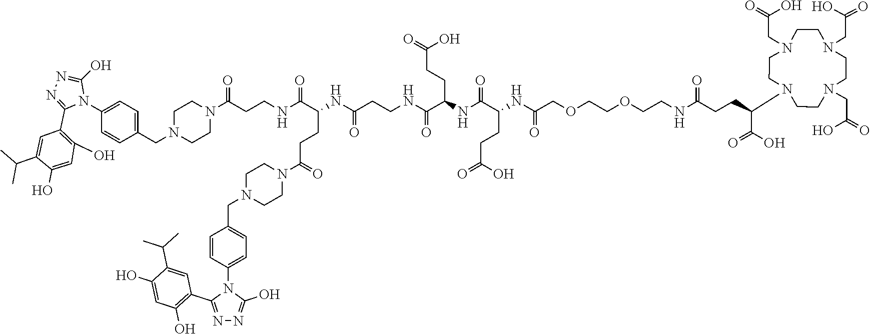 [SMILES: CC(C)c1cc(-c2nnc(O)n2-c2ccc(CN3CCN(C(=O)CCNC(=O)[C@@H](CCC(=O)N4CCN(Cc5ccc(-n6c(O)nnc6-c6cc(C(C)C)c(O)cc6O)cc5)CC4)NC(=O)CCNC(=O)[C@@H](CCC(=O)O)NC(=O)[C@@H](CCC(=O)O)NC(=O)COCCOCCNC(=O)CC[C@H](C(=O)O)N4CCN(CC(=O)O)CCN(CC(=O)O)CCN(CC(=O)O)CC4)CC3)cc2)c(O)cc1O]

CMP13′: comprising 2 TM2s; encompassed by A2

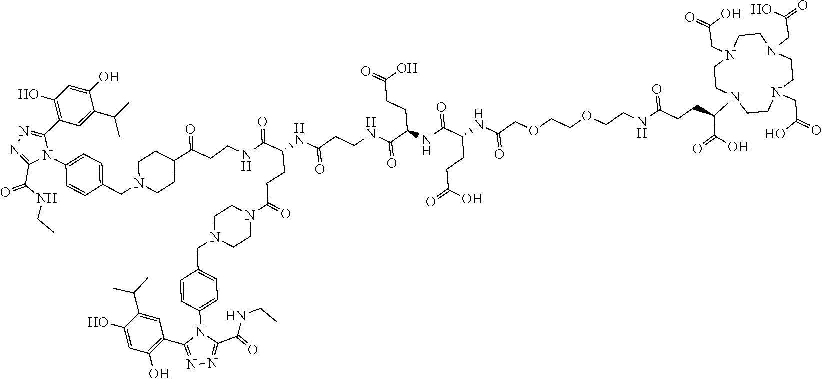 [SMILES: CCNC(=O)c1nnc(-c2cc(C(C)C)c(O)cc2O)n1-c1ccc(CN2CCC(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)[C@@H](CCC(=O)O)NC(=O)[C@@H](CCC(=O)O)NC(=O)COCCOCCNC(=O)CC[C@H](C(=O)O)N3CCN(CC(=O)O)CCN(CC(=O)O)CCN(CC(=O)O)CC3)CC2)cc1]

CMP14′: comprising 2 TM5s; encompassed by A2

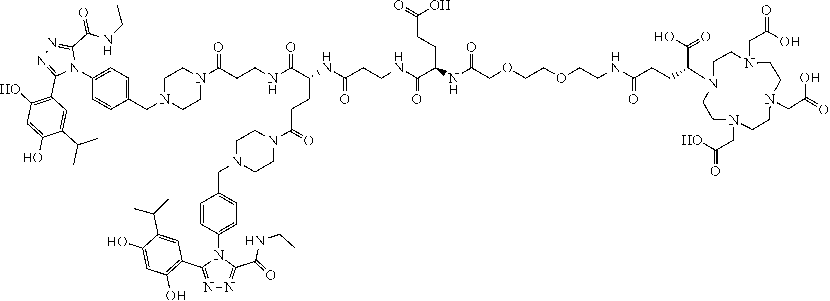 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)[C@@H](CCC(=O)O)NC(=O)COCCOCCNC(=O)CC[C@H](C(=O)O)N3CCN(CC(=O)O)CCN(CC(=O)O)CCN(CC(=O)O)CC3)CC2)cc1]

CMP15′: comprising 2 TM5s; encompassed by A2

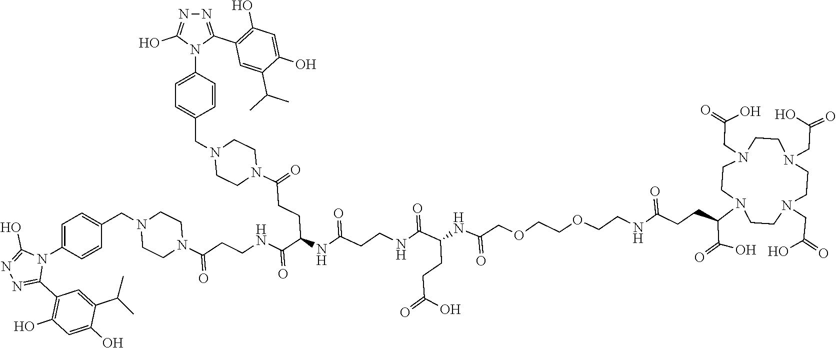 [SMILES: CC(C)c1cc(-c2nnc(O)n2-c2ccc(CN3CCN(C(=O)CCNC(=O)[C@@H](CCC(=O)N4CCN(Cc5ccc(-n6c(O)nnc6-c6cc(C(C)C)c(O)cc6O)cc5)CC4)NC(=O)CCNC(=O)[C@@H](CCC(=O)O)NC(=O)COCCOCCNC(=O)CC[C@H](C(=O)O)N4CCN(CC(=O)O)CCN(CC(=O)O)CCN(CC(=O)O)CC4)CC3)cc2)c(O)cc1O]

CMP16′: comprising 2 TM2s; encompassed by A2

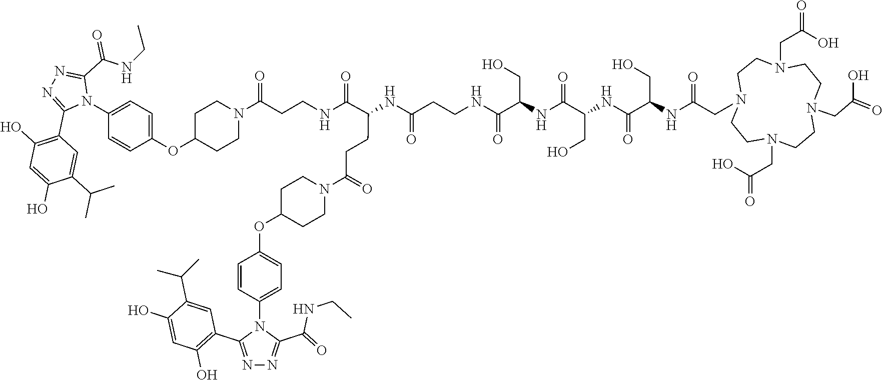 [SMILES: CCNC(=O)c1nnc(-c2cc(C(C)C)c(O)cc2O)n1-c1ccc(OC2CCN(C(=O)CCNC(=O)[C@@H](CCC(=O)N3CCC(Oc4ccc(-n5c(C(=O)NCC)nnc5-c5cc(C(C)C)c(O)cc5O)cc4)CC3)NC(=O)CCNC(=O)[C@@H](CO)NC(=O)[C@@H](CO)NC(=O)[C@@H](CO)NC(=O)CN3CCN(CC(=O)O)CCN(CC(=O)O)CCN(CC(=O)O)CC3)CC2)cc1]

CMP37′: comprising 2 TM10s; encompassed by Formula A1

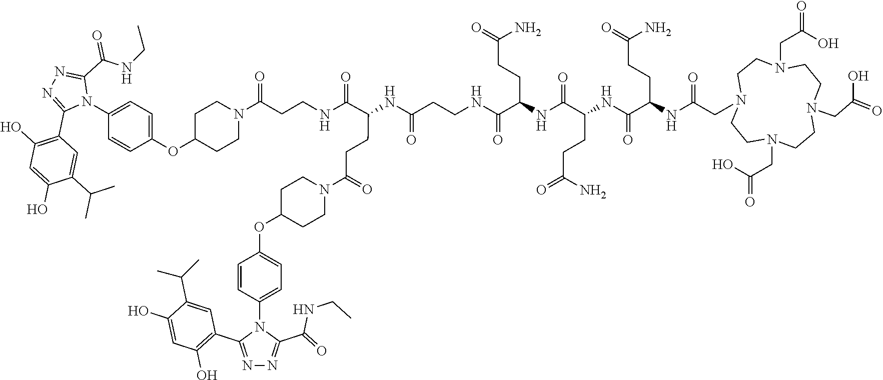 [SMILES: CCNC(=O)c1nnc(-c2cc(C(C)C)c(O)cc2O)n1-c1ccc(OC2CCN(C(=O)CCNC(=O)[C@@H](CCC(=O)N3CCC(Oc4ccc(-n5c(C(=O)NCC)nnc5-c5cc(C(C)C)c(O)cc5O)cc4)CC3)NC(=O)CCNC(=O)[C@@H](CCC(N)=O)NC(=O)[C@@H](CCC(N)=O)NC(=O)[C@@H](CCC(N)=O)NC(=O)CN3CCN(CC(=O)O)CCN(CC(=O)O)CCN(CC(=O)O)CC3)CC2)cc1]

CMP38′: comprising 2 TM10s; encompassed by Formula A1

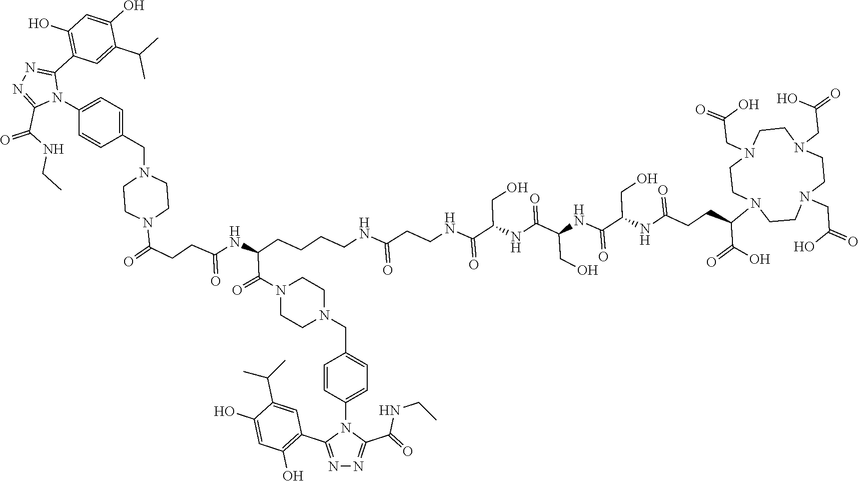 [SMILES: CCNC(=O)c1nnc(-c2cc(C(C)C)c(O)cc2O)n1-c1ccc(CN2CCN(C(=O)CCC(=O)N[C@@H](CCCCNC(=O)CCNC(=O)[C@H](CO)NC(=O)[C@H](CO)NC(=O)[C@H](CO)NC(=O)CC[C@H](C(=O)O)N3CCN(CC(=O)O)CCN(CC(=O)O)CCN(CC(=O)O)CC3)C(=O)N3CCN(Cc4ccc(-n5c(C(=O)NCC)nnc5-c5cc(C(C)C)c(O)cc5O)cc4)CC3)CC2)cc1]

CMP43′: comprising 2 TM5s; encompassed by Formula A2

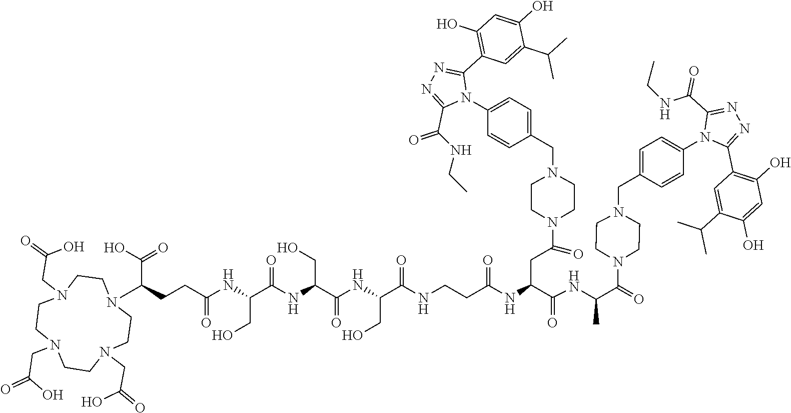 [SMILES: CCNC(=O)c1nnc(-c2cc(C(C)C)c(O)cc2O)n1-c1ccc(CN2CCN(C(=O)C[C@H](NC(=O)CCNC(=O)[C@H](CO)NC(=O)[C@H](CO)NC(=O)[C@H](CO)NC(=O)CC[C@H](C(=O)O)N3CCN(CC(=O)O)CCN(CC(=O)O)CCN(CC(=O)O)CC3)C(=O)N[C@H](C)C(=O)N3CCN(Cc4ccc(-n5c(C(=O)NCC)nnc5-c5cc(C(C)C)c(O)cc5O)cc4)CC3)CC2)cc1]

CMP44′: comprising 2 TM5s; encompassed by Formula A2

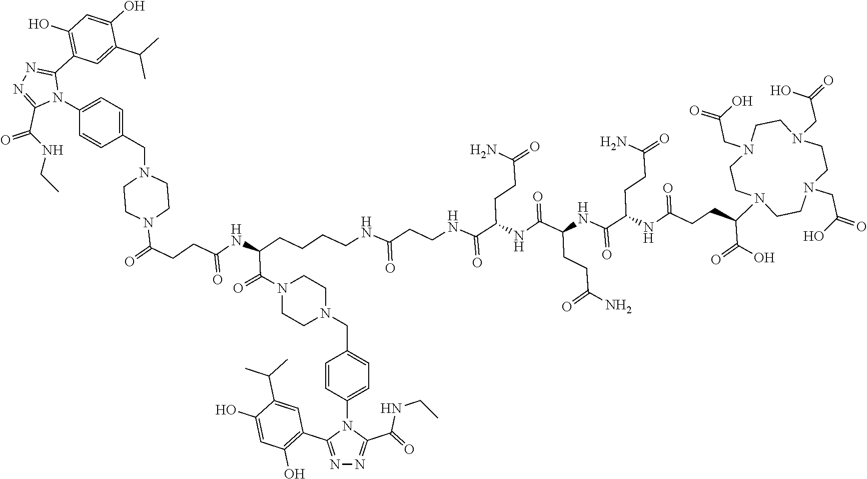 [SMILES: CCNC(=O)c1nnc(-c2cc(C(C)C)c(O)cc2O)n1-c1ccc(CN2CCN(C(=O)CCC(=O)N[C@@H](CCCCNC(=O)CCNC(=O)[C@H](CCC(N)=O)NC(=O)[C@H](CCC(N)=O)NC(=O)[C@H](CCC(N)=O)NC(=O)CC[C@H](C(=O)O)N3CCN(CC(=O)O)CCN(CC(=O)O)CCN(CC(=O)O)CC3)C(=O)N3CCN(Cc4ccc(-n5c(C(=O)NCC)nnc5-c5cc(C(C)C)c(O)cc5O)cc4)CC3)CC2)cc1]

CMP45′: comprising 2 TM5s; encompassed by Formula A2

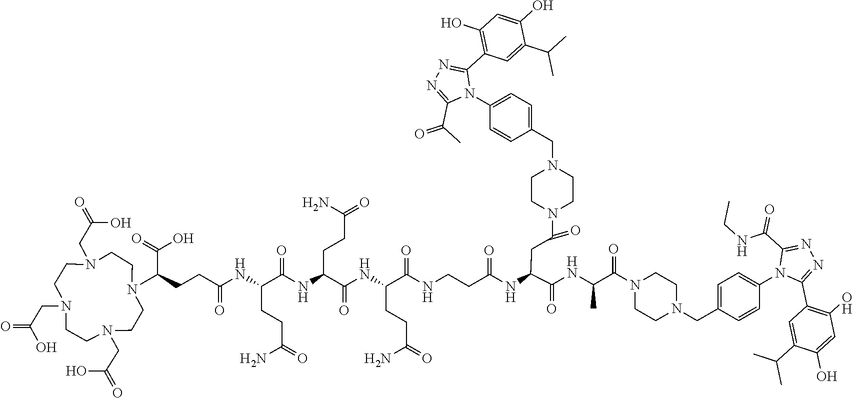 [SMILES: CCNC(=O)c1nnc(-c2cc(C(C)C)c(O)cc2O)n1-c1ccc(CN2CCN(C(=O)[C@@H](C)NC(=O)[C@H](CC(=O)N3CCN(Cc4ccc(-n5c(C(C)=O)nnc5-c5cc(C(C)C)c(O)cc5O)cc4)CC3)NC(=O)CCNC(=O)[C@H](CCC(N)=O)NC(=O)[C@H](CCC(N)=O)NC(=O)[C@H](CCC(N)=O)NC(=O)CC[C@H](C(=O)O)N3CCN(CC(=O)O)CCN(CC(=O)O)CCN(CC(=O)O)CC3)CC2)cc1]

CMP46′: comprising 2 TM5s; encompassed by Formula A2

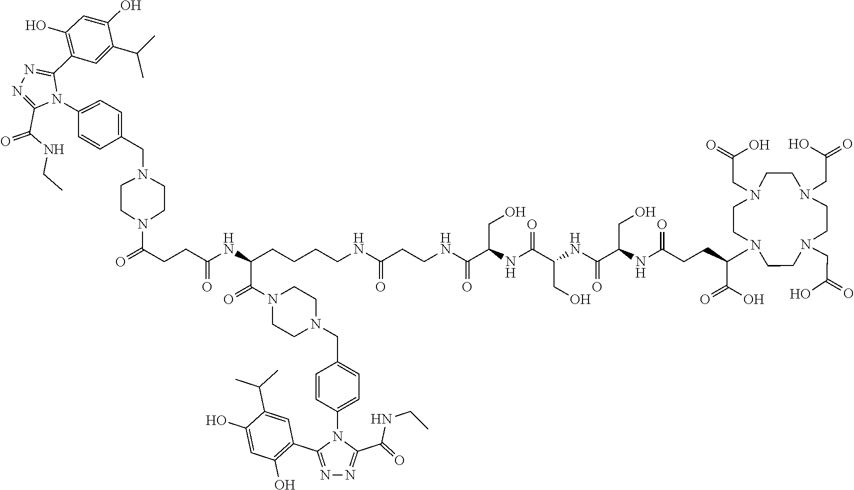 [SMILES: CCNC(=O)c1nnc(-c2cc(C(C)C)c(O)cc2O)n1-c1ccc(CN2CCN(C(=O)CCC(=O)N[C@@H](CCCCNC(=O)CCNC(=O)[C@@H](CO)NC(=O)[C@@H](CO)NC(=O)[C@@H](CO)NC(=O)CC[C@H](C(=O)O)N3CCN(CC(=O)O)CCN(CC(=O)O)CCN(CC(=O)O)CC3)C(=O)N3CCN(Cc4ccc(-n5c(C(=O)NCC)nnc5-c5cc(C(C)C)c(O)cc5O)cc4)CC3)CC2)cc1]

CMP47′: comprising 2 TM5s; encompassed by Formula A2

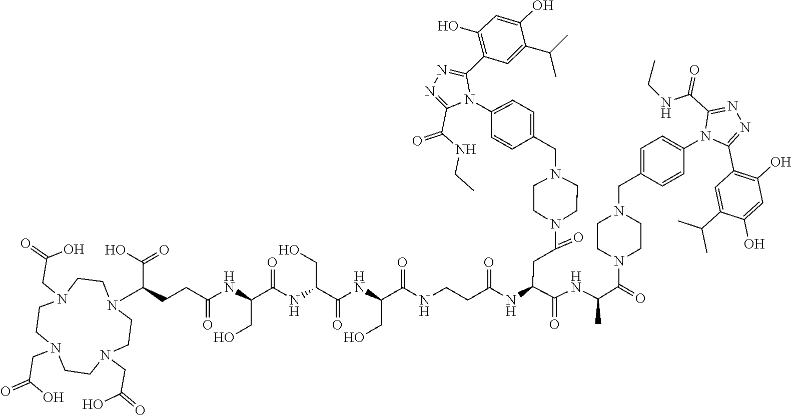 [SMILES: CCNC(=O)c1nnc(-c2cc(C(C)C)c(O)cc2O)n1-c1ccc(CN2CCN(C(=O)C[C@H](NC(=O)CCNC(=O)[C@@H](CO)NC(=O)[C@@H](CO)NC(=O)[C@@H](CO)NC(=O)CC[C@H](C(=O)O)N3CCN(CC(=O)O)CCN(CC(=O)O)CCN(CC(=O)O)CC3)C(=O)N[C@H](C)C(=O)N3CCN(Cc4ccc(-n5c(C(=O)NCC)nnc5-c5cc(C(C)C)c(O)cc5O)cc4)CC3)CC2)cc1]

CMP48′: comprising 2 TM5s; encompassed by Formula A2

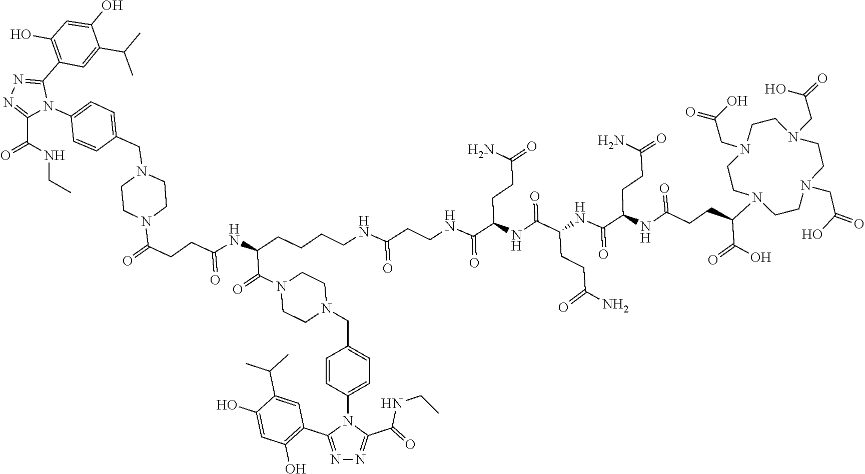 [SMILES: CCNC(=O)c1nnc(-c2cc(C(C)C)c(O)cc2O)n1-c1ccc(CN2CCN(C(=O)CCC(=O)N[C@@H](CCCCNC(=O)CCNC(=O)[C@@H](CCC(N)=O)NC(=O)[C@@H](CCC(N)=O)NC(=O)[C@@H](CCC(N)=O)NC(=O)CC[C@H](C(=O)O)N3CCN(CC(=O)O)CCN(CC(=O)O)CCN(CC(=O)O)CC3)C(=O)N3CCN(Cc4ccc(-n5c(C(=O)NCC)nnc5-c5cc(C(C)C)c(O)cc5O)cc4)CC3)CC2)cc1]

CMP49′: comprising 2 TM5s; encompassed by Formula A2

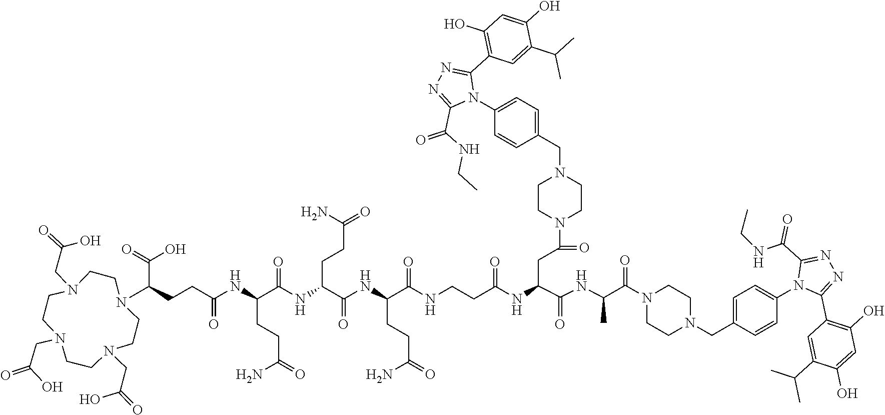 [SMILES: CCNC(=O)c1nnc(-c2cc(C(C)C)c(O)cc2O)n1-c1ccc(CN2CCN(C(=O)C[C@H](NC(=O)CCNC(=O)[C@@H](CCC(N)=O)NC(=O)[C@@H](CCC(N)=O)NC(=O)[C@@H](CCC(N)=O)NC(=O)CC[C@H](C(=O)O)N3CCN(CC(=O)O)CCN(CC(=O)O)CCN(CC(=O)O)CC3)C(=O)N[C@H](C)C(=O)N3CCN(Cc4ccc(-n5c(C(=O)NCC)nnc5-c5cc(C(C)C)c(O)cc5O)cc4)CC3)CC2)cc1]

CMP50′: comprising 2 TM5s; encompassed by Formula A2

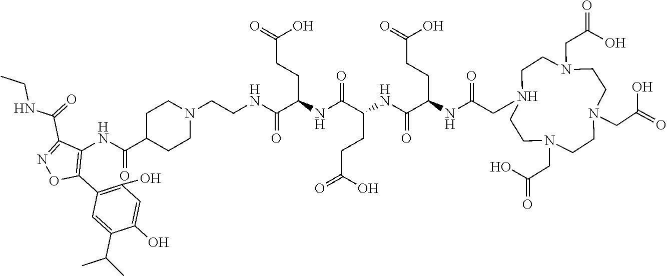 [SMILES: CCNC(=O)c1noc(-c2cc(C(C)C)c(O)cc2O)c1NC(=O)C1CCN(CCNC(=O)[C@@H](CCC(=O)O)NC(=O)[C@@H](CCC(=O)O)NC(=O)[C@@H](CCC(=O)O)NC(=O)CN2CCN(CC(=O)O)CCN(CC(=O)O)CCN(CC(=O)O)CC2)CC1]

CMP23′: comprising TM15; encompassed by A1

In some embodiments, the conjugate comprises at least one amino acid spacer and comprises at least one TM1 as a ligand. In some embodiments, the conjugate comprises at least one amino acid spacer and comprises 1 TM1 as a ligand. In some embodiments, the conjugate comprises at least one amino acid spacer and comprises two TM1s as ligands. Non-limiting examples include T10, T27, and T28.

In some embodiments, the conjugate comprises at least one amino acid spacer and comprises at least one TM2 as a ligand. In some embodiments, the conjugate comprises at least one amino acid spacer and comprises 1 TM2 as a ligand. In some embodiments, the conjugate comprises at least one amino acid spacer and comprises two TM2s as ligands. Non-limiting examples include CMP13 and CMP16.

In some embodiments, the conjugate comprises at least one amino acid spacer and comprises at least one TM5 as a ligand. In some embodiments, the conjugate comprises at least one amino acid spacer and comprises 1 TM5 as a ligand. In some embodiments, the conjugate comprises at least one amino acid spacer and comprises two TM5s as ligands. Non-limiting examples include T1, T2, T4, T5, T8, T9, T39, CMP14, CMP15, CMP43, CMP44, CMP45, CMP46, CMP47, CMP48, CMP49 and CMP50.

In some embodiments, the conjugate comprises at least one amino acid spacer and comprises at least one TM10 as a ligand. In some embodiments, the conjugate comprises at least one amino acid spacer and comprises 1 TM10 as a ligand. In some embodiments, the conjugate comprises at least one amino acid spacer and comprises two TM10s as ligands. Non-limiting examples include T6, T29, CMP37, and CMP38.

In some embodiments, the conjugate comprises at least one amino acid spacer and comprises at least one TM15 as a ligand. In some embodiments, the conjugate comprises at least one amino acid spacer and comprises 1 TM15 as a ligand. In some embodiments, the conjugate comprises at least one amino acid spacer and comprises two TM10s as ligands. Non-limiting examples include CMP23.

### PEG Spacers

In some embodiments, the conjugate comprises an HSP90 targeting moiety attached to a chelating agent for a radioactive agent with a linker. The HSP90 targeting moiety may be a ganetespib analog or derivative (such as TM1, TM2, TM3, TM4, TM5, TM8, TM9, TM10, TM11, TM12, TM13, or TM14), an onalespib analog or derivative (such as TM6 or TM7), or TM15. The linker may comprise a spacer comprising polyethylene glycol (PEG). The PEG spacers can be constructed from (PEG)ₙ, wherein n is an integer between 1 to 20. In some embodiments, the PEG spacer is (PEG)4. In some embodiments, the PEG spacer is (PEG)12. HSP90 binding affinity is maintained regardless of what charges are present on the spacer.

In some embodiments, the conjugate has a structure of Formula B:

wherein TM(s) refers to at least one targeting moiety that binds to HSP90. In some embodiments, the conjugate has 2 targeting moieties. In some embodiments, the conjugate has 3 targeting moieties. In some embodiments, the conjugate has 4 targeting moieties.

In some embodiments, the conjugate has a structure of Formula B10:

TM is selected from TM1, TM2, TM3, TM4, TM5, TM8, TM9, TM10, TM11, TM12, TM13, TM6, TM7, or TM14.

In some embodiments, the conjugate has a structure of Formula B1:

TM is selected from TM1, TM2, TM3, TM4, TM5, TM8, TM9, TM10, TM11, TM12, TM13, TM6, TM7, or TM14. Non-limiting examples of the conjugates encompassed by Formula B1 include CMP1, CMP29, CMP30, CMP31, CMP32, CMP33, CMP34, CMP35, CMP36, CMP39, CMP40, CMP41, and CMP42.

In some embodiments, the conjugate has a structure of Formula B20:

TM is selected from TM1, TM2, TM3, TM4, TM5, TM8, TM9, TM10, TM11, TM12, TM13, TM6, TM7, or TM14.

In some embodiments, the conjugate has a structure of Formula B2:

TM is selected from TM1, TM2, TM3, TM4, TM5, TM8, TM9, TM10, TM11, TM12, TM13, TM6, TM7, or TM14. Non-limiting examples of the conjugates encompassed by Formula B2 include CMP2.

Non-limiting examples of conjugates of the current disclosure that are encompassed by Formula B include CMP1, CMP2, CMP29, CMP30, CMP31, CMP32, CMP33, CMP34, CMP35, CMP36, CMP39, CMP40, CMP41, CMP42, CMP52:

TABLE 3
Non-Limiting Examples of Conjugates Comprising 1 TM and PEG
Spacers
Structures

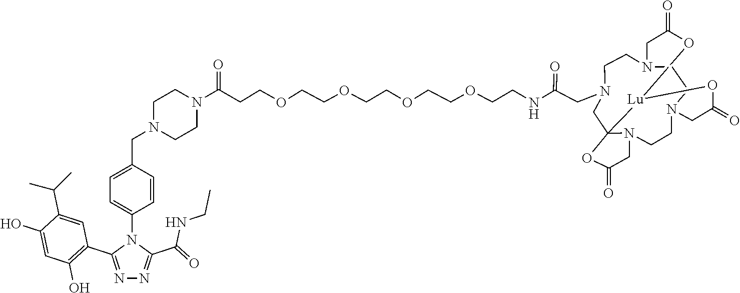

CMP1: comprising TM5 ligand and (PEG)4 spacer; encompassed by Formula B1

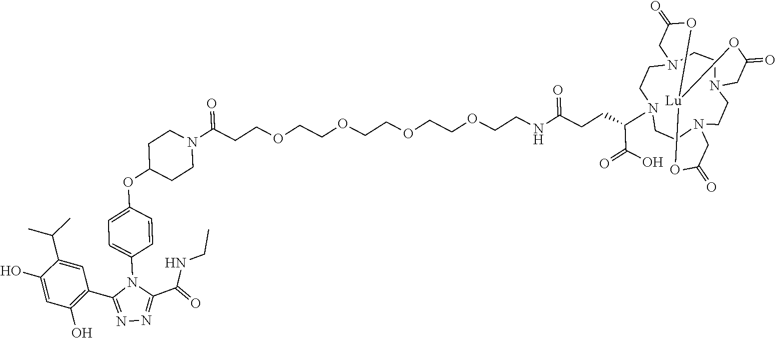

CMP2: comprising TM10 ligand and (PEG)4 spacer; encompassed by Formula B2

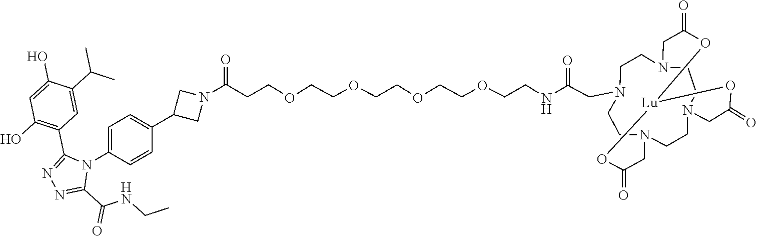

CMP29: comprising TM11 ligand and (PEG)4 spacer; encompassed by Formula B1

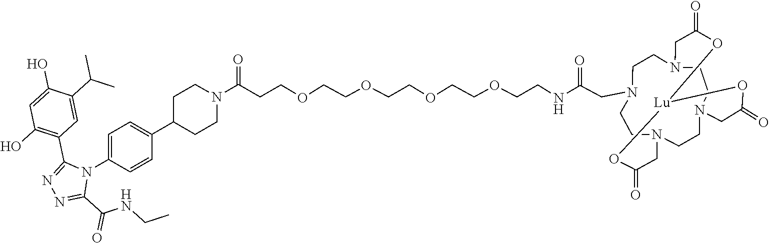

CMP30: comprising TM12 ligand and (PEG)4 spacer; encompassed by Formula B1

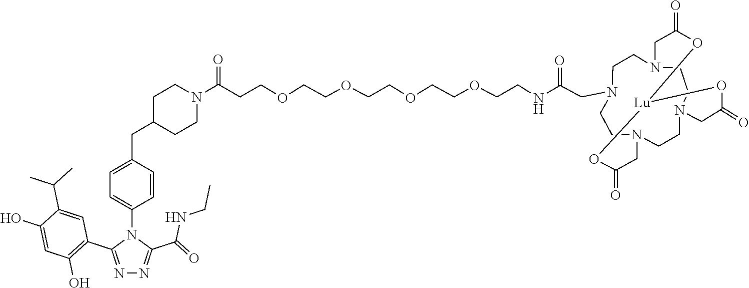

CMP31: comprising TM9 ligand and (PEG)4 spacer; encompassed by Formula B1

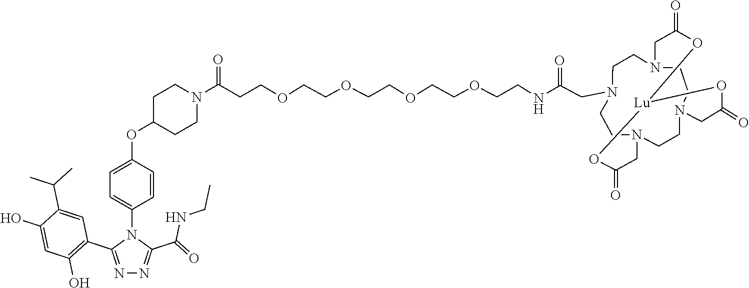

CMP39: comprising TM10 ligand and (PEG)4 spacer; encompassed by Formula B1

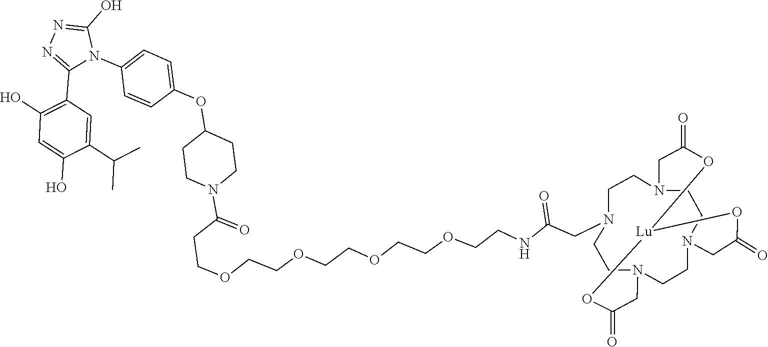

CMP40: comprising TM13 ligand and (PEG)4 spacer; encompassed by Formula B1

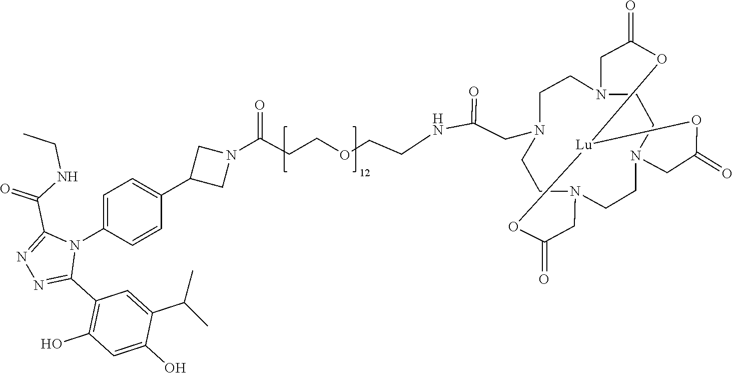

CMP32: comprising TM11 ligand and (PEG)12 spacer; encompassed by Formula B1

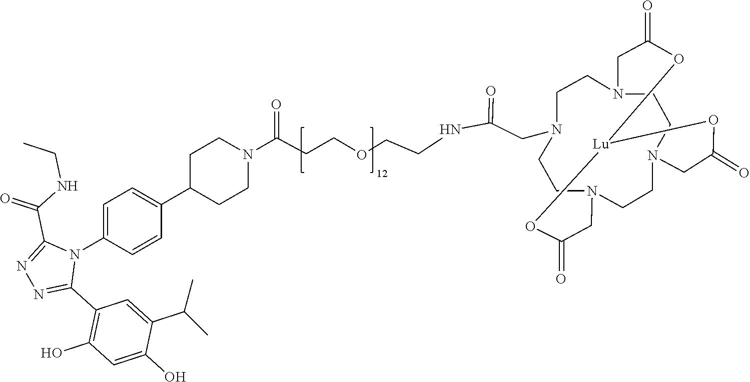

CMP33: comprising TM9 ligand and (PEG)12 spacer; encompassed by Formula B1

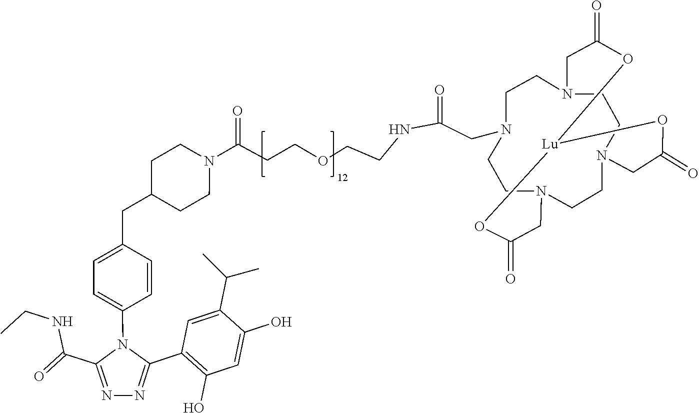

CMP34: comprising TM9 ligand and (PEG)12 spacer; encompassed by Formula B1

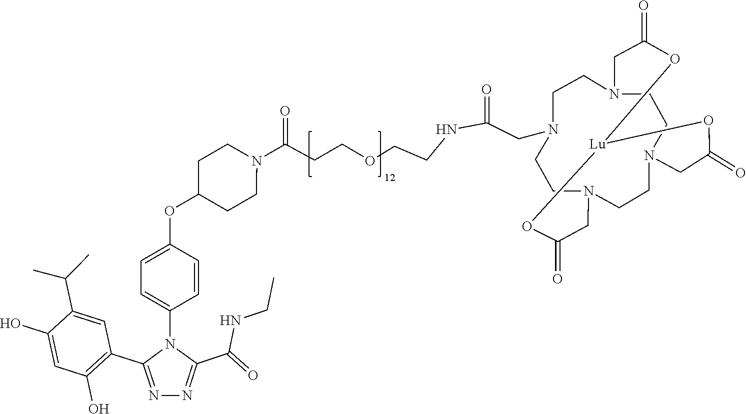

CMP41: comprising TM10 ligand and (PEG)12 spacer; encompassed by Formula B1

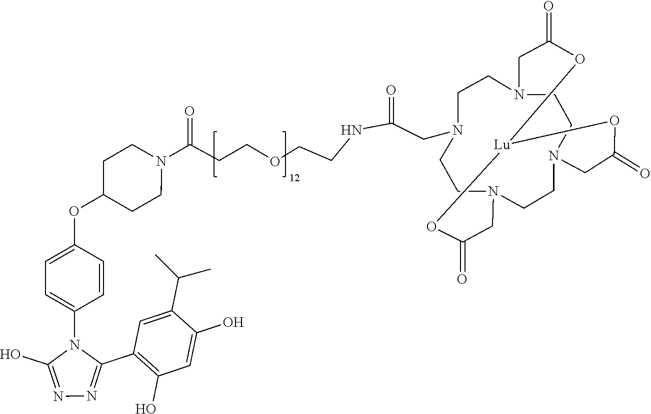

CMP42: comprising TM13 ligand and (PEG)12 spacer; encompassed by Formula B1

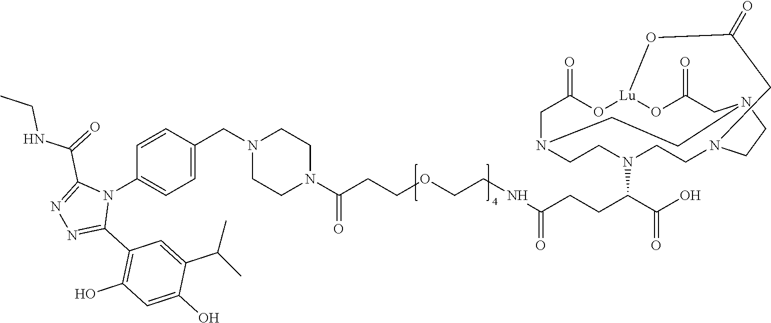

CMP52: comprising TM5 ligand and (PEG)4 spacer, encompassed by Formula B2

The conjugate analog structures without the radioactive agent are included in Table 3′. The conjugates may comprise any radioisotope, such as Y-90, Y-86, I-131, Re-186, Re-188, Y-90, Bi-212, At-211, Zr-89, Sr-89, Ho-166, Sm-153, Cu-67, Cu-64, Lu-177, Ac-225, Pb-203, Bi-213, Th-227, Pb-212, Ra-223, P-32, Sc-47, Br-77, Rh-105, Pd-103, Ag-111, Pr-142, Pm-149, Gd-159, Ir-194 and Pt-199.

TABLE 3′
Non-Limiting Examples of Conjugates Comprising 1 TM and PEG
Spacers (without radioactive agent)
Structures

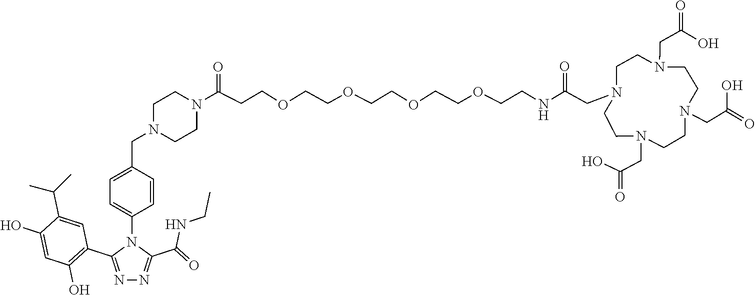 [SMILES: CCNC(=O)c1nnc(-c2cc(C(C)C)c(O)cc2O)n1-c1ccc(CN2CCN(C(=O)CCOCCOCCOCCOCCNC(=O)CN3CCN(CC(=O)O)CCN(CC(=O)O)CCN(CC(=O)O)CC3)CC2)cc1]

CMP1′: comprising TM5 ligand and (PEG)4 spacer; encompassed by Formula B1

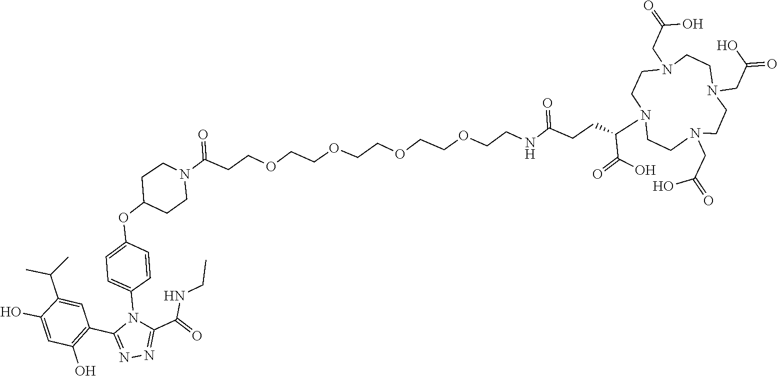 [SMILES: CCNC(=O)c1nnc(-c2cc(C(C)C)c(O)cc2O)n1-c1ccc(OC2CCN(C(=O)CCOCCOCCOCCOCCNC(=O)CC[C@@H](C(=O)O)N3CCN(CC(=O)O)CCN(CC(=O)O)CCN(CC(=O)O)CC3)CC2)cc1]

CMP2′: comprising TM10 ligand and (PEG)4 spacer; encompassed by Formula B2

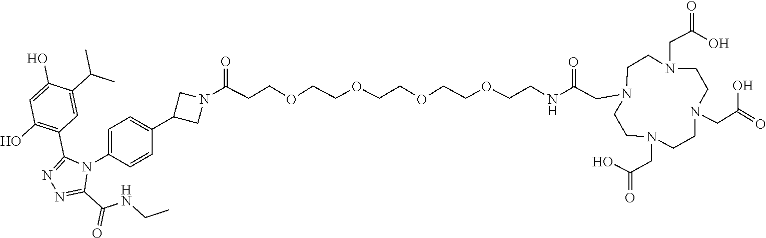 [SMILES: CCNC(=O)c1nnc(-c2cc(C(C)C)c(O)cc2O)n1-c1ccc(C2CN(C(=O)CCOCCOCCOCCOCCNC(=O)CN3CCN(CC(=O)O)CCN(CC(=O)O)CCN(CC(=O)O)CC3)C2)cc1]

CMP29′: comprising TM11 ligand and (PEG)4 spacer; encompassed by Formula B1

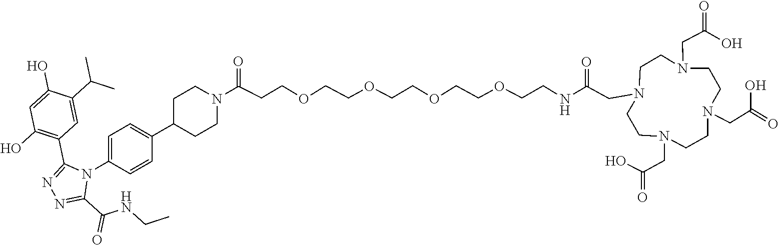 [SMILES: CCNC(=O)c1nnc(-c2cc(C(C)C)c(O)cc2O)n1-c1ccc(C2CCN(C(=O)CCOCCOCCOCCOCCNC(=O)CN3CCN(CC(=O)O)CCN(CC(=O)O)CCN(CC(=O)O)CC3)CC2)cc1]

CMP30′: comprising TM12 ligand and (PEG)4 spacer; encompassed by Formula B1

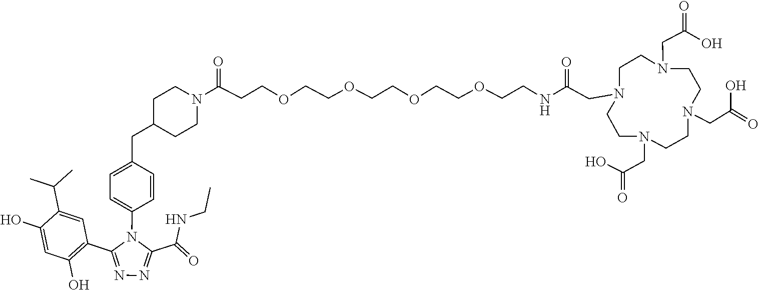 [SMILES: CCNC(=O)c1nnc(-c2cc(C(C)C)c(O)cc2O)n1-c1ccc(CC2CCN(C(=O)CCOCCOCCOCCOCCNC(=O)CN3CCN(CC(=O)O)CCN(CC(=O)O)CCN(CC(=O)O)CC3)CC2)cc1]

CMP31′: comprising TM9 ligand and (PEG)4 spacer; encompassed by Formula B1

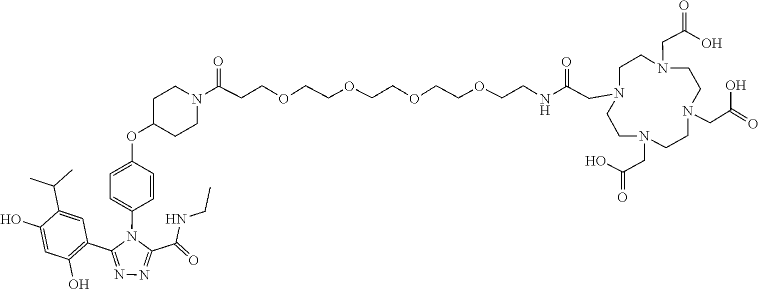 [SMILES: CCNC(=O)c1nnc(-c2cc(C(C)C)c(O)cc2O)n1-c1ccc(OC2CCN(C(=O)CCOCCOCCOCCOCCNC(=O)CN3CCN(CC(=O)O)CCN(CC(=O)O)CCN(CC(=O)O)CC3)CC2)cc1]

CMP39′: comprising TM10 ligand and (PEG)4 spacer; encompassed by Formula B1

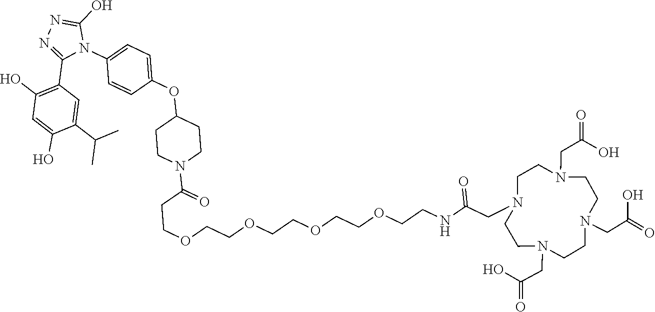 [SMILES: CC(C)c1cc(-c2nnc(O)n2-c2ccc(OC3CCN(C(=O)CCOCCOCCOCCOCCNC(=O)CN4CCN(CC(=O)O)CCN(CC(=O)O)CCN(CC(=O)O)CC4)CC3)cc2)c(O)cc1O]

CMP40′: comprising TM13 ligand and (PEG)4 spacer; encompassed by Formula B1

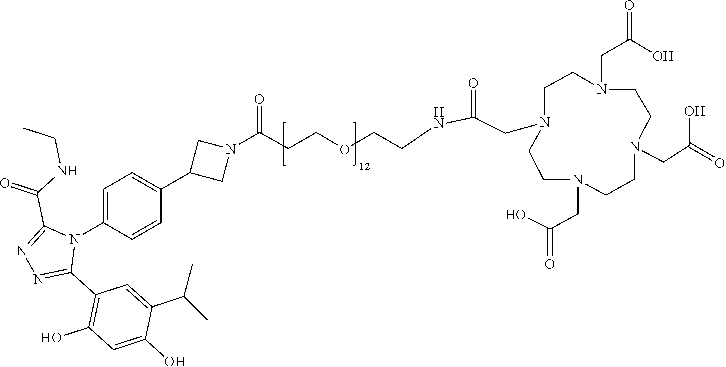

CMP32′: comprising TM11 ligand and (PEG)12 spacer; encompassed by Formula B1

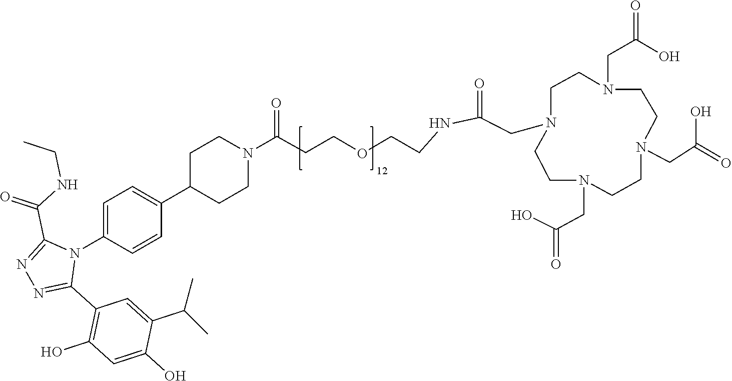

CMP33′: comprising TM9 ligand and (PEG)12 spacer; encompassed by Formula B1

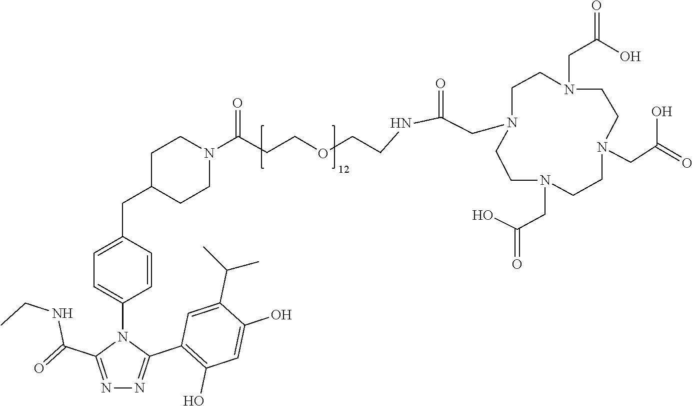

CMP34′: comprising TM9 ligand and (PEG)12 spacer; encompassed by Formula B1

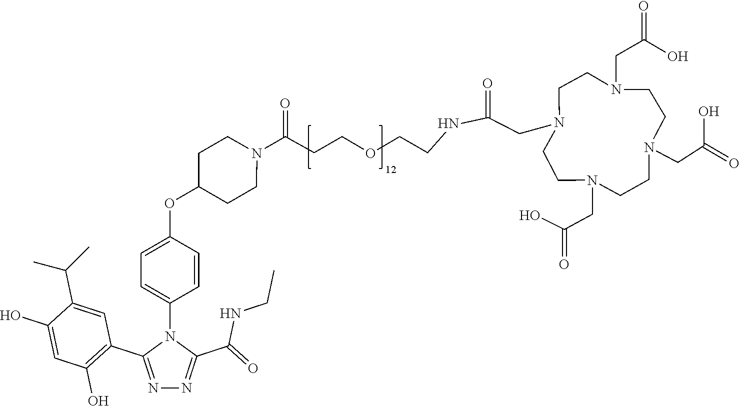

CMP41′: comprising TM10 ligand and (PEG)12 spacer; encompassed by Formula B1

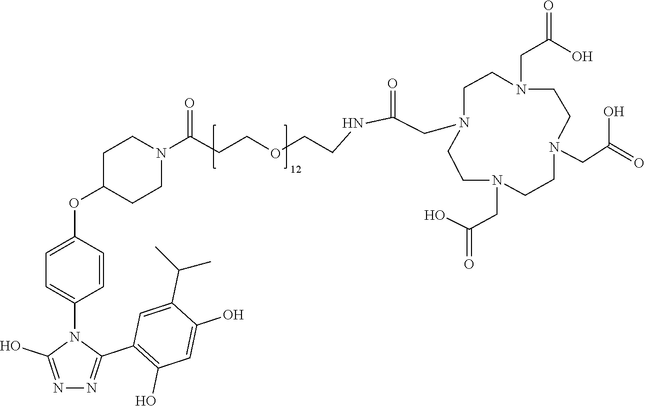

CMP42′: comprising TM13 ligand and (PEG)12 spacer; encompassed by Formula B1

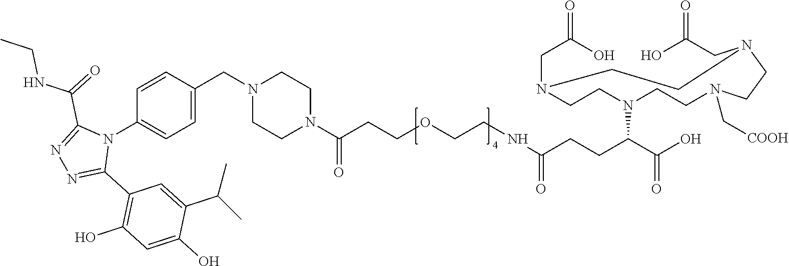

CMP52′: comprising TM5 ligand and (PEG)4 spacer, encompassed by Formula B2

TABLE 4
Non-Limiting Examples of Conjugates Comprising PEG Spacers and
More Than 1 TM
Structures

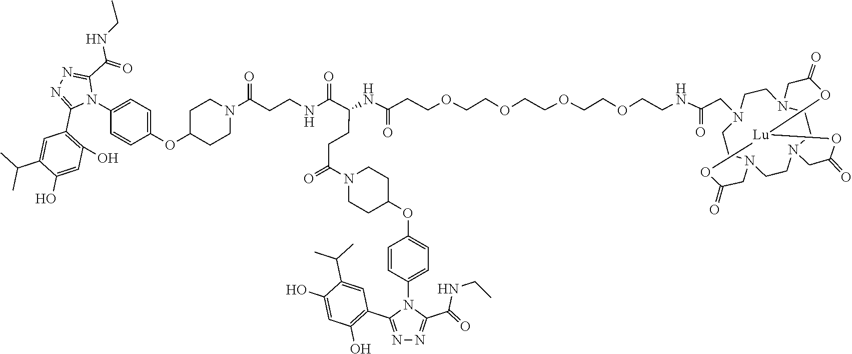

CMP35: comprising 2 TM10s and (PEG)4 spacer; encompassed by Formula B1

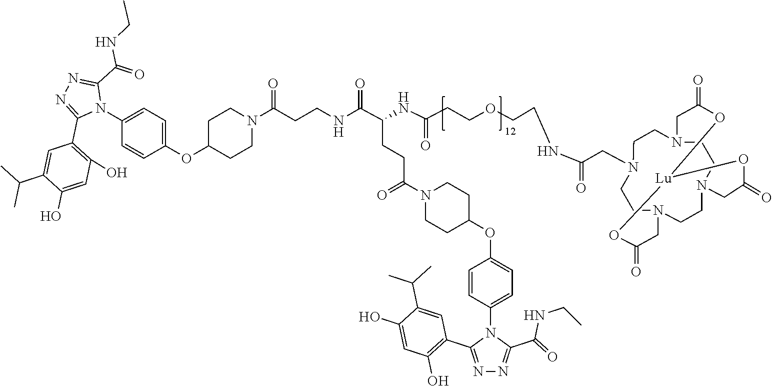

CMP36: comprising 2 TM10s and (PEG)12 spacer; encompassed by Formula B1

The conjugate analog structures without the radioactive agent are included in Table 4′. The conjugates may comprise any radioisotope, such as Y-90, Y-86, I-131, Re-186, Re-188, Y-90, Bi-212, At-211, Zr-89, Sr-89, Ho-166, Sm-153, Cu-67, Cu-64, Lu-177, Ac-225, Pb-203, Bi-213, Th-227, Pb-212, Ra-223, P-32, Sc-47, Br-77, Rh-105, Pd-103, Ag-111, Pr-142, Pm-149, Gd-159, Ir-194 and Pt-199.

TABLE 4′
Non-Limiting Examples of Conjugates Comprising PEG Spacers and
More Than 1 TM (without radioactive agent)
Structures

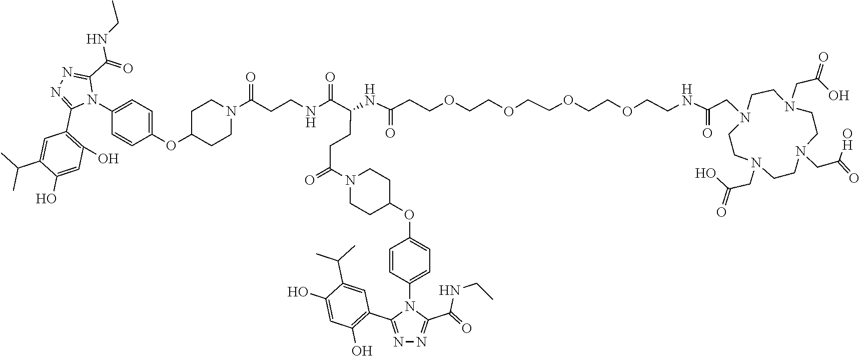 [SMILES: CCNC(=O)c1nnc(-c2cc(C(C)C)c(O)cc2O)n1-c1ccc(OC2CCN(C(=O)CCNC(=O)[C@@H](CCC(=O)N3CCC(Oc4ccc(-n5c(C(=O)NCC)nnc5-c5cc(C(C)C)c(O)cc5O)cc4)CC3)NC(=O)CCOCCOCCOCCOCCNC(=O)CN3CCN(CC(=O)O)CCN(CC(=O)O)CCN(CC(=O)O)CC3)CC2)cc1]

CMP35′: comprising 2 TM10s and (PEG)4 spacer; encompassed by Formula B1

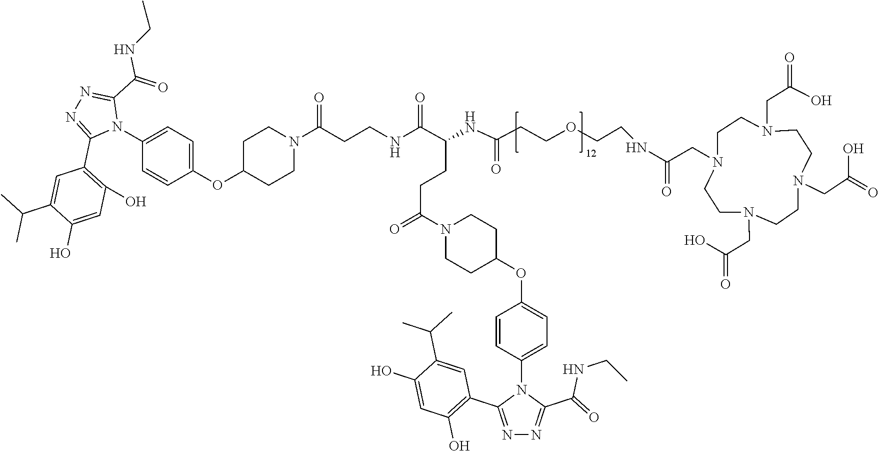

CMP36′: comprising 2 TM10s and (PEG)12 spacer; encompassed by Formula B1

In some embodiments, the conjugate comprises at least one (PEG)4 spacer. Non-limiting examples include CMP1, CMP2, CMP29, CMP30, CMP31, CMP39, CMP40, and CMP35.

In some embodiments, the conjugate comprises at least one (PEG)12 spacer. Non-limiting examples include CMP32, CMP33, CMP34, CMP36, CPM41, and CMP42.

### Albumin-Binding Pharmacokinetic Modulating Units

In some embodiments, the conjugate comprises an HSP90 targeting moiety attached to a chelating agent for a radioactive agent with a linker, and at least one pharmacokinetic modulating unit (PMU). The PMU may be any group that binds to albumin. The HSP90 targeting moiety may be a ganetespib analog or derivative (such as TM1, TM2, TM3, TM4, TM5, TM8, TM9, TM10, TM11, TM12, TM13 or TM14), an onalespib analog or derivative (such as TM6 or TM7), or TM15. In some embodiments, the linker may comprise a spacer comprising polyethylene glycol (PEG), such as (PEG)4 or (PEG)12. In some embodiments, the linker may comprise at least one amino acid. HSP90 binding affinity is maintained regardless of what charges are present on the spacer and/or PMU.

In some embodiments, the PMU comprises a functional group that binds to albumin, such as

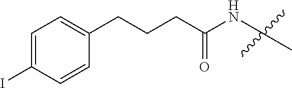 [SMILES: *NC(=O)CCCc1ccc(I)cc1]

(4-(4-iodophenyl) butanamido group).

In some embodiments, the conjugate has a structure of Formula C:

wherein TM(s) refers to at least one targeting moiety that binds to HSP90. In some embodiments, the conjugate has 2 targeting moieties. In some embodiments, the conjugate has 3 targeting moieties. In some embodiments, the conjugate has 4 targeting moieties. In some embodiments, the linker comprises a PEG spacer. In some embodiments, the linker comprises an amino acid spacer.

In some embodiments, the conjugate has a structure of Formula C10:

TM is selected from TM1, TM2, TM3, TM4, TM5, TM8, TM9, TM10, TM11, TM12, TM13, TM14, TM6, TM7, or TM15.

In some embodiments, the conjugate has a structure of Formula C1:

TM is selected from TM1, TM2, TM3, TM4, TM5, TM8, TM9, TM10, TM11, TM12, TM13, TM14, TM6, TM7, or TM15. Non-limiting examples of conjugates encompassed by Formula C1 include CMP3, CMP4, CMP5, CMP6.

In some embodiments, the conjugate has a structure of Formula C20:

TM is selected from TM1, TM2, TM3, TM4, TM5, TM8, TM9, TM10, TM11, TM12, TM13, TM14, TM6, TM7, or TM15.

In some embodiments, the conjugate has a structure of Formula C2:

TM is selected from TM1, TM2, TM3, TM4, TM5, TM8, TM9, TM10, TM11, TM12, TM13, TM14, TM6, TM7, or TM15. Non-limiting examples of conjugates encompassed by Formula C1 include T3, T7, T11, T12, T13, T14, T15, T16, T17, T22, T23, T32, T24, T33, T34, T35, T36, T37, T38, T41, and T42.

Non-limiting examples of conjugates of the current disclosure that are encompassed by Formula C include CMP3, CMP4, CMP5, CMP6, T3, T7, T11, T12, T13, T14, T15, T16, T17, T22, T23, T32, T24, T33, T34, T35, T36, T37, T38, T41, or T42:

TABLE 5
Non-Limiting Examples of Conjugates Comprising
Albumin-binding PMU
Structure

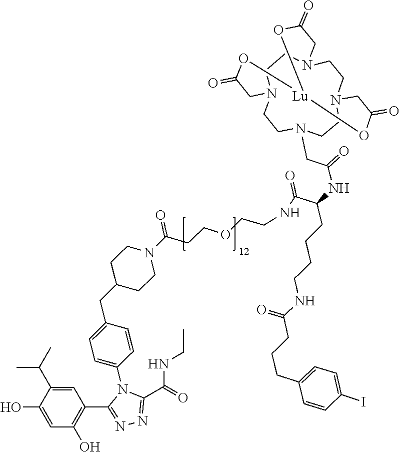

CMP3: comprising a TM9 ligand and a (PEG)12 spacer; encompassed by Formula C1

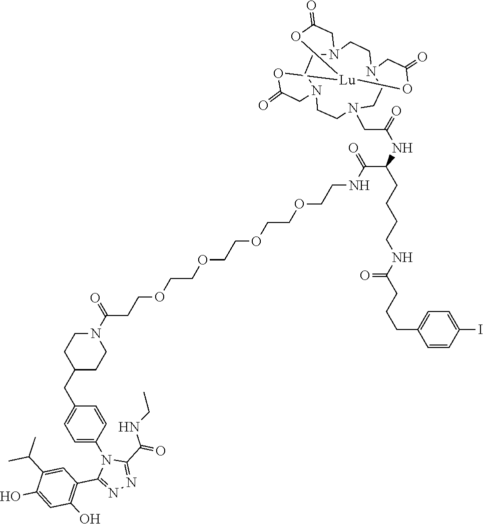

CMP4: comprising a TM9 ligand and a (PEG)4 spacer; encompassed by Formula C1

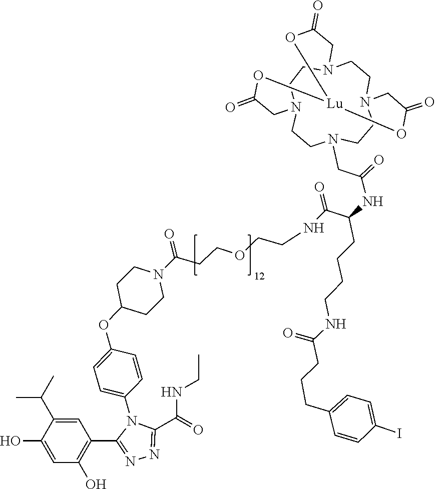

CMP5: comprising a TM10 ligand and a (PEG)12 spacer; encompassed by Formula C1

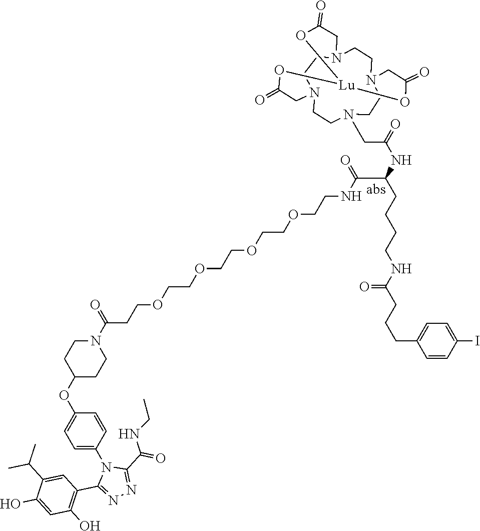

CMP6: comprising a TM10 ligand and a (PEG)4 spacer; encompassed by Formula C1

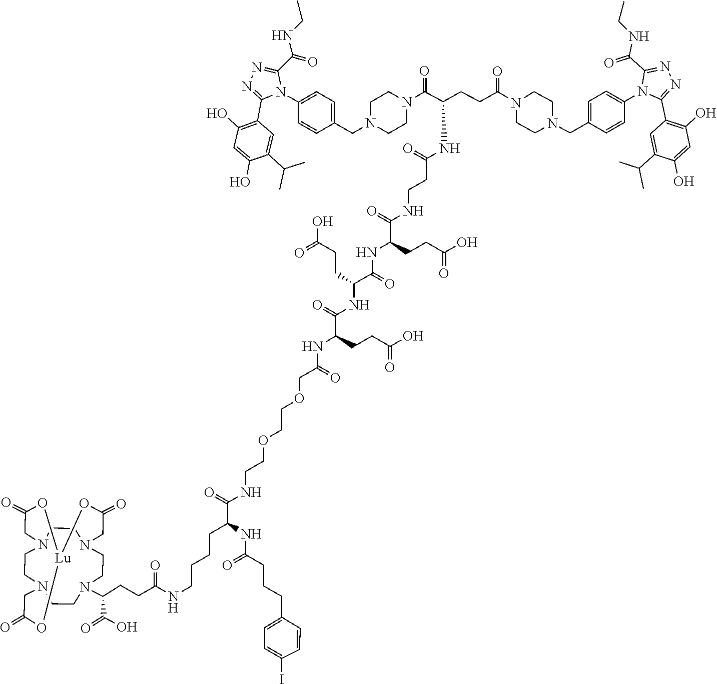

T3: comprising two TM5 ligands and an amino acid spacer; encompassed by Formula C2

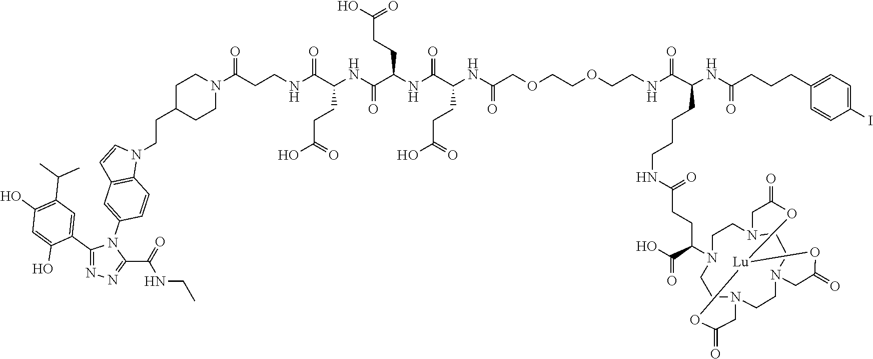

T7: comprising a TM5 ligand and an amino acid spacer; encompassed by Formula C2

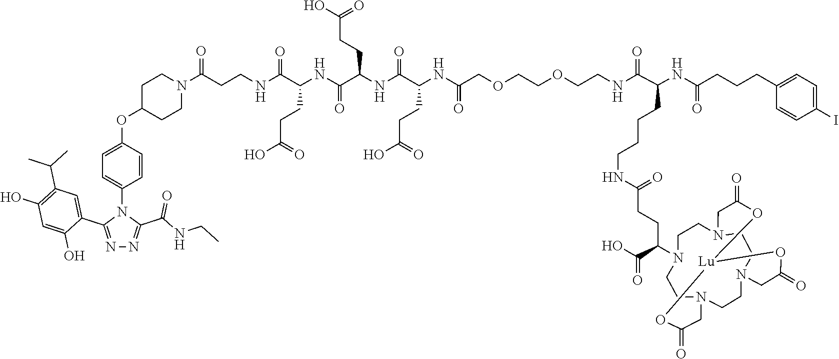

T11: comprising a TM10 ligand and an amino acid spacer; encompassed by Formula C2

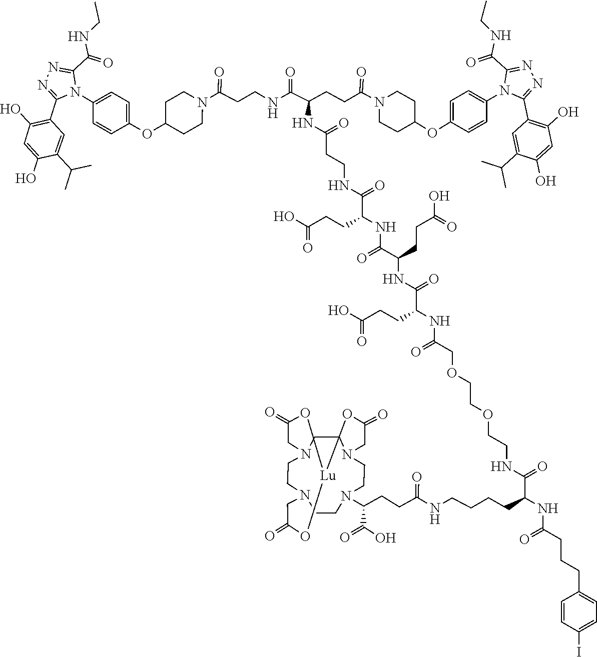

T12: comprising 2 TM10 ligands and an amino acid spacer; encompassed by Formula C2

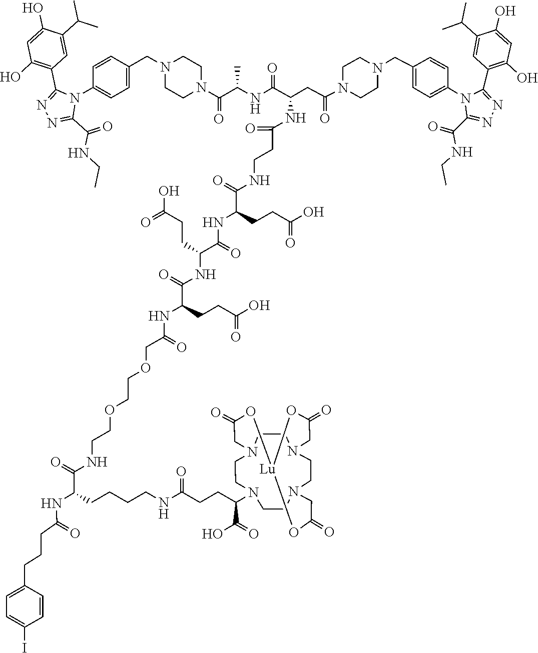

T13: comprising two TM2 ligands and an amino acid spacer; encompassed by Formula C2

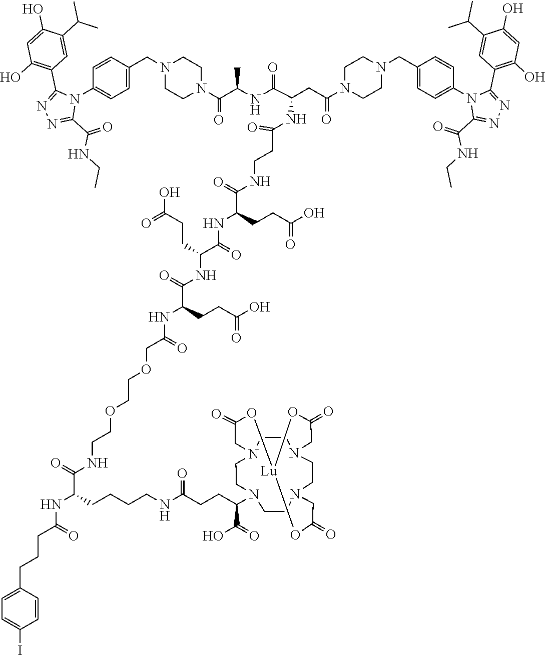

T14: comprising two TM5 ligands and an amino acid spacer; encompassed by Formula C2

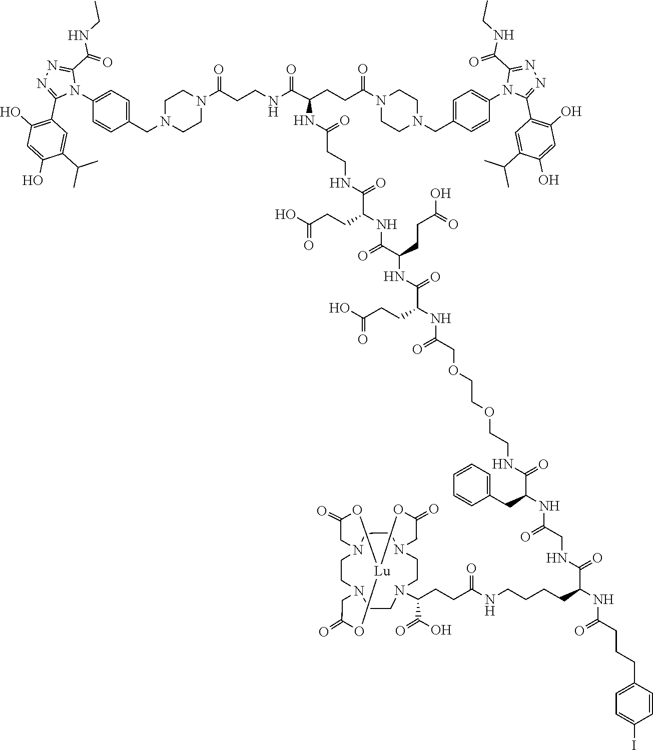

T15: comprising two TM5 ligands and an amino acid spacer; encompassed by Formula C2

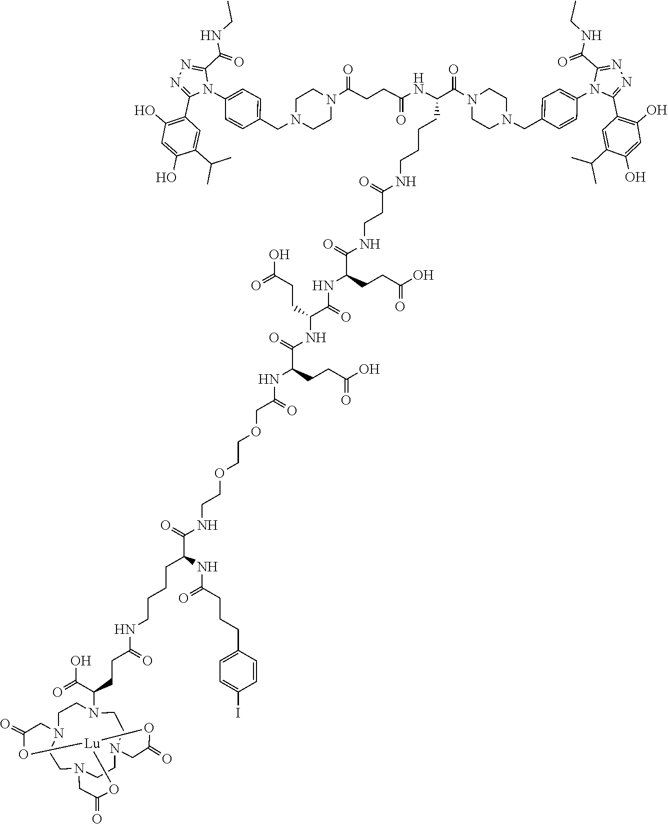

T16: comprising two TM5 ligands and amino acid spacer; encompassed by Formula C2

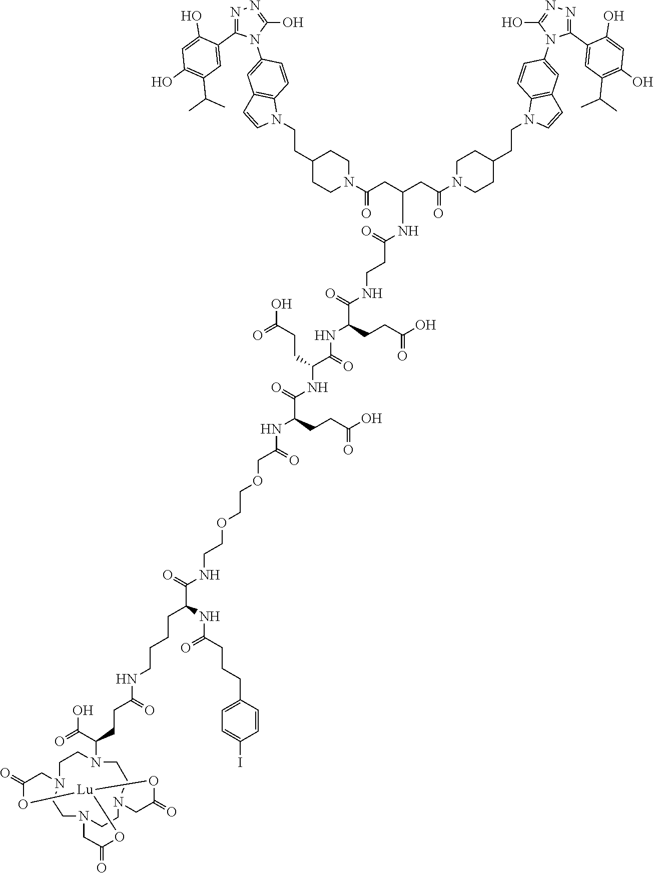

T17: comprising two TM1 ligands and an amino acid spacer; encompassed by Formula C2

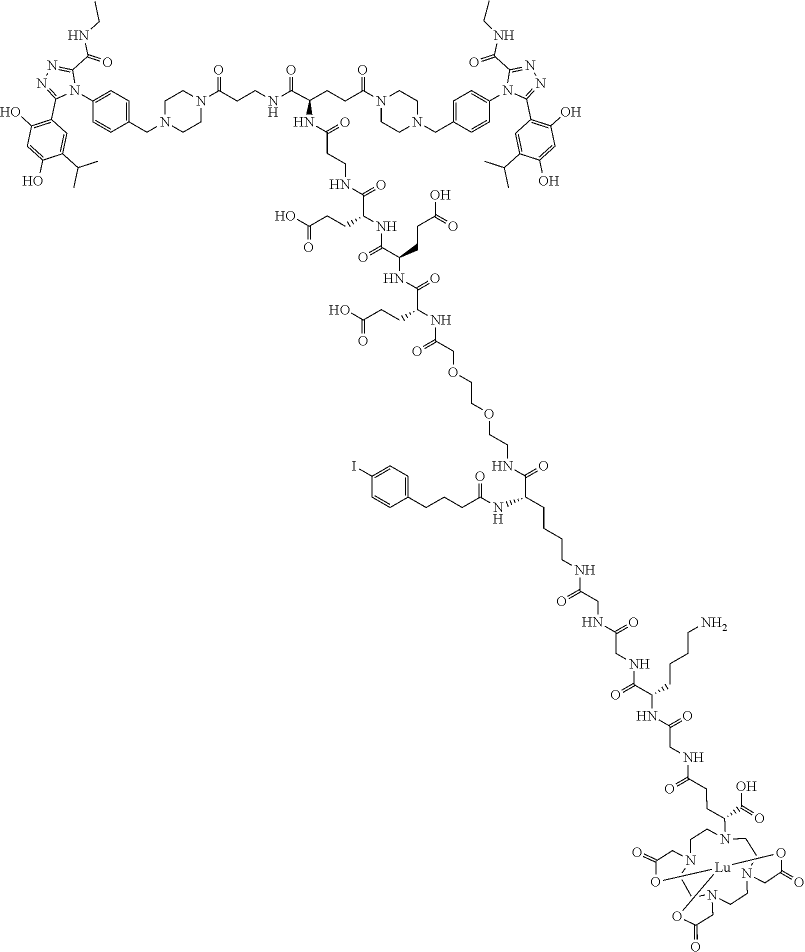

T22: comprising two TM5 ligands and an amino acid spacer; encompassed by Formula C2

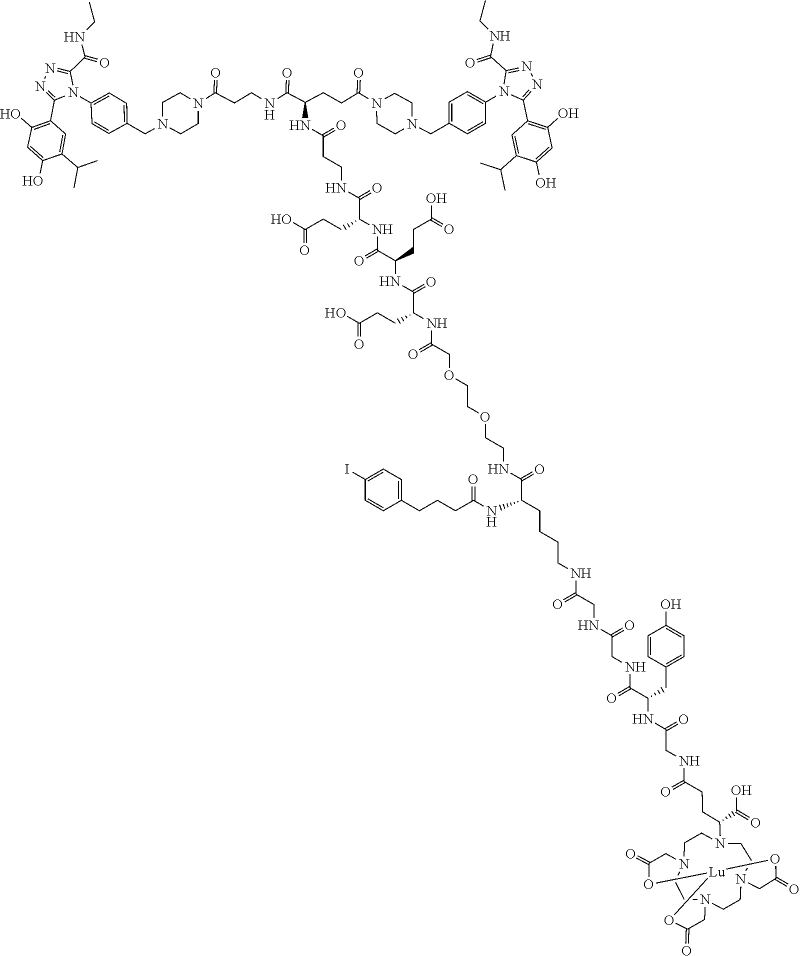

T23: comprising two TM5 ligands and an amino acid spacer; encompassed by Formula C2

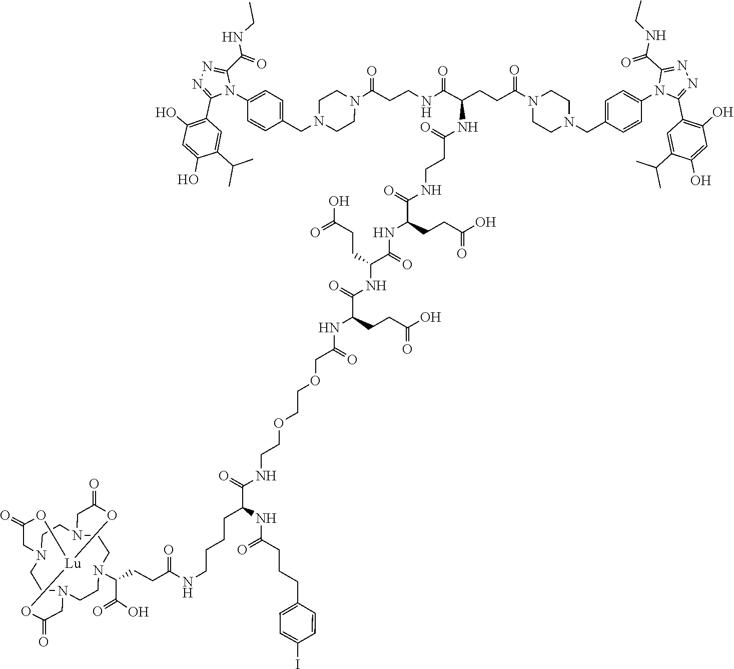

T32: comprising two TM5 ligands and an amino acid spacer; encompassed by Formula C2

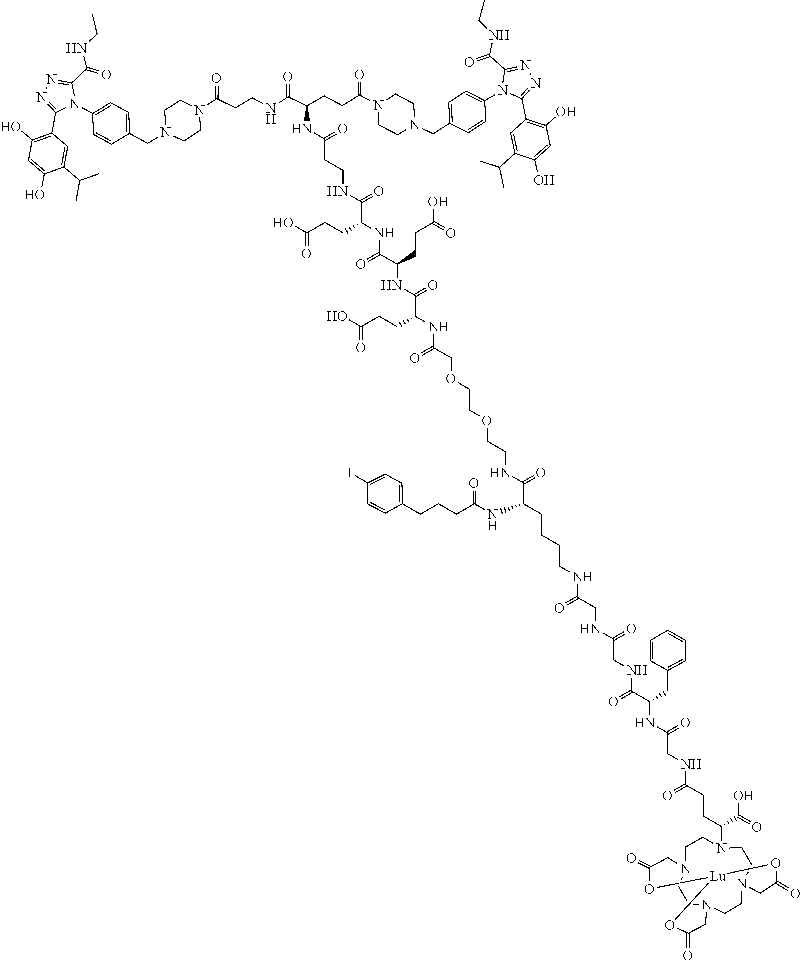

T24: comprising two TM5 ligands and an amino acid spacer; encompassed by Formula C2

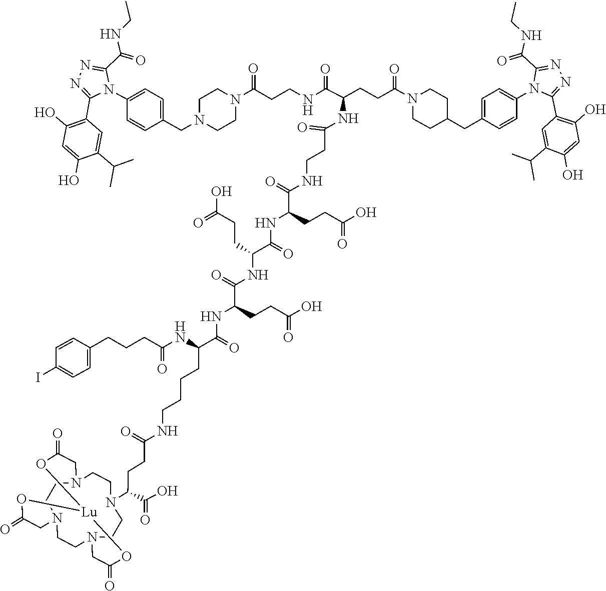

T33: comprising two TM5 ligands and an amino acid spacer; encompassed by Formula C2

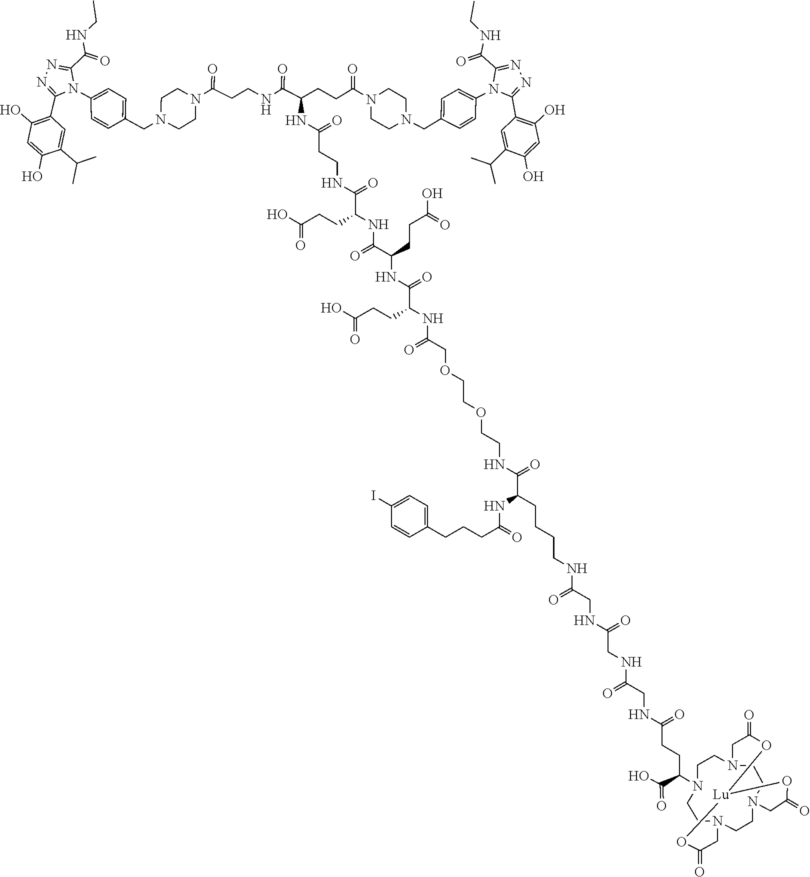

T34: comprising two TM5 ligands and an amino acid spacer; encompassed by Formula C2

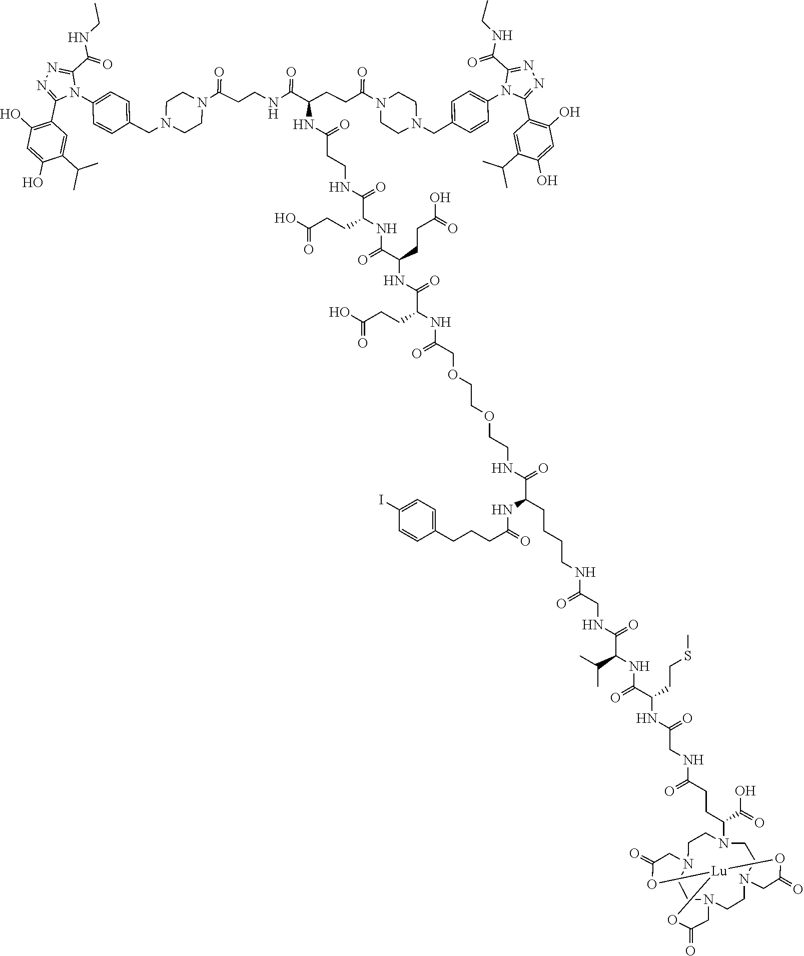

T35: comprising two TM5 ligands and an amino acid spacer; encompassed by Formula C2

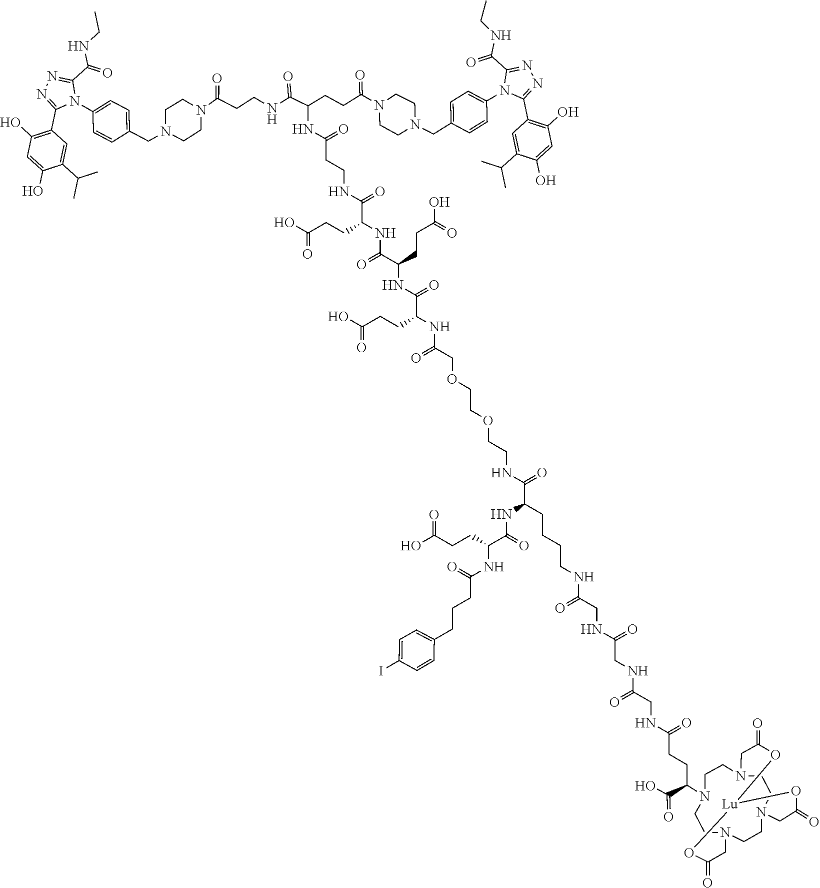

T36: comprising two TM5 ligands and an amino acid spacer; encompassed by Formula C2

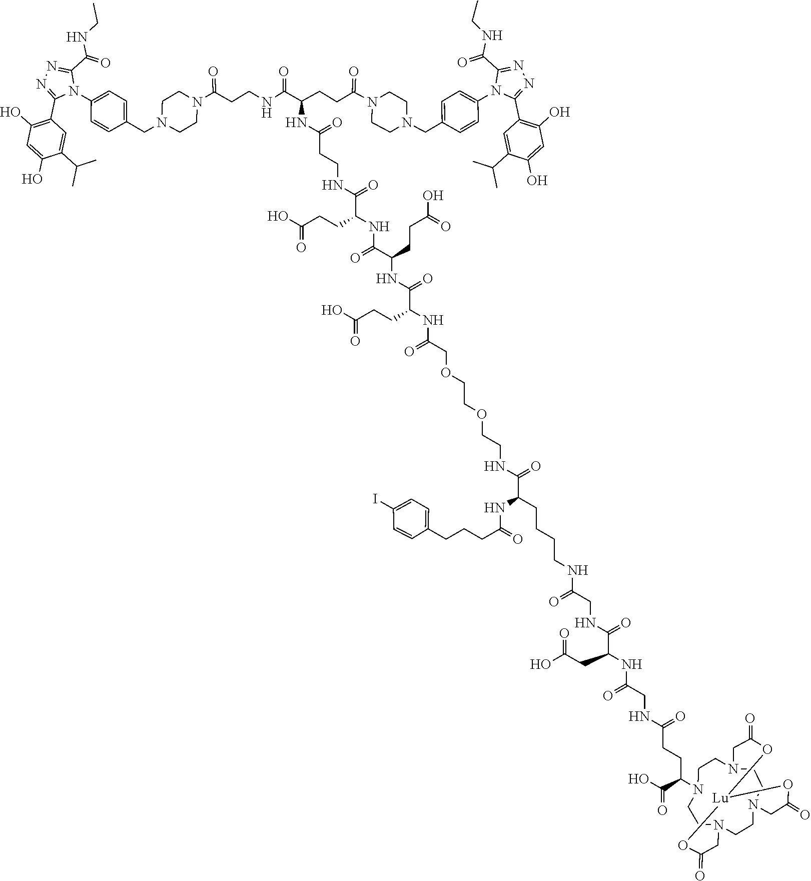

T37: comprising two TM5 ligands and an amino acid spacer; encompassed by Formula C2

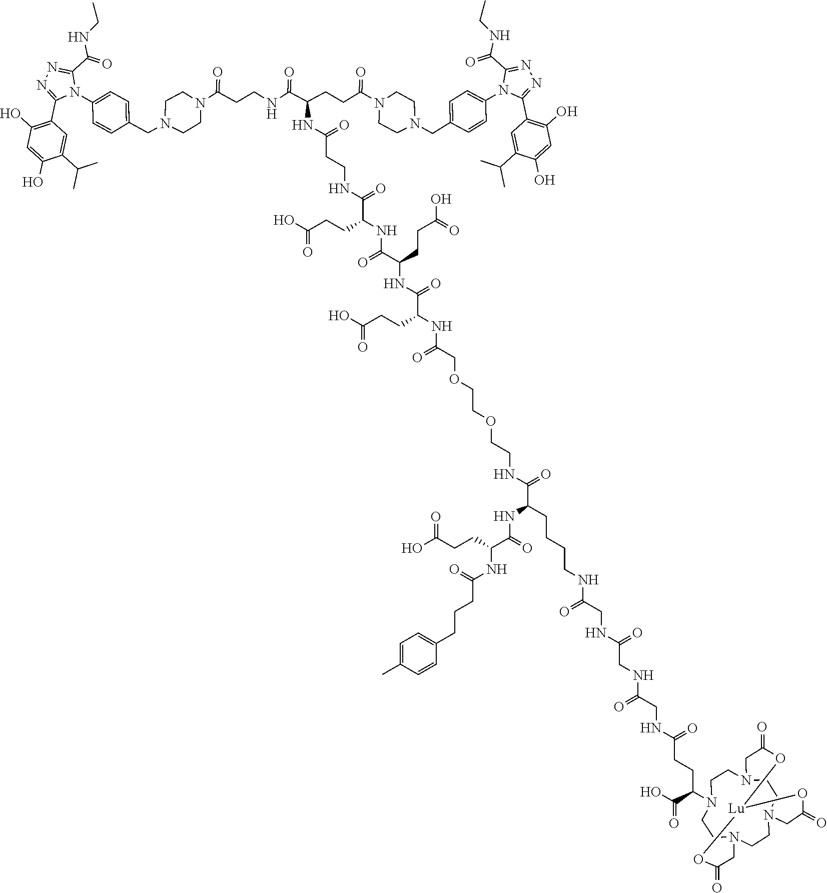

T38: comprising two TM5 ligands and an amino acid spacer; encompassed by Formula C2

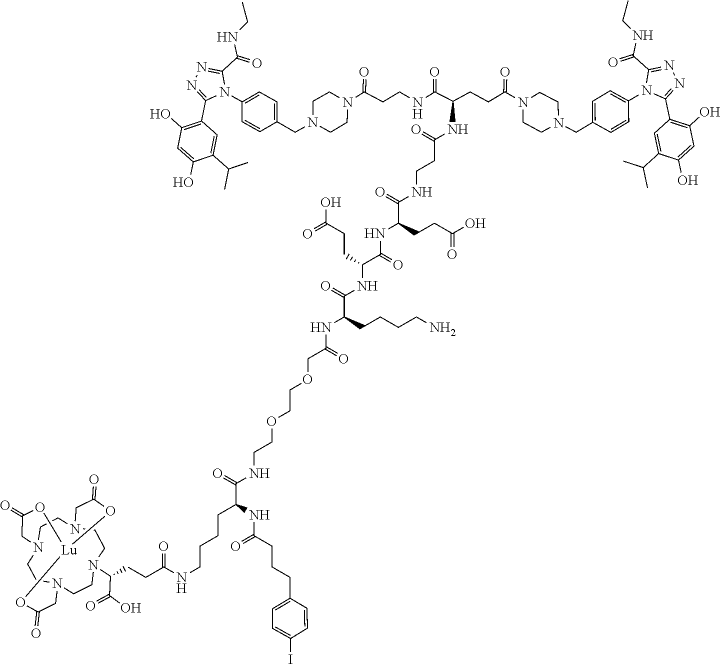

T41: comprising two TM5 ligands and an amino acid spacer; encompassed by Formula C2

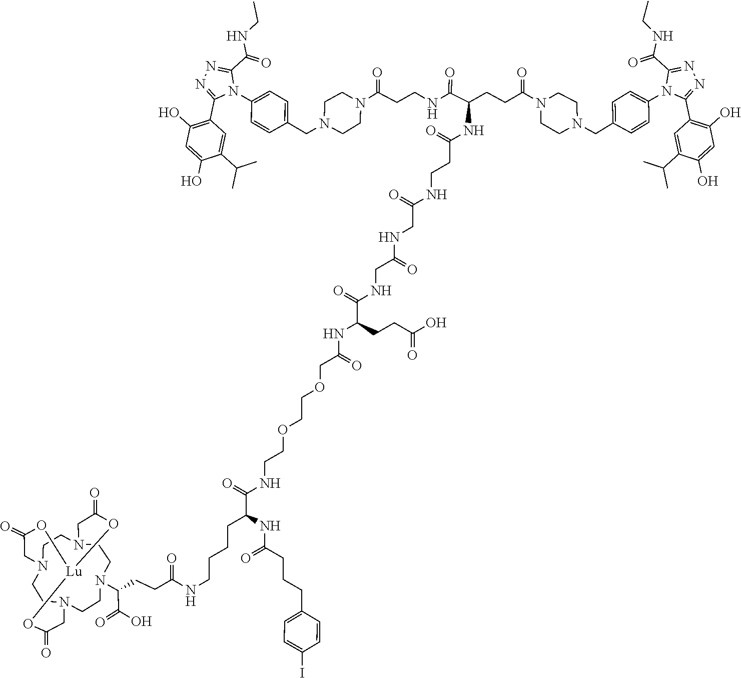

T42: comprising two TM5 ligands and an amino acid spacer; encompassed by Formula C2

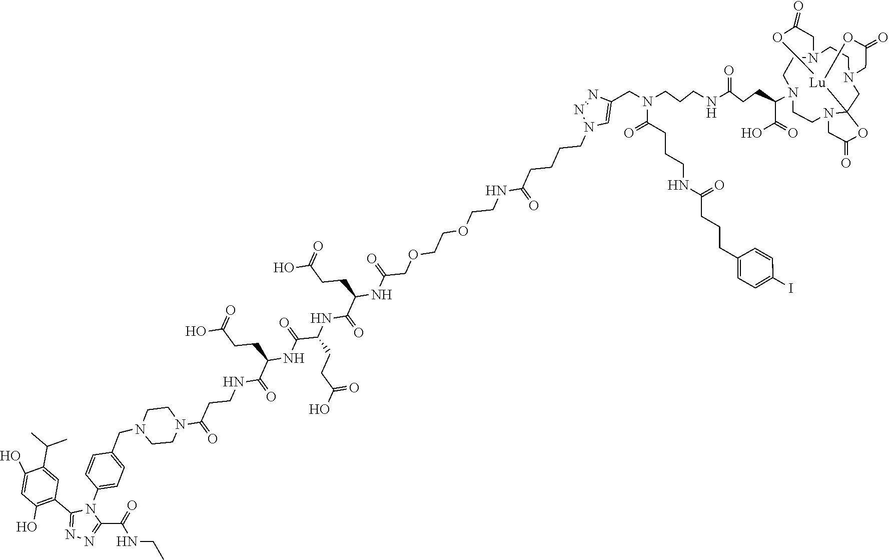

CMP51: comprising one TM5 ligand and an amino acid spacer; encompassed by Formula C2

The conjugate analog structures without the radioactive agent are included in Table 5′. The conjugates may comprise any radioisotope, such as Y-90, Y-86, I-131, Re-186, Re-188, Y-90, Bi-212, At-211, Zr-89, Sr-89, Ho-166, Sm-153, Cu-67, Cu-64, Lu-177, Ac-225, Pb-203, Bi-213, Th-227, Pb-212, Ra-223, P-32, Sc-47, Br-77, Rh-105, Pd-103, Ag-111, Pr-142, Pm-149, Gd-159, Ir-194 and Pt-199.

TABLE 5′
Non-Limiting Examples of Conjugates Comprising Albumin-binding
PMU (without radioactive agent)
Structure

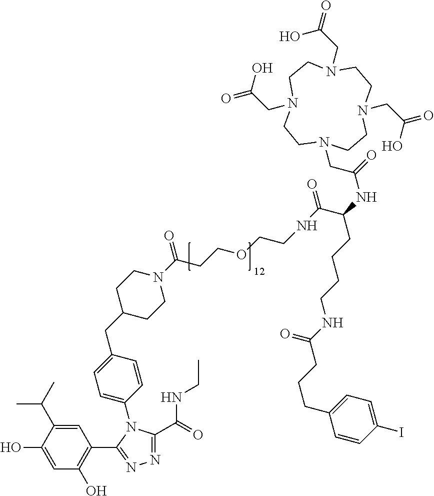

CMP3′: comprising a TM9 ligand and a (PEG)12 spacer; encompassed by Formula C1

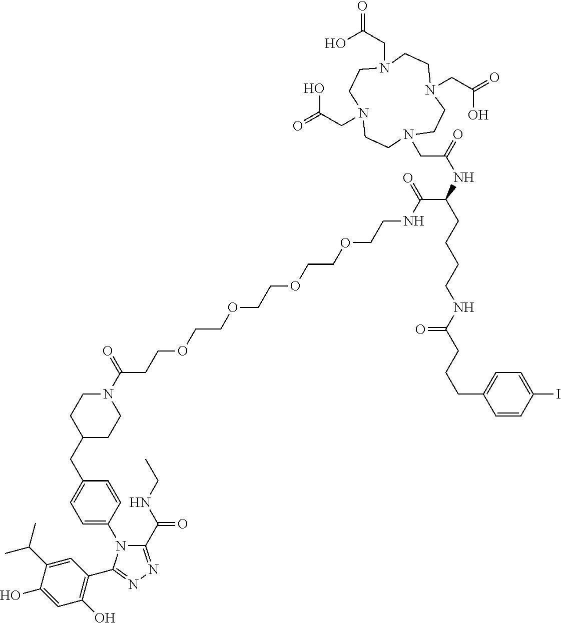 [SMILES: CCNC(=O)c1nnc(-c2cc(C(C)C)c(O)cc2O)n1-c1ccc(CC2CCN(C(=O)CCOCCOCCOCCOCCNC(=O)[C@H](CCCCNC(=O)CCCc3ccc(I)cc3)NC(=O)CN3CCN(CC(=O)O)CCN(CC(=O)O)CCN(CC(=O)O)CC3)CC2)cc1]

CMP4′: comprising a TM9 ligand and a (PEG)4 spacer; encompassed by Formula C1

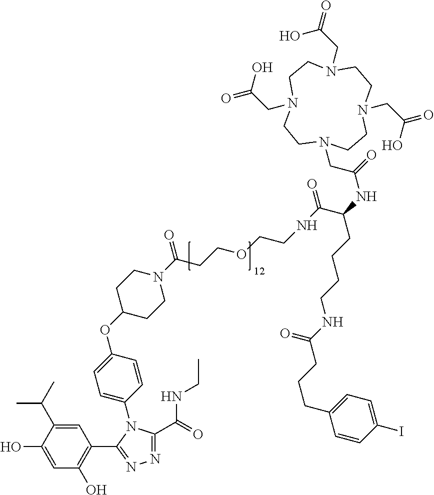

CMP5′: comprising a TM10 ligand and a (PEG)12 spacer; encompassed by Formula C1

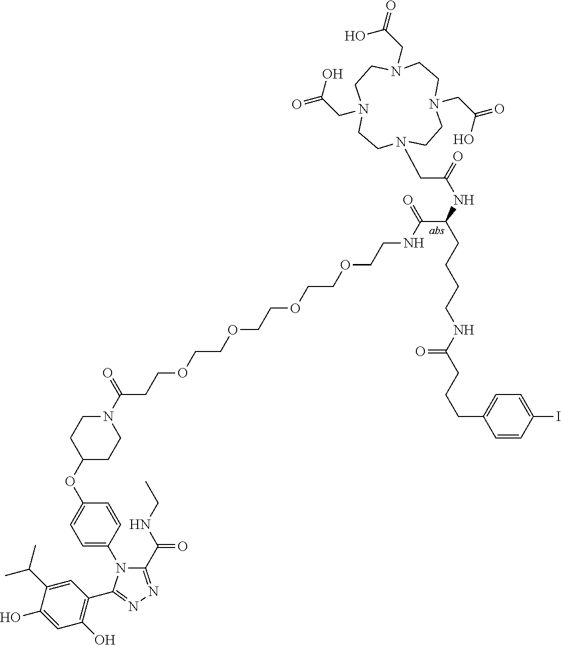 [SMILES: CCNC(=O)c1nnc(-c2cc(C(C)C)c(O)cc2O)n1-c1ccc(OC2CCN(C(=O)CCOCCOCCOCCOCCNC(=O)[C@H](CCCCNC(=O)CCCc3ccc(I)cc3)NC(=O)CN3CCN(CC(=O)O)CCN(CC(=O)O)CCN(CC(=O)O)CC3)CC2)cc1]

CMP6′: comprising a TM10 ligand and a (PEG)4 spacer; encompassed by Formula C1

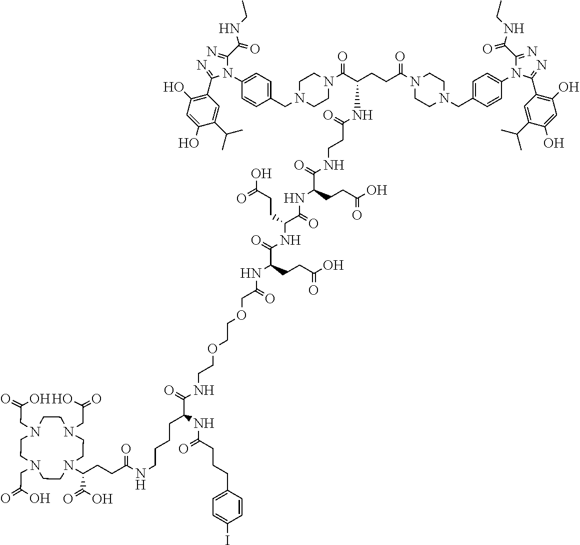 [SMILES: CCNC(=O)c1nnc(-c2cc(C(C)C)c(O)cc2O)n1-c1ccc(CN2CCN(C(=O)CC[C@H](NC(=O)CCNC(=O)[C@@H](CCC(=O)O)NC(=O)[C@@H](CCC(=O)O)NC(=O)[C@@H](CCC(=O)O)NC(=O)COCCOCCNC(=O)[C@H](CCCCNC(=O)CC[C@H](C(=O)O)N3CCN(CC(=O)O)CCN(CC(=O)O)CCN(CC(=O)O)CC3)NC(=O)CCCc3ccc(I)cc3)C(=O)N3CCN(Cc4ccc(-n5c(C(=O)NCC)nnc5-c5cc(C(C)C)c(O)cc5O)cc4)CC3)CC2)cc1]

T3′: comprising two TM5 ligands and an amino acid spacer; encompassed by Formula C2

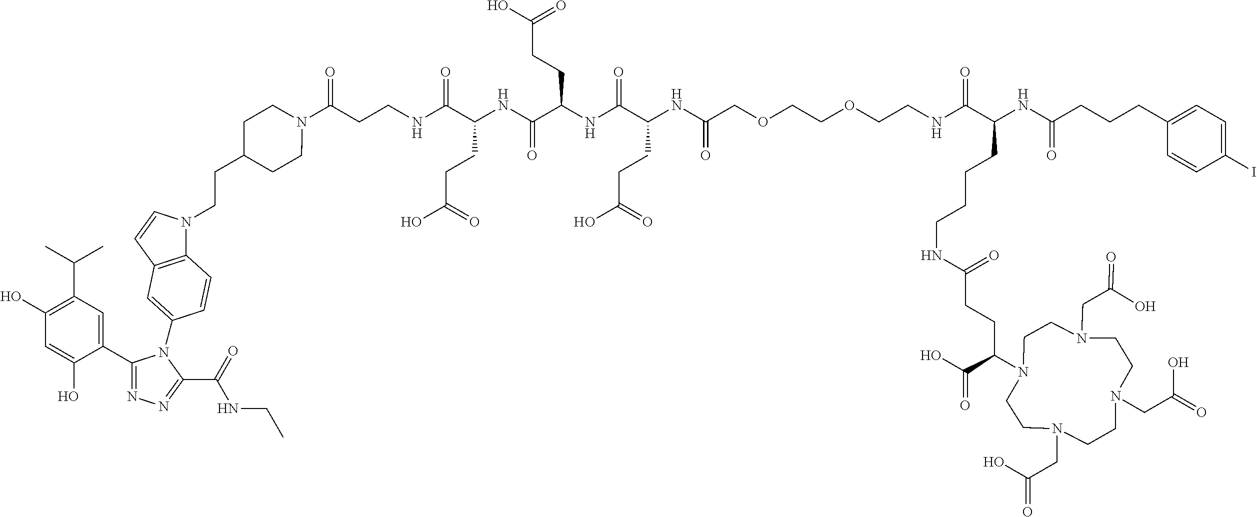 [SMILES: CCNC(=O)c1nnc(-c2cc(C(C)C)c(O)cc2O)n1-c1ccc2c(ccn2CCC2CCN(C(=O)CCNC(=O)[C@@H](CCC(=O)O)NC(=O)[C@@H](CCC(=O)O)NC(=O)[C@@H](CCC(=O)O)NC(=O)COCCOCCNC(=O)[C@H](CCCCNC(=O)CC[C@H](C(=O)O)N3CCN(CC(=O)O)CCN(CC(=O)O)CCN(CC(=O)O)CC3)NC(=O)CCCc3ccc(I)cc3)CC2)c1]

T7′: comprising a TM5 ligand and an amino acid spacer; encompassed by Formula C2

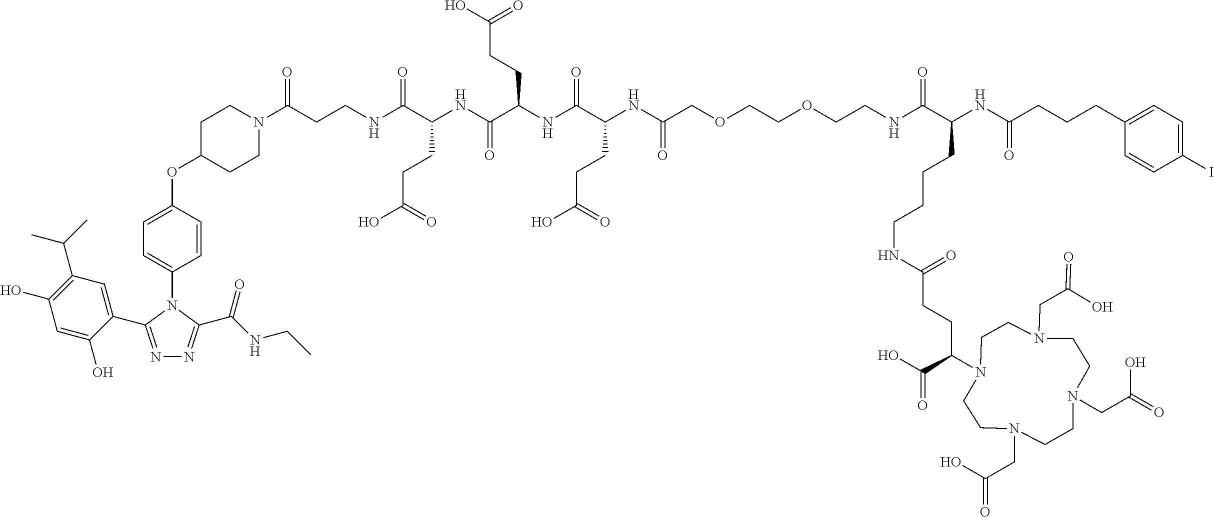 [SMILES: CCNC(=O)c1nnc(-c2cc(C(C)C)c(O)cc2O)n1-c1ccc(OC2CCN(C(=O)CCNC(=O)[C@@H](CCC(=O)O)NC(=O)[C@@H](CCC(=O)O)NC(=O)[C@@H](CCC(=O)O)NC(=O)COCCOCCNC(=O)[C@H](CCCCNC(=O)CC[C@H](C(=O)O)N3CCN(CC(=O)O)CCN(CC(=O)O)CCN(CC(=O)O)CC3)NC(=O)CCCc3ccc(I)cc3)CC2)cc1]

T11′: comprising a TM10 ligand and an amino acid spacer; encompassed by Formula C2

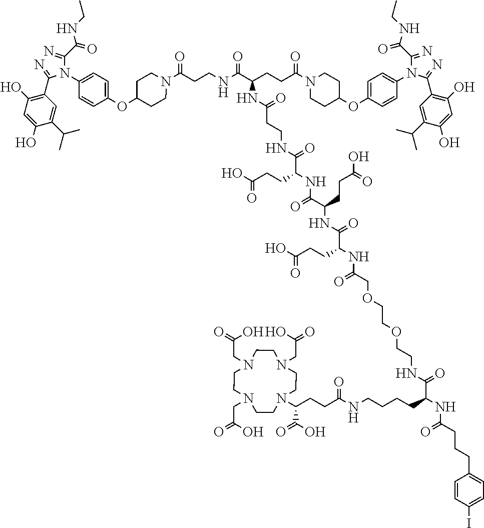 [SMILES: CCNC(=O)c1nnc(-c2cc(C(C)C)c(O)cc2O)n1-c1ccc(OC2CCN(C(=O)CCNC(=O)[C@@H](CCC(=O)N3CCC(Oc4ccc(-n5c(C(=O)NCC)nnc5-c5cc(C(C)C)c(O)cc5O)cc4)CC3)NC(=O)CCNC(=O)[C@@H](CCC(=O)O)NC(=O)[C@@H](CCC(=O)O)NC(=O)[C@@H](CCC(=O)O)NC(=O)COCCOCCNC(=O)[C@H](CCCCNC(=O)CC[C@H](C(=O)O)N3CCN(CC(=O)O)CCN(CC(=O)O)CCN(CC(=O)O)CC3)NC(=O)CCCc3ccc(I)cc3)CC2)cc1]

T12′: comprising 2 TM10 ligands and an amino acid spacer; encompassed by Formula C2

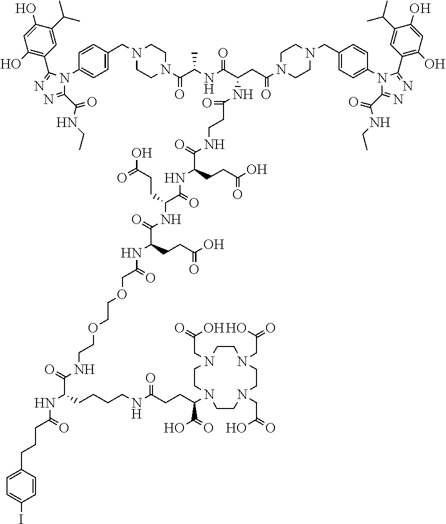 [SMILES: CCNC(=O)c1nnc(-c2cc(C(C)C)c(O)cc2O)n1-c1ccc(CN2CCN(C(=O)C[C@H](NC(=O)CCNC(=O)[C@@H](CCC(=O)O)NC(=O)[C@@H](CCC(=O)O)NC(=O)[C@@H](CCC(=O)O)NC(=O)COCCOCCNC(=O)[C@H](CCCCNC(=O)CC[C@H](C(=O)O)N3CCN(CC(=O)O)CCN(CC(=O)O)CCN(CC(=O)O)CC3)NC(=O)CCCc3ccc(I)cc3)C(=O)N[C@@H](C)C(=O)N3CCN(Cc4ccc(-n5c(C(=O)NCC)nnc5-c5cc(C(C)C)c(O)cc5O)cc4)CC3)CC2)cc1]

T13′: comprising two TM2 ligands and an amino acid spacer; encompassed by Formula C2

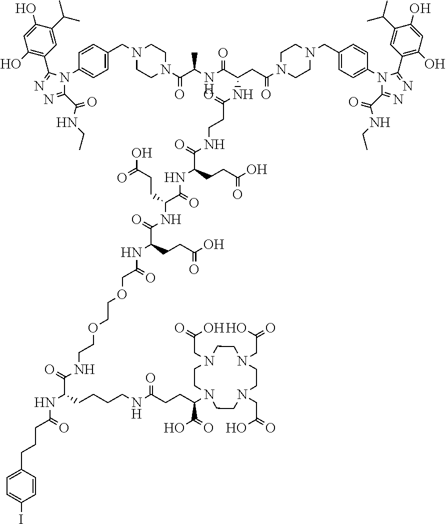 [SMILES: CCNC(=O)c1nnc(-c2cc(C(C)C)c(O)cc2O)n1-c1ccc(CN2CCN(C(=O)C[C@H](NC(=O)CCNC(=O)[C@@H](CCC(=O)O)NC(=O)[C@@H](CCC(=O)O)NC(=O)[C@@H](CCC(=O)O)NC(=O)COCCOCCNC(=O)[C@H](CCCCNC(=O)CC[C@H](C(=O)O)N3CCN(CC(=O)O)CCN(CC(=O)O)CCN(CC(=O)O)CC3)NC(=O)CCCc3ccc(I)cc3)C(=O)N[C@H](C)C(=O)N3CCN(Cc4ccc(-n5c(C(=O)NCC)nnc5-c5cc(C(C)C)c(O)cc5O)cc4)CC3)CC2)cc1]

T14′: comprising two TM5 ligands and an amino acid spacer; encompassed by Formula C2

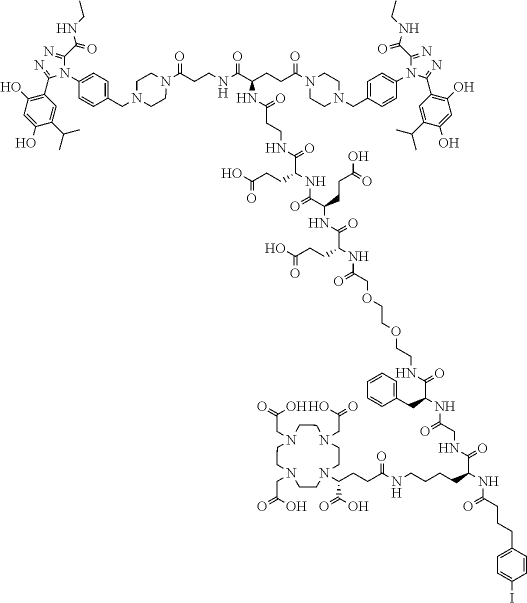 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)[C@@H](CCC(=O)O)NC(=O)[C@@H](CCC(=O)O)NC(=O)[C@@H](CCC(=O)O)NC(=O)COCCOCCNC(=O)[C@H](Cc3ccccc3)NC(=O)CNC(=O)[C@H](CCCCNC(=O)CC[C@H](C(=O)O)N3CCN(CC(=O)O)CCN(CC(=O)O)CCN(CC(=O)O)CC3)NC(=O)CCCc3ccc(I)cc3)CC2)cc1]

T15′: comprising two TM5 ligands and an amino acid spacer; encompassed by Formula C2

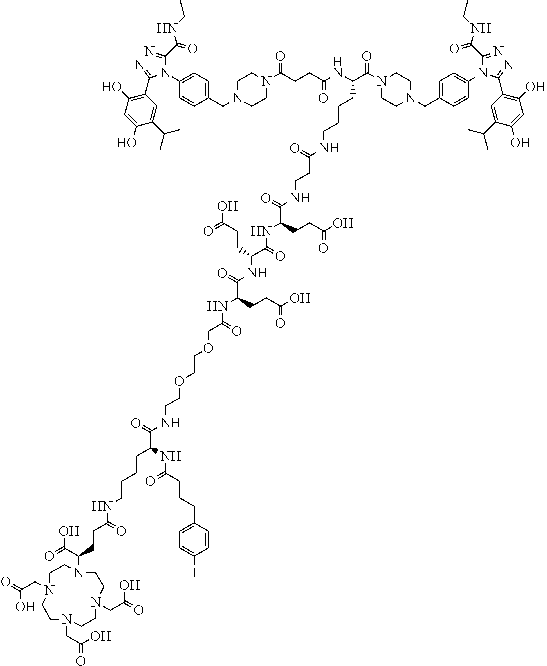 [SMILES: CCNC(=O)c1nnc(-c2cc(C(C)C)c(O)cc2O)n1-c1ccc(CN2CCN(C(=O)CCC(=O)N[C@@H](CCCCNC(=O)CCNC(=O)[C@@H](CCC(=O)O)NC(=O)[C@@H](CCC(=O)O)NC(=O)[C@@H](CCC(=O)O)NC(=O)COCCOCCNC(=O)[C@H](CCCCNC(=O)CC[C@H](C(=O)O)N3CCN(CC(=O)O)CCN(CC(=O)O)CCN(CC(=O)O)CC3)NC(=O)CCCc3ccc(I)cc3)C(=O)N3CCN(Cc4ccc(-n5c(C(=O)NCC)nnc5-c5cc(C(C)C)c(O)cc5O)cc4)CC3)CC2)cc1]

T16′: comprising two TM5 ligands and amino acid spacer; encompassed by Formula C2

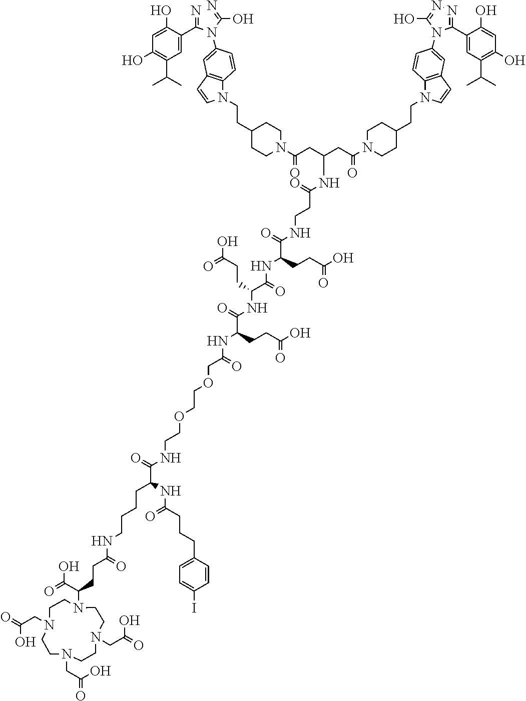 [SMILES: CC(C)c1cc(-c2nnc(O)n2-c2ccc3c(ccn3CCC3CCN(C(=O)CC(CC(=O)N4CCC(CCn5ccc6cc(-n7c(O)nnc7-c7cc(C(C)C)c(O)cc7O)ccc65)CC4)NC(=O)CCNC(=O)[C@@H](CCC(=O)O)NC(=O)[C@@H](CCC(=O)O)NC(=O)[C@@H](CCC(=O)O)NC(=O)COCCOCCNC(=O)[C@H](CCCCNC(=O)CC[C@H](C(=O)O)N4CCN(CC(=O)O)CCN(CC(=O)O)CCN(CC(=O)O)CC4)NC(=O)CCCc4ccc(I)cc4)CC3)c2)c(O)cc1O]

T17′: comprising two TM1 ligands and an amino acid spacer; encompassed by Formula C2

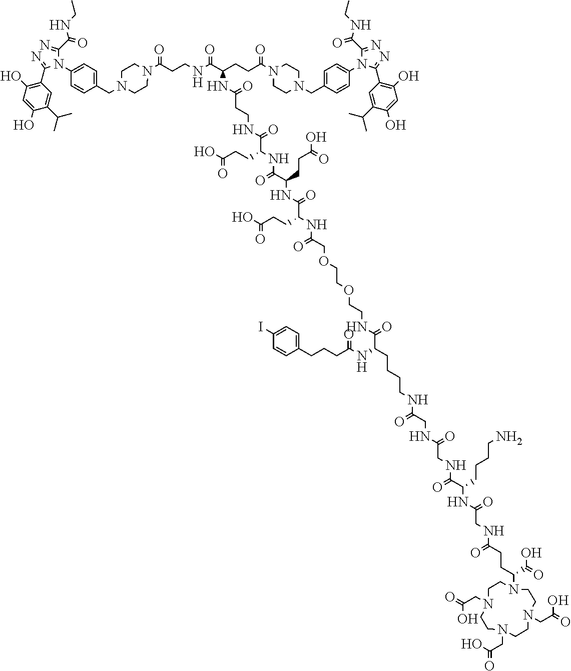 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)[C@@H](CCC(=O)O)NC(=O)[C@@H](CCC(=O)O)NC(=O)[C@@H](CCC(=O)O)NC(=O)COCCOCCNC(=O)[C@H](CCCCNC(=O)CNC(=O)CNC(=O)[C@H](CCCCN)NC(=O)CNC(=O)CC[C@H](C(=O)O)N3CCN(CC(=O)O)CCN(CC(=O)O)CCN(CC(=O)O)CC3)NC(=O)CCCc3ccc(I)cc3)CC2)cc1]

T22′: comprising two TM5 ligands and an amino acid spacer; encompassed by Formula C2

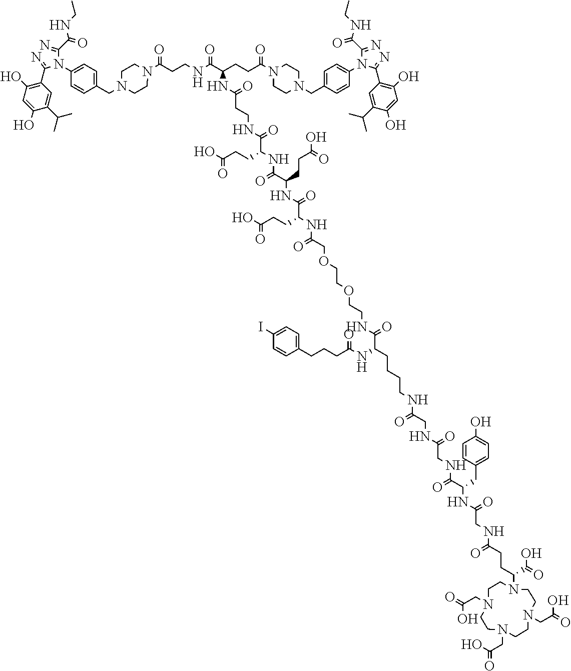 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)[C@@H](CCC(=O)O)NC(=O)[C@@H](CCC(=O)O)NC(=O)[C@@H](CCC(=O)O)NC(=O)COCCOCCNC(=O)[C@H](CCCCNC(=O)CNC(=O)CNC(=O)[C@H](Cc3ccc(O)cc3)NC(=O)CNC(=O)CC[C@H](C(=O)O)N3CCN(CC(=O)O)CCN(CC(=O)O)CCN(CC(=O)O)CC3)NC(=O)CCCc3ccc(I)cc3)CC2)cc1]

T23′: comprising two TM5 ligands and an amino acid spacer; encompassed by Formula C2

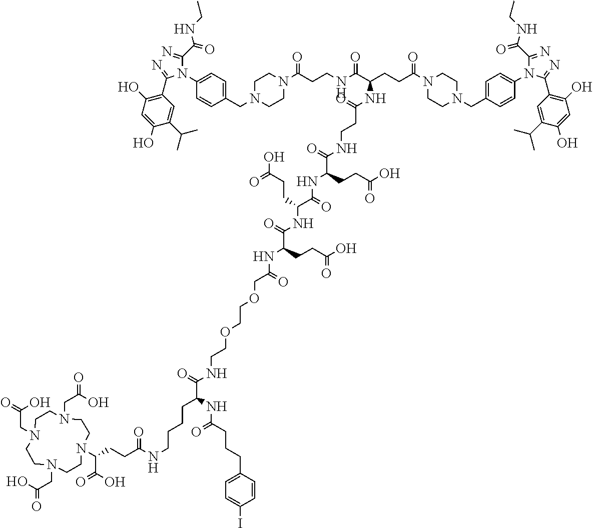 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)[C@@H](CCC(=O)O)NC(=O)[C@@H](CCC(=O)O)NC(=O)[C@@H](CCC(=O)O)NC(=O)COCCOCCNC(=O)[C@H](CCCCNC(=O)CC[C@H](C(=O)O)N3CCN(CC(=O)O)CCN(CC(=O)O)CCN(CC(=O)O)CC3)NC(=O)CCCc3ccc(I)cc3)CC2)cc1]

T32′: comprising two TM5 ligands and an amino acid spacer; encompassed by Formula C2

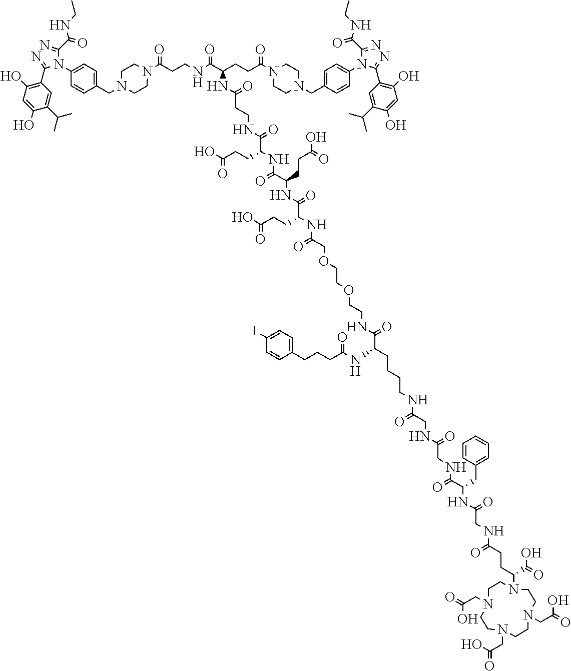 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)[C@@H](CCC(=O)O)NC(=O)[C@@H](CCC(=O)O)NC(=O)[C@@H](CCC(=O)O)NC(=O)COCCOCCNC(=O)[C@H](CCCCNC(=O)CNC(=O)CNC(=O)[C@H](Cc3ccccc3)NC(=O)CNC(=O)CC[C@H](C(=O)O)N3CCN(CC(=O)O)CCN(CC(=O)O)CCN(CC(=O)O)CC3)NC(=O)CCCc3ccc(I)cc3)CC2)cc1]

T24′: comprising two TM5 ligands and an amino acid spacer; encompassed by Formula C2

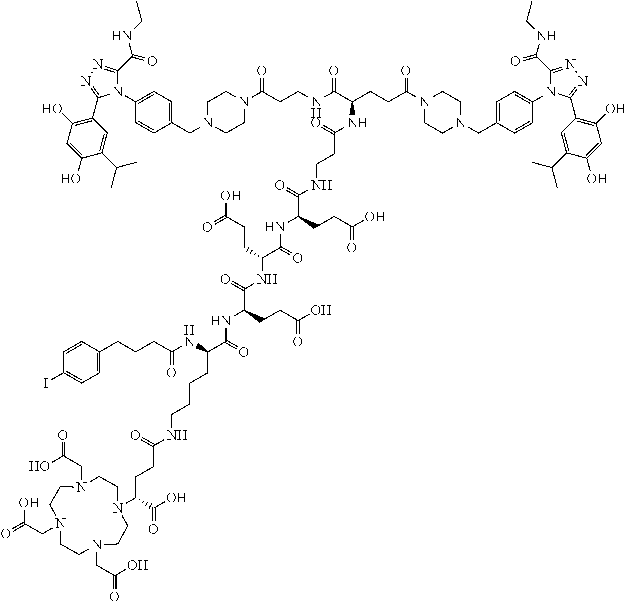 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)[C@@H](CCC(=O)O)NC(=O)[C@@H](CCC(=O)O)NC(=O)[C@@H](CCC(=O)O)NC(=O)[C@@H](CCCCNC(=O)CC[C@H](C(=O)O)N3CCN(CC(=O)O)CCN(CC(=O)O)CCN(CC(=O)O)CC3)NC(=O)CCCc3ccc(I)cc3)CC2)cc1]

T33′: comprising two TM5 ligands and an amino acid spacer; encompassed by Formula C2

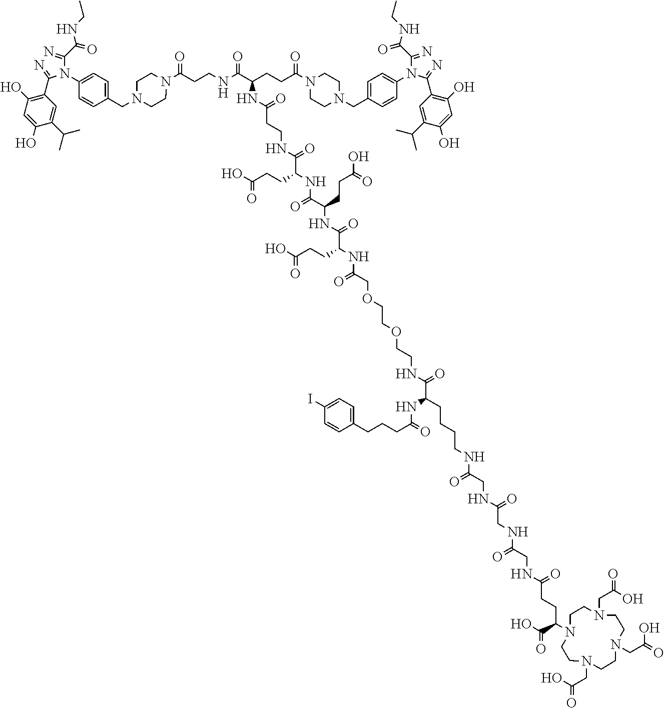 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)[C@@H](CCC(=O)O)NC(=O)[C@@H](CCC(=O)O)NC(=O)[C@@H](CCC(=O)O)NC(=O)COCCOCCNC(=O)[C@@H](CCCCNC(=O)CNC(=O)CNC(=O)CNC(=O)CC[C@H](C(=O)O)N3CCN(CC(=O)O)CCN(CC(=O)O)CCN(CC(=O)O)CC3)NC(=O)CCCc3ccc(I)cc3)CC2)cc1]

T34′: comprising two TM5 ligands and an amino acid spacer; encompassed by Formula C2

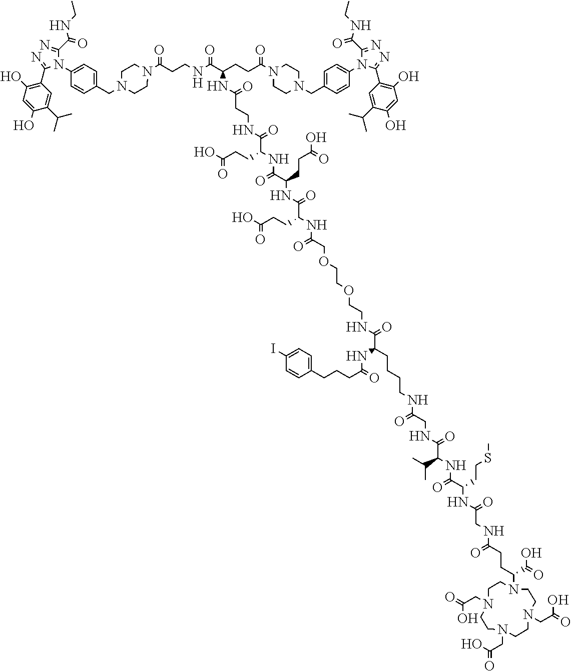 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)[C@@H](CCC(=O)O)NC(=O)[C@@H](CCC(=O)O)NC(=O)[C@@H](CCC(=O)O)NC(=O)COCCOCCNC(=O)[C@@H](CCCCNC(=O)CNC(=O)[C@@H](NC(=O)[C@H](CCSC)NC(=O)CNC(=O)CC[C@H](C(=O)O)N3CCN(CC(=O)O)CCN(CC(=O)O)CCN(CC(=O)O)CC3)C(C)C)NC(=O)CCCc3ccc(I)cc3)CC2)cc1]

T35′: comprising two TM5 ligands and an amino acid spacer; encompassed by Formula C2

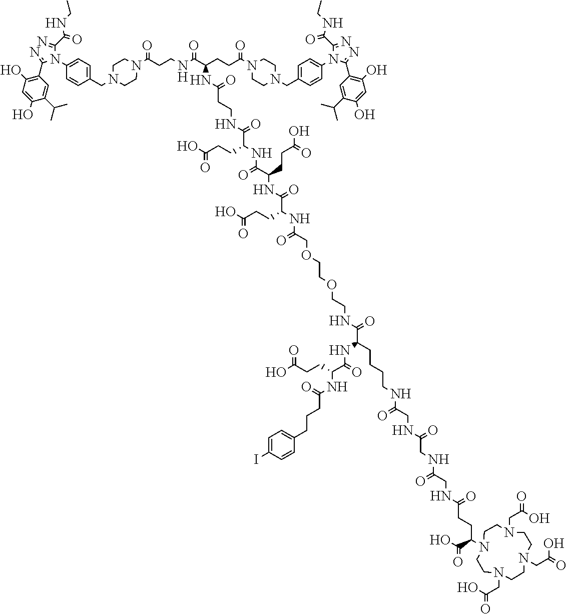 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)[C@@H](CCC(=O)O)NC(=O)[C@@H](CCC(=O)O)NC(=O)[C@@H](CCC(=O)O)NC(=O)COCCOCCNC(=O)[C@@H](CCCCNC(=O)CNC(=O)CNC(=O)CNC(=O)CC[C@H](C(=O)O)N3CCN(CC(=O)O)CCN(CC(=O)O)CCN(CC(=O)O)CC3)NC(=O)[C@@H](CCC(=O)O)NC(=O)CCCc3ccc(I)cc3)CC2)cc1]

T36′: comprising two TM5 ligands and an amino acid spacer; encompassed by Formula C2

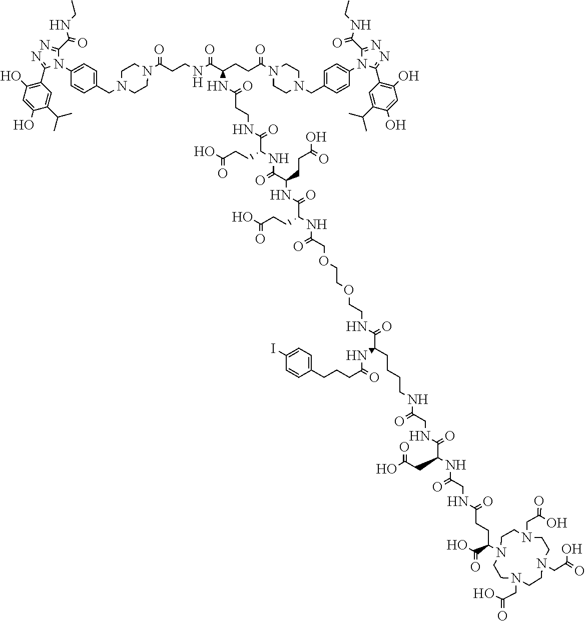 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)[C@@H](CCC(=O)O)NC(=O)[C@@H](CCC(=O)O)NC(=O)[C@@H](CCC(=O)O)NC(=O)COCCOCCNC(=O)[C@@H](CCCCNC(=O)CNC(=O)[C@H](CC(=O)O)NC(=O)CNC(=O)CC[C@H](C(=O)O)N3CCN(CC(=O)O)CCN(CC(=O)O)CCN(CC(=O)O)CC3)NC(=O)CCCc3ccc(I)cc3)CC2)cc1]

T37′: comprising two TM5 ligands and an amino acid spacer; encompassed by Formula C2

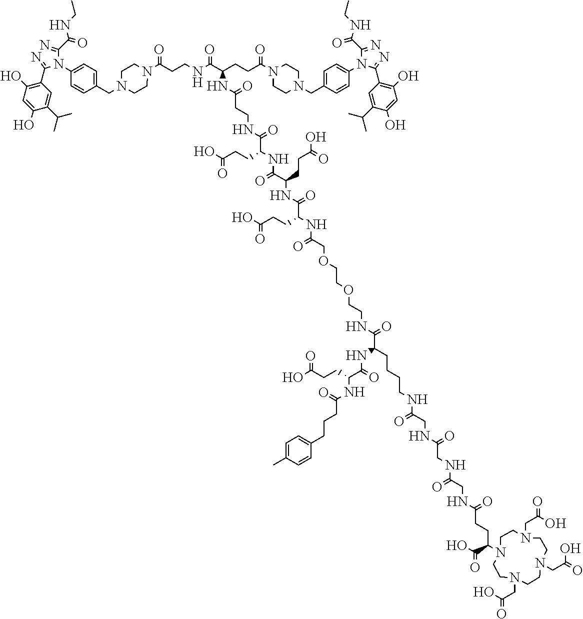 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)[C@@H](CCC(=O)O)NC(=O)[C@@H](CCC(=O)O)NC(=O)[C@@H](CCC(=O)O)NC(=O)COCCOCCNC(=O)[C@@H](CCCCNC(=O)CNC(=O)CNC(=O)CNC(=O)CC[C@H](C(=O)O)N3CCN(CC(=O)O)CCN(CC(=O)O)CCN(CC(=O)O)CC3)NC(=O)[C@@H](CCC(=O)O)NC(=O)CCCc3ccc(C)cc3)CC2)cc1]

T38′: comprising two TM5 ligands and an amino acid spacer; encompassed by Formula C2

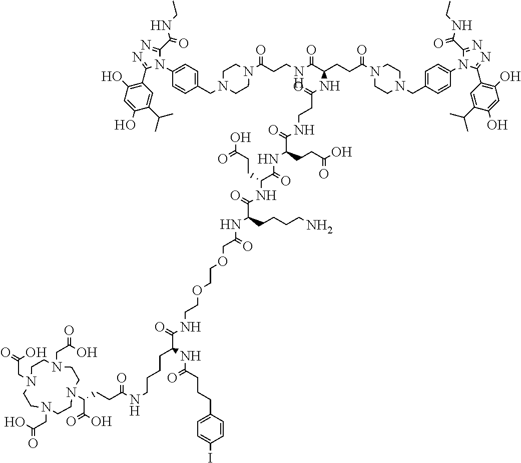 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)[C@@H](CCC(=O)O)NC(=O)[C@@H](CCC(=O)O)NC(=O)[C@@H](CCCCN)NC(=O)COCCOCCNC(=O)[C@H](CCCCNC(=O)CC[C@H](C(=O)O)N3CCN(CC(=O)O)CCN(CC(=O)O)CCN(CC(=O)O)CC3)NC(=O)CCCc3ccc(I)cc3)CC2)cc1]

T41′: comprising two TM5 ligands and an amino acid spacer; encompassed by Formula C2

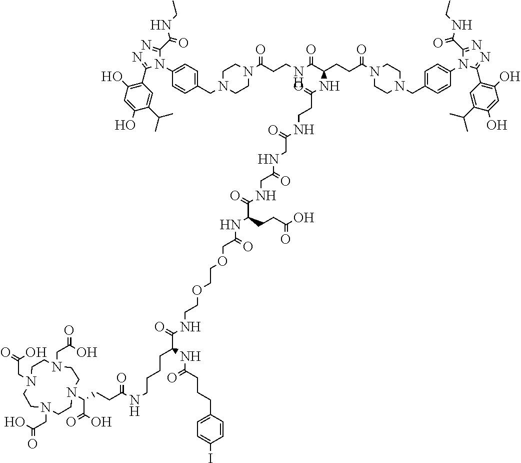 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)CNC(=O)CNC(=O)[C@@H](CCC(=O)O)NC(=O)COCCOCCNC(=O)[C@H](CCCCNC(=O)CC[C@H](C(=O)O)N3CCN(CC(=O)O)CCN(CC(=O)O)CCN(CC(=O)O)CC3)NC(=O)CCCc3ccc(I)cc3)CC2)cc1]

T42′: comprising two TM5 ligands and an amino acid spacer; encompassed by Formula C2

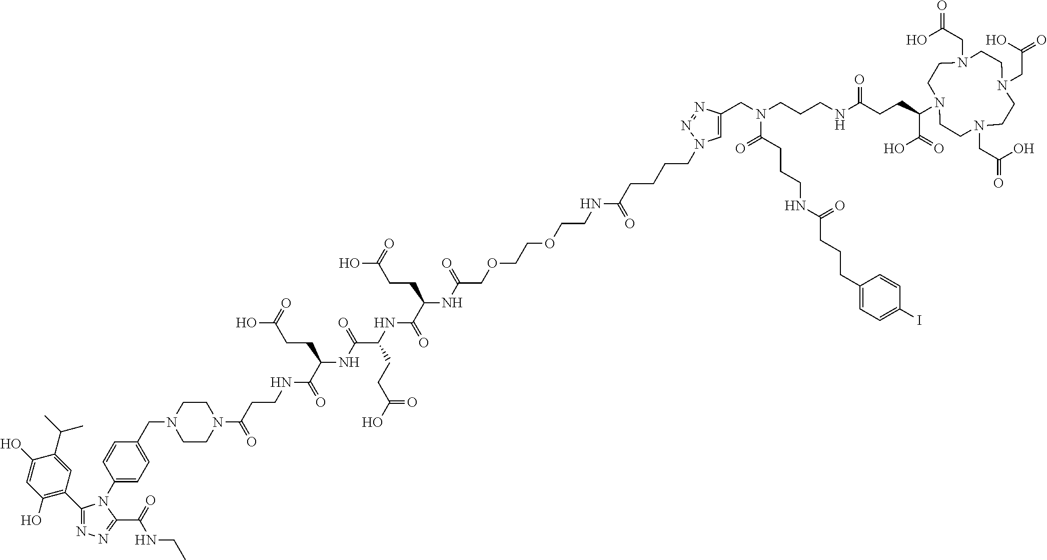 [SMILES: CCNC(=O)c1nnc(-c2cc(C(C)C)c(O)cc2O)n1-c1ccc(CN2CCN(C(=O)CCNC(=O)[C@@H](CCC(=O)O)NC(=O)[C@@H](CCC(=O)O)NC(=O)[C@@H](CCC(=O)O)NC(=O)COCCOCCNC(=O)CCCCn3cc(CN(CCCNC(=O)CC[C@H](C(=O)O)N4CCN(CC(=O)O)CCN(CC(=O)O)CCN(CC(=O)O)CC4)C(=O)CCCNC(=O)CCCc4ccc(I)cc4)nn3)CC2)cc1]

CMP51′: comprising one TM5 ligand and an amino acid spacer; encompassed by Formula C2

In some embodiments, the conjugate comprises at least one albumin-binding PMU and comprises at least one TM1 as a ligand. In some embodiments, the conjugate comprises at least one albumin-binding PMU and comprises 1 TM1 as a ligand. In some embodiments, the conjugate comprises at least one albumin-binding PMU and comprises two TM1s as ligands, such as T17.

In some embodiments, the conjugate comprises at least one albumin-binding PMU and comprises at least one TM2 as a ligand. In some embodiments, the conjugate comprises at least one albumin-binding PMU and comprises 1 TM2 as a ligand. In some embodiments, the conjugate comprises at least one albumin-binding PMU and comprises two TM2s as ligands, such as T13.

In some embodiments, the conjugate comprises at least one albumin-binding PMU and comprises at least one TM5 as a ligand. In some embodiments, the conjugate comprises at least one albumin-binding PMU and comprises 1 TM5 as a ligand, such as T7. In some embodiments, the conjugate comprises at least one albumin-binding PMU and comprises two TM5s as ligands. Non-limiting examples include T3, T14, T15, T16, T22, T23, T32, T24, T33, T34, T35, T36, T37, T38, T41, and T42.

In some embodiments, the conjugate comprises at least one albumin-binding PMU and comprises at least one TM9 as a ligand. In some embodiments, the conjugate comprises at least one albumin-binding PMU and comprises 1 TM9 as a ligand, such as CMP3 and CMP4.

In some embodiments, the conjugate comprises at least one albumin-binding PMU and comprises at least one TM10 as a ligand. In some embodiments, the conjugate comprises at least one albumin-binding PMU and comprises 1 TM10 as a ligand, such as CMP5, CMP6, and T11. In some embodiments, the conjugate comprises at least one amino acid spacer and comprises two TM10s as ligands, such as T12.

Lutetium (Lu) in Conjugate T1, T2, T3, T4, T5, T6, T7, T8, T9, T10, T11, T12, T13, T14, T15, T16, T17, T18, T19, T20, T21, T22, T23, T24, T25, T26, T27, T28, T29, T30, T31, T32, T33, T34, T35, T36, T37, T38, T39, T40, T41, T42, T43, CMP1, CMP2, CMP3, CMP4, CMP5, CMP6, CMP7, CMP8, CMP9, CMP10, CMP11, CMP12, CMP13, CMP14, CMP15, CMP16, CMP17, CMP18, CMP19, CMP20, CMP21, CMP22, CMP23, CMP24, CMP25, CMP26, CMP27, CMP28, CMP29, CMP30, CMP31, CMP32, CMP33, CMP34, CMP35, CMP36, CMP37, CMP38, CMP39, CMP40, CMP41, CMP42, CMP43, CMP44, CMP45, CMP46, CMP47, CMP48, C4MP9, CMP50, or CMP52 may be replaced with Lu177 (¹⁷⁷Lu) or any other radioisotope (such as Y-90, Y-86, I-131, Re-186, Re-188, Y-90, Bi-212, At-211, Zr-89, Sr-89, Ho-166, Sm-153, Cu-67, Cu-64, Ac-225, Pb-203, Bi-213, Th-227, Pb-212, Ra-223, P-32, Sc-47, Br-77, Rh-105, Pd-103, Ag-111, Pr-142, Pm-149, Gd-159, Ir-194 and Pt-199) to render a radioactive analog of the conjugate.

### II. Formulations

In some embodiments, compositions are administered to humans, human patients or subjects. For the purposes of the present disclosure, the phrase “active ingredient” generally refers to the conjugate as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 100%, e.g., between 0.5 and 50%, between 1-30%, between 5-80%, at least 80% (w/w) active ingredient.

The conjugates of the present invention can be formulated using one or more excipients to: (1) increase stability; (2) permit the sustained or delayed release (e.g., from a depot formulation of the monomaleimide); (3) alter the biodistribution (e.g., target the monomaleimide compounds to specific tissues or cell types); (4) alter the release profile of the monomaleimide compounds in vivo. Non-limiting examples of the excipients include any and all solvents, dispersion media, diluents, or other liquid vehicles, dispersion or suspension aids, surface active agents, isotonic agents, thickening or emulsifying agents, and preservatives. Excipients of the present invention may also include, without limitation, lipidoids, liposomes, lipid nanoparticles, polymers, lipoplexes, core-shell nanoparticles, peptides, proteins, hyaluronidase, nanoparticle mimics and combinations thereof. Accordingly, the formulations of the invention may include one or more excipients, each in an amount that together increases the stability of the monomaleimide compounds.

### Excipients

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro (Lippincott, Williams & Wilkins, Baltimore, Md., 2006; incorporated herein by reference in its entirety) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEEN®60], polyoxyethylene sorbitan monooleate [TWEEN®80], sorbitan monopalmitate [SPAN®40], sorbitan monostearate [SPAN®60], sorbitan tristearate [SPAN®65], glyceryl monooleate, sorbitan monooleate [SPAN®80]), polyoxyethylene esters (e.g. polyoxyethylene monostearate [MYRJ®45], polyoxyethylene hydrogenated castor oil, polyethoxylated castor oil, polyoxymethylene stearate, and Kolliphor® (SOLUTOL®)), sucrose fatty acid esters, polyethylene glycol fatty acid esters (e.g. CREMOPHOR®), polyoxyethylene ethers, (e.g. polyoxyethylene lauryl ether [BRIJ®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLUORINC®F 68, POLOXAMER®188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol,); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and/or other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid. Exemplary alcohol preservatives include, but are not limited to, ethanol, polyethylene glycol, phenol, phenolic compounds, bisphenol, chlorobutanol, hydroxybenzoate, and/or phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL®115, GERMABEN®II, NEOLONE™, KATHON™, and/or EUXYL®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Excipients such as cocoa butter and suppository waxes, coloring agents, coating agents, sweetening, flavoring, and/or perfuming agents can be present in the composition, according to the judgment of the formulator.

### Administration

The conjugates of the present invention may be administered by any route which results in a therapeutically effective outcome. These include, but are not limited to enteral, gastroenteral, epidural, oral, transdermal, epidural (peridural), intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection, (into the base of the penis), intravaginal administration, intrauterine, extra-amniotic administration, transdermal (diffusion through the intact skin for systemic distribution), transmucosal (diffusion through a mucous membrane), insufflation (snorting), sublingual, sublabial, enema, eye drops (onto the conjunctiva), or in ear drops. In specific embodiments, compositions may be administered in a way which allows them cross the blood-brain barrier, vascular barrier, or other epithelial barrier.

The formulations described herein contain an effective amount of conjugates in a pharmaceutical carrier appropriate for administration to an individual in need thereof. The formulations may be administered parenterally (e.g., by injection or infusion). The formulations or variations thereof may be administered in any manner including enterally, topically (e.g., to the eye), or via pulmonary administration. In some embodiments the formulations are administered topically.

### Dosing

The present invention provides methods comprising administering conjugates as described herein to a subject in need thereof. Conjugates as described herein may be administered to a subject using any amount and any route of administration effective for preventing or treating or imaging a disease, disorder, and/or condition (e.g., a disease, disorder, and/or condition relating to working memory deficits). The exact amount required will vary from subject to subject, depending on the species, age, and general condition of the subject, the severity of the disease, the particular composition, its mode of administration, its mode of activity, and the like.

Compositions in accordance with the invention are typically formulated in dosage unit form for ease of administration and uniformity of dosage. It will be understood, however, that the total daily usage of the compositions of the present invention may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In some embodiments, compositions in accordance with the present invention may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, from about 25 mg/kg to about 50 mg/kg, from about 50 mg/kg to about 100 mg/kg, from about 100 mg/kg to about 125 mg/kg, from about 125 mg/kg to about 150 mg/kg, from about 150 mg/to about 175 mg/kg, from about 175 mg/kg to about 200 mg/kg, from about 200 mg/kg to about 250 mg/kg of subject body weight per day, one or more times a day, to obtain the desired therapeutic, diagnostic, prophylactic, or imaging effect. The desired dosage may be delivered three times a day, two times a day, once a day, every other day, every third day, every week, every two weeks, every three weeks, or every four weeks. In some embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used.

The concentration of the conjugates may be between about 0.01 mg/mL to about 50 mg/mL, about 0.1 mg/mL to about 25 mg/mL, about 0.5 mg/mL to about 10 mg/mL, or about 1 mg/mL to about 5 mg/mL in the pharmaceutical composition.

As used herein, a “split dose” is the division of single unit dose or total daily dose into two or more doses, e.g, two or more administrations of the single unit dose. As used herein, a “single unit dose” is a dose of any therapeutic administered in one dose/at one time/single route/single point of contact, i.e., single administration event. As used herein, a “total daily dose” is an amount given or prescribed in 24 hr period. It may be administered as a single unit dose.

### Dosage Forms

A pharmaceutical composition described herein can be formulated into a dosage form described herein, such as a topical, intranasal, intratracheal, or injectable (e.g., intravenous, intraocular, intravitreal, intramuscular, intracardiac, intraperitoneal, and subcutaneous).

### VI. Methods of Using the Conjugates

The conjugates as described herein can be administered to treat any hyperproliferative disease, metabolic disease, infectious disease, or cancer, as appropriate. Formulations may be administered by injection, orally, or topically, typically to a mucosal surface (lung, nasal, oral, buccal, sublingual, vaginally, rectally) or to the eye (intraocularly or transocularly).

In various embodiments, methods for treating a subject having a cancer are provided, wherein the method comprises administering a therapeutically-effective amount of the conjugates, salt forms thereof, as described herein, to a subject having a cancer, suspected of having cancer, or having a predisposition to a cancer. According to the present invention, cancer embraces any disease or malady characterized by uncontrolled cell proliferation, e.g., hyperproliferation. Cancers may be characterized by tumors, e.g., solid tumors or any neoplasm.

In some embodiments, the cancer is a solid tumor. Large drug molecules have limited penetration in solid tumors. The penetration of large drug molecules is slow. On the other hand, small molecules such as conjugates of the present invention may penetrate solid tumors rapidly and more deeply. Regarding penetration depth of the drugs, larger molecules penetrate less, despite having more durable pharmacokinetics. Small molecules such as conjugates of the present invention penetrate deeper. Dreher et al. (Dreher et al., JNCI, vol. 98(5):335 (2006), the contents of which are incorporated herein by reference in their entirety) studied penetration of dextrans with different sizes into a tumor xenograft.

In one embodiment, conjugates of the present invention reach at least about 25 μm, about 30 μm, about 35 μm, about 40 μm, about 45 μm, about 50 μm, about 75 μm, about 100 μm, about 150 μm, about 200 μm, about 250 μm, about 300 μm, about 400 μm, about 500 μm, about 600 μm, about 700 μm, about 800 μm, about 900 μm, about 1000 μm, about 1100 μm, about 1200 μm, about 1300 μm, about 1400 μm or about 1500 μm into the solid tumor from the vascular surface of the tumor. Zero distance is defined as the vascular surface of the tumor, and every distance greater than zero is defined as the distance measured in three dimensions to the nearest vascular surface.

In another embodiment, conjugates of the present invention penetrate to the core of the tumor. “Core” of the tumor, as used herein, refers to the central area of the tumor. The distance from any part of the core area of the tumor to the vascular surface of the tumor is between about 30% to about 50% of the length or width of the tumor. The distance from any part of the core area of the tumor to the center point of the tumor is less than about 20% of the length or width of the tumor. The core area of the tumor is roughly the center ⅓ of the tumor.

In another embodiment, conjugates of the present invention conjugates of the present invention penetrate to the middle of the solid tumor. “Middle” of the tumor, as sued herein, refers to the middle area of the tumor. The distance from any part of the middle area of the tumor to the vascular surface of the tumor is between about 15% and about 30% of the length or the width of the tumor. The distance from any part of the middle area of the tumor to the center point of the tumor is between about 20% to about 35% of the length or width of the tumor. The middle area of the tumor is roughly between the center ⅓ of the tumor and the outer ⅓ of the tumor.

In some embodiments, the subject may be otherwise free of indications for treatment with the conjugates. In some embodiments, methods include use of cancer cells, including but not limited to mammalian cancer cells. In some instances, the mammalian cancer cells are human cancer cells.

In some embodiments, the conjugates of the present teachings have been found to inhibit cancer and/or tumor growth. They may also reduce, including cell proliferation, invasiveness, and/or metastasis, thereby rendering them useful for the treatment of a cancer.

In some embodiments, the conjugates of the present teachings may be used to prevent the growth of a tumor or cancer, and/or to prevent the metastasis of a tumor or cancer. In some embodiments, compositions of the present teachings may be used to shrink or destroy a cancer.

In some embodiments, the conjugates provided herein are useful for inhibiting proliferation of a cancer cell. In some embodiments, the conjugates provided herein are useful for inhibiting cellular proliferation, e.g., inhibiting the rate of cellular proliferation, preventing cellular proliferation, and/or inducing cell death. In general, the conjugates as described herein can inhibit cellular proliferation of a cancer cell or both inhibiting proliferation and/or inducing cell death of a cancer cell. In some embodiments, cell proliferation is reduced by at least about 25%, about 50%, about 75%, or about 90% after treatment with conjugates of the present invention compared with cells with no treatment. In some embodiments, cell cycle arrest marker phospho histone H3 (PH3 or PHH3) is increased by at least about 50%, about 75%, about 100%, about 200%, about 400% or about 600% after treatment with conjugates of the present invention compared with cells with no treatment. In some embodiments, cell apoptosis marker cleaved caspase-3 (CC3) is increased by at least 50%, about 75%, about 100%, about 200%, about 400% or about 600% after treatment with conjugates of the present invention compared with cells with no treatment.

Furthermore, in some embodiments, conjugates of the present invention are effective for inhibiting tumor growth, whether measured as a net value of size (weight, surface area or volume) or as a rate over time, in multiple types of tumors.

In some embodiments the size of a tumor is reduced by about 60% or more after treatment with conjugates of the present invention. In some embodiments, the size of a tumor is reduced by at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 100%, by a measure of weight, and/or area and/or volume.

The cancers treatable by methods of the present teachings generally occur in mammals. Mammals include, for example, humans, non-human primates, dogs, cats, rats, mice, rabbits, ferrets, guinea pigs horses, pigs, sheep, goats, and cattle. In various embodiments, Cancers include, but are not limited to, acoustic neuroma, acute leukemia, acute lymphocytic leukemia, acute myelocytic leukemia (monocytic, myeloblastic, adenocarcinoma, angiosarcoma, astrocytoma, myelomonocytic and promyelocytic), acute T-cell leukemia, basal cell carcinoma, bile duct carcinoma, bladder cancer, brain cancer, breast cancer, bronchogenic carcinoma, cervical cancer, chondrosarcoma, chordoma, choriocarcinoma, chronic leukemia, chronic lymphocytic leukemia, chronic myelocytic (granulocytic) leukemia, chronic myelogenous leukemia, colon cancer, colorectal cancer, craniopharyngioma, cystadenocarcinoma, diffuse large B-cell lymphoma, Burkitt's lymphoma, dysproliferative changes (dysplasias and metaplasias), embryonal carcinoma, endometrial cancer, endotheliosarcoma, ependymoma, epithelial carcinoma, erythroleukemia, esophageal cancer, estrogen-receptor positive breast cancer, essential thrombocythemia, Ewing's tumor, fibrosarcoma, follicular lymphoma, germ cell testicular cancer, glioma, heavy chain disease, hemangioblastoma, hepatoma, hepatocellular cancer, hormone insensitive prostate cancer, leiomyosarcoma, liposarcoma, lung cancer, lymphagioendotheliosarcoma, lymphangiosarcoma, lymphoblastic leukemia, lymphoma (Hodgkin's and non-Hodgkin's), malignancies and hyperproliferative disorders of the bladder, breast, colon, lung, ovaries, pancreas, prostate, skin, and uterus, lymphoid malignancies of T-cell or B-cell origin, leukemia, lymphoma, medullary carcinoma, medulloblastoma, melanoma, meningioma, mesothelioma, multiple myeloma, myelogenous leukemia, myeloma, myxosarcoma, neuroblastoma, non-small cell lung cancer, oligodendroglioma, oral cancer, osteogenic sarcoma, ovarian cancer, pancreatic cancer, papillary adenocarcinomas, papillary carcinoma, pinealoma, polycythemia vera, prostate cancer, rectal cancer, renal cell carcinoma, retinoblastoma, rhabdomyosarcoma, sarcoma, sebaceous gland carcinoma, seminoma, skin cancer, small cell lung carcinoma, solid tumors (carcinomas and sarcomas), small cell lung cancer, stomach cancer, squamous cell carcinoma, synovioma, sweat gland carcinoma, thyroid cancer, Waldenstrom's macroglobulinemia, testicular tumors, uterine cancer, and Wilms' tumor. Other cancers include primary cancer, metastatic cancer, oropharyngeal cancer, hypopharyngeal cancer, liver cancer, gall bladder cancer, bile duct cancer, small intestine cancer, urinary tract cancer, kidney cancer, urothelium cancer, female genital tract cancer, uterine cancer, gestational trophoblastic disease, male genital tract cancer, seminal vesicle cancer, testicular cancer, germ cell tumors, endocrine gland tumors, thyroid cancer, adrenal cancer, pituitary gland cancer, hemangioma, sarcoma arising from bone and soft tissues, Kaposi's sarcoma, nerve cancer, ocular cancer, meningial cancer, glioblastomas, neuromas, neuroblastomas, Schwannomas, solid tumors arising from hematopoietic malignancies such as leukemias, metastatic melanoma, recurrent or persistent ovarian epithelial cancer, fallopian tube cancer, primary peritoneal cancer, gastrointestinal stromal tumors, colorectal cancer, gastric cancer, melanoma, glioblastoma multiforme, non-squamous non-small-cell lung cancer, malignant glioma, epithelial ovarian cancer, primary peritoneal serous cancer, metastatic liver cancer, neuroendocrine carcinoma, refractory malignancy, triple negative breast cancer, HER2-amplified breast cancer, nasopharageal cancer, oral cancer, biliary tract, hepatocellular carcinoma, squamous cell carcinomas of the head and neck (SCCHN), non-medullary thyroid carcinoma, recurrent glioblastoma multiforme, neurofibromatosis type 1, CNS cancer, liposarcoma, leiomyosarcoma, salivary gland cancer, mucosal melanoma, acral/lentiginous melanoma, paraganglioma, pheochromocytoma, advanced metastatic cancer, solid tumor, triple negative breast cancer, colorectal cancer, sarcoma, melanoma, renal carcinoma, endometrial cancer, thyroid cancer, rhabdomysarcoma, multiple myeloma, ovarian cancer, glioblastoma, gastrointestinal stromal tumor, mantle cell lymphoma, and refractory malignancy.

In one embodiment, the conjugates as described herein or formulations containing the conjugates as described herein are used to treat small cell lung cancer. About 12%-15% of patients having lung cancer have small cell lung cancer. Survival in metastatic small cell lung cancer is poor. Survival rate is below 5% five years after diagnosis. US incidence of small cell lung cancer is about 26K-30K.

In some embodiments, the conjugates as described herein or formulations containing the conjugates as described herein are used to treat patients with tumors that express or over-express the HSP90.

A feature of conjugates of the present invention is relatively low toxicity to an organism while maintaining efficacy at inhibiting, e.g. slowing or stopping tumor growth. As used herein, “toxicity” refers to the capacity of a substance or composition to be harmful or poisonous to a cell, tissue organism or cellular environment. Low toxicity refers to a reduced capacity of a substance or composition to be harmful or poisonous to a cell, tissue organism or cellular environment. Such reduced or low toxicity may be relative to a standard measure, relative to a treatment or relative to the absence of a treatment. For example, conjugates of the present invention may have lower toxicity than the active agent moiety Z administered alone. For conjugates comprising DM1, their toxicity is lower than DM1 administered alone.

Toxicity may further be measured relative to a subject's weight loss where weight loss over 15%, over 20% or over 30% of the body weight is indicative of toxicity. Other metrics of toxicity may also be measured such as patient presentation metrics including lethargy and general malaise. Neutropenia, thrombocytopenia, white blood cell (WBC) count, complete blood cell (CBC) count may also be metrics of toxicity. Pharmacologic indicators of toxicity include elevated aminotransferases (AST/ALT) levels, neurotoxicity, kidney damage, GI damage and the like. In one embodiment, conjugates of the present invention do not cause a significant change of a subject's body weight. The body weight loss of a subject is less about 30%, about 20%, about 15%, about 10%, or about 5% after treatment with conjugates of the present invention. In another embodiment, conjugates of the present invention do not cause a significant increase of a subject's AST/ALT levels. The AST or ALT level of a subject is increased by less than about 30%, about 20%, about 15%, about 10%, or about 5% after treatment with conjugates of the present invention. In yet another embodiment, conjugates of the present invention do not cause a significant change of a subject's CBC or WBC count after treatment with conjugates of the present invention. The CBC or WBC level of a subject is decreased by less than about 30%, about 20%, about 15%, about 10%, or about 5% after treatment with conjugates of the present invention.

### Combination Therapies

In some embodiments, conjugates of the present invention are combined with at least one additional active agent. The active agent may be any suitable drug. The conjugates and the at least one additional active agent may be administered simultaneously, sequentially, or at any order. The conjugates and the at least one additional active agent may be administered at different dosages, with different dosing frequencies, or via different routes, whichever is suitable.

In some embodiments, the additional active agents affect the biodistribution (i.e., tissue distribution) of the conjugates of the current invention. For example, radioactive agents may accumulate in kidneys and may pose a potential radiotoxicity problem to kidneys and surrounding organs. The additional active agent may reduce renal accumulation or retention time. Preferably, kidney update of the conjugates is reduced, while tumor uptake of the conjugates is not affected. Kidney and surrounding organs are protected without reducing the efficacy of the conjugates. In one non-limiting example, conjugates of the current invention may be administered in combination with at least one amino acid or analog(s) thereof. The amino acid or analog(s) thereof may be positively charged basic amino acids such as lysine (L-lysine or D-lysine) or arginine, or a combination thereof. In another non-limiting example, conjugates of the current invention may be administered in combination with an active agent that binds to HSP90, such as an HSP90 inhibitor. Any ligand discussed in the “HSP90 Targeting Moieties” section, such as ganetespib or its derivative/analog thereof, may be used. In another non-limiting example, conjugates of the current invention may be administered in combination with monosodium glutamate (MSG) or glutamic acid. In yet another non-limiting example, conjugates of the current invention may be administered in combination with amifostine (Ethyol, WR-2721), the bovine gelatin-containing solution Gelofusine or albumin fragments. The albumin fragments may have a molecular weight between 3 and 50 kDa.

The additional active agent may also be selected from any active agent described herein such as a drug for treating cancer. It may also be a cancer symptom relief drug. Non-limiting examples of symptom relief drugs include: octreotide or lanreotide; interferon, cypoheptadine or any other antihistamines. In some embodiments, conjugates of the present invention do not have drug-drug interference with the additional active agent. In one embodiment, conjugates of the present invention do not inhibit cytochrome P450 (CYP) isozymes. CYP isozymes may include CYP3A4 Midazolam, CYP3A4 Testosterone, CYP2C9, CYP2D6, CYP1A2, CYP2C8, CYP2B6, and CYP2C19. The additional active agent may be administered concomitantly with conjugates of the present invention.

In another example, conjugates of the present invention may be combined with a moderate dose of chemotherapy agents such as mitomycin C, vinblastine and cisplatin (see Ellis et al., Br J Cancer, vol. 71(2): 366-370 (1995), the contents of which are incorporated herein by reference in their entirety).

In yet another example, a patient may first receive a pharmaceutically effective dose of an unconjugated active agent, followed by a pharmaceutically effective dose of a conjugate comprising the same active agent.

In some embodiments, a non-radioactive conjugate of the present invention may be combined with an radioactive analog of this conjugate. For example, the non-radioactive conjugate can be administered prior to the radioactive analog. In another example, a subject may receive a mixture of the non-radioactive conjugate and its radioactive analog. In yet another example, a subject may receive the non-radioactive conjugate treatment first, followed by a mixture of the non-radioactive conjugate and its radioactive analog.

In some embodiments, a conjugate of the present invention comprising one radiolabel may be combined with at least one other conjugate of the present invention comprising one or more different radiolabels. For example, conjugates comprising an imaging radiolabel may be combined with conjugates comprising a non-imaging radiolabel. In one embodiment, conjugates comprising lutetium (Lu) may be combined with conjugates comprising gallium (Ga).

The conjugates as described herein or formulations containing the conjugates as described herein can be used for the selective tissue delivery of a therapeutic, prophylactic, or diagnostic agent to an individual or patient in need thereof. For example, conjugates of the present invention are used to deliver radioactive agents to selective tissues. These tissues may be tumor tissues. Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. Dosage unit form as used herein refers to physically discrete units suited as unitary dosages for the mammalian subjects to be treated; each unit containing a predetermined quantity of active compound calculated to produce the desired therapeutic.

### V. Kits and Devices

The invention provides a variety of kits and devices for conveniently and/or effectively carrying out methods of the present invention. Typically kits will comprise sufficient amounts and/or numbers of components to allow a user to perform multiple treatments of a subject(s) and/or to perform multiple experiments.

In one embodiment, the present invention provides kits for inhibiting tumor cell growth in vitro or in vivo, comprising a conjugate of the present invention or a combination of conjugates of the present invention, optionally in combination with any other active agents.

The kit may further comprise packaging and instructions and/or a delivery agent to form a formulation composition. The delivery agent may comprise a saline, a buffered solution, or any delivery agent disclosed herein. The amount of each component may be varied to enable consistent, reproducible higher concentration saline or simple buffer formulations. The components may also be varied in order to increase the stability of the conjugates in the buffer solution over a period of time and/or under a variety of conditions.

The present invention provides for devices which may incorporate conjugates of the present invention. These devices contain in a stable formulation available to be immediately delivered to a subject in need thereof, such as a human patient. In some embodiments, the subject has cancer.

Non-limiting examples of the devices include a pump, a catheter, a needle, a transdermal patch, a pressurized olfactory delivery device, iontophoresis devices, multi-layered microfluidic devices. The devices may be employed to deliver conjugates of the present invention according to single, multi- or split-dosing regiments. The devices may be employed to deliver conjugates of the present invention across biological tissue, intradermal, subcutaneously, or intramuscularly.

### VI. Definitions

The term “compound”, as used herein, is meant to include all stereoisomers, geometric isomers, tautomers, and isotopes of the structures depicted. In the present application, compound is used interchangeably with conjugate. Therefore, conjugate, as used herein, is also meant to include all stereoisomers, geometric isomers, tautomers, and isotopes of the structures depicted.

The compounds described herein can be asymmetric (e.g., having one or more stereocenters). All stereoisomers, such as enantiomers and diastereomers, are intended unless otherwise indicated. Compounds of the present disclosure that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present disclosure. Cis and trans geometric isomers of the compounds of the present disclosure are described and may be isolated as a mixture of isomers or as separated isomeric forms.

Compounds of the present disclosure also include tautomeric forms. Tautomeric forms result from the swapping of a single bond with an adjacent double bond and the concomitant migration of a proton. Tautomeric forms include prototropic tautomers which are isomeric protonation states having the same empirical formula and total charge. Examples prototropic tautomers include ketone-enol pairs, amide-imidic acid pairs, lactam-lactim pairs, amide-imidic acid pairs, enamine-imine pairs, and annular forms where a proton can occupy two or more positions of a heterocyclic system, such as, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

Compounds of the present disclosure also include all of the isotopes of the atoms occurring in the intermediate or final compounds. “Isotopes” refers to atoms having the same atomic number but different mass numbers resulting from a different number of neutrons in the nuclei. For example, isotopes of hydrogen include tritium and deuterium.

The compounds and salts of the present disclosure can be prepared in combination with solvent or water molecules to form solvates and hydrates by routine methods.

The terms “subject” or “patient”, as used herein, refer to any organism to which the conjugates may be administered, e.g., for experimental, therapeutic, diagnostic, and/or prophylactic purposes. Typical subjects include animals (e.g., mammals such as mice, rats, rabbits, guinea pigs, cattle, pigs, sheep, horses, dogs, cats, hamsters, lamas, non-human primates, and humans).

The terms “treating” or “preventing”, as used herein, can include preventing a disease, disorder or condition from occurring in an animal that may be predisposed to the disease, disorder and/or condition but has not yet been diagnosed as having the disease, disorder or condition; inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, such as treating the pain of a subject by administration of an analgesic agent even though such agent does not treat the cause of the pain.

A “target”, as used herein, shall mean a site to which targeted constructs bind. A target may be either in vivo or in vitro. In certain embodiments, a target may be cancer cells found in leukemias or tumors (e.g., tumors of the brain, lung (small cell and non-small cell), ovary, prostate, breast and colon as well as other carcinomas and sarcomas). In still other embodiments, a target may refer to a molecular structure to which a targeting moiety or ligand binds, such as a hapten, epitope, receptor, dsDNA fragment, carbohydrate or enzyme. A target may be a type of tissue, e.g., neuronal tissue, intestinal tissue, pancreatic tissue, liver, kidney, prostate, ovary, lung, bone marrow, or breast tissue.

The “target cells” that may serve as the target for the method or conjugates, are generally animal cells, e.g., mammalian cells. The present method may be used to modify cellular function of living cells in vitro, i.e., in cell culture, or in vivo, in which the cells form part of or otherwise exist in animal tissue. Thus, the target cells may include, for example, the blood, lymph tissue, cells lining the alimentary canal, such as the oral and pharyngeal mucosa, cells forming the villi of the small intestine, cells lining the large intestine, cells lining the respiratory system (nasal passages/lungs) of an animal (which may be contacted by inhalation of the subject invention), dermal/epidermal cells, cells of the vagina and rectum, cells of internal organs including cells of the placenta and the so-called blood/brain barrier, etc. In general, a target cell expresses at least one type of HSP90. In some embodiments, a target cell can be a cell that expresses an HSP90 and is targeted by a conjugate described herein, and is near a cell that is affected by release of the active agent of the conjugate. For example, a blood vessel expressing an HSP90 that is in proximity to a tumor may be the target, while the active agent released at the site will affect the tumor.

The term “therapeutic effect” is art-recognized and refers to a local or systemic effect in animals, particularly mammals, and more particularly humans caused by a pharmacologically active substance. The term thus means any substance intended for use in the diagnosis, cure, mitigation, treatment or prevention of disease, disorder or condition in the enhancement of desirable physical or mental development and conditions in an animal, e.g., a human.

The term “modulation” is art-recognized and refers to up regulation (i.e., activation or stimulation), down regulation (i.e., inhibition or suppression) of a response, or the two in combination or apart. The modulation is generally compared to a baseline or reference that can be internal or external to the treated entity.

“Parenteral administration”, as used herein, means administration by any method other than through the digestive tract (enteral) or non-invasive topical routes. For example, parenteral administration may include administration to a patient intravenously, intradermally, intraperitoneally, intrapleurally, intratracheally, intraossiously, intracerebrally, intrathecally, intramuscularly, subcutaneously, subjunctivally, by injection, and by infusion.

“Topical administration”, as used herein, means the non-invasive administration to the skin, orifices, or mucosa. Topical administration can be delivered locally, i.e., the therapeutic can provide a local effect in the region of delivery without systemic exposure or with minimal systemic exposure. Some topical formulations can provide a systemic effect, e.g., via adsorption into the blood stream of the individual. Topical administration can include, but is not limited to, cutaneous and transdermal administration, buccal administration, intranasal administration, intravaginal administration, intravesical administration, ophthalmic administration, and rectal administration.

“Enteral administration”, as used herein, means administration via absorption through the gastrointestinal tract. Enteral administration can include oral and sublingual administration, gastric administration, or rectal administration.

“Pulmonary administration”, as used herein, means administration into the lungs by inhalation or endotracheal administration. As used herein, the term “inhalation” refers to intake of air to the alveoli. The intake of air can occur through the mouth or nose.

The terms “sufficient” and “effective”, as used interchangeably herein, refer to an amount (e.g., mass, volume, dosage, concentration, and/or time period) needed to achieve one or more desired result(s). A “therapeutically effective amount” is at least the minimum concentration required to effect a measurable improvement or prevention of at least one symptom or a particular condition or disorder, to effect a measurable enhancement of life expectancy, or to generally improve patient quality of life. The therapeutically effective amount is thus dependent upon the specific biologically active molecule and the specific condition or disorder to be treated. Therapeutically effective amounts of many active agents, such as antibodies, are known in the art. The therapeutically effective amounts of compounds and compositions described herein, e.g., for treating specific disorders may be determined by techniques that are well within the craft of a skilled artisan, such as a physician.

The terms “bioactive agent” and “active agent”, as used interchangeably herein, include, without limitation, physiologically or pharmacologically active substances that act locally or systemically in the body. A bioactive agent is a substance used for the treatment (e.g., therapeutic agent), prevention (e.g., prophylactic agent), diagnosis (e.g., diagnostic agent), cure or mitigation of disease or illness, a substance which affects the structure or function of the body, or pro-drugs, which become biologically active or more active after they have been placed in a predetermined physiological environment.

The term “prodrug” refers to an agent, including a small organic molecule, peptide, nucleic acid or protein, that is converted into a biologically active form in vitro and/or in vivo. Prodrugs can be useful because, in some situations, they may be easier to administer than the parent compound (the active compound). For example, a prodrug may be bioavailable by oral administration whereas the parent compound is not. The prodrug may also have improved solubility in pharmaceutical compositions compared to the parent drug. A prodrug may also be less toxic than the parent. A prodrug may be converted into the parent drug by various mechanisms, including enzymatic processes and metabolic hydrolysis. Harper, N.J. (1962) Drug Latentiation in Jucker, ed. Progress in Drug Research, 4:221-294; Morozowich et al. (1977) Application of Physical Organic Principles to Prodrug Design in E. B. Roche ed. Design of Biopharmaceutical Properties through Prodrugs and Analogs, APhA; Acad. Pharm. Sci.; E. B. Roche, ed. (1977) Bioreversible Carriers in Drug in Drug Design, Theory and Application, APhA; H. Bundgaard, ed. (1985) Design of Prodrugs, Elsevier; Wang et al. (1999) Prodrug approaches to the improved delivery of peptide drug, Curr. Pharm. Design. 5(4):265-287; Pauletti et al. (1997) Improvement in peptide bioavailability: Peptidomimetics and Prodrug Strategies, Adv. Drug. Delivery Rev. 27:235-256; Mizen et al. (1998). The Use of Esters as Prodrugs for Oral Delivery of β-Lactam antibiotics, Pharm. Biotech. 11:345-365; Gaignault et al. (1996) Designing Prodrugs and Bioprecursors I. Carrier Prodrugs, Pract. Med. Chem. 671-696; M. Asgharnejad (2000). Improving Oral Drug Transport Via Prodrugs, in G. L. Amidon, P. I. Lee and E. M. Topp, Eds., Transport Processes in Pharmaceutical Systems, Marcell Dekker, p. 185-218; Balant et al. (1990) Prodrugs for the improvement of drug absorption via different routes of administration, Eur. J. Drug Metab. Pharmacokinet., 15(2): 143-53; Balimane and Sinko (1999). Involvement of multiple transporters in the oral absorption of nucleoside analogues, Adv. Drug Delivery Rev., 39(1-3):183-209; Browne (1997). Fosphenytoin (Cerebyx), Clin. Neuropharmacol. 20(1): 1-12; Bundgaard (1979). Bioreversible derivatization of drugs—principle and applicability to improve the therapeutic effects of drugs, Arch. Pharm. Chemi. 86(1): 1-39; H. Bundgaard, ed. (1985) Design of Prodrugs, New York: Elsevier; Fleisher et al. (1996) Improved oral drug delivery: solubility limitations overcome by the use of prodrugs, Adv. Drug Delivery Rev. 19(2): 115-130; Fleisher et al. (1985) Design of prodrugs for improved gastrointestinal absorption by intestinal enzyme targeting, Methods Enzymol. 112: 360-81; Farquhar D, et al. (1983) Biologically Reversible Phosphate-Protective Groups, J. Pharm. Sci., 72(3): 324-325; Han, H. K. et al. (2000) Targeted prodrug design to optimize drug delivery, AAPS PharmSci., 2(1): E6; Sadzuka Y. (2000) Effective prodrug liposome and conversion to active metabolite, Curr. Drug Metab., 1(1):31-48; D. M. Lambert (2000) Rationale and applications of lipids as prodrug carriers, Eur. J. Pharm. Sci., 11 Suppl. 2:S15-27; Wang, W. et al. (1999) Prodrug approaches to the improved delivery of peptide drugs. Curr. Pharm. Des., 5(4):265-87.

The term “biocompatible”, as used herein, refers to a material that along with any metabolites or degradation products thereof that are generally non-toxic to the recipient and do not cause any significant adverse effects to the recipient. Generally speaking, biocompatible materials are materials which do not elicit a significant inflammatory or immune response when administered to a patient.

The term “biodegradable” as used herein, generally refers to a material that will degrade or erode under physiologic conditions to smaller units or chemical species that are capable of being metabolized, eliminated, or excreted by the subject. The degradation time is a function of composition and morphology. Degradation times can be from hours to weeks.

The term “pharmaceutically acceptable”, as used herein, refers to compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio, in accordance with the guidelines of agencies such as the U.S. Food and Drug Administration. A “pharmaceutically acceptable carrier”, as used herein, refers to all components of a pharmaceutical formulation that facilitate the delivery of the composition in vivo. Pharmaceutically acceptable carriers include, but are not limited to, diluents, preservatives, binders, lubricants, disintegrators, swelling agents, fillers, stabilizers, and combinations thereof.

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

The term “small molecule”, as used herein, generally refers to an organic molecule that is less than 2000 g/mol in molecular weight, less than 1500 g/mol, less than 1000 g/mol, less than 800 g/mol, or less than 500 g/mol. Small molecules are non-polymeric and/or non-oligomeric.

The term “hydrophilic”, as used herein, refers to substances that have strongly polar groups that readily interact with water.

The term “hydrophobic”, as used herein, refers to substances that lack an affinity for water; tending to repel and not absorb water as well as not dissolve in or mix with water.

The term “lipophilic”, as used herein, refers to compounds having an affinity for lipids.

The term “amphiphilic”, as used herein, refers to a molecule combining hydrophilic and lipophilic (hydrophobic) properties. “Amphiphilic material” as used herein refers to a material containing a hydrophobic or more hydrophobic oligomer or polymer (e.g., biodegradable oligomer or polymer) and a hydrophilic or more hydrophilic oligomer or polymer.

The term “targeting moiety”, as used herein, refers to a moiety that binds to or localizes to a specific locale. The moiety may be, for example, a protein, nucleic acid, nucleic acid analog, carbohydrate, or small molecule. The locale may be a tissue, a particular cell type, or a subcellular compartment. In some embodiments, a targeting moiety can specifically bind to a selected molecule.

The term “reactive coupling group”, as used herein, refers to any chemical functional group capable of reacting with a second functional group to form a covalent bond. The selection of reactive coupling groups is within the ability of those in the art. Examples of reactive coupling groups can include primary amines (—NH₂) and amine-reactive linking groups such as isothiocyanates, isocyanates, acyl azides, NHS esters, sulfonyl chlorides, aldehydes, glyoxals, epoxides, oxiranes, carbonates, aryl halides, imidoesters, carbodiimides, anhydrides, and fluorophenyl esters. Most of these conjugate to amines by either acylation or alkylation. Examples of reactive coupling groups can include aldehydes (—COH) and aldehyde reactive linking groups such as hydrazides, alkoxyamines, and primary amines. Examples of reactive coupling groups can include thiol groups (—SH) and sulfhydryl reactive groups such as maleimides, haloacetyls, and pyridyl disulfides. Examples of reactive coupling groups can include photoreactive coupling groups such as aryl azides or diazirines. The coupling reaction may include the use of a catalyst, heat, pH buffers, light, or a combination thereof.

The term “protective group”, as used herein, refers to a functional group that can be added to and/or substituted for another desired functional group to protect the desired functional group from certain reaction conditions and selectively removed and/or replaced to deprotect or expose the desired functional group. Protective groups are known to the skilled artisan. Suitable protective groups may include those described in Greene and Wuts, Protective Groups in Organic Synthesis, (1991). Acid sensitive protective groups include dimethoxytrityl (DMT), tert-butylcarbamate (tBoc) and trifluoroacetyl (tFA). Base sensitive protective groups include 9-fluorenylmethoxycarbonyl (Fmoc), isobutyrl (iBu), benzoyl (Bz) and phenoxyacetyl (pac). Other protective groups include acetamidomethyl, acetyl, tert-amyloxycarbonyl, benzyl, benzyloxycarbonyl, 2-(4-biphεnylyl)-2-propy!oxycarbonyl, 2-bromobenzyloxycarbonyl, tert-butyl₇ tert-butyloxycarbonyl, l-carbobenzoxamido-2,2.2-trifluoroethyl, 2,6-dichlorobenzyl, 2-(3,5-dimethoxyphenyl)-2-propyloxycarbonyl, 2,4-dinitrophenyl, dithiasuccinyl, formyl, 4-methoxybenzenesulfonyl, 4-methoxybenzyl, 4-methylbenzyl, o-nitrophenylsulfenyl, 2-phenyl-2-propyloxycarbonyl, α-2,4,5-tetramethylbenzyloxycarbonyl, p-toluenesulfonyl, xanthenyl, benzyl ester, N-hydroxysuccinimide ester, p-nitrobenzyl ester, p-nitrophenyl ester, phenyl ester, p-nitrocarbonate, p-nitrobenzylcarbonate, trimethylsilyl and pentachlorophenyl ester.

The term “activated ester”, as used herein, refers to alkyl esters of carboxylic acids where the alkyl is a good leaving group rendering the carbonyl susceptible to nucleophilic attack by molecules bearing amino groups. Activated esters are therefore susceptible to aminolysis and react with amines to form amides. Activated esters contain a carboxylic acid ester group —CO₂R where R is the leaving group.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups.

In some embodiments, a straight chain or branched chain alkyl has 30 or fewer carbon atoms in its backbone (e.g., C₁-C₃₀ for straight chains, C₃-C₃₀ for branched chains), 20 or fewer, 12 or fewer, or 7 or fewer. Likewise, in some embodiments cycloalkyls have from 3-10 carbon atoms in their ring structure, e.g., have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone. Such substituents include, but are not limited to, halogen, hydroxyl, carbonyl (such as a carboxyl, alkoxycarbonyl, formyl, or an acyl), thiocarbonyl (such as a thioester, a thioacetate, or a thioformate), alkoxyl, phosphoryl, phosphate, phosphonate, a hosphinate, amino, amido, amidine, imine, cyano, nitro, azido, sulfhydryl, alkylthio, sulfate, sulfonate, sulfamoyl, sulfonamido, sulfonyl, heterocyclyl, aralkyl, or an aromatic or heteroaromatic moiety.

Unless the number of carbons is otherwise specified, “lower alkyl” as used herein means an alkyl group, as defined above, but having from one to ten carbons, or from one to six carbon atoms in its backbone structure. Likewise, “lower alkenyl” and “lower alkynyl” have similar chain lengths. In some embodiments, alkyl groups are lower alkyls. In some embodiments, a substituent designated herein as alkyl is a lower alkyl.

It will be understood by those skilled in the art that the moieties substituted on the hydrocarbon chain can themselves be substituted, if appropriate. For instance, the substituents of a substituted alkyl may include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Cycloalkyls can be substituted in the same manner.

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined above for alkyl groups.

The term “alkylthio” refers to an alkyl group, as defined above, having a sulfur radical attached thereto. In some embodiments, the “alkylthio” moiety is represented by one of —S-alkyl, —S-alkenyl, and —S-alkynyl. Representative alkylthio groups include methylthio, and ethylthio. The term “alkylthio” also encompasses cycloalkyl groups, alkene and cycloalkene groups, and alkyne groups. “Arylthio” refers to aryl or heteroaryl groups. Alkylthio groups can be substituted as defined above for alkyl groups.

The terms “alkenyl” and “alkynyl”, refer to unsaturated aliphatic groups analogous in length and possible substitution to the alkyls described above, but that contain at least one double or triple bond respectively.

The terms “alkoxyl” or “alkoxy” as used herein refers to an alkyl group, as defined above, having an oxygen radical attached thereto. Representative alkoxyl groups include methoxy, ethoxy, propyloxy, and tert-butoxy. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O-alkynyl. Aroxy can be represented by —O-aryl or O-heteroaryl, wherein aryl and heteroaryl are as defined below. The alkoxy and aroxy groups can be substituted as described above for alkyl.

The terms “amine” and “amino” are art-recognized and refer to both unsubstituted and substituted amines, e.g., a moiety that can be represented by the general formula:

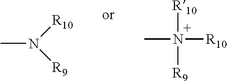

wherein R₉, R₁₀, and R′₁₀ each independently represent a hydrogen, an alkyl, an alkenyl, —(CH₂)ₘ—R₈ or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and m is zero or an integer in the range of 1 to 8. In some embodiments, only one of R₉ or R₁₀ can be a carbonyl, e.g., R₉, R₁₀ and the nitrogen together do not form an imide. In still other embodiments, the term “amine” does not encompass amides, e.g., wherein one of R₉ and R₁₀ represents a carbonyl. In additional embodiments, R₉ and R₁₀ (and optionally R′₁₀) each independently represent a hydrogen, an alkyl or cycloalkly, an alkenyl or cycloalkenyl, or alkynyl. Thus, the term “alkylamine” as used herein means an amine group, as defined above, having a substituted (as described above for alkyl) or unsubstituted alkyl attached thereto, i.e., at least one of R₉ and R₁₀ is an alkyl group.

The term “amido” is art-recognized as an amino-substituted carbonyl and includes a moiety that can be represented by the general formula:

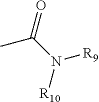

wherein R₉ and R10 are as defined above.

“Aryl”, as used herein, refers to C₅-C₁₀-membered aromatic, heterocyclic, fused aromatic, fused heterocyclic, biaromatic, or bihetereocyclic ring systems. Broadly defined, “aryl”, as used herein, includes 5-, 6-, 7-, 8-, 9-, and 10-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with one or more substituents including, but not limited to, halogen, azide, alkyl, aralkyl, alkenyl, alkynyl, cycloalkyl, hydroxyl, alkoxyl, amino (or quaternized amino), nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN; and combinations thereof.

The term “aryl” also includes polycyclic ring systems having two or more cyclic rings in which two or more carbons are common to two adjoining rings (i.e., “fused rings”) wherein at least one of the rings is aromatic, e.g., the other cyclic ring or rings can be cycloalkyls, cycloalkenyls, cycloalkynyls, aryls and/or heterocycles. Examples of heterocyclic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3 b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined above for “aryl”.

The term “aralkyl”, as used herein, refers to an alkyl group substituted with an aryl group (e.g., an aromatic or heteroaromatic group).

The term “carbocycle”, as used herein, refers to an aromatic or nonaromatic ring in which each atom of the ring is carbon.

“Heterocycle” or “heterocyclic”, as used herein, refers to a cyclic radical attached via a ring carbon or nitrogen of a monocyclic or bicyclic ring containing 3-10 ring atoms, for example, from 5-6 ring atoms, consisting of carbon and one to four heteroatoms each selected from the group consisting of non-peroxide oxygen, sulfur, and N(Y) wherein Y is absent or is H, O, (C₁-C₁₀) alkyl, phenyl or benzyl, and optionally containing 1-3 double bonds and optionally substituted with one or more substituents. Examples of heterocyclic rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Heterocyclic groups can optionally be substituted with one or more substituents at one or more positions as defined above for alkyl and aryl, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF3, and —CN.

The term “carbonyl” is art-recognized and includes such moieties as can be represented by the general formula:

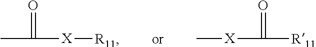

wherein X is a bond or represents an oxygen or a sulfur, and R₁₁ represents a hydrogen, an alkyl, a cycloalkyl, an alkenyl, a cycloalkenyl, or an alkynyl, R′₁₁ represents a hydrogen, an alkyl, a cycloalkyl, an alkenyl, a cycloalkenyl, or an alkynyl. Where X is an oxygen and R₁₁ or R′₁₁ is not hydrogen, the formula represents an “ester”. Where X is an oxygen and R₁₁ is as defined above, the moiety is referred to herein as a carboxyl group, and particularly when R₁₁ is a hydrogen, the formula represents a “carboxylic acid”. Where X is an oxygen and R′₁₁ is hydrogen, the formula represents a “formate”. In general, where the oxygen atom of the above formula is replaced by sulfur, the formula represents a “thiocarbonyl” group. Where X is a sulfur and R₁₁ or R′₁₁ is not hydrogen, the formula represents a “thioester.” Where X is a sulfur and R₁₁ is hydrogen, the formula represents a “thiocarboxylic acid.” Where X is a sulfur and R′₁₁ is hydrogen, the formula represents a “thioformate.” On the other hand, where X is a bond, and R₁₁ is not hydrogen, the above formula represents a “ketone” group. Where X is a bond, and R₁₁ is hydrogen, the above formula represents an “aldehyde” group.

The term “monoester” as used herein refers to an analog of a dicarboxylic acid wherein one of the carboxylic acids is functionalized as an ester and the other carboxylic acid is a free carboxylic acid or salt of a carboxylic acid. Examples of monoesters include, but are not limited to, to monoesters of succinic acid, glutaric acid, adipic acid, suberic acid, sebacic acid, azelaic acid, oxalic and maleic acid.

The term “heteroatom” as used herein means an atom of any element other than carbon or hydrogen. Examples of heteroatoms are boron, nitrogen, oxygen, phosphorus, sulfur and selenium. Other useful heteroatoms include silicon and arsenic.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂—.

The term “substituted” as used herein, refers to all permissible substituents of the compounds described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, for example, 1-14 carbon atoms, and optionally include one or more heteroatoms such as oxygen, sulfur, or nitrogen grouping in linear, branched, or cyclic structural formats. Representative substituents include alkyl, substituted alkyl, alkenyl, substituted alkenyl, alkynyl, substituted alkynyl, phenyl, substituted phenyl, aryl, substituted aryl, heteroaryl, substituted heteroaryl, halo, hydroxyl, alkoxy, substituted alkoxy, phenoxy, substituted phenoxy, aroxy, substituted aroxy, alkylthio, substituted alkylthio, phenylthio, substituted phenylthio, arylthio, substituted arylthio, cyano, isocyano, substituted isocyano, carbonyl, substituted carbonyl, carboxyl, substituted carboxyl, amino, substituted amino, amido, substituted amido, sulfonyl, substituted sulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, aminoacid, peptide, and polypeptide groups.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e., a compound that does not spontaneously undergo transformation, for example, by rearrangement, cyclization, or elimination.

In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents can be one or more and the same or different for appropriate organic compounds. The heteroatoms such as nitrogen may have hydrogen substituents and/or any permissible substituents of organic compounds described herein which satisfy the valencies of the heteroatoms.

In various embodiments, the substituent is selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone, each of which optionally is substituted with one or more suitable substituents. In some embodiments, the substituent is selected from alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone, wherein each of the alkoxy, aryloxy, alkyl, alkenyl, alkynyl, amide, amino, aryl, arylalkyl, carbamate, carboxy, cycloalkyl, ester, ether, formyl, haloalkyl, heteroaryl, heterocyclyl, ketone, phosphate, sulfide, sulfinyl, sulfonyl, sulfonic acid, sulfonamide, and thioketone can be further substituted with one or more suitable substituents.

Examples of substituents include, but are not limited to, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, thioketone, ester, heterocyclyl, —CN, aryl, aryloxy, perhaloalkoxy, aralkoxy, heteroaryl, heteroaryloxy, heteroarylalkyl, heteroaralkoxy, azido, alkylthio, oxo, acylalkyl, carboxy esters, carboxamido, acyloxy, aminoalkyl, alkylaminoaryl, alkylaryl, alkylaminoalkyl, alkoxyaryl, arylamino, aralkylamino, alkylsulfonyl, carboxamidoalkylaryl, carboxamidoaryl, hydroxyalkyl, haloalkyl, alkylaminoalkylcarboxy, aminocarboxamidoalkyl, cyano, alkoxyalkyl, perhaloalkyl, arylalkyloxyalkyl, and the like. In some embodiments, the substituent is selected from cyano, halogen, hydroxyl, and nitro.

The term “copolymer” as used herein, generally refers to a single polymeric material that is comprised of two or more different monomers. The copolymer can be of any form, for example, random, block, or graft. The copolymers can have any end-group, including capped or acid end groups.

The terms “polypeptide,” “peptide” and “protein” generally refer to a polymer of amino acid residues. As used herein, the term also applies to amino acid polymers in which one or more amino acids are chemical analogs or modified derivatives of corresponding naturally-occurring amino acids or are unnatural amino acids. The term “protein”, as generally used herein, refers to a polymer of amino acids linked to each other by peptide bonds to form a polypeptide for which the chain length is sufficient to produce tertiary and/or quaternary structure. The term “protein” excludes small peptides by definition, the small peptides lacking the requisite higher-order structure necessary to be considered a protein.

The terms “nucleic acid,” “polynucleotide,” and “oligonucleotide” are used interchangeably to refer to a deoxyribonucleotide or ribonucleotide polymer, in linear or circular conformation, and in either single- or double-stranded form. These terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general and unless otherwise specified, an analog of a particular nucleotide has the same base-pairing specificity; i.e., an analog of A will base-pair with T. The term “nucleic acid” is a term of art that refers to a string of at least two base-sugar-phosphate monomeric units. Nucleotides are the monomeric units of nucleic acid polymers. The term includes deoxyribonucleic acid (DNA) and ribonucleic acid (RNA) in the form of a messenger RNA, antisense, plasmid DNA, parts of a plasmid DNA or genetic material derived from a virus. An antisense nucleic acid is a polynucleotide that interferes with the expression of a DNA and/or RNA sequence. The term nucleic acids refer to a string of at least two base-sugar-phosphate combinations. Natural nucleic acids have a phosphate backbone. Artificial nucleic acids may contain other types of backbones, but contain the same bases as natural nucleic acids. The term also includes PNAs (peptide nucleic acids), phosphorothioates, and other variants of the phosphate backbone of native nucleic acids.

A “functional fragment” of a protein, polypeptide or nucleic acid is a protein, polypeptide or nucleic acid whose sequence is not identical to the full-length protein, polypeptide or nucleic acid, yet retains at least one function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA binding function of a polypeptide can be determined, for example, by filter-binding, electrophoretic mobility shift, or immunoprecipitation assays. DNA cleavage can be assayed by gel electrophoresis. The ability of a protein to interact with another protein can be determined, for example, by co-immunoprecipitation, two-hybrid assays or complementation, e.g., genetic or biochemical. See, for example, Fields et al. (1989) Nature 340:245-246; U.S. Pat. No. 5,585,245 and PCT WO 98/44350.

As used herein, the term “linker” refers to a carbon chain that can contain heteroatoms (e.g., nitrogen, oxygen, sulfur, etc.) and which may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 atoms long. Linkers may be substituted with various substituents including, but not limited to, hydrogen atoms, alkyl, alkenyl, alkynl, amino, alkylamino, dialkylamino, trialkylamino, hydroxyl, alkoxy, halogen, aryl, heterocyclic, aromatic heterocyclic, cyano, amide, carbamoyl, carboxylic acid, ester, thioether, alkylthioether, thiol, and ureido groups. Those of skill in the art will recognize that each of these groups may in turn be substituted. Examples of linkers include, but are not limited to, pH-sensitive linkers, protease cleavable peptide linkers, nuclease sensitive nucleic acid linkers, lipase sensitive lipid linkers, glycosidase sensitive carbohydrate linkers, hypoxia sensitive linkers, photo-cleavable linkers, heat-labile linkers, enzyme cleavable linkers (e.g., esterase cleavable linker), ultrasound-sensitive linkers, and x-ray cleavable linkers.

The term “pharmaceutically acceptable counter ion” refers to a pharmaceutically acceptable anion or cation. In various embodiments, the pharmaceutically acceptable counter ion is a pharmaceutically acceptable ion. For example, the pharmaceutically acceptable counter ion is selected from citrate, malate, acetate, oxalate, chloride, bromide, iodide, nitrate, sulfate, bisulfate, phosphate, acid phosphate, isonicotinate, acetate, lactate, salicylate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)). In some embodiments, the pharmaceutically acceptable counter ion is selected from chloride, bromide, iodide, nitrate, sulfate, bisulfate, phosphate, acid phosphate, citrate, malate, acetate, oxalate, acetate, and lactate. In particular embodiments, the pharmaceutically acceptable counter ion is selected from chloride, bromide, iodide, nitrate, sulfate, bisulfate, and phosphate.

The term “pharmaceutically acceptable salt(s)” refers to salts of acidic or basic groups that may be present in compounds used in the present compositions. Compounds included in the present compositions that are basic in nature are capable of forming a variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfate, citrate, malate, acetate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions, that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

If the compounds described herein are obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

A pharmaceutically acceptable salt can be derived from an acid selected from 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid, aspartic acid, benzenesulfonic acid, benzoic acid, camphoric acid, camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid, gluconic acid, glucuronic acid, glutamic acid, glutaric acid, glycerophosphoric acid, glycolic acid, hippuric acid, hydrobromic acid, hydrochloric acid, isethionic, isobutyric acid, lactic acid, lactobionic acid, lauric acid, maleic acid, malic acid, malonic acid, mandelic acid, methanesulfonic acid, mucic, naphthalene-1,5-disulfonic acid, naphthalene-2-sulfonic acid, nicotinic acid, nitric acid, oleic acid, oxalic acid, palmitic acid, pamoic acid, pantothenic, phosphoric acid, proprionic acid, pyroglutamic acid, salicylic acid, sebacic acid, stearic acid, succinic acid, sulfuric acid, tartaric acid, thiocyanic acid, toluenesulfonic acid, trifluoroacetic, and undecylenic acid.

The term “bioavailable” is art-recognized and refers to a form of the subject invention that allows for it, or a portion of the amount administered, to be absorbed by, incorporated to, or otherwise physiologically available to a subject or patient to whom it is administered.

It will be appreciated that the following examples are intended to illustrate but not to limit the present invention. Various other examples and modifications of the foregoing description and examples will be apparent to a person skilled in the art after reading the disclosure without departing from the spirit and scope of the invention, and it is intended that all such examples or modifications be included within the scope of the appended claims. All publications and patents referenced herein are hereby incorporated by reference in their entirety.

It will be appreciated that in the following examples, some conjugates were prepared and characterized using non-radioactive metals such as Lu-175. It will be apparent to a person skilled in that art that the corresponding radioactive Lu-177 analogs can be readily prepared using known methods and that the distribution data for the Lu-175 conjugates may be representative of the Lu-177 analogs.

### EXAMPLES

### Example 1: Synthesis of the Conjugates

The conjugates of the invention may be prepared using any convenient methodology. In a rational approach, the conjugates are constructed from their individual components, targeting moiety, in some cases a linker, and active agent moiety. The components can be covalently bonded to one another through functional groups, as is known in the art, where such functional groups may be present on the components or introduced onto the components using one or more steps, e.g., oxidation reactions, reduction reactions, cleavage reactions and the like. Functional groups that may be used in covalently bonding the components together to produce the pharmaceutical conjugate include: hydroxy, sulfhydryl, amino, and the like. The particular portion of the different components that are modified to provide for covalent linkage will be chosen so as not to substantially adversely interfere with that components desired binding activity, e.g., for the active agent moiety, a region that does not affect the target binding activity will be modified, such that a sufficient amount of the desired drug activity is preserved. Where necessary and/or desired, certain moieties on the components may be protected using blocking groups, as is known in the art, see, e.g., Green & Wuts, Protective Groups in Organic Synthesis (John Wiley & Sons) (1991).

Alternatively, the conjugate can be produced using known combinatorial methods to produce large libraries of potential conjugates which may then be screened for identification of a bifunctional, molecule with the pharmacokinetic profile. Alternatively, the conjugates may be produced using medicinal chemistry and known structure-activity relationships for the targeting moiety and the active agent moiety. In particular, this approach will provide insight as to where to join the two moieties to the linker.

### Peptide Synthesis

Peptide 1 was synthesized on a Liberty Blue peptide synthesizer starting with Fmoc-beta-alanine loaded onto 2-chlorotrityl resin (1.33 g, 0.75 mmol/g loading, 1.00 mmol) and appending Fmoc-D-Glu(OtBu) (×3), Fmoc-AEEA, Fmoc-Lys(Boc), and 4-(p-iodophenyl)butyric acid. The crude peptide was cleaved by treating the resin with 2% TFA in dichloromethane, and the crude peptide purified by preparative HPLC to yield 624 mg (0.483 mmol, 48% yield) of peptide 1.

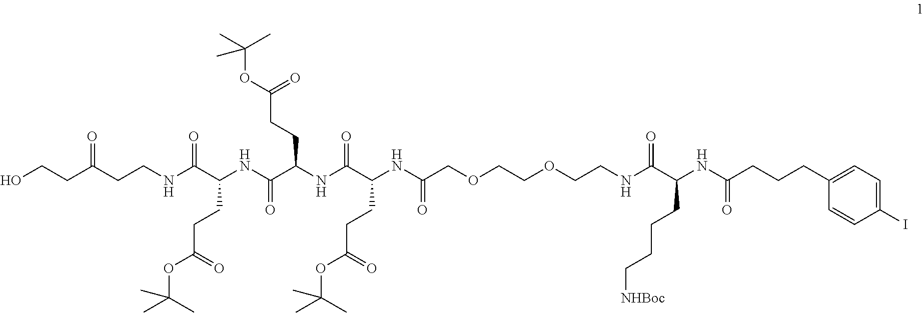 [SMILES: CC(C)(C)OC(=O)CC[C@@H](NC(=O)COCCOCCNC(=O)[C@H](CCCCNC(=O)OC(C)(C)C)NC(=O)CCCc1ccc(I)cc1)C(=O)N[C@H](CCC(=O)OC(C)(C)C)C(=O)N[C@H](CCC(=O)OC(C)(C)C)C(=O)NCCC(=O)CCO]

Peptide 2 was synthesized on a Liberty Blue peptide synthesizer starting with Fmoc-beta-alanine loaded onto 2-chlorotrityl resin (4.00 g, 0.75 mmol/g loading, 3.00 mmol) and appending Fmoc-D-Glu(OtBu) (×3) and Fmoc-AEEA. The crude peptide was cleaved by treating the resin with 2% TFA in dichloromethane, and the crude peptide purified by preparative HPLC to yield 1.28 g (1.26 mmol, 42% yield) of peptide 2. Peptides 3 and 4 were synthesized in a similar manner.

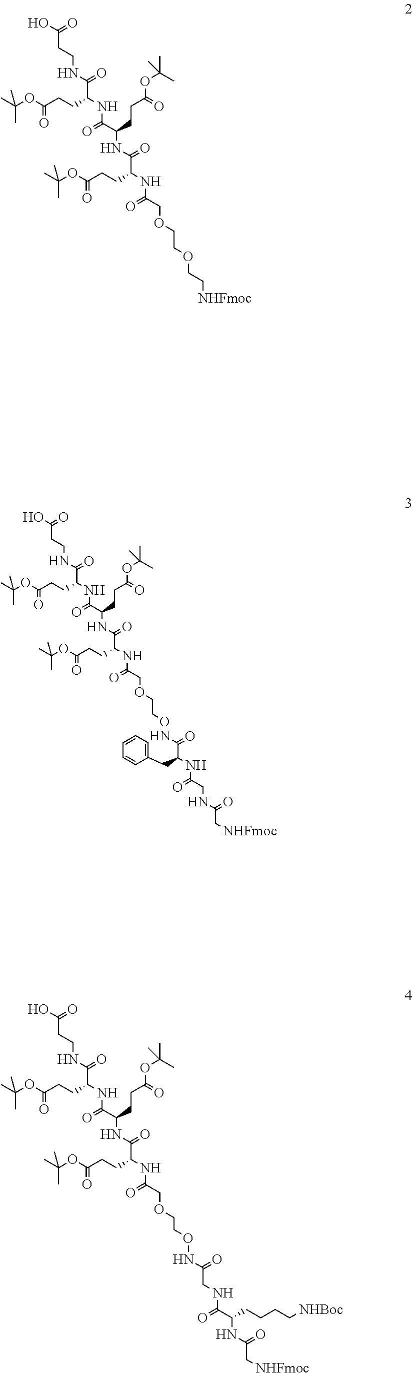

Peptide 5 was synthesized on a Liberty Blue peptide synthesizer starting with Fmoc-beta-alanine loaded onto 2-chlorotrityl resin (0.4 g, 0.5 mmol/g loading, 0.2 mmol) and appending Fmoc-D-Glu(OtBu) (×3), Fmoc-AEEA, Dde-Lys(Fmoc), (R)-tert-Bu4-DOTAGA, then treating with 5% hydrazine in DMF, and appending 4-(p-iodophenyl)butyric acid. The crude peptide was cleaved by treating the resin with 2% TFA in dichloromethane, and the crude peptide purified by preparative HPLC to yield 50 mg (0.027 mmol, 13% yield) of peptide 5. Peptides 6 and 7 were synthesized in a similar manner.

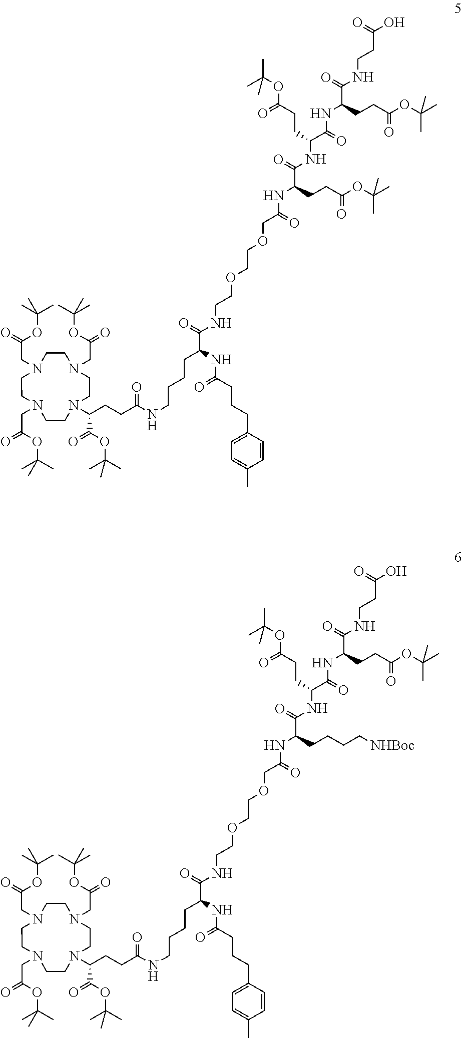 [SMILES: Cc1ccc(CCCC(=O)N[C@@H](CCCCNC(=O)CC[C@H](C(=O)OC(C)(C)C)N2CCN(CC(=O)OC(C)(C)C)CCN(CC(=O)OC(C)(C)C)CCN(CC(=O)OC(C)(C)C)CC2)C(=O)NCCOCCOCC(=O)N[C@H](CCC(=O)OC(C)(C)C)C(=O)N[C@H](CCC(=O)OC(C)(C)C)C(=O)N[C@H](CCC(=O)OC(C)(C)C)C(=O)NCCC(=O)O)cc1 ; Cc1ccc(CCCC(=O)N[C@@H](CCCCNC(=O)CC[C@H](C(=O)OC(C)(C)C)N2CCN(CC(=O)OC(C)(C)C)CCN(CC(=O)OC(C)(C)C)CCN(CC(=O)OC(C)(C)C)CC2)C(=O)NCCOCCOCC(=O)N[C@H](CCCCNC(=O)OC(C)(C)C)C(=O)N[C@H](CCC(=O)OC(C)(C)C)C(=O)N[C@H](CCC(=O)OC(C)(C)C)C(=O)NCCC(=O)O)cc1]

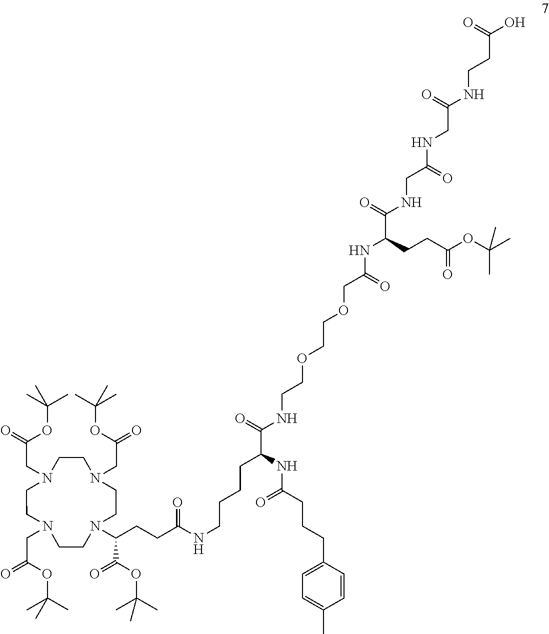 [SMILES: Cc1ccc(CCCC(=O)N[C@@H](CCCCNC(=O)CC[C@H](C(=O)OC(C)(C)C)N2CCN(CC(=O)OC(C)(C)C)CCN(CC(=O)OC(C)(C)C)CCN(CC(=O)OC(C)(C)C)CC2)C(=O)NCCOCCOCC(=O)N[C@H](CCC(=O)OC(C)(C)C)C(=O)NCC(=O)NCC(=O)NCCC(=O)O)cc1]

Peptide 8 was synthesized on a Liberty Blue peptide synthesizer starting with Fmoc-beta-alanine loaded onto 2-chlorotrityl resin (0.5 g, 0.75 mmol/g loading, 0.375 mmol) and appending Fmoc-D-Glu(OtBu) (×3), Fmoc-AEEA, Fmoc-D-Lys(Dde), 4-(p-iodophenyl)butyric acid, treating with 5% hydrazine in DMF, and then Fmoc-Gly, Fmoc-Val, Fmoc-Met, and Fmoc-Gly. The crude peptide was cleaved from the resin with 2% TFA in dichloromethane, and all solvent removed. The crude peptide was dissolved in acetonitrile (5 mL) and triethylamine (2 mL), and the solution heated to 70° C. for 1 h. All solvent was removed under vacuum, and the crude material treated with (R)-tert-Bu4-DOTAGA NHS ester (172 mg, 0.215 mmol) in DMF (5 mL) and diisopropylethylamine (0.5 mL). The solution was stirred at 50° C. for 1 h then purified by preparative HPLC to give 8 (124 mg, 55.9 mmol, 15% yield). Peptides 9-16 were made in a similar manner.

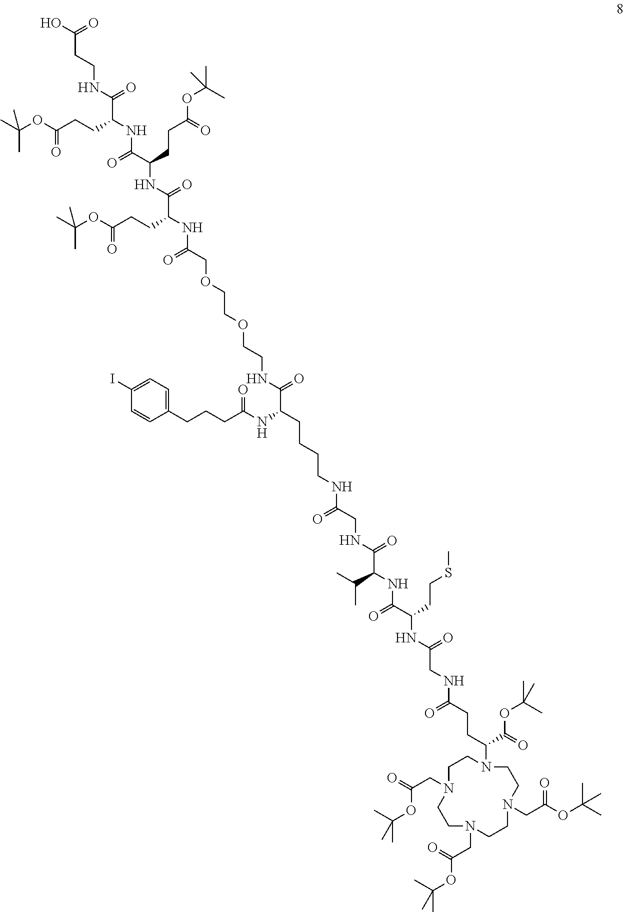 [SMILES: CSCC[C@H](NC(=O)CNC(=O)CC[C@H](C(=O)OC(C)(C)C)N1CCN(CC(=O)OC(C)(C)C)CCN(CC(=O)OC(C)(C)C)CCN(CC(=O)OC(C)(C)C)CC1)C(=O)N[C@H](C(=O)NCC(=O)NCCCC[C@H](NC(=O)CCCc1ccc(I)cc1)C(=O)NCCOCCOCC(=O)N[C@H](CCC(=O)OC(C)(C)C)C(=O)N[C@H](CCC(=O)OC(C)(C)C)C(=O)N[C@H](CCC(=O)OC(C)(C)C)C(=O)NCCC(=O)O)C(C)C]

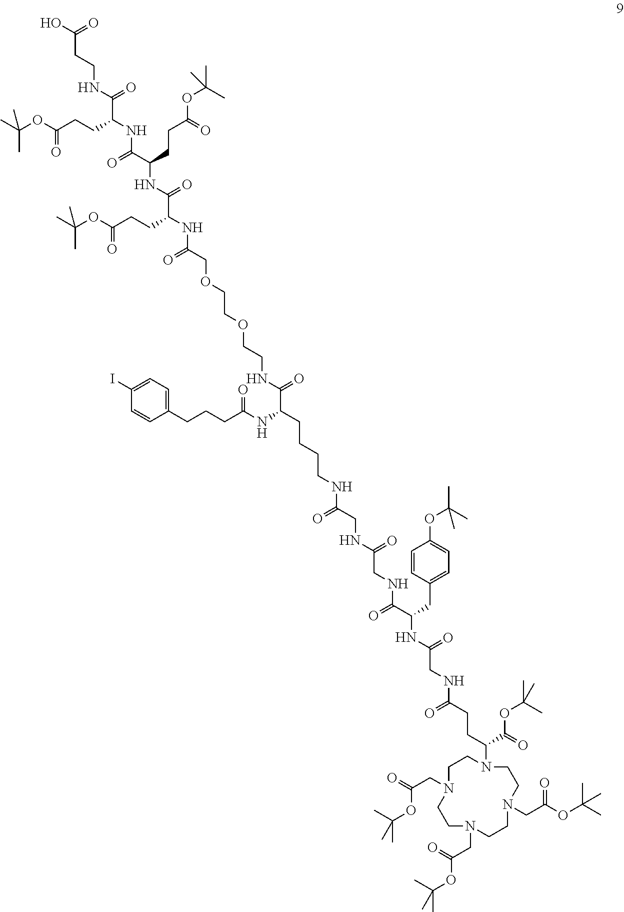 [SMILES: CC(C)(C)OC(=O)CC[C@@H](NC(=O)COCCOCCNC(=O)[C@H](CCCCNC(=O)CNC(=O)CNC(=O)[C@H](Cc1ccc(OC(C)(C)C)cc1)NC(=O)CNC(=O)CC[C@H](C(=O)OC(C)(C)C)N1CCN(CC(=O)OC(C)(C)C)CCN(CC(=O)OC(C)(C)C)CCN(CC(=O)OC(C)(C)C)CC1)NC(=O)CCCc1ccc(I)cc1)C(=O)N[C@H](CCC(=O)OC(C)(C)C)C(=O)N[C@H](CCC(=O)OC(C)(C)C)C(=O)NCCC(=O)O]

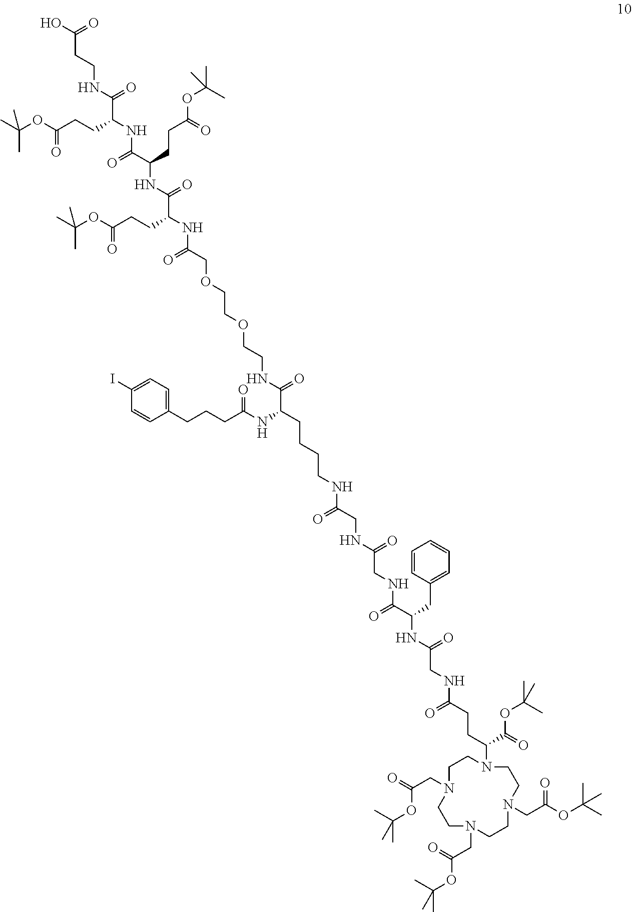 [SMILES: CC(C)(C)OC(=O)CC[C@@H](NC(=O)COCCOCCNC(=O)[C@H](CCCCNC(=O)CNC(=O)CNC(=O)[C@H](Cc1ccccc1)NC(=O)CNC(=O)CC[C@H](C(=O)OC(C)(C)C)N1CCN(CC(=O)OC(C)(C)C)CCN(CC(=O)OC(C)(C)C)CCN(CC(=O)OC(C)(C)C)CC1)NC(=O)CCCc1ccc(I)cc1)C(=O)N[C@H](CCC(=O)OC(C)(C)C)C(=O)N[C@H](CCC(=O)OC(C)(C)C)C(=O)NCCC(=O)O]

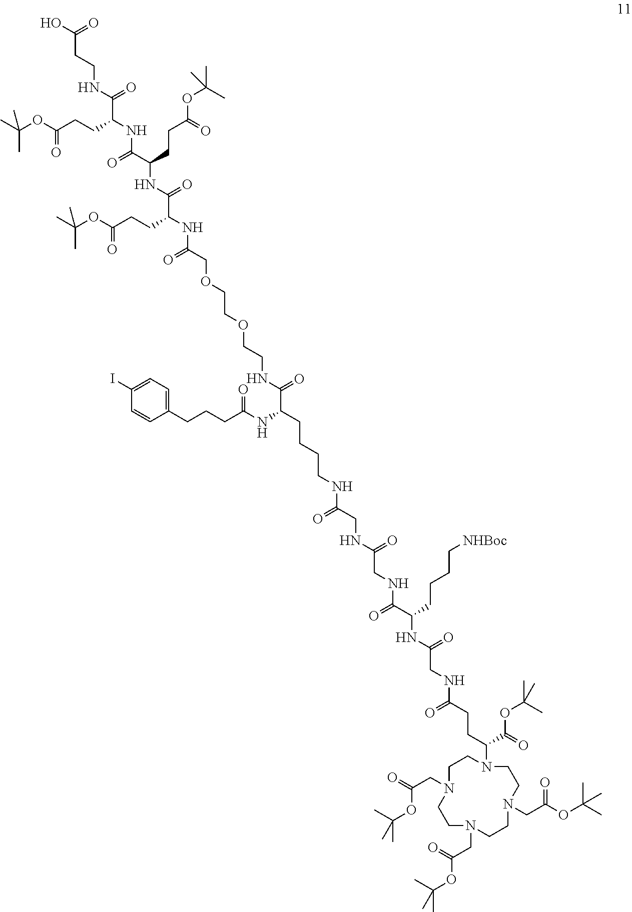 [SMILES: CC(C)(C)OC(=O)CC[C@@H](NC(=O)COCCOCCNC(=O)[C@H](CCCCNC(=O)CNC(=O)CNC(=O)[C@H](CCCCNC(=O)OC(C)(C)C)NC(=O)CNC(=O)CC[C@H](C(=O)OC(C)(C)C)N1CCN(CC(=O)OC(C)(C)C)CCN(CC(=O)OC(C)(C)C)CCN(CC(=O)OC(C)(C)C)CC1)NC(=O)CCCc1ccc(I)cc1)C(=O)N[C@H](CCC(=O)OC(C)(C)C)C(=O)N[C@H](CCC(=O)OC(C)(C)C)C(=O)NCCC(=O)O]

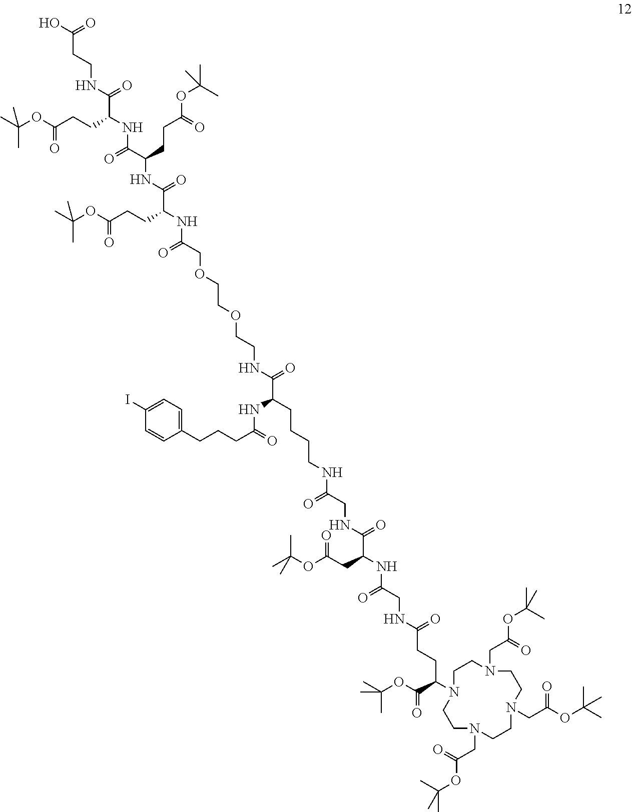 [SMILES: CC(C)(C)OC(=O)CC[C@@H](NC(=O)COCCOCCNC(=O)[C@@H](CCCCNC(=O)CNC(=O)[C@H](CC(=O)OC(C)(C)C)NC(=O)CNC(=O)CC[C@H](C(=O)OC(C)(C)C)N1CCN(CC(=O)OC(C)(C)C)CCN(CC(=O)OC(C)(C)C)CCN(CC(=O)OC(C)(C)C)CC1)NC(=O)CCCc1ccc(I)cc1)C(=O)N[C@H](CCC(=O)OC(C)(C)C)C(=O)N[C@H](CCC(=O)OC(C)(C)C)C(=O)NCCC(=O)O]

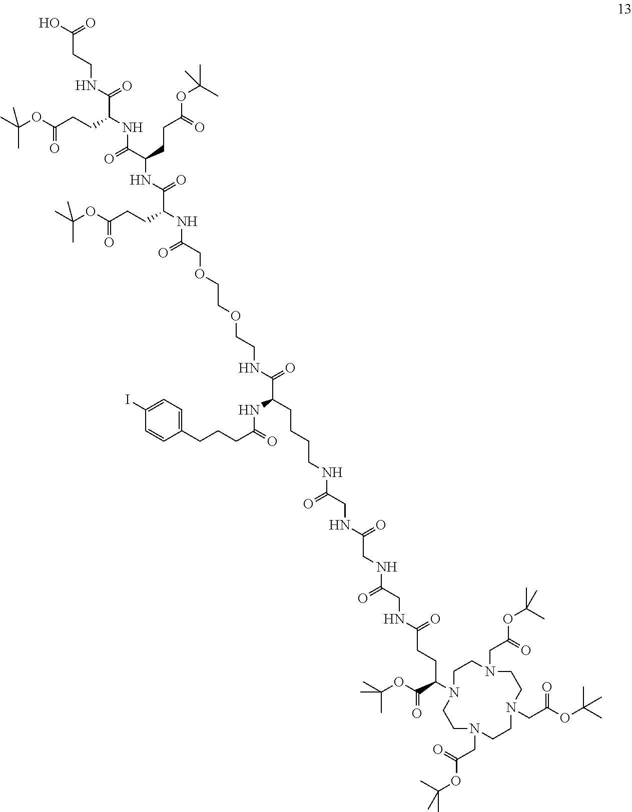 [SMILES: CC(C)(C)OC(=O)CC[C@@H](NC(=O)COCCOCCNC(=O)[C@@H](CCCCNC(=O)CNC(=O)CNC(=O)CNC(=O)CC[C@H](C(=O)OC(C)(C)C)N1CCN(CC(=O)OC(C)(C)C)CCN(CC(=O)OC(C)(C)C)CCN(CC(=O)OC(C)(C)C)CC1)NC(=O)CCCc1ccc(I)cc1)C(=O)N[C@H](CCC(=O)OC(C)(C)C)C(=O)N[C@H](CCC(=O)OC(C)(C)C)C(=O)NCCC(=O)O]

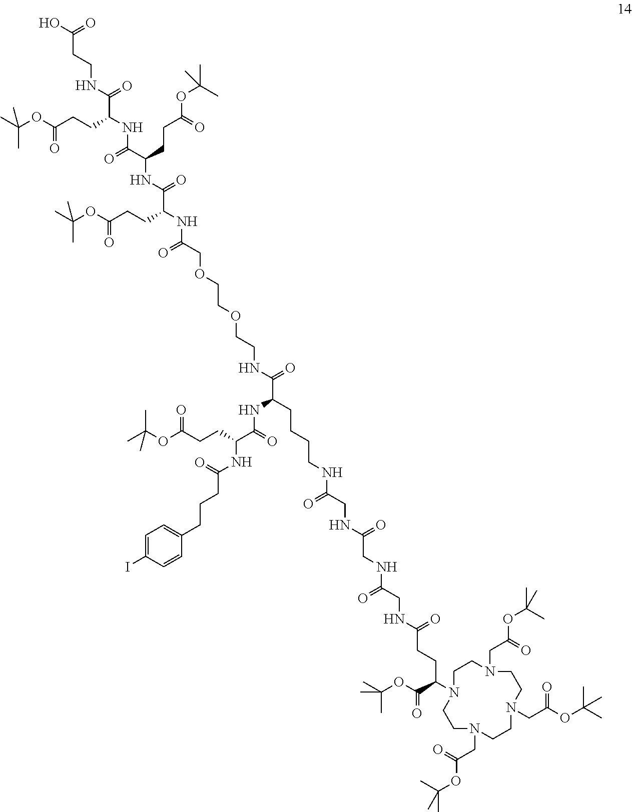 [SMILES: CC(C)(C)OC(=O)CC[C@@H](NC(=O)CCCc1ccc(I)cc1)C(=O)N[C@H](CCCCNC(=O)CNC(=O)CNC(=O)CNC(=O)CC[C@H](C(=O)OC(C)(C)C)N1CCN(CC(=O)OC(C)(C)C)CCN(CC(=O)OC(C)(C)C)CCN(CC(=O)OC(C)(C)C)CC1)C(=O)NCCOCCOCC(=O)N[C@H](CCC(=O)OC(C)(C)C)C(=O)N[C@H](CCC(=O)OC(C)(C)C)C(=O)N[C@H](CCC(=O)OC(C)(C)C)C(=O)NCCC(=O)O]

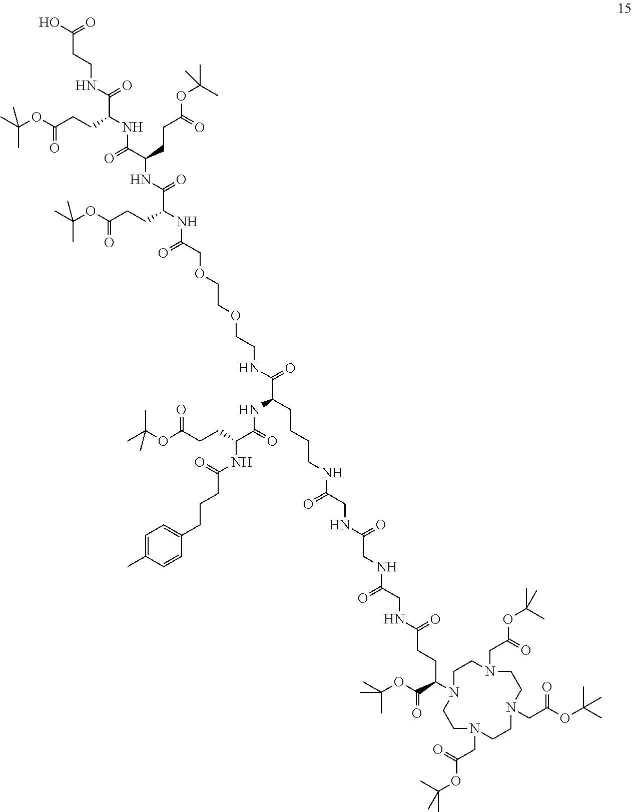 [SMILES: Cc1ccc(CCCC(=O)N[C@H](CCC(=O)OC(C)(C)C)C(=O)N[C@H](CCCCNC(=O)CNC(=O)CNC(=O)CNC(=O)CC[C@H](C(=O)OC(C)(C)C)N2CCN(CC(=O)OC(C)(C)C)CCN(CC(=O)OC(C)(C)C)CCN(CC(=O)OC(C)(C)C)CC2)C(=O)NCCOCCOCC(=O)N[C@H](CCC(=O)OC(C)(C)C)C(=O)N[C@H](CCC(=O)OC(C)(C)C)C(=O)N[C@H](CCC(=O)OC(C)(C)C)C(=O)NCCC(=O)O)cc1]

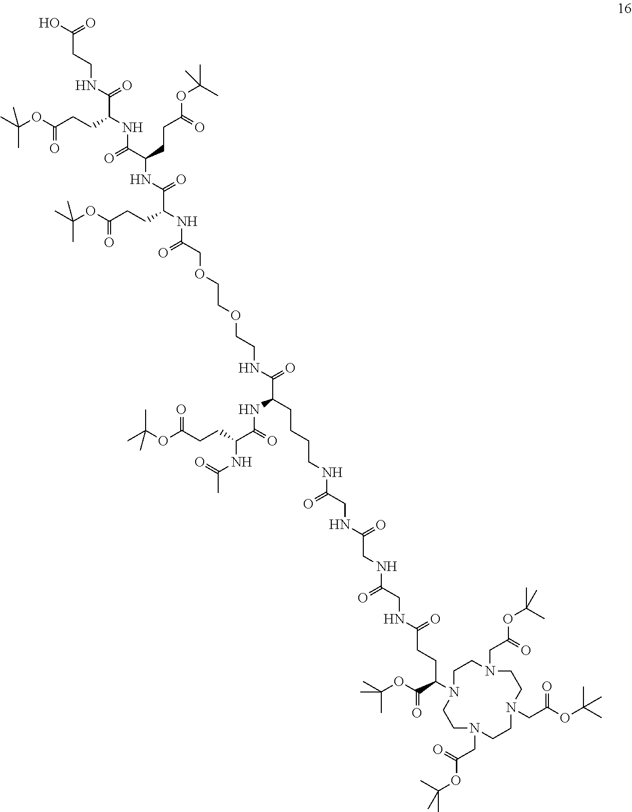 [SMILES: CC(=O)N[C@H](CCC(=O)OC(C)(C)C)C(=O)N[C@H](CCCCNC(=O)CNC(=O)CNC(=O)CNC(=O)CC[C@H](C(=O)OC(C)(C)C)N1CCN(CC(=O)OC(C)(C)C)CCN(CC(=O)OC(C)(C)C)CCN(CC(=O)OC(C)(C)C)CC1)C(=O)NCCOCCOCC(=O)N[C@H](CCC(=O)OC(C)(C)C)C(=O)N[C@H](CCC(=O)OC(C)(C)C)C(=O)N[C@H](CCC(=O)OC(C)(C)C)C(=O)NCCC(=O)O]

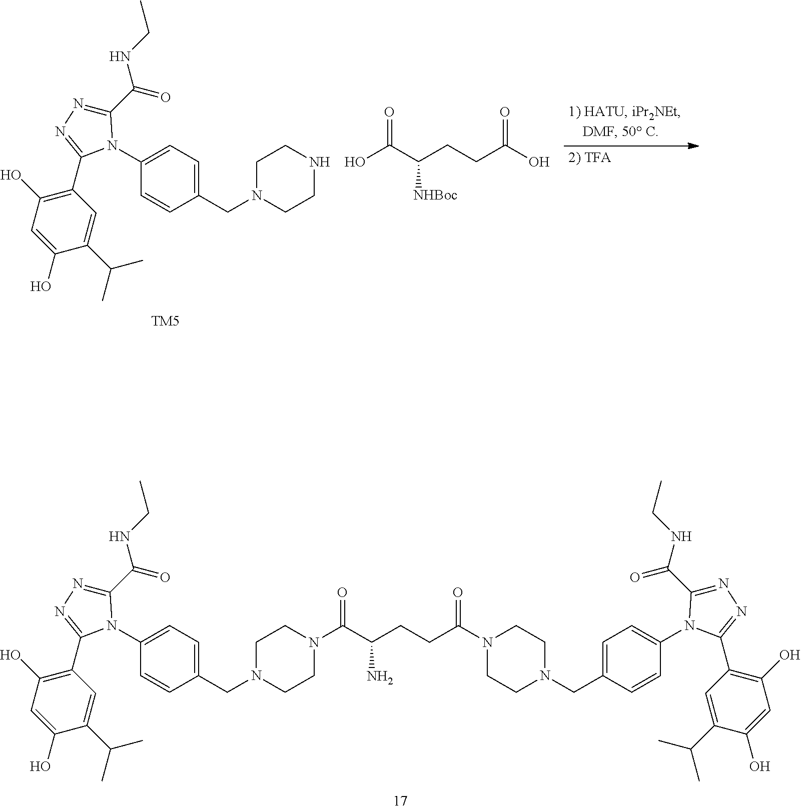

To a vial charged with TM5 HCl salt (170 mg, 0.340 mmol) and Boc-glutamic acid (42 mg, 0.170 mmol) was added a solution of HATU (128 mg, 0.340 mmol) in DMF (3 mL), followed by diisopropylethylamine (0.60 mL). The solution was stirred at 50° C. for 1 h, then purified by preparative HPLC. The product-containing fractions were concentrated to dryness, and then trifluoroacetic acid (2 mL) was added. The solution was stirred at room temperature for 15 min, then excess trifluoroacetic acid removed under vacuum. The remaining material was redissolved in 1:1 acetonitrile:water (10 mL), frozen, and lyophilized to give 17 as the trifluoroacetate salt (192 mg, 0.138 mmol, 81% yield). Compounds 18 and 19 were made in a similar manner.

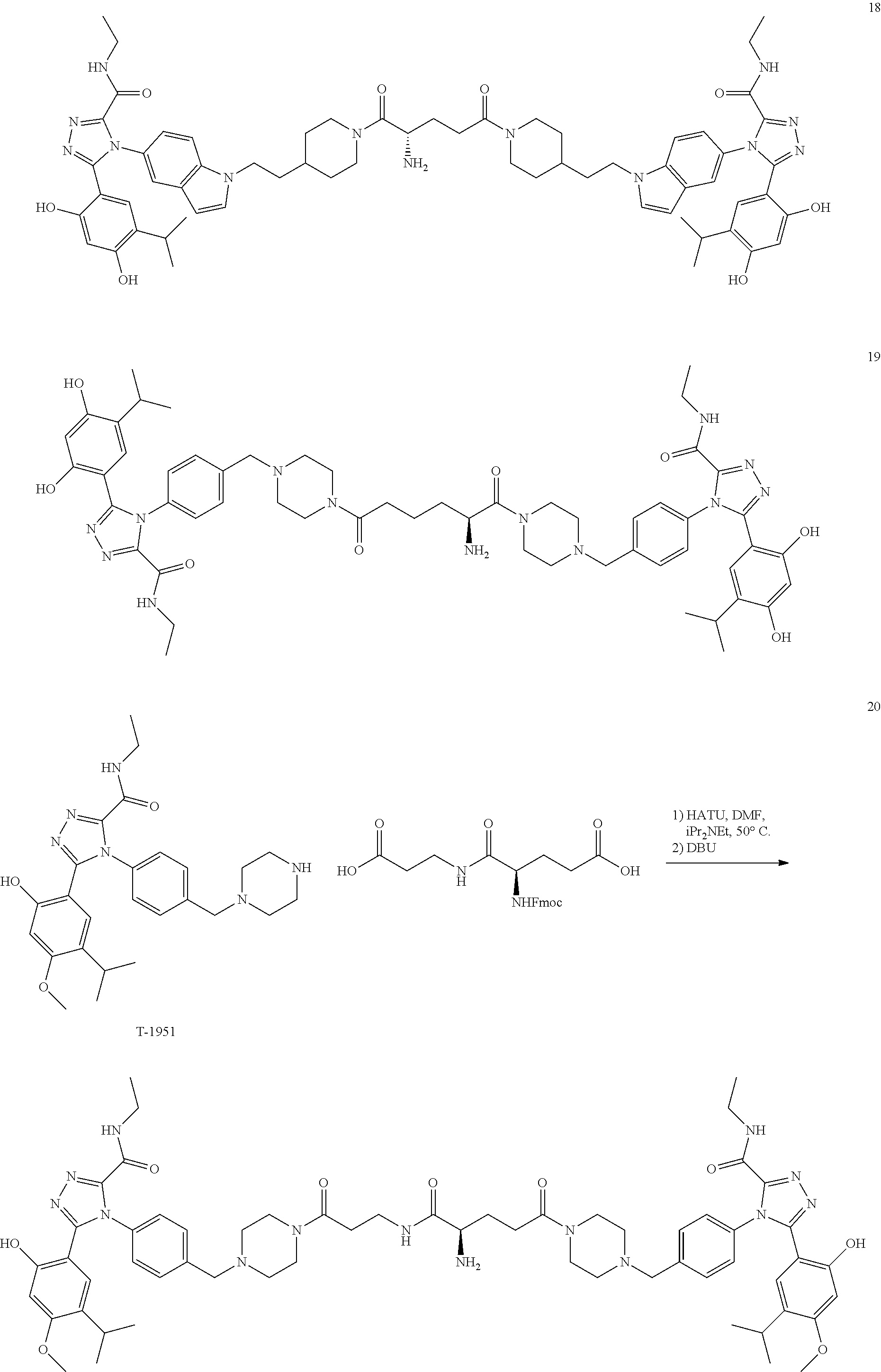

A vial was charged with T-1951 HCl salt (460 mg, 0.893 mmol) and Fmoc-DGlu-bAla (200 mg, 0.454 mmol). To this was added a solution of HATU (343 mg, 0.909 mmol) in DMF (5 mL). Diisopropylethylamine (0.50 mL) was added, and the solution stirred at 50° C. for 1 h, then DBU (0.50 mL) was added. The reaction was stirred at 50° C. for another 1 h, then the reaction acidified by adding 1 mL TFA, and the reaction mixture purified by preparative HPLC to give 20 as the trifluoroacetate salt (443 mg, 0.300 mmol, 65% yield). Compounds 21-32 were made in a similar manner.

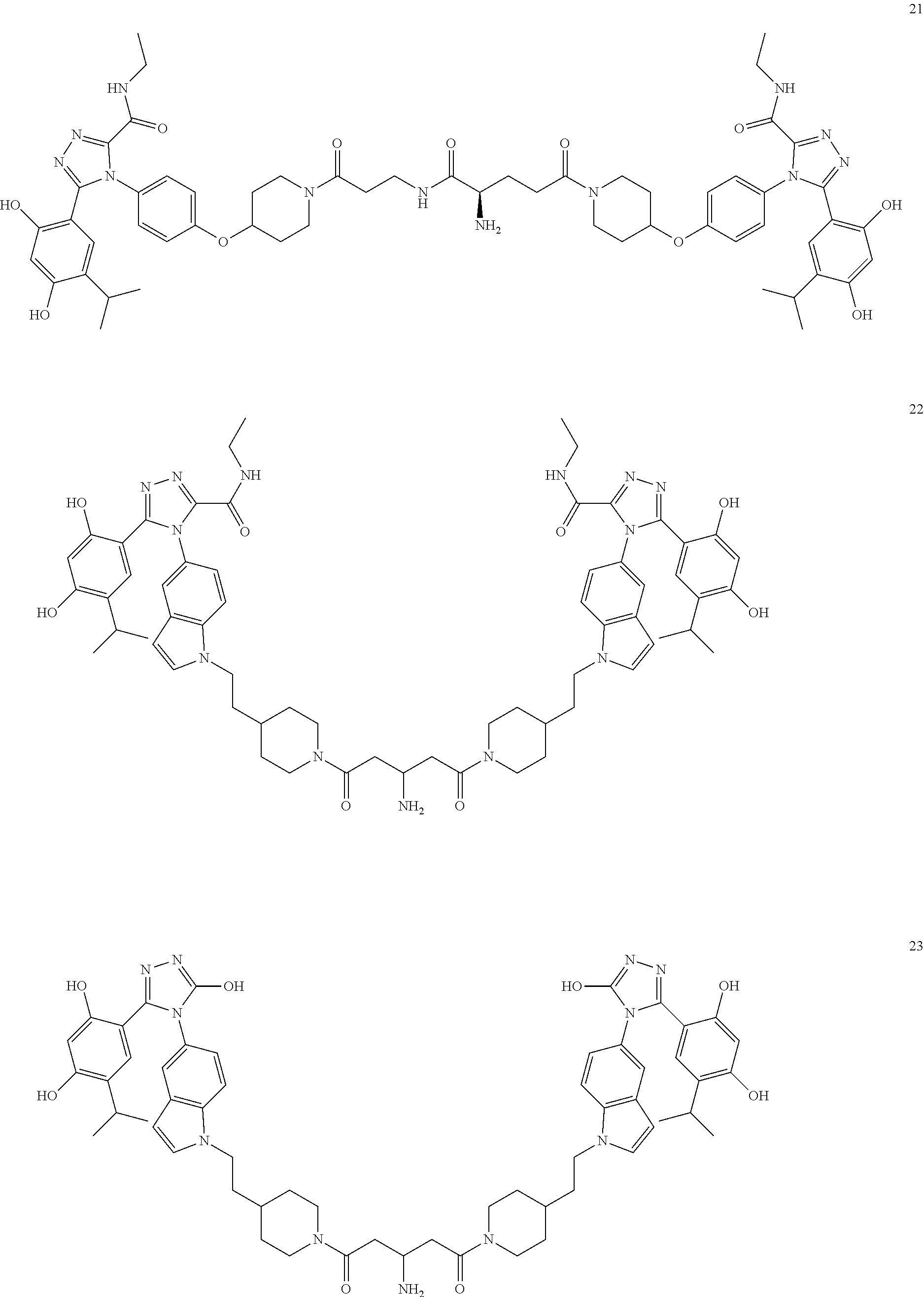 [SMILES: CCNC(=O)c1nnc(-c2cc(C(C)C)c(O)cc2O)n1-c1ccc(OC2CCN(C(=O)CCNC(=O)[C@H](N)CCC(=O)N3CCC(Oc4ccc(-n5c(C(=O)NCC)nnc5-c5cc(C(C)C)c(O)cc5O)cc4)CC3)CC2)cc1 ; CCNC(=O)c1nnc(-c2cc(C(C)C)c(O)cc2O)n1-c1ccc2c(ccn2CCC2CCN(C(=O)CC(N)CC(=O)N3CCC(CCn4ccc5cc(-n6c(C(=O)NCC)nnc6-c6cc(C(C)C)c(O)cc6O)ccc54)CC3)CC2)c1 ; CC(C)c1cc(-c2nnc(O)n2-c2ccc3c(ccn3CCC3CCN(C(=O)CC(N)CC(=O)N4CCC(CCn5ccc6cc(-n7c(O)nnc7-c7cc(C(C)C)c(O)cc7O)ccc65)CC4)CC3)c2)c(O)cc1O]

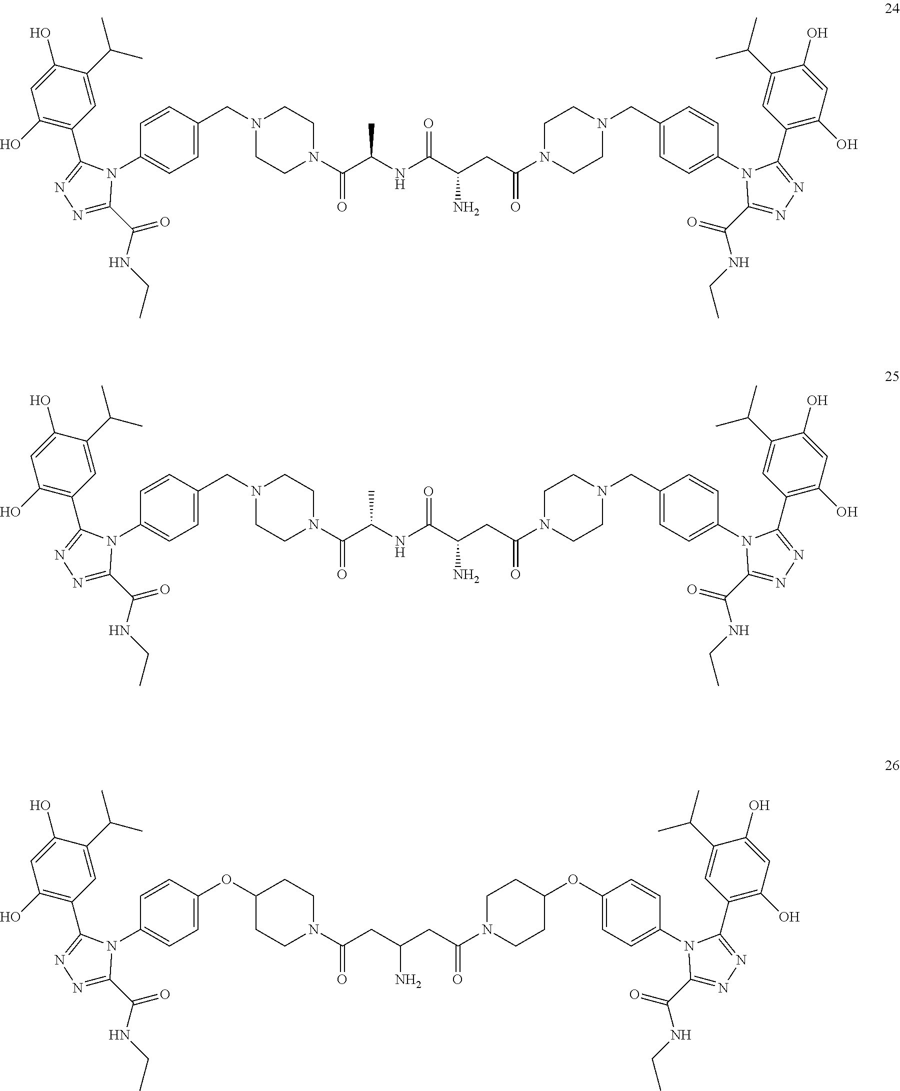 [SMILES: CCNC(=O)c1nnc(-c2cc(C(C)C)c(O)cc2O)n1-c1ccc(CN2CCN(C(=O)C[C@H](N)C(=O)N[C@H](C)C(=O)N3CCN(Cc4ccc(-n5c(C(=O)NCC)nnc5-c5cc(C(C)C)c(O)cc5O)cc4)CC3)CC2)cc1 ; CCNC(=O)c1nnc(-c2cc(C(C)C)c(O)cc2O)n1-c1ccc(CN2CCN(C(=O)C[C@H](N)C(=O)N[C@@H](C)C(=O)N3CCN(Cc4ccc(-n5c(C(=O)NCC)nnc5-c5cc(C(C)C)c(O)cc5O)cc4)CC3)CC2)cc1 ; CCNC(=O)c1nnc(-c2cc(C(C)C)c(O)cc2O)n1-c1ccc(OC2CCN(C(=O)CC(N)CC(=O)N3CCC(Oc4ccc(-n5c(C(=O)NCC)nnc5-c5cc(C(C)C)c(O)cc5O)cc4)CC3)CC2)cc1]

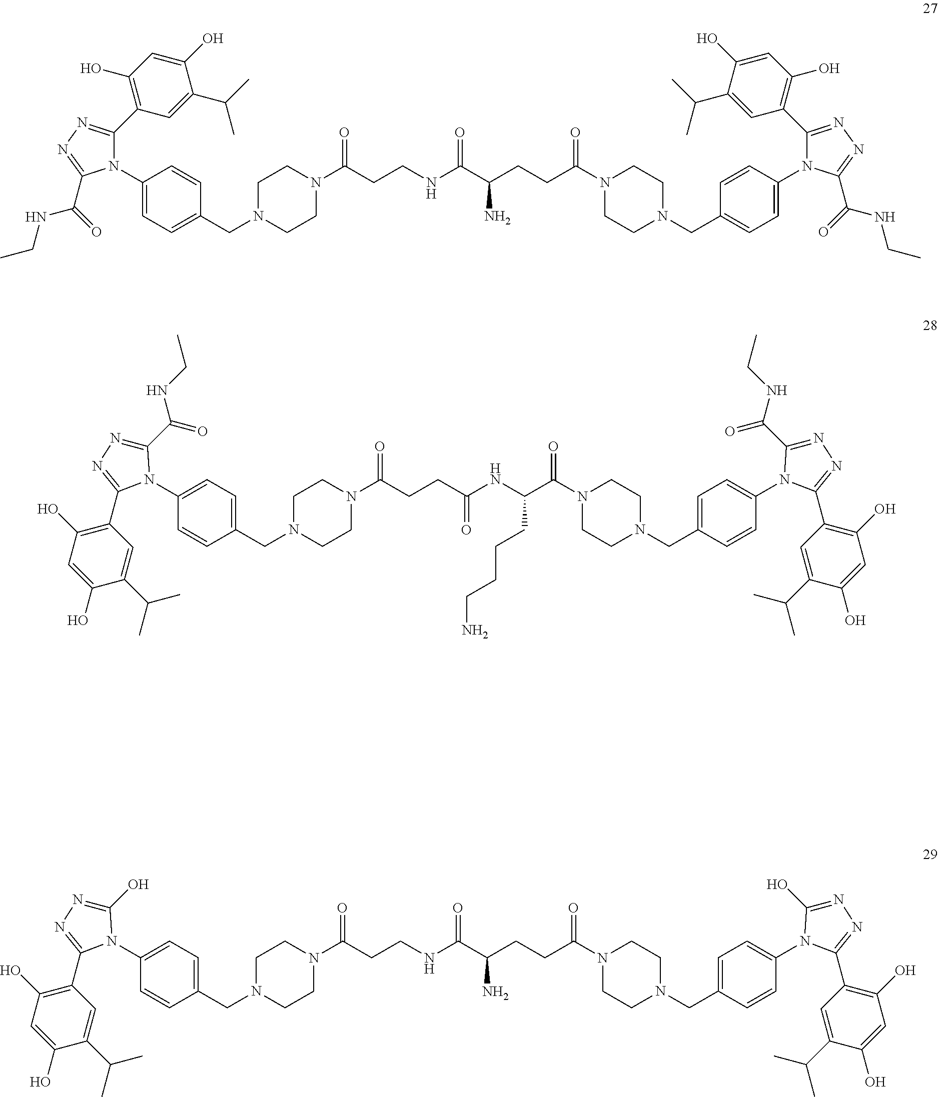 [SMILES: CCNC(=O)c1nnc(-c2cc(C(C)C)c(O)cc2O)n1-c1ccc(CN2CCN(C(=O)CCNC(=O)[C@H](N)CCC(=O)N3CCN(Cc4ccc(-n5c(C(=O)NCC)nnc5-c5cc(C(C)C)c(O)cc5O)cc4)CC3)CC2)cc1 ; CCNC(=O)c1nnc(-c2cc(C(C)C)c(O)cc2O)n1-c1ccc(CN2CCN(C(=O)CCC(=O)N[C@@H](CCCCN)C(=O)N3CCN(Cc4ccc(-n5c(C(=O)NCC)nnc5-c5cc(C(C)C)c(O)cc5O)cc4)CC3)CC2)cc1 ; CC(C)c1cc(-c2nnc(O)n2-c2ccc(CN3CCN(C(=O)CCNC(=O)[C@H](N)CCC(=O)N4CCN(Cc5ccc(-n6c(O)nnc6-c6cc(C(C)C)c(O)cc6O)cc5)CC4)CC3)cc2)c(O)cc1O]

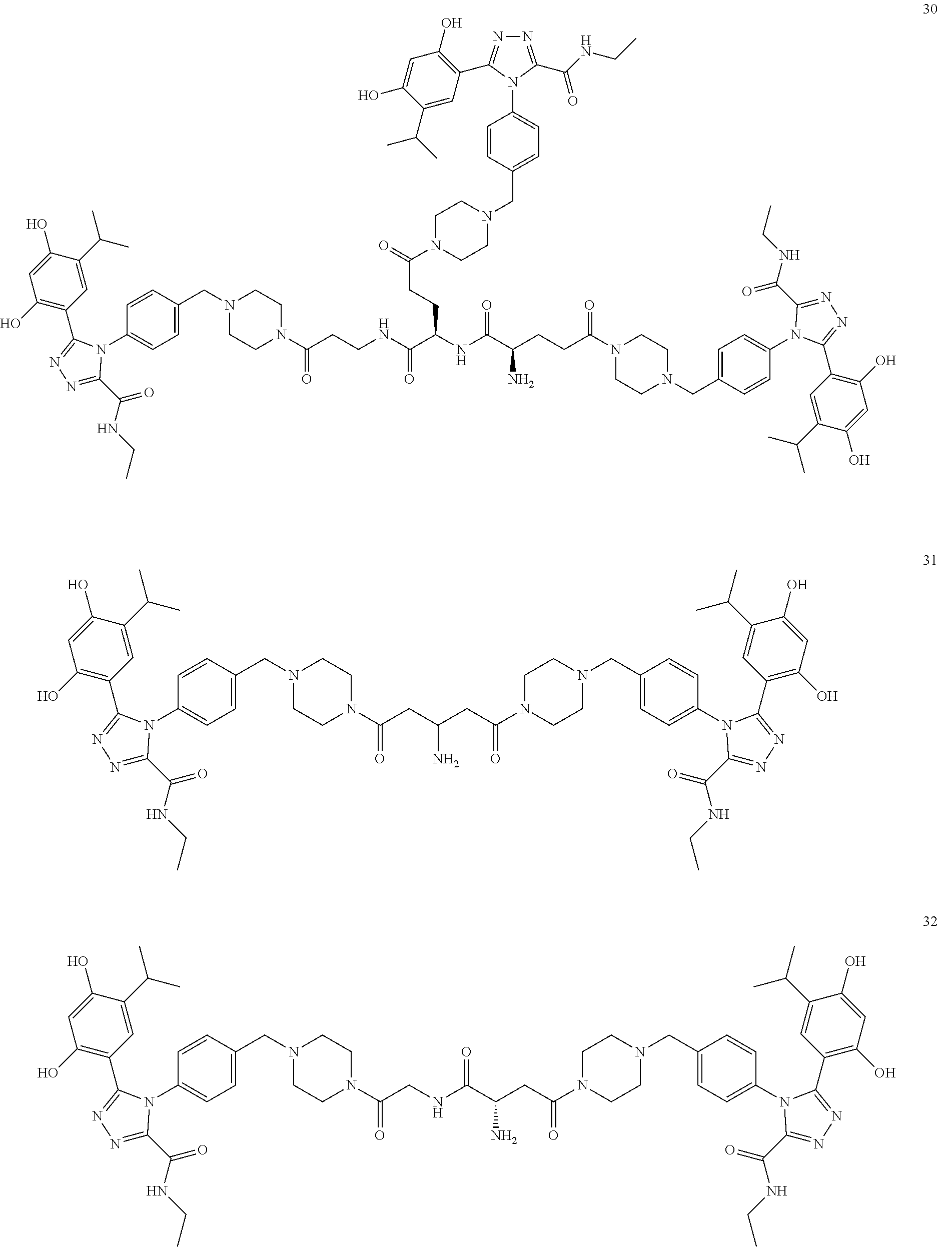

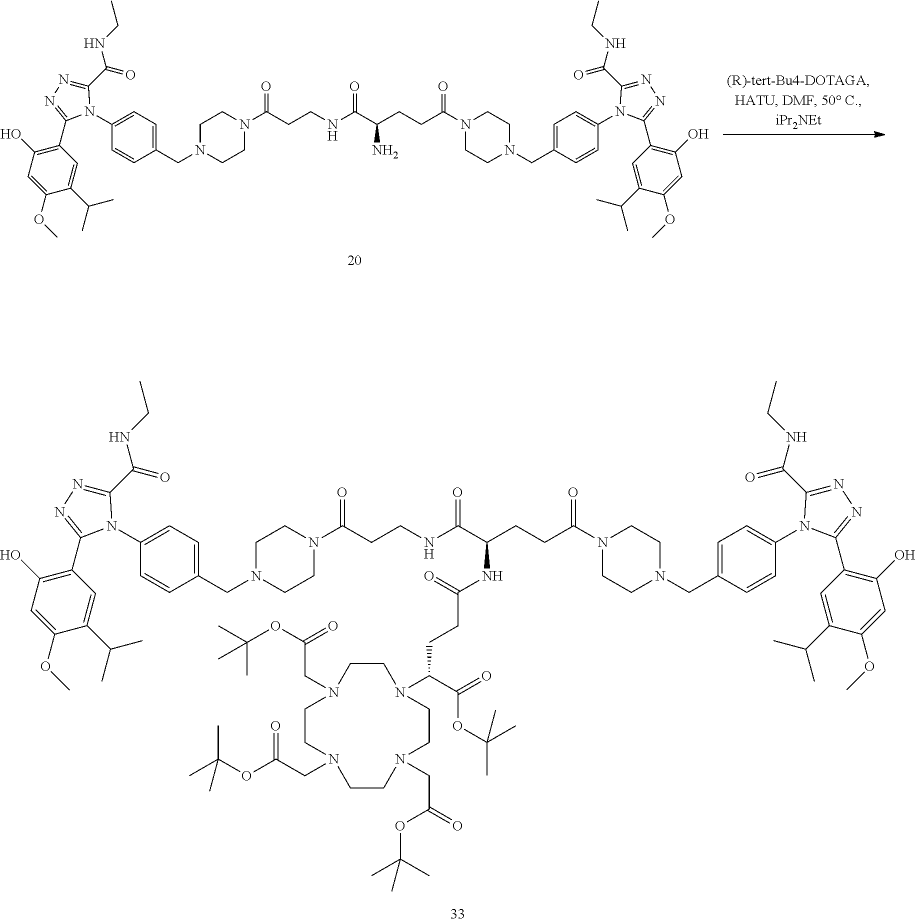 [SMILES: CCNC(=O)c1nnc(-c2cc(C(C)C)c(OC)cc2O)n1-c1ccc(CN2CCN(C(=O)CCNC(=O)[C@H](N)CCC(=O)N3CCN(Cc4ccc(-n5c(C(=O)NCC)nnc5-c5cc(C(C)C)c(OC)cc5O)cc4)CC3)CC2)cc1 ; CCNC(=O)c1nnc(-c2cc(C(C)C)c(OC)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(OC)cc5O)cc4)CC3)NC(=O)CC[C@H](C(=O)OC(C)(C)C)N3CCN(CC(=O)OC(C)(C)C)CCN(CC(=O)OC(C)(C)C)CCN(CC(=O)OC(C)(C)C)CC3)CC2)cc1]

To a vial containing 20 trifluoroacetate salt (64.9 mg, 43.8 μmol), (R)-tert-Bu4-DOTAGA (74.9 mg, 109 μmol), and HATU (31.9 mg, 84.7 μmol) was added DMF (2 mL) and diisopropylethylamine (0.20 mL). The solution was stirred at 50° C. for 1 h, then purified by preparative HPLC to give 33 (43 mg, 23.6 μmol), 53% yield). Compounds 34-46 were made in a similar manner.

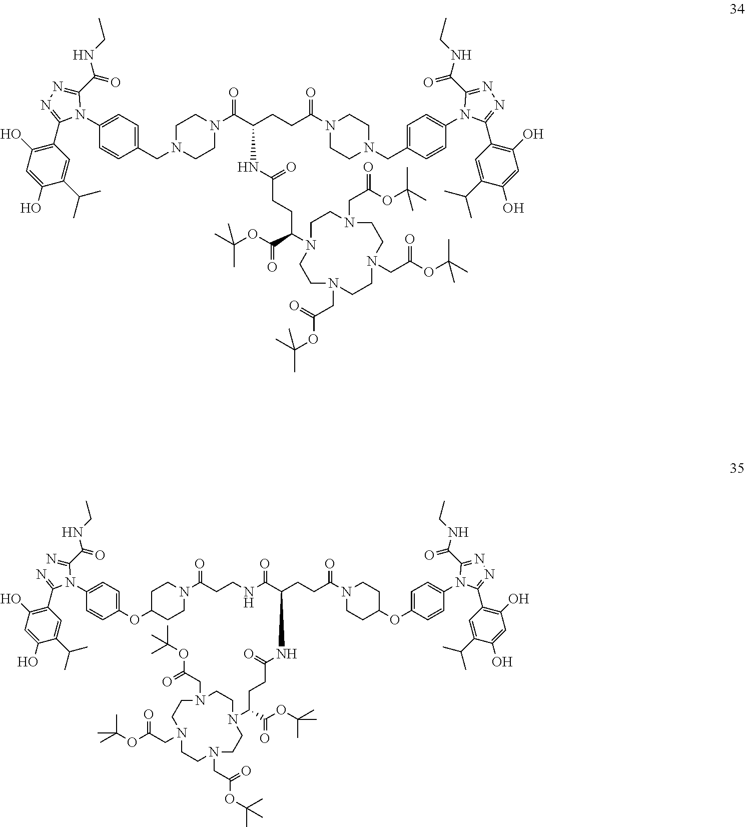 [SMILES: CCNC(=O)c1nnc(-c2cc(C(C)C)c(O)cc2O)n1-c1ccc(CN2CCN(C(=O)CC[C@H](NC(=O)CC[C@H](C(=O)OC(C)(C)C)N3CCN(CC(=O)OC(C)(C)C)CCN(CC(=O)OC(C)(C)C)CCN(CC(=O)OC(C)(C)C)CC3)C(=O)N3CCN(Cc4ccc(-n5c(C(=O)NCC)nnc5-c5cc(C(C)C)c(O)cc5O)cc4)CC3)CC2)cc1 ; CCNC(=O)c1nnc(-c2cc(C(C)C)c(O)cc2O)n1-c1ccc(OC2CCN(C(=O)CCNC(=O)[C@@H](CCC(=O)N3CCC(Oc4ccc(-n5c(C(=O)NCC)nnc5-c5cc(C(C)C)c(O)cc5O)cc4)CC3)NC(=O)CC[C@H](C(=O)OC(C)(C)C)N3CCN(CC(=O)OC(C)(C)C)CCN(CC(=O)OC(C)(C)C)CCN(CC(=O)OC(C)(C)C)CC3)CC2)cc1]

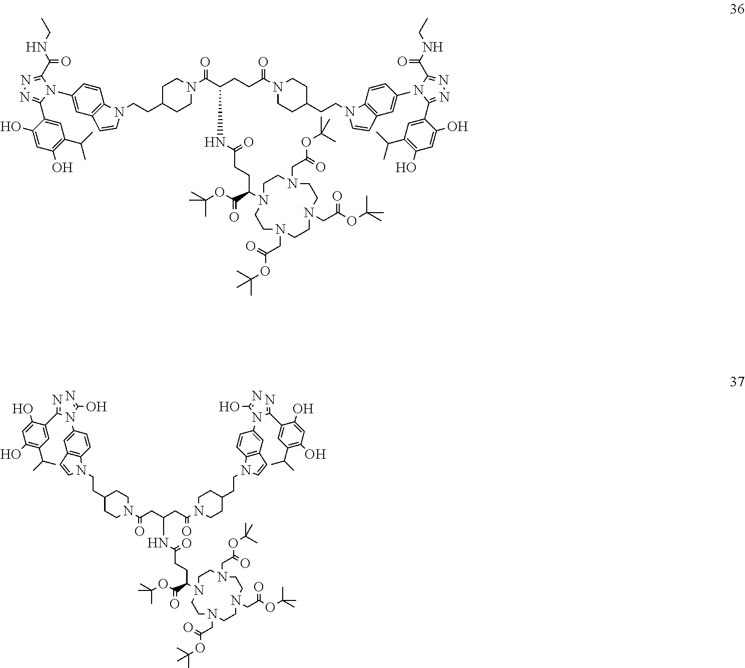 [SMILES: CCNC(=O)c1nnc(-c2cc(C(C)C)c(O)cc2O)n1-c1ccc2c(ccn2CCC2CCN(C(=O)CC[C@H](NC(=O)CC[C@H](C(=O)OC(C)(C)C)N3CCN(CC(=O)OC(C)(C)C)CCN(CC(=O)OC(C)(C)C)CCN(CC(=O)OC(C)(C)C)CC3)C(=O)N3CCC(CCn4ccc5cc(-n6c(C(=O)NCC)nnc6-c6cc(C(C)C)c(O)cc6O)ccc54)CC3)CC2)c1 ; CC(C)c1cc(-c2nnc(O)n2-c2ccc3c(ccn3CCC3CCN(C(=O)CC(CC(=O)N4CCC(CCn5ccc6cc(-n7c(O)nnc7-c7cc(C(C)C)c(O)cc7O)ccc65)CC4)NC(=O)CC[C@H](C(=O)OC(C)(C)C)N4CCN(CC(=O)OC(C)(C)C)CCN(CC(=O)OC(C)(C)C)CCN(CC(=O)OC(C)(C)C)CC4)CC3)c2)c(O)cc1O]

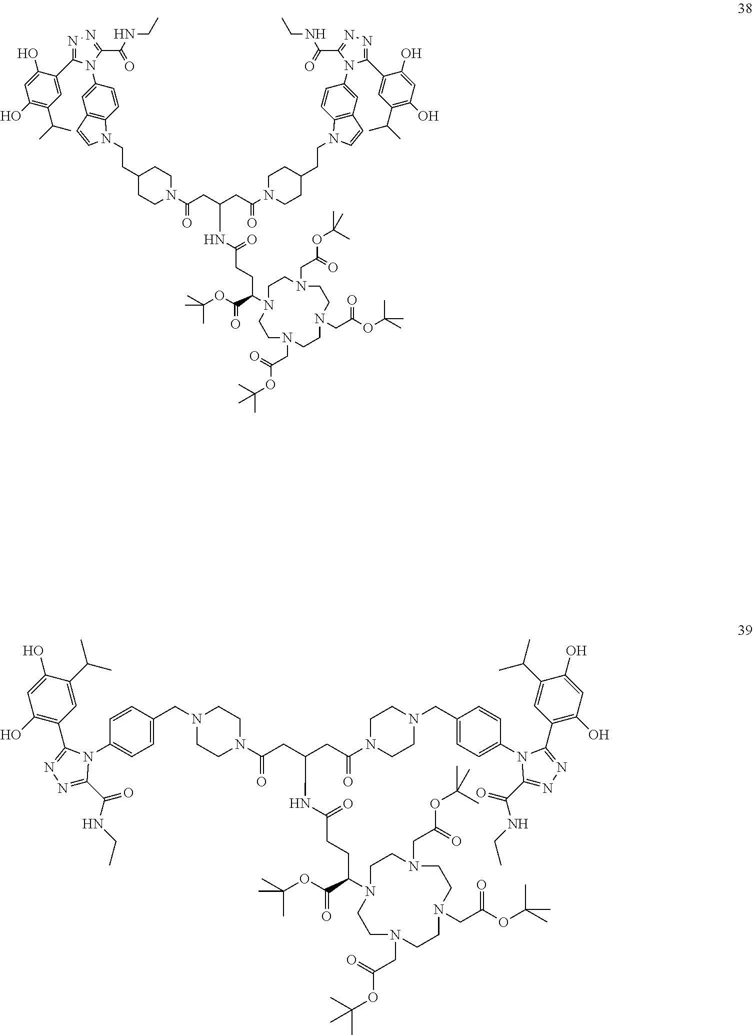 [SMILES: CCNC(=O)c1nnc(-c2cc(C(C)C)c(O)cc2O)n1-c1ccc2c(ccn2CCC2CCN(C(=O)CC(CC(=O)N3CCC(CCn4ccc5cc(-n6c(C(=O)NCC)nnc6-c6cc(C(C)C)c(O)cc6O)ccc54)CC3)NC(=O)CC[C@H](C(=O)OC(C)(C)C)N3CCN(CC(=O)OC(C)(C)C)CCN(CC(=O)OC(C)(C)C)CCN(CC(=O)OC(C)(C)C)CC3)CC2)c1 ; CCNC(=O)c1nnc(-c2cc(C(C)C)c(O)cc2O)n1-c1ccc(CN2CCN(C(=O)CC(CC(=O)N3CCN(Cc4ccc(-n5c(C(=O)NCC)nnc5-c5cc(C(C)C)c(O)cc5O)cc4)CC3)NC(=O)CC[C@H](C(=O)OC(C)(C)C)N3CCN(CC(=O)OC(C)(C)C)CCN(CC(=O)OC(C)(C)C)CCN(CC(=O)OC(C)(C)C)CC3)CC2)cc1]

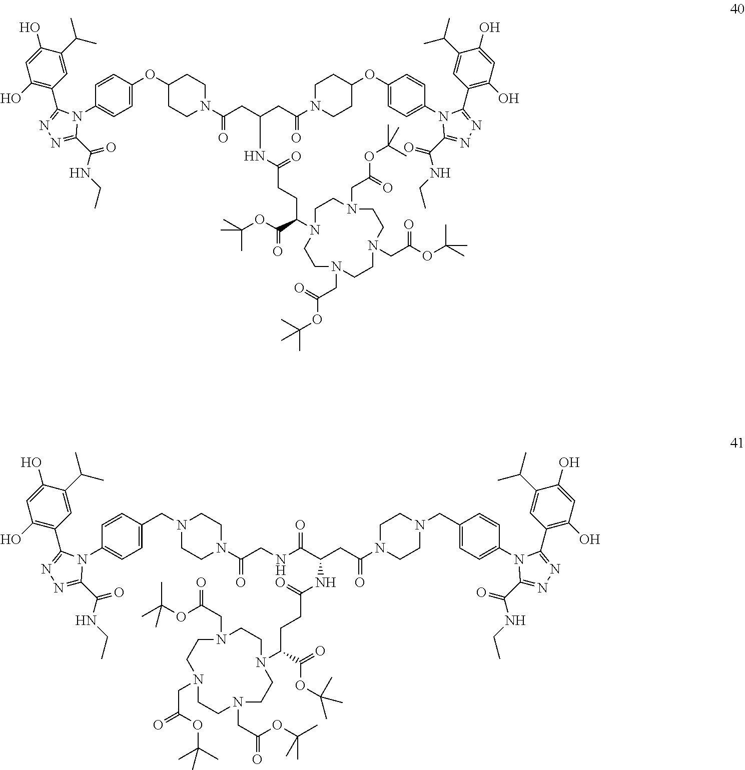 [SMILES: CCNC(=O)c1nnc(-c2cc(C(C)C)c(O)cc2O)n1-c1ccc(OC2CCN(C(=O)CC(CC(=O)N3CCC(Oc4ccc(-n5c(C(=O)NCC)nnc5-c5cc(C(C)C)c(O)cc5O)cc4)CC3)NC(=O)CC[C@H](C(=O)OC(C)(C)C)N3CCN(CC(=O)OC(C)(C)C)CCN(CC(=O)OC(C)(C)C)CCN(CC(=O)OC(C)(C)C)CC3)CC2)cc1 ; CCNC(=O)c1nnc(-c2cc(C(C)C)c(O)cc2O)n1-c1ccc(CN2CCN(C(=O)CNC(=O)[C@H](CC(=O)N3CCN(Cc4ccc(-n5c(C(=O)NCC)nnc5-c5cc(C(C)C)c(O)cc5O)cc4)CC3)NC(=O)CC[C@H](C(=O)OC(C)(C)C)N3CCN(CC(=O)OC(C)(C)C)CCN(CC(=O)OC(C)(C)C)CCN(CC(=O)OC(C)(C)C)CC3)CC2)cc1]

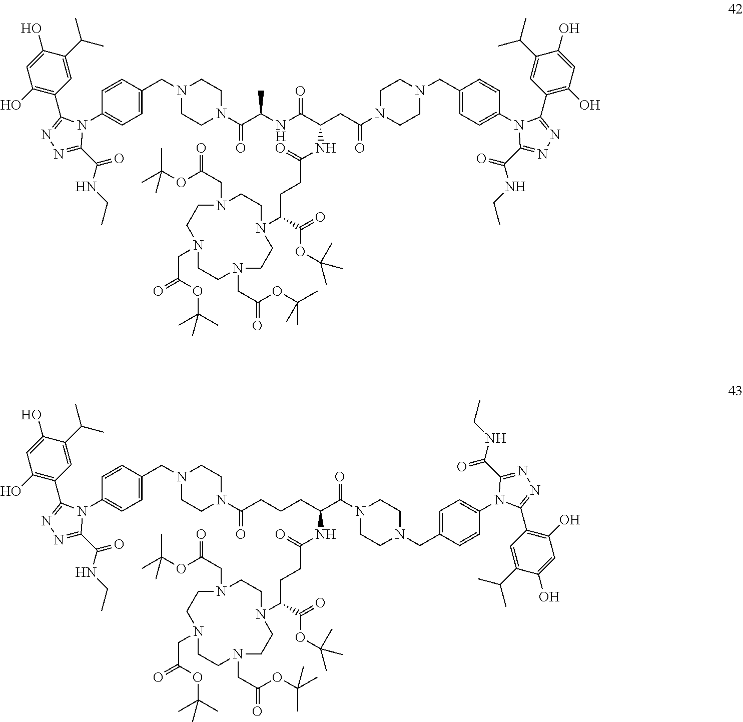 [SMILES: CCNC(=O)c1nnc(-c2cc(C(C)C)c(O)cc2O)n1-c1ccc(CN2CCN(C(=O)C[C@H](NC(=O)CC[C@H](C(=O)OC(C)(C)C)N3CCN(CC(=O)OC(C)(C)C)CCN(CC(=O)OC(C)(C)C)CCN(CC(=O)OC(C)(C)C)CC3)C(=O)N[C@H](C)C(=O)N3CCN(Cc4ccc(-n5c(C(=O)NCC)nnc5-c5cc(C(C)C)c(O)cc5O)cc4)CC3)CC2)cc1 ; CCNC(=O)c1nnc(-c2cc(C(C)C)c(O)cc2O)n1-c1ccc(CN2CCN(C(=O)CCC[C@H](NC(=O)CC[C@H](C(=O)OC(C)(C)C)N3CCN(CC(=O)OC(C)(C)C)CCN(CC(=O)OC(C)(C)C)CCN(CC(=O)OC(C)(C)C)CC3)C(=O)N3CCN(Cc4ccc(-n5c(C(=O)NCC)nnc5-c5cc(C(C)C)c(O)cc5O)cc4)CC3)CC2)cc1]

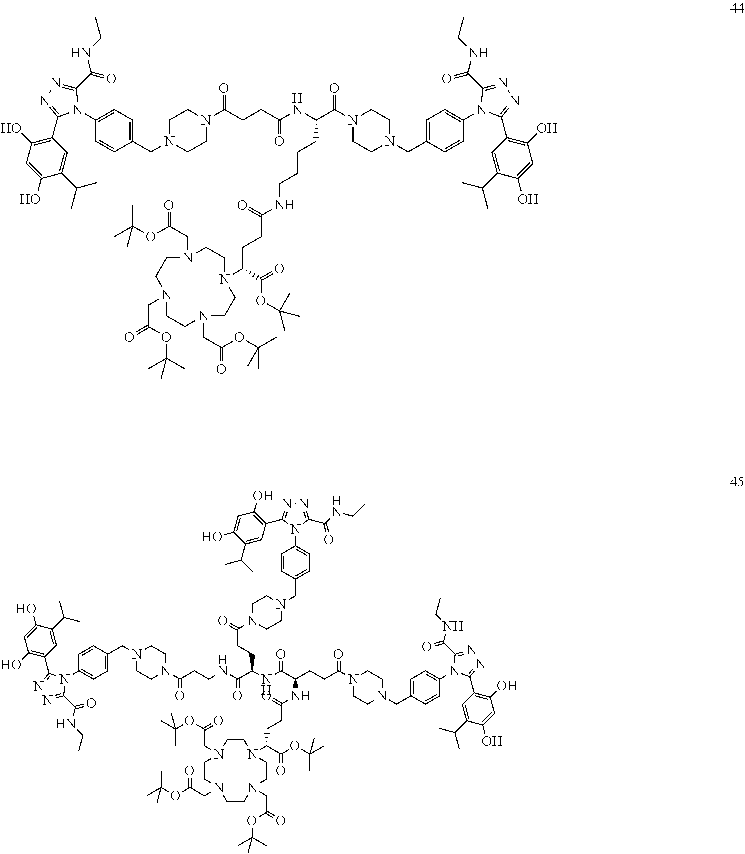

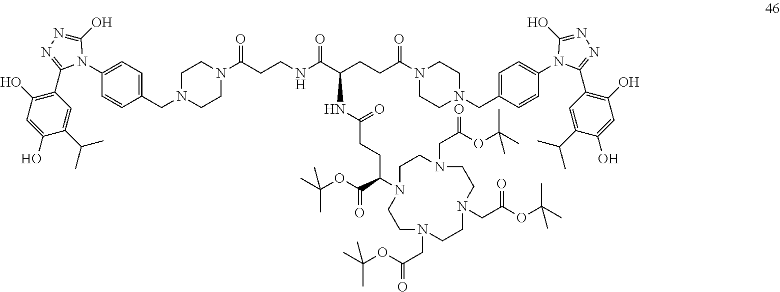 [SMILES: CC(C)c1cc(-c2nnc(O)n2-c2ccc(CN3CCN(C(=O)CCNC(=O)[C@@H](CCC(=O)N4CCN(Cc5ccc(-n6c(O)nnc6-c6cc(C(C)C)c(O)cc6O)cc5)CC4)NC(=O)CC[C@H](C(=O)OC(C)(C)C)N4CCN(CC(=O)OC(C)(C)C)CCN(CC(=O)OC(C)(C)C)CCN(CC(=O)OC(C)(C)C)CC4)CC3)cc2)c(O)cc1O]

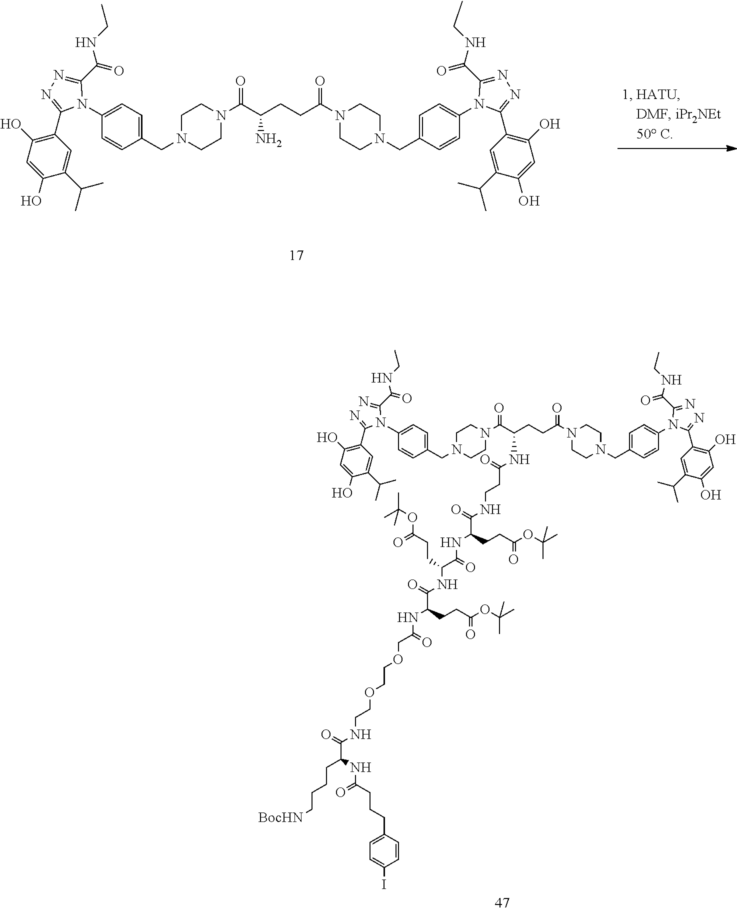 [SMILES: CCNC(=O)c1nnc(-c2cc(C(C)C)c(O)cc2O)n1-c1ccc(CN2CCN(C(=O)CC[C@H](N)C(=O)N3CCN(Cc4ccc(-n5c(C(=O)NCC)nnc5-c5cc(C(C)C)c(O)cc5O)cc4)CC3)CC2)cc1 ; "17" ; CCNC(=O)c1nnc(-c2cc(C(C)C)c(O)cc2O)n1-c1ccc(CN2CCN(C(=O)CC[C@H](NC(=O)CCNC(=O)[C@@H](CCC(=O)OC(C)(C)C)NC(=O)[C@@H](CCC(=O)OC(C)(C)C)NC(=O)[C@@H](CCC(=O)OC(C)(C)C)NC(=O)COCCOCCNC(=O)[C@H](CCCCNC(=O)OC(C)(C)C)NC(=O)CCCc3ccc(I)cc3)C(=O)N3CCN(Cc4ccc(-n5c(C(=O)NCC)nnc5-c5cc(C(C)C)c(O)cc5O)cc4)CC3)CC2)cc1]

A vial was charged with 17 trifluoroacetate salt (120 mg, 86.8 μmol) and 1 (131 mg, 101 μmol). To this was added a solution of HATU (36.0 mg, 95.5 μmol) in DMF (4 mL). Diisopropylethylamine (0.40 mL) was added, and the reaction stirred at 50° C. for 1 h. The reaction mixture was purified by preparative HPLC to provide 47 (38.0 mg, 16.4 μmol, 18% yield). Compounds 48-53 were prepared in a similar manner.

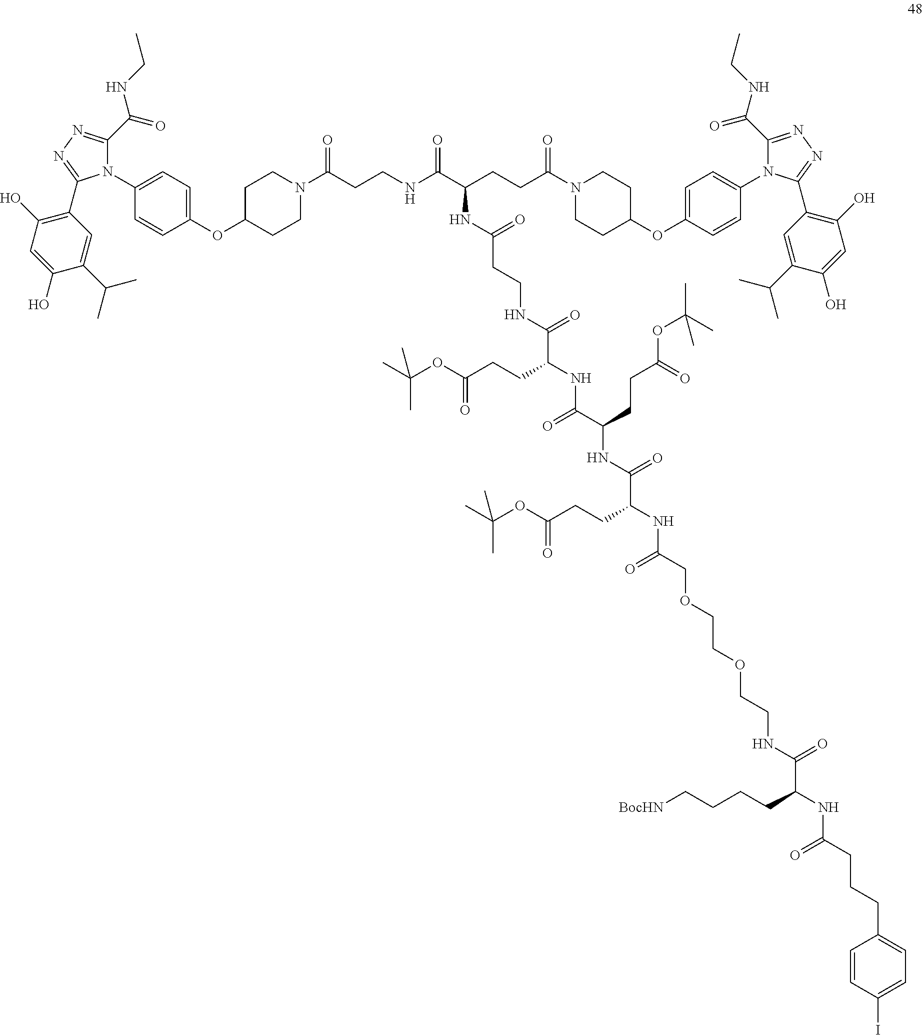 [SMILES: CCNC(=O)c1nnc(-c2cc(C(C)C)c(O)cc2O)n1-c1ccc(OC2CCN(C(=O)CCNC(=O)[C@@H](CCC(=O)N3CCC(Oc4ccc(-n5c(C(=O)NCC)nnc5-c5cc(C(C)C)c(O)cc5O)cc4)CC3)NC(=O)CCNC(=O)[C@@H](CCC(=O)OC(C)(C)C)NC(=O)[C@@H](CCC(=O)OC(C)(C)C)NC(=O)[C@@H](CCC(=O)OC(C)(C)C)NC(=O)COCCOCCNC(=O)[C@H](CCCCNC(=O)OC(C)(C)C)NC(=O)CCCc3ccc(I)cc3)CC2)cc1]

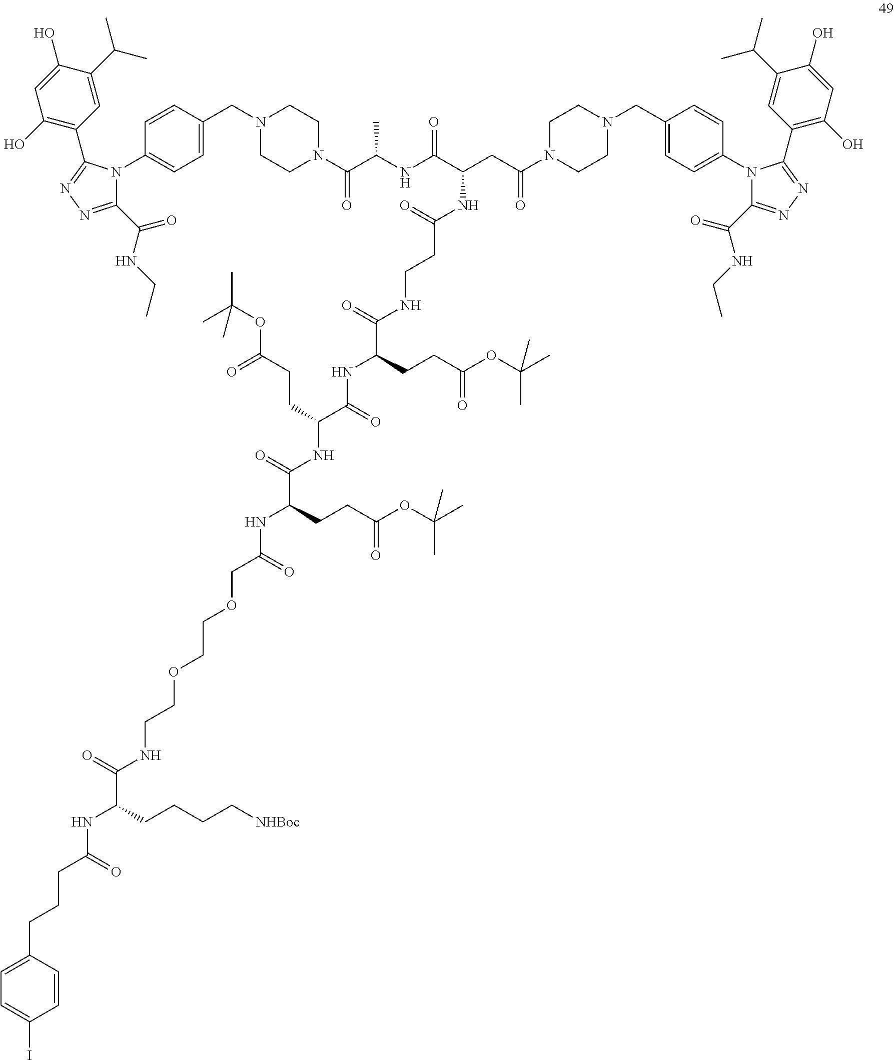 [SMILES: CCNC(=O)c1nnc(-c2cc(C(C)C)c(O)cc2O)n1-c1ccc(CN2CCN(C(=O)C[C@H](NC(=O)CCNC(=O)[C@@H](CCC(=O)OC(C)(C)C)NC(=O)[C@@H](CCC(=O)OC(C)(C)C)NC(=O)[C@@H](CCC(=O)OC(C)(C)C)NC(=O)COCCOCCNC(=O)[C@H](CCCCNC(=O)OC(C)(C)C)NC(=O)CCCc3ccc(I)cc3)C(=O)N[C@@H](C)C(=O)N3CCN(Cc4ccc(-n5c(C(=O)NCC)nnc5-c5cc(C(C)C)c(O)cc5O)cc4)CC3)CC2)cc1]

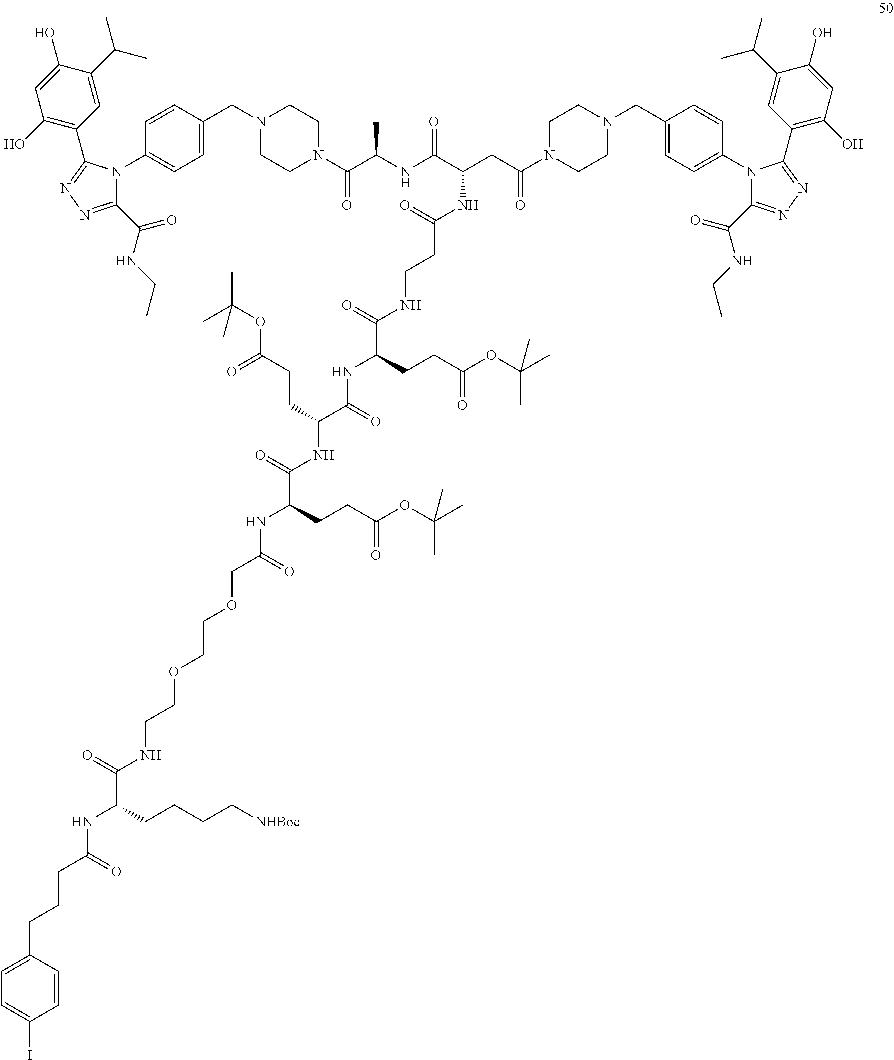 [SMILES: CCNC(=O)c1nnc(-c2cc(C(C)C)c(O)cc2O)n1-c1ccc(CN2CCN(C(=O)C[C@H](NC(=O)CCNC(=O)[C@@H](CCC(=O)OC(C)(C)C)NC(=O)[C@@H](CCC(=O)OC(C)(C)C)NC(=O)[C@@H](CCC(=O)OC(C)(C)C)NC(=O)COCCOCCNC(=O)[C@H](CCCCNC(=O)OC(C)(C)C)NC(=O)CCCc3ccc(I)cc3)C(=O)N[C@H](C)C(=O)N3CCN(Cc4ccc(-n5c(C(=O)NCC)nnc5-c5cc(C(C)C)c(O)cc5O)cc4)CC3)CC2)cc1]

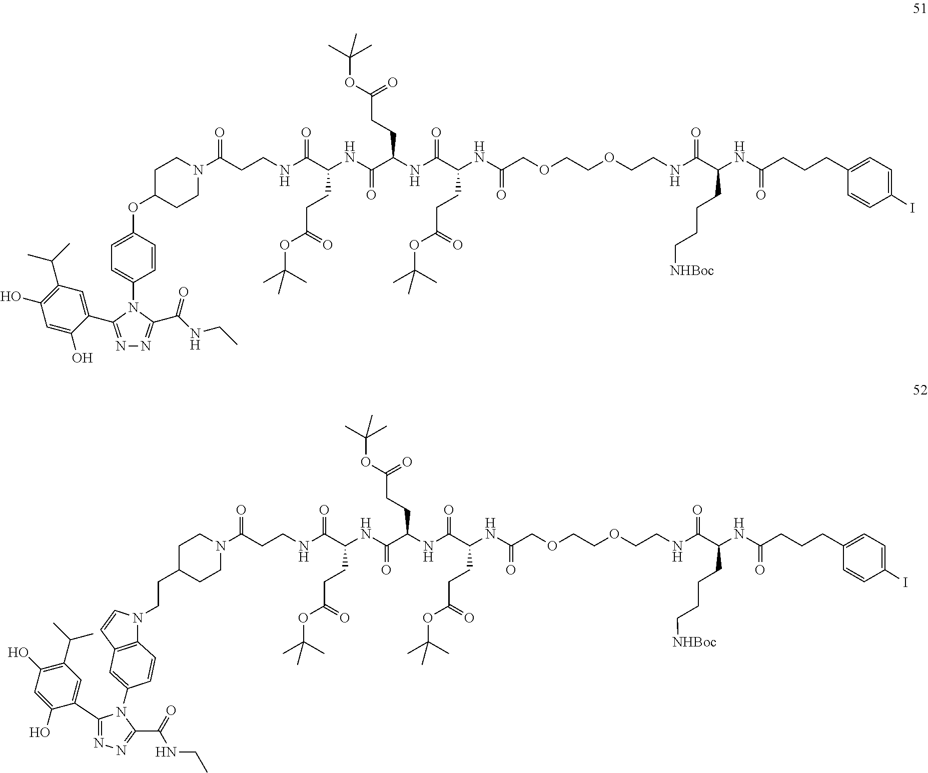 [SMILES: CCNC(=O)c1nnc(-c2cc(C(C)C)c(O)cc2O)n1-c1ccc(OC2CCN(C(=O)CCNC(=O)[C@@H](CCC(=O)OC(C)(C)C)NC(=O)[C@@H](CCC(=O)OC(C)(C)C)NC(=O)[C@@H](CCC(=O)OC(C)(C)C)NC(=O)COCCOCCNC(=O)[C@H](CCCCNC(=O)OC(C)(C)C)NC(=O)CCCc3ccc(I)cc3)CC2)cc1 ; CCNC(=O)c1nnc(-c2cc(C(C)C)c(O)cc2O)n1-c1ccc2c(ccn2CCC2CCN(C(=O)CCNC(=O)[C@@H](CCC(=O)OC(C)(C)C)NC(=O)[C@@H](CCC(=O)OC(C)(C)C)NC(=O)[C@@H](CCC(=O)OC(C)(C)C)NC(=O)COCCOCCNC(=O)[C@H](CCCCNC(=O)OC(C)(C)C)NC(=O)CCCc3ccc(I)cc3)CC2)c1]

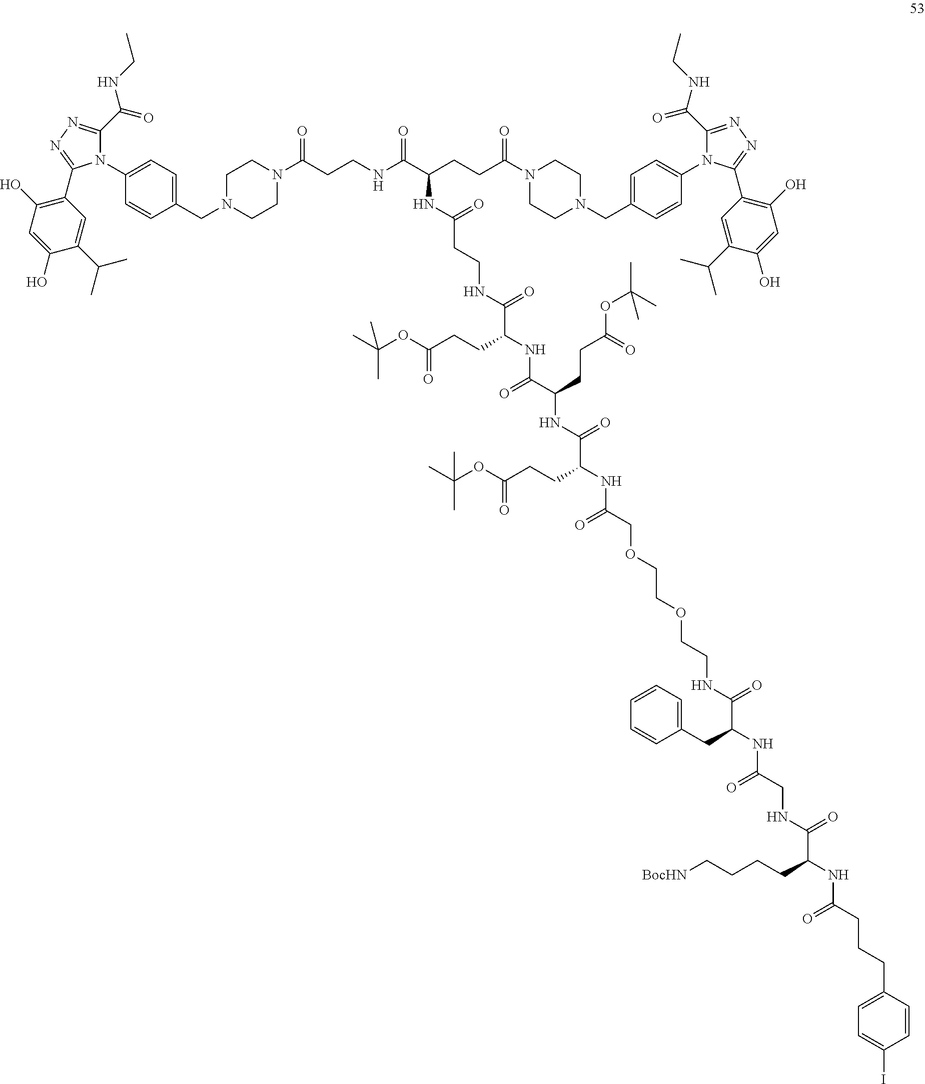 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)[C@@H](CCC(=O)OC(C)(C)C)NC(=O)[C@@H](CCC(=O)OC(C)(C)C)NC(=O)[C@@H](CCC(=O)OC(C)(C)C)NC(=O)COCCOCCNC(=O)[C@H](Cc3ccccc3)NC(=O)CNC(=O)[C@H](CCCCNC(=O)OC(C)(C)C)NC(=O)CCCc3ccc(I)cc3)CC2)cc1]

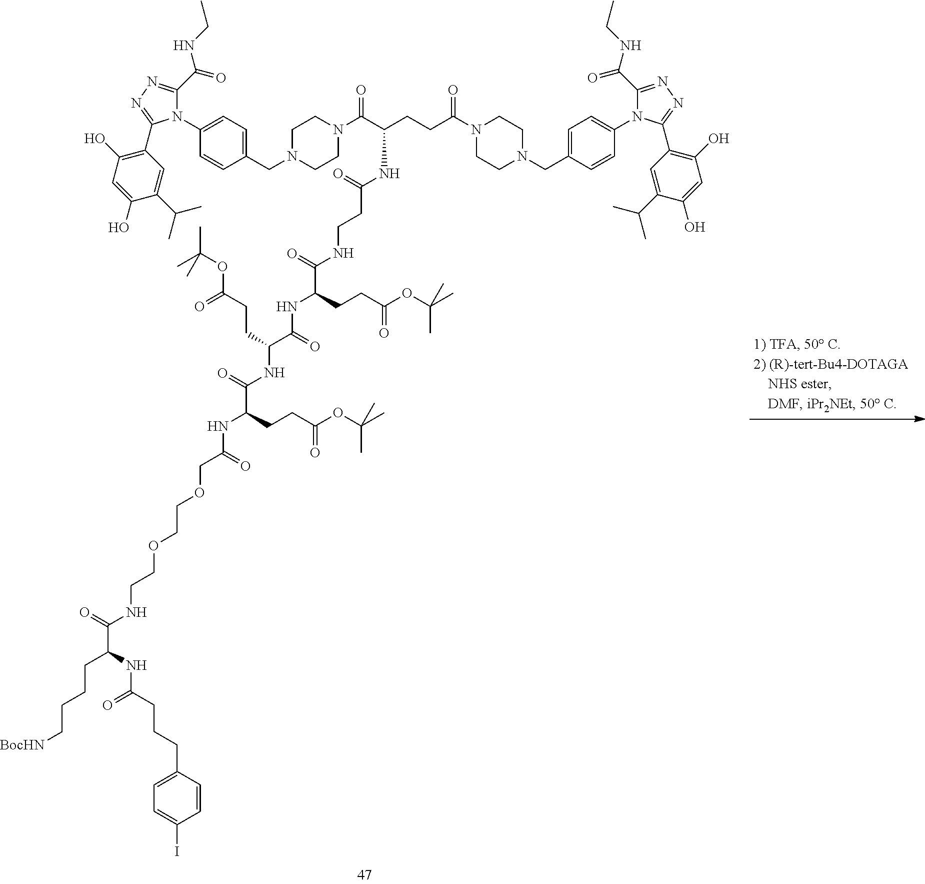 [SMILES: CCNC(=O)c1nnc(-c2cc(C(C)C)c(O)cc2O)n1-c1ccc(CN2CCN(C(=O)CC[C@H](NC(=O)CCNC(=O)[C@@H](CCC(=O)OC(C)(C)C)NC(=O)[C@@H](CCC(=O)OC(C)(C)C)NC(=O)[C@@H](CCC(=O)OC(C)(C)C)NC(=O)COCCOCCNC(=O)[C@H](CCCCNC(=O)OC(C)(C)C)NC(=O)CCCc3ccc(I)cc3)C(=O)N3CCN(Cc4ccc(-n5c(C(=O)NCC)nnc5-c5cc(C(C)C)c(O)cc5O)cc4)CC3)CC2)cc1]

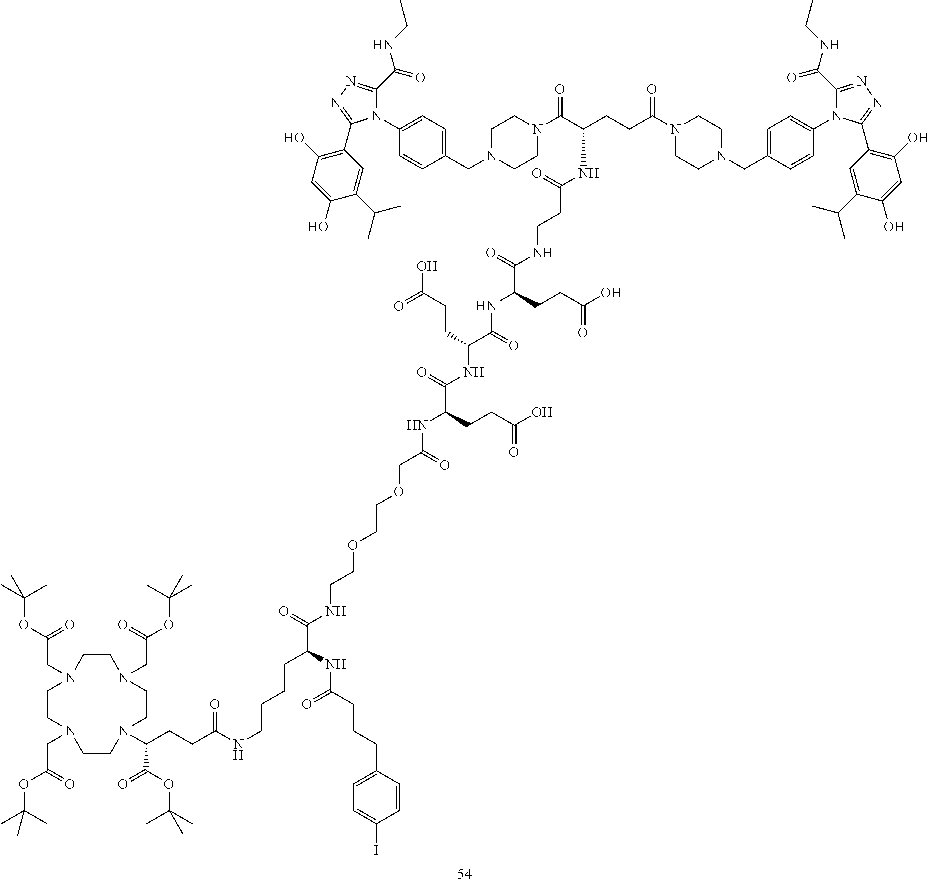 [SMILES: CCNC(=O)c1nnc(-c2cc(C(C)C)c(O)cc2O)n1-c1ccc(CN2CCN(C(=O)CC[C@H](NC(=O)CCNC(=O)[C@@H](CCC(=O)O)NC(=O)[C@@H](CCC(=O)O)NC(=O)[C@@H](CCC(=O)O)NC(=O)COCCOCCNC(=O)[C@H](CCCCNC(=O)CC[C@H](C(=O)OC(C)(C)C)N3CCN(CC(=O)OC(C)(C)C)CCN(CC(=O)OC(C)(C)C)CCN(CC(=O)OC(C)(C)C)CC3)NC(=O)CCCc3ccc(I)cc3)C(=O)N3CCN(Cc4ccc(-n5c(C(=O)NCC)nnc5-c5cc(C(C)C)c(O)cc5O)cc4)CC3)CC2)cc1]

A vial was charged with 47 (18.0 mg, 7.78 μmol) and TFA (1 mL) was added. The reaction was stirred at 50° C. for 1 h, then the TFA was removed under vacuum. To the remaining material was added a solution of (R)-tert-Bu4-DOTAGA NHS ester (18.6 mg, 23.3 μmol) in DMF (3 mL). Diisopropylethylamine (0.5 mL) was added, and the solution stirred at 50° C. for 30 min, then purified by preparative HPLC to give 54 (11.2 mg, 4.11 μmol, 52% yield). Compounds 55-60 were made in a similar manner.

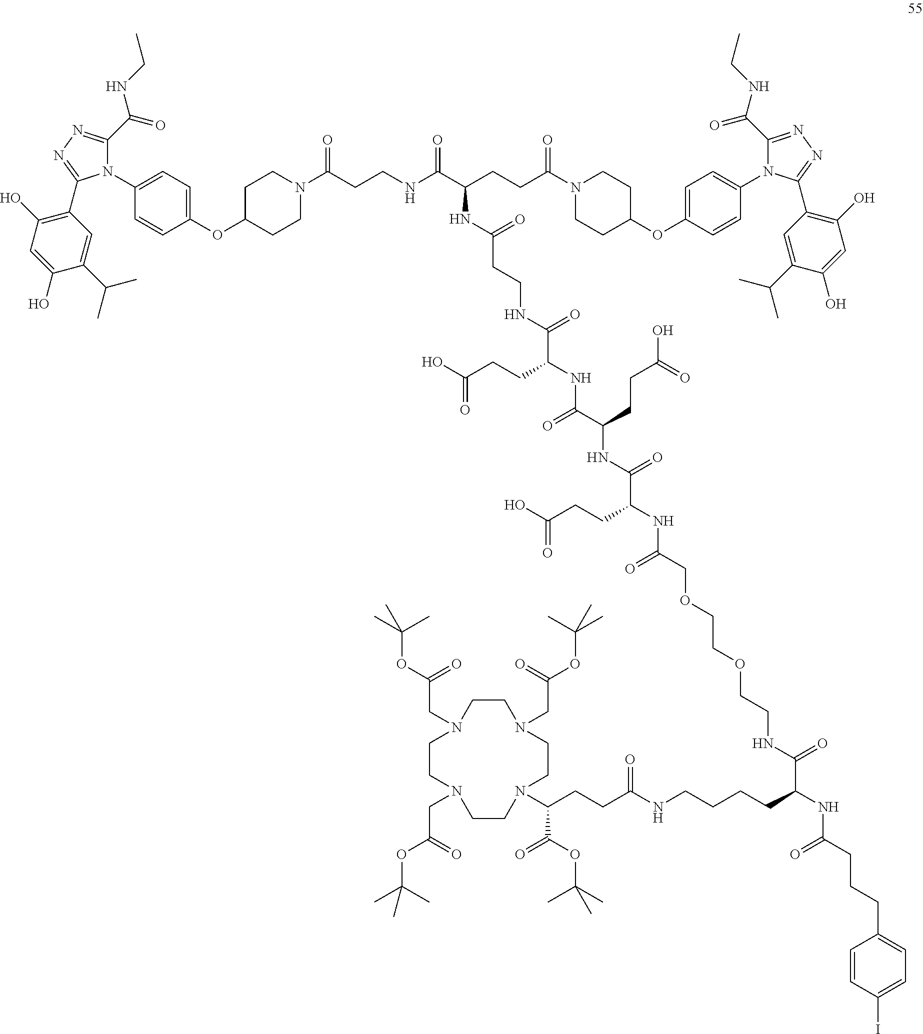 [SMILES: CCNC(=O)c1nnc(-c2cc(C(C)C)c(O)cc2O)n1-c1ccc(OC2CCN(C(=O)CCNC(=O)[C@@H](CCC(=O)N3CCC(Oc4ccc(-n5c(C(=O)NCC)nnc5-c5cc(C(C)C)c(O)cc5O)cc4)CC3)NC(=O)CCNC(=O)[C@@H](CCC(=O)O)NC(=O)[C@@H](CCC(=O)O)NC(=O)[C@@H](CCC(=O)O)NC(=O)COCCOCCNC(=O)[C@H](CCCCNC(=O)CC[C@H](C(=O)OC(C)(C)C)N3CCN(CC(=O)OC(C)(C)C)CCN(CC(=O)OC(C)(C)C)CCN(CC(=O)OC(C)(C)C)CC3)NC(=O)CCCc3ccc(I)cc3)CC2)cc1]

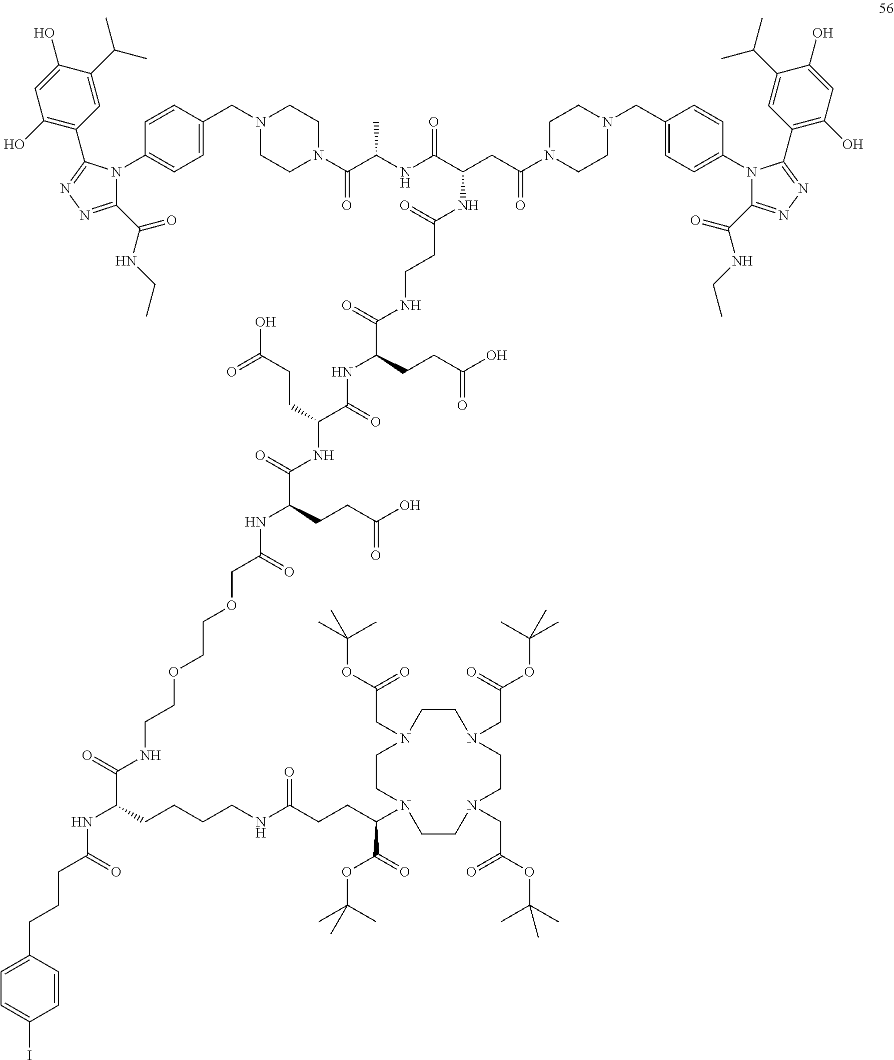 [SMILES: CCNC(=O)c1nnc(-c2cc(C(C)C)c(O)cc2O)n1-c1ccc(CN2CCN(C(=O)C[C@H](NC(=O)CCNC(=O)[C@@H](CCC(=O)O)NC(=O)[C@@H](CCC(=O)O)NC(=O)[C@@H](CCC(=O)O)NC(=O)COCCOCCNC(=O)[C@H](CCCCNC(=O)CC[C@H](C(=O)OC(C)(C)C)N3CCN(CC(=O)OC(C)(C)C)CCN(CC(=O)OC(C)(C)C)CCN(CC(=O)OC(C)(C)C)CC3)NC(=O)CCCc3ccc(I)cc3)C(=O)N[C@@H](C)C(=O)N3CCN(Cc4ccc(-n5c(C(=O)NCC)nnc5-c5cc(C(C)C)c(O)cc5O)cc4)CC3)CC2)cc1]

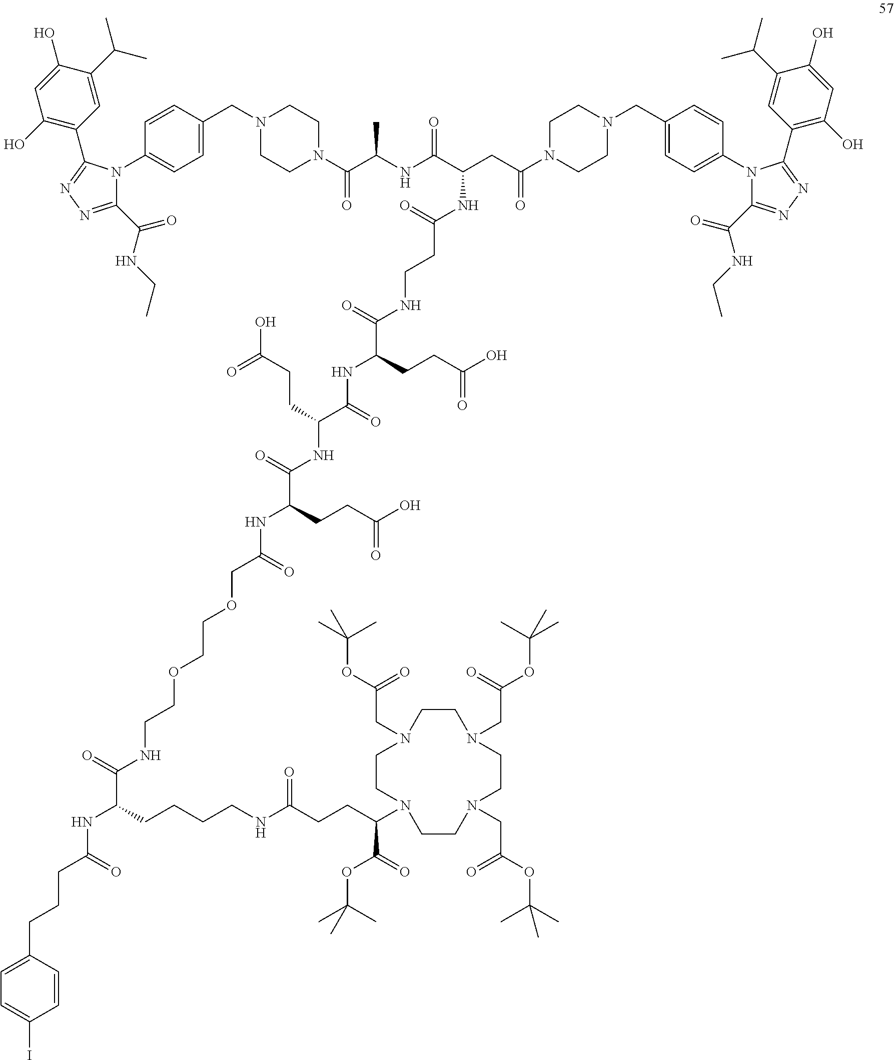 [SMILES: CCNC(=O)c1nnc(-c2cc(C(C)C)c(O)cc2O)n1-c1ccc(CN2CCN(C(=O)C[C@H](NC(=O)CCNC(=O)[C@@H](CCC(=O)O)NC(=O)[C@@H](CCC(=O)O)NC(=O)[C@@H](CCC(=O)O)NC(=O)COCCOCCNC(=O)[C@H](CCCCNC(=O)CC[C@H](C(=O)OC(C)(C)C)N3CCN(CC(=O)OC(C)(C)C)CCN(CC(=O)OC(C)(C)C)CCN(CC(=O)OC(C)(C)C)CC3)NC(=O)CCCc3ccc(I)cc3)C(=O)N[C@H](C)C(=O)N3CCN(Cc4ccc(-n5c(C(=O)NCC)nnc5-c5cc(C(C)C)c(O)cc5O)cc4)CC3)CC2)cc1]

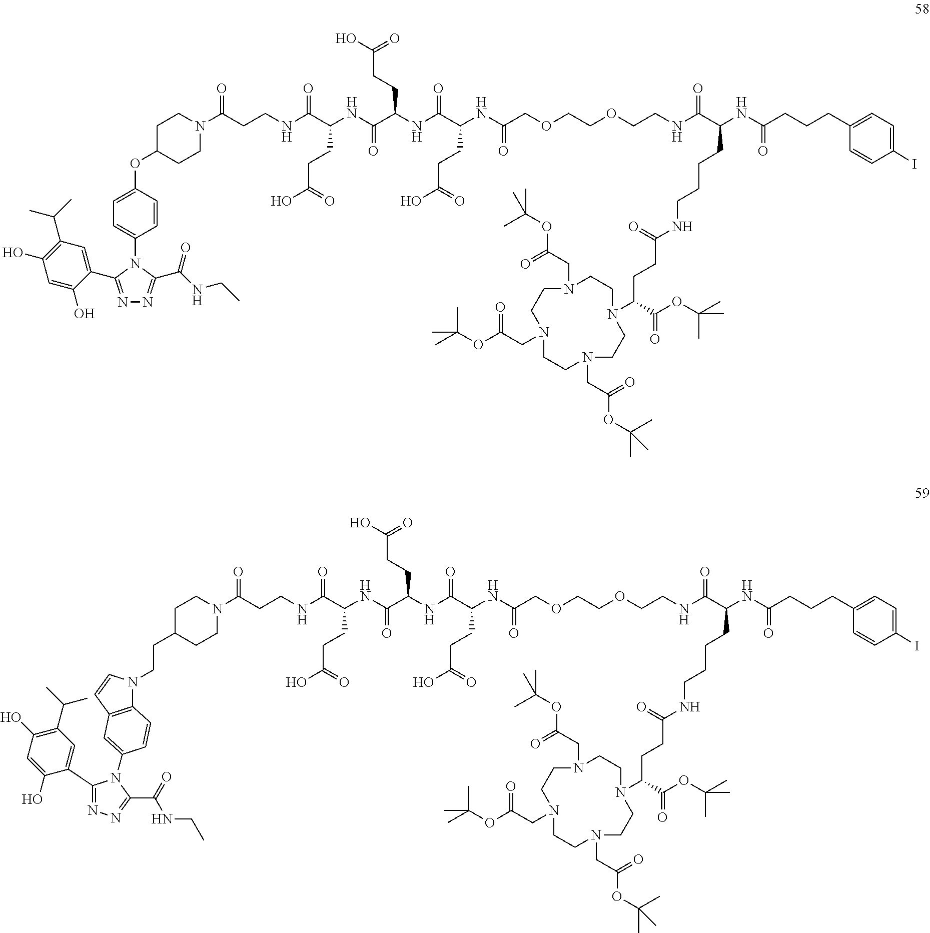

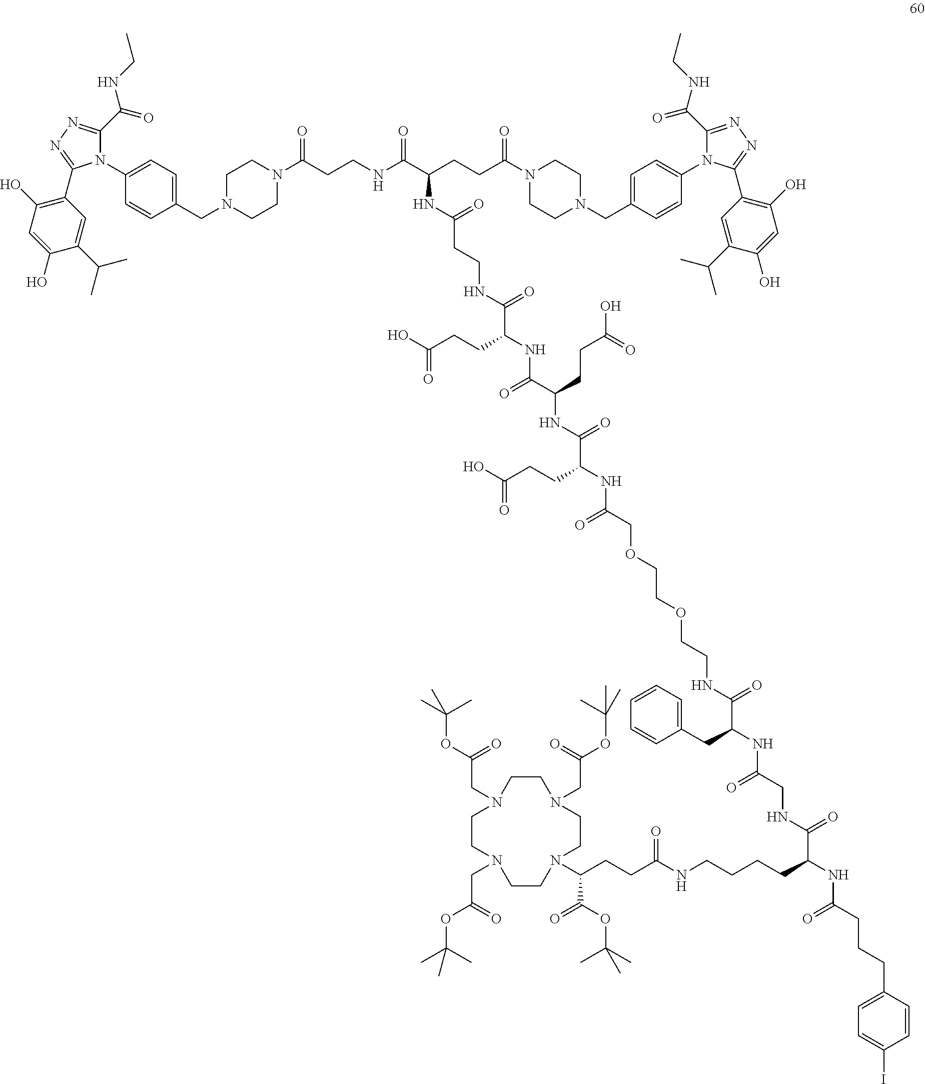 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)[C@@H](CCC(=O)O)NC(=O)[C@@H](CCC(=O)O)NC(=O)[C@@H](CCC(=O)O)NC(=O)COCCOCCNC(=O)[C@H](Cc3ccccc3)NC(=O)CNC(=O)[C@H](CCCCNC(=O)CC[C@H](C(=O)OC(C)(C)C)N3CCN(CC(=O)OC(C)(C)C)CCN(CC(=O)OC(C)(C)C)CCN(CC(=O)OC(C)(C)C)CC3)NC(=O)CCCc3ccc(I)cc3)CC2)cc1]

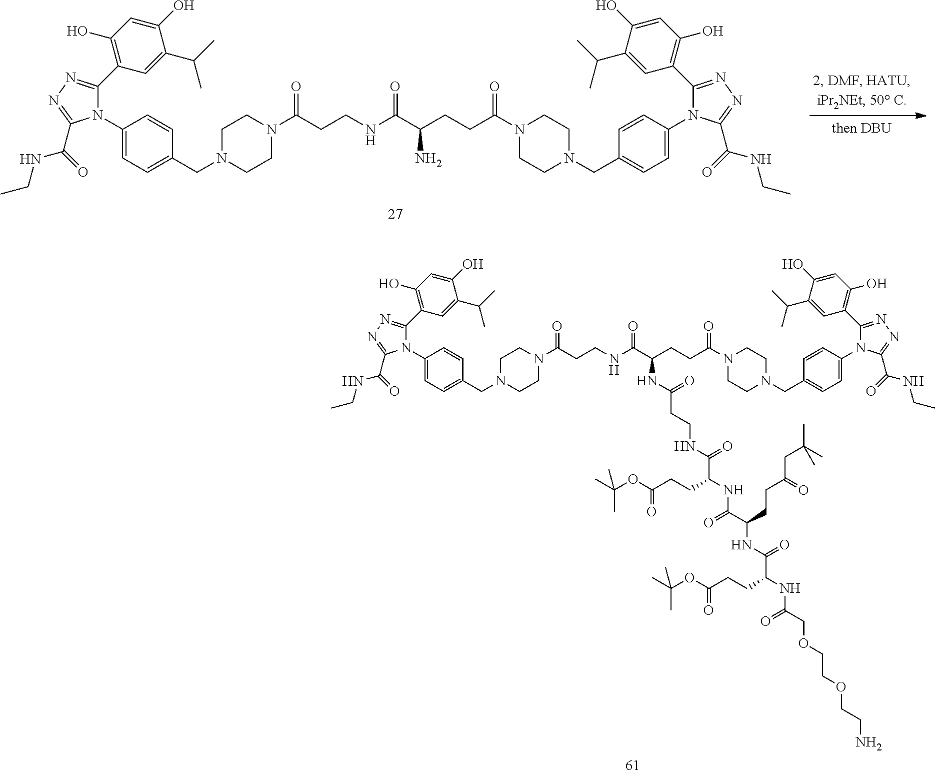

A vial was charged with 2 (41.0 mg, 40.5 μmop, 27 trifluoroacetate salt (43.8 mg, 31.7 μmop and HATU (15.5 mg, 41.0 μmop. DMF (2 mL) and diisopropylethylamine (0.2 mL) were added, and the reaction stirred at 50° C. for 1 h, and then DBU (0.2 mL) was added, and the reaction stirred at 50° C. for another 10 min. The reaction mixture was purified by preparative HPLC to give 61 trifluoroacetate salt (23.0 mg, 10.6 μmol, 33% yield). Compounds 62-67 were made in a similar manner.

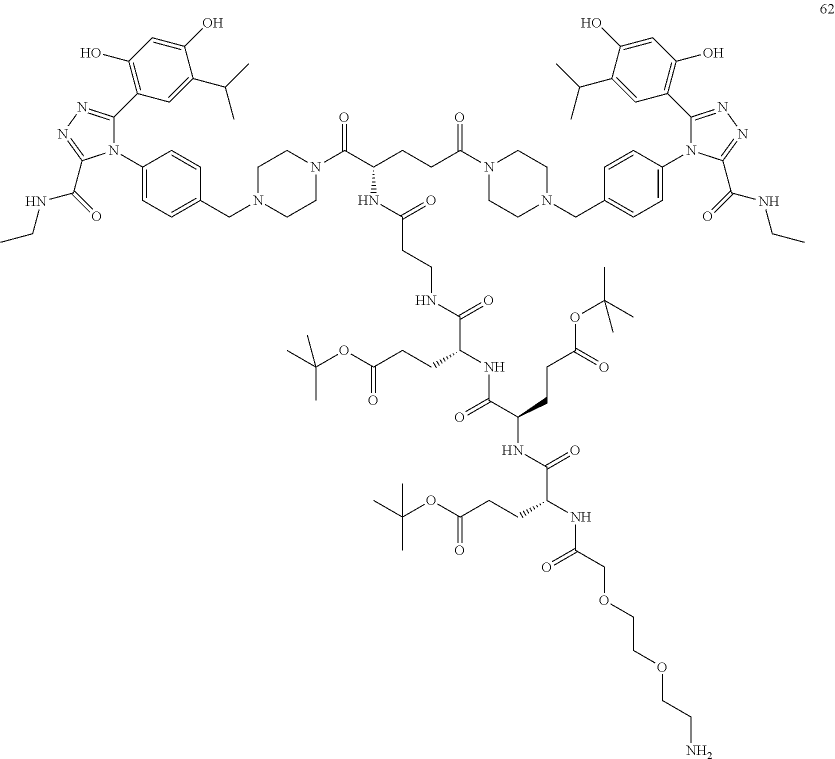 [SMILES: CCNC(=O)c1nnc(-c2cc(C(C)C)c(O)cc2O)n1-c1ccc(CN2CCN(C(=O)CC[C@H](NC(=O)CCNC(=O)[C@@H](CCC(=O)OC(C)(C)C)NC(=O)[C@@H](CCC(=O)OC(C)(C)C)NC(=O)[C@@H](CCC(=O)OC(C)(C)C)NC(=O)COCCOCCN)C(=O)N3CCN(Cc4ccc(-n5c(C(=O)NCC)nnc5-c5cc(C(C)C)c(O)cc5O)cc4)CC3)CC2)cc1]

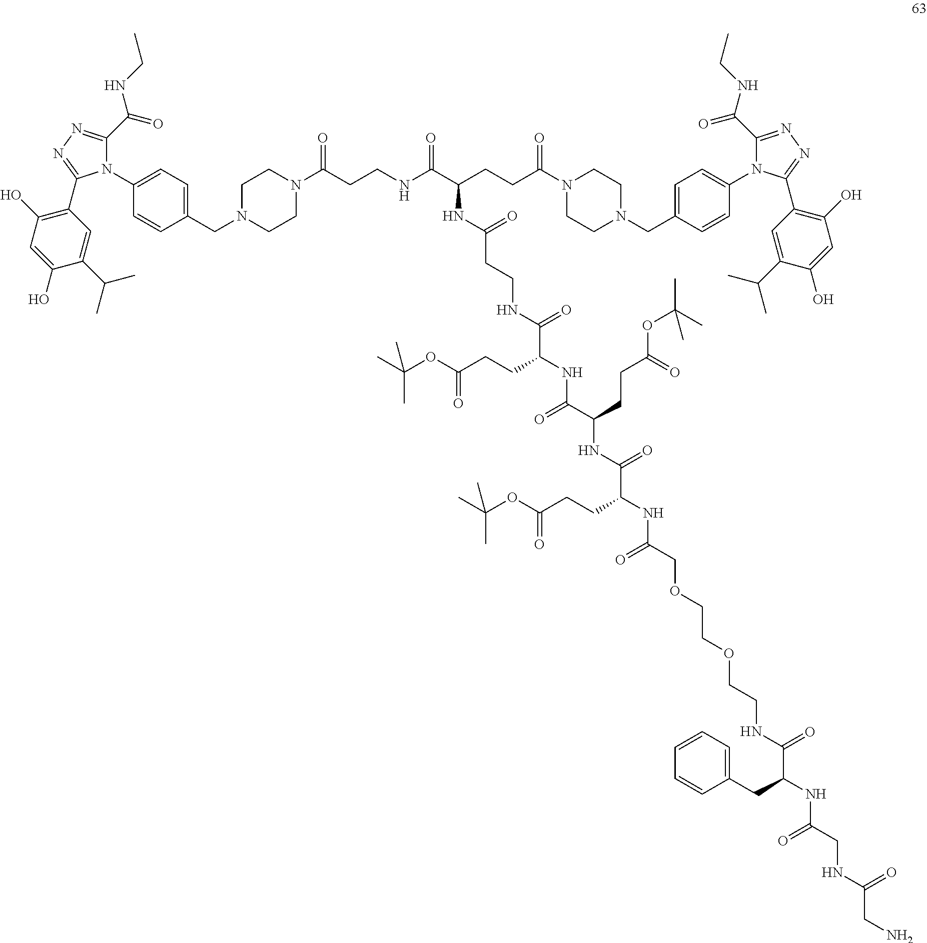 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)[C@@H](CCC(=O)OC(C)(C)C)NC(=O)[C@@H](CCC(=O)OC(C)(C)C)NC(=O)[C@@H](CCC(=O)OC(C)(C)C)NC(=O)COCCOCCNC(=O)[C@H](Cc3ccccc3)NC(=O)CNC(=O)CN)CC2)cc1]

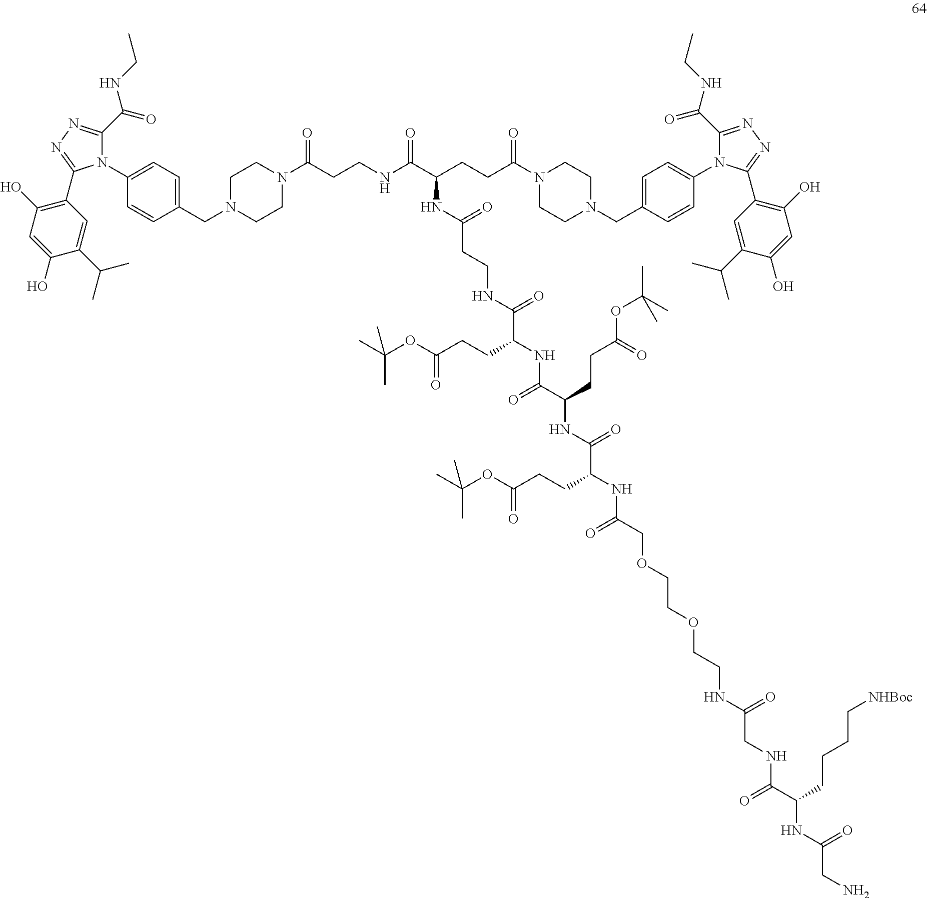 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)[C@@H](CCC(=O)OC(C)(C)C)NC(=O)[C@@H](CCC(=O)OC(C)(C)C)NC(=O)[C@@H](CCC(=O)OC(C)(C)C)NC(=O)COCCOCCNC(=O)CNC(=O)[C@H](CCCCNC(=O)OC(C)(C)C)NC(=O)CN)CC2)cc1]

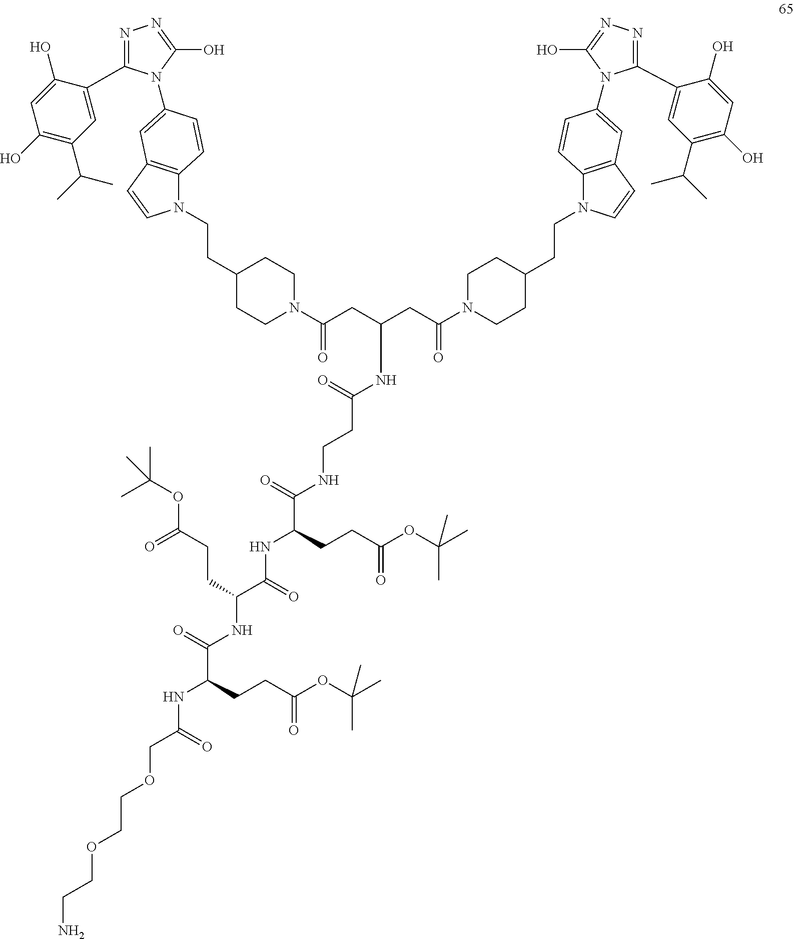 [SMILES: CC(C)c1cc(-c2nnc(O)n2-c2ccc3c(ccn3CCC3CCN(C(=O)CC(CC(=O)N4CCC(CCn5ccc6cc(-n7c(O)nnc7-c7cc(C(C)C)c(O)cc7O)ccc65)CC4)NC(=O)CCNC(=O)[C@@H](CCC(=O)OC(C)(C)C)NC(=O)[C@@H](CCC(=O)OC(C)(C)C)NC(=O)[C@@H](CCC(=O)OC(C)(C)C)NC(=O)COCCOCCN)CC3)c2)c(O)cc1O]

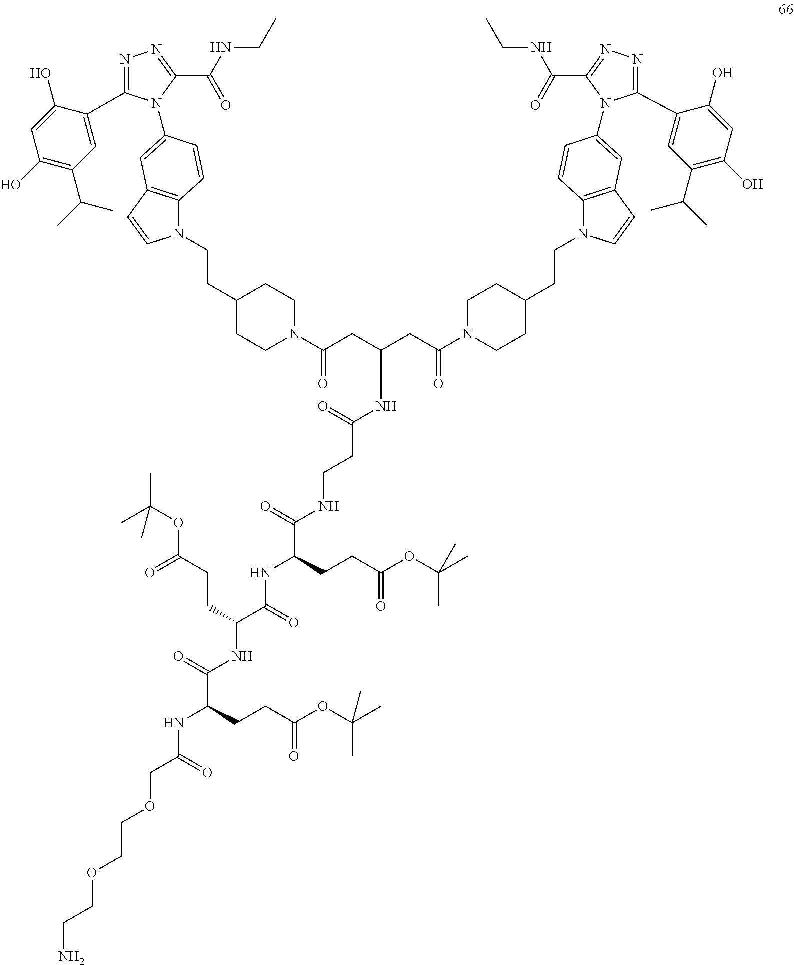 [SMILES: CCNC(=O)c1nnc(-c2cc(C(C)C)c(O)cc2O)n1-c1ccc2c(ccn2CCC2CCN(C(=O)CC(CC(=O)N3CCC(CCn4ccc5cc(-n6c(C(=O)NCC)nnc6-c6cc(C(C)C)c(O)cc6O)ccc54)CC3)NC(=O)CCNC(=O)[C@@H](CCC(=O)OC(C)(C)C)NC(=O)[C@@H](CCC(=O)OC(C)(C)C)NC(=O)[C@@H](CCC(=O)OC(C)(C)C)NC(=O)COCCOCCN)CC2)c1]

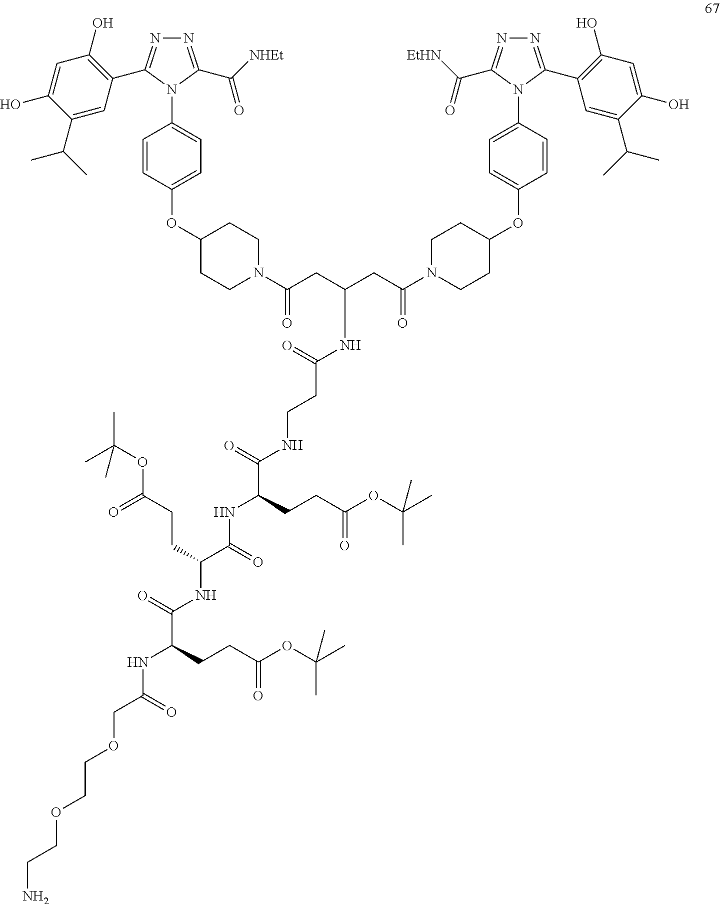

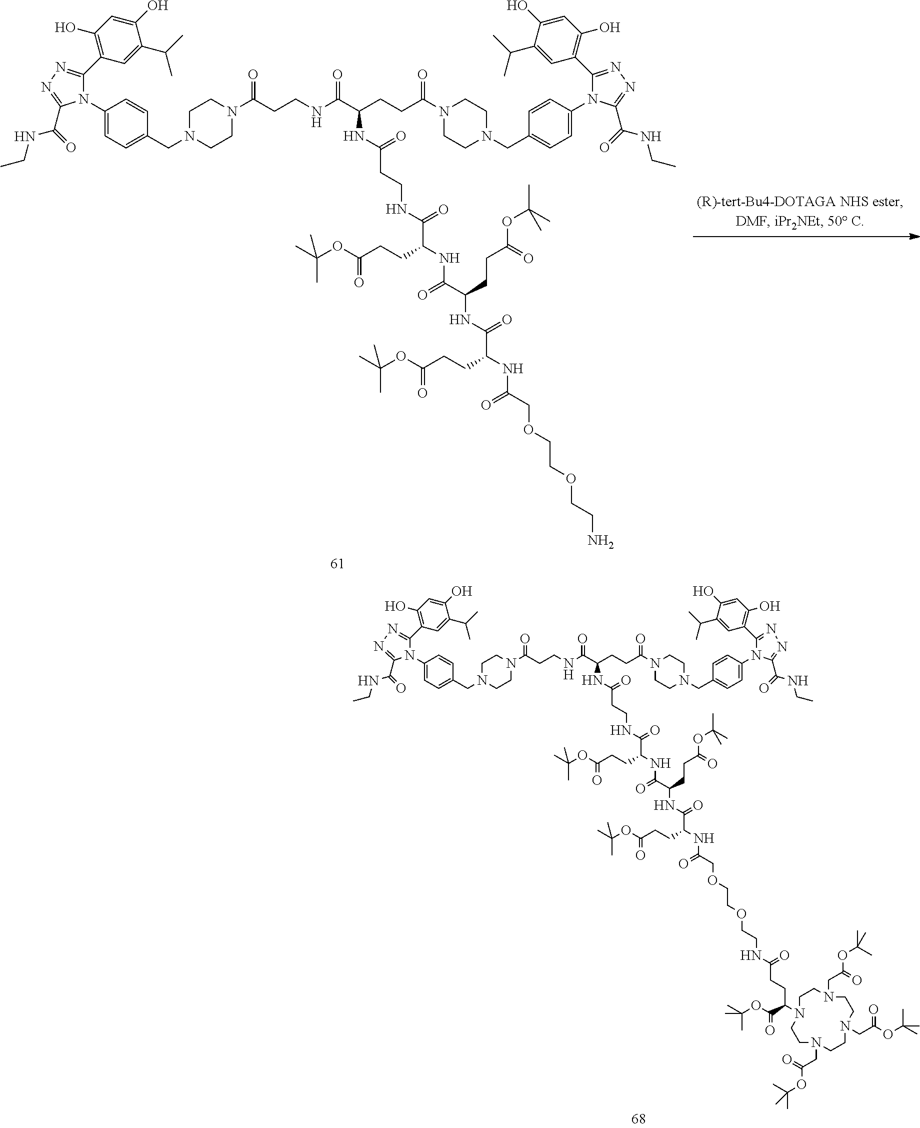

A vial was charged with 61 trifluoroacetate salt (29.0 mg, 14.5 μmol), and a solution of (R)-tert-Bu4-DOTAGA NHS ester (16.3 mg, 20.4 μmol) in DMF (2 mL) was added. Diiisopropylethylamine (0.2 mL) was added, and the reaction was stirred at room temperature for 18 h, then purified by preparative HPLC to give 68 (18.0 mg, 7.01 μmol, 48% yield). Compounds 69-74 were made in a similar manner.

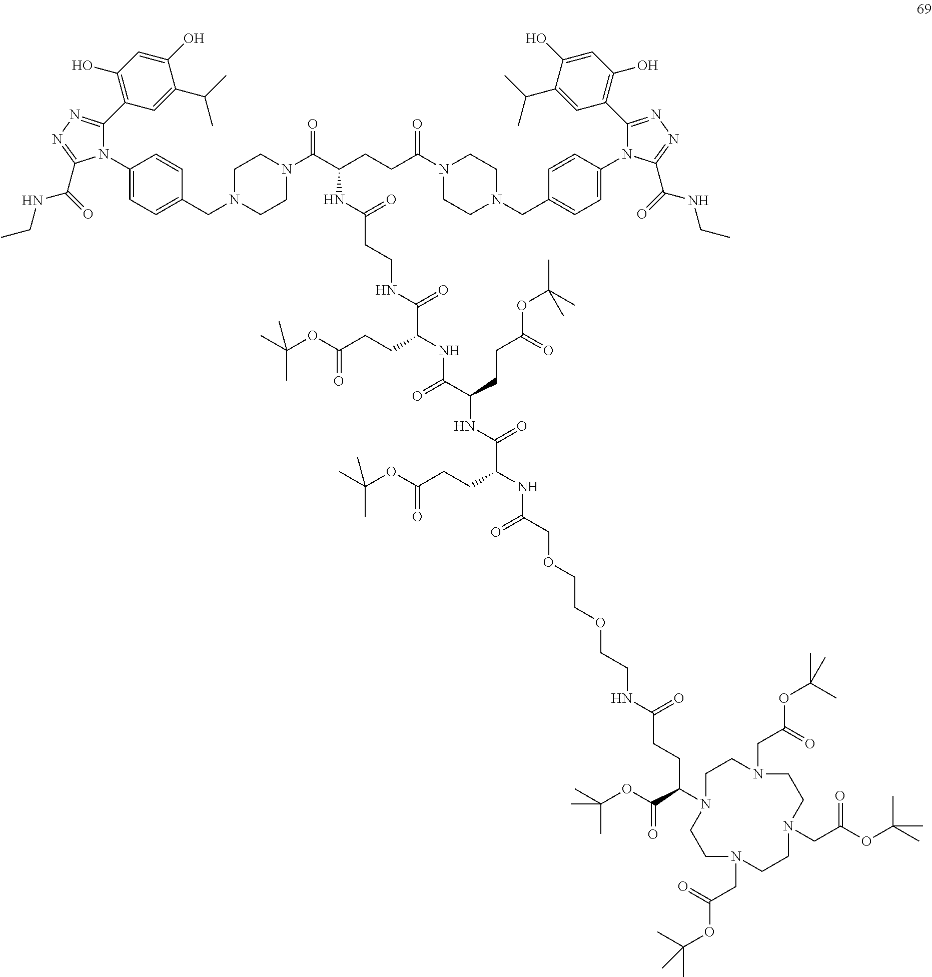 [SMILES: CCNC(=O)c1nnc(-c2cc(C(C)C)c(O)cc2O)n1-c1ccc(CN2CCN(C(=O)CC[C@H](NC(=O)CCNC(=O)[C@@H](CCC(=O)OC(C)(C)C)NC(=O)[C@@H](CCC(=O)OC(C)(C)C)NC(=O)[C@@H](CCC(=O)OC(C)(C)C)NC(=O)COCCOCCNC(=O)CC[C@H](C(=O)OC(C)(C)C)N3CCN(CC(=O)OC(C)(C)C)CCN(CC(=O)OC(C)(C)C)CCN(CC(=O)OC(C)(C)C)CC3)C(=O)N3CCN(Cc4ccc(-n5c(C(=O)NCC)nnc5-c5cc(C(C)C)c(O)cc5O)cc4)CC3)CC2)cc1]

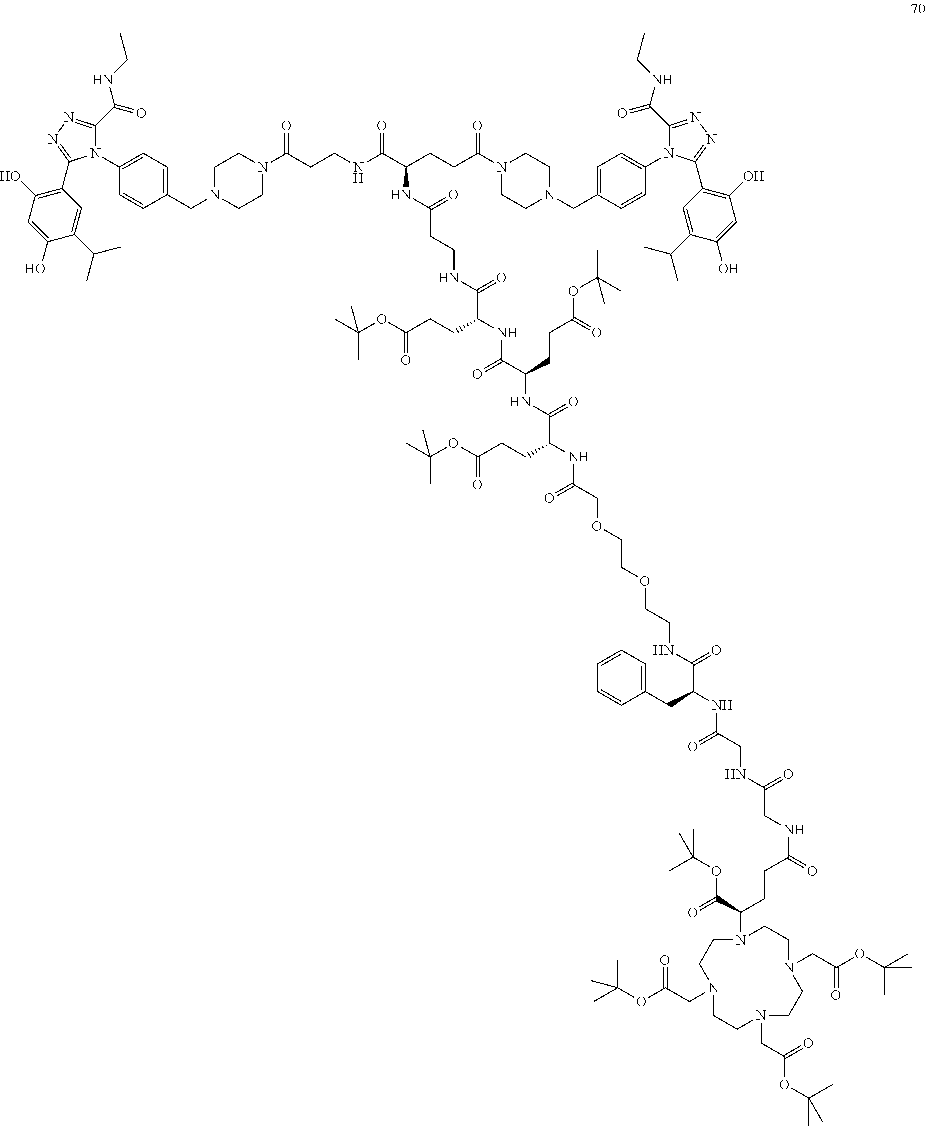 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)[C@@H](CCC(=O)OC(C)(C)C)NC(=O)[C@@H](CCC(=O)OC(C)(C)C)NC(=O)[C@@H](CCC(=O)OC(C)(C)C)NC(=O)COCCOCCNC(=O)[C@H](Cc3ccccc3)NC(=O)CNC(=O)CNC(=O)CC[C@H](C(=O)OC(C)(C)C)N3CCN(CC(=O)OC(C)(C)C)CCN(CC(=O)OC(C)(C)C)CCN(CC(=O)OC(C)(C)C)CC3)CC2)cc1]

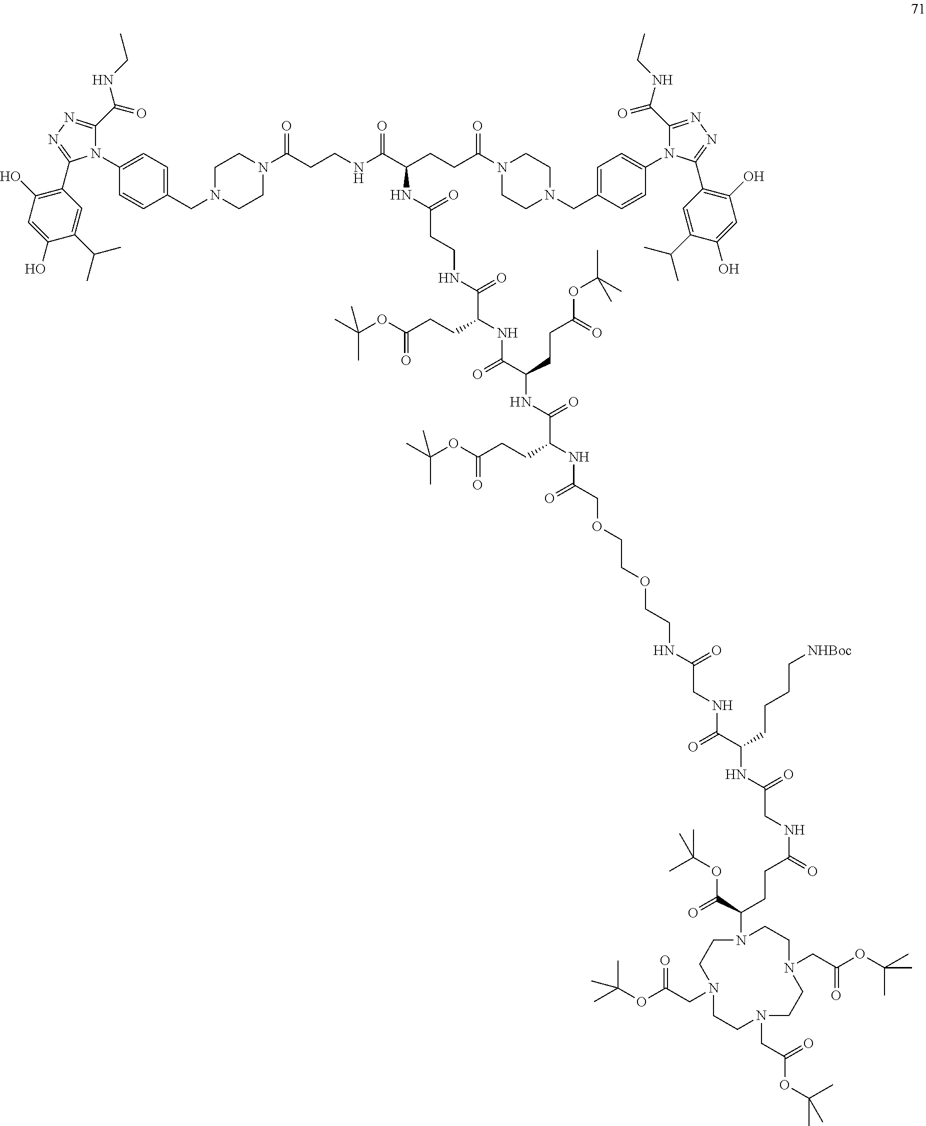 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)[C@@H](CCC(=O)OC(C)(C)C)NC(=O)[C@@H](CCC(=O)OC(C)(C)C)NC(=O)[C@@H](CCC(=O)OC(C)(C)C)NC(=O)COCCOCCNC(=O)CNC(=O)[C@H](CCCCNC(=O)OC(C)(C)C)NC(=O)CNC(=O)CC[C@H](C(=O)OC(C)(C)C)N3CCN(CC(=O)OC(C)(C)C)CCN(CC(=O)OC(C)(C)C)CCN(CC(=O)OC(C)(C)C)CC3)CC2)cc1]

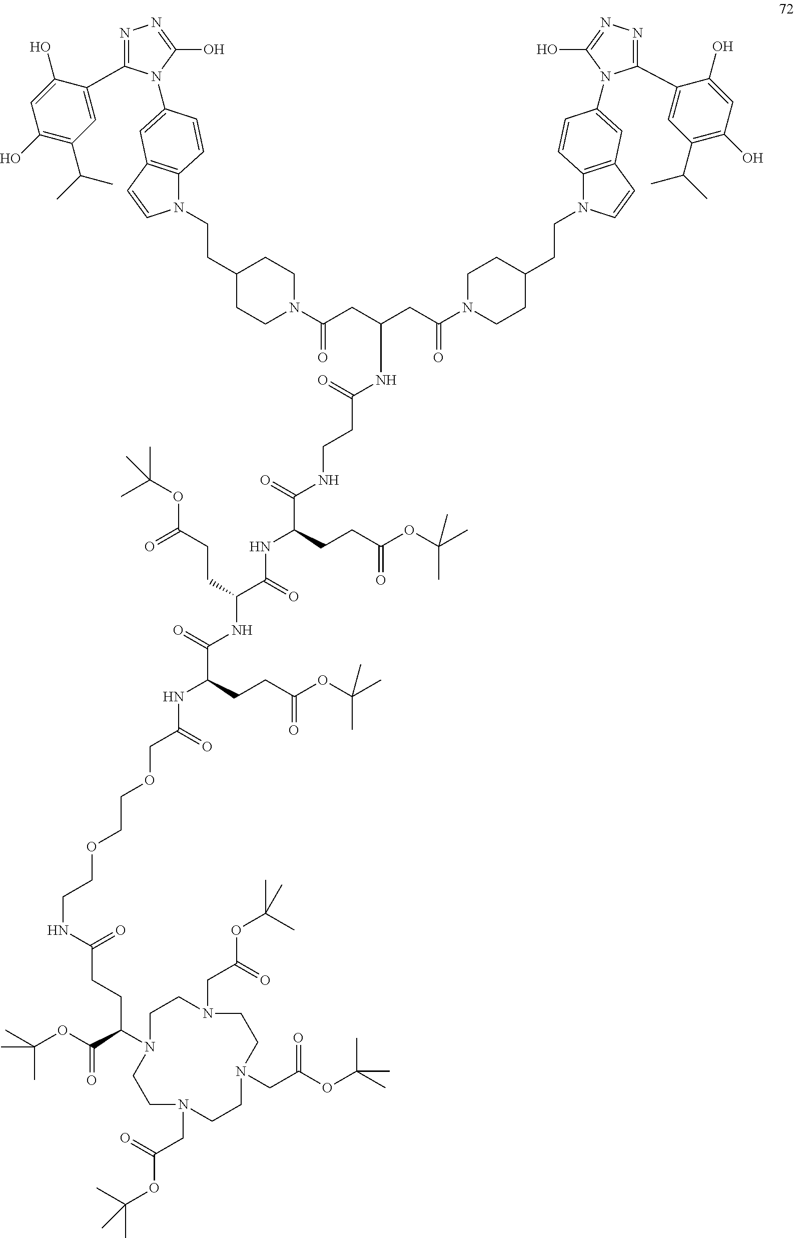 [SMILES: CC(C)c1cc(-c2nnc(O)n2-c2ccc3c(ccn3CCC3CCN(C(=O)CC(CC(=O)N4CCC(CCn5ccc6cc(-n7c(O)nnc7-c7cc(C(C)C)c(O)cc7O)ccc65)CC4)NC(=O)CCNC(=O)[C@@H](CCC(=O)OC(C)(C)C)NC(=O)[C@@H](CCC(=O)OC(C)(C)C)NC(=O)[C@@H](CCC(=O)OC(C)(C)C)NC(=O)COCCOCCNC(=O)CC[C@H](C(=O)OC(C)(C)C)N4CCN(CC(=O)OC(C)(C)C)CCN(CC(=O)OC(C)(C)C)CCN(CC(=O)OC(C)(C)C)CC4)CC3)c2)c(O)cc1O]

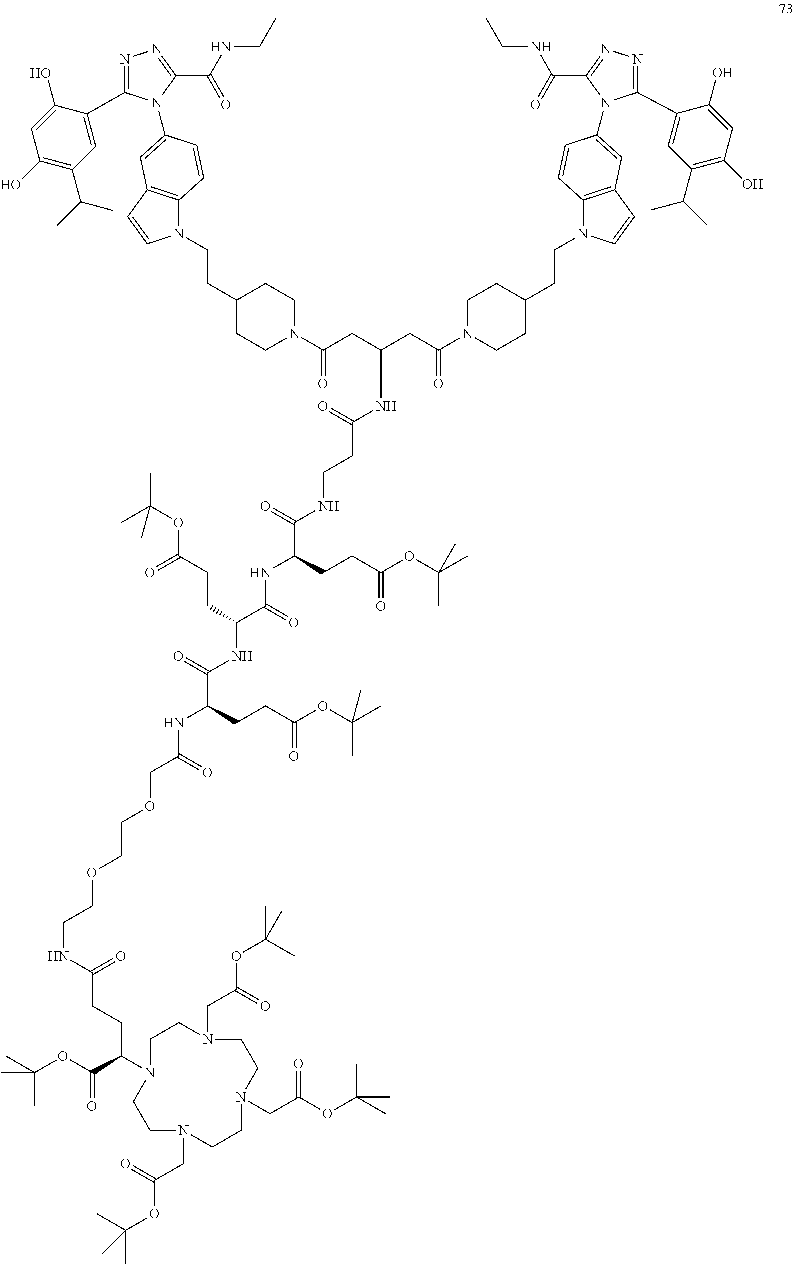 [SMILES: CCNC(=O)c1nnc(-c2cc(C(C)C)c(O)cc2O)n1-c1ccc2c(ccn2CCC2CCN(C(=O)CC(CC(=O)N3CCC(CCn4ccc5cc(-n6c(C(=O)NCC)nnc6-c6cc(C(C)C)c(O)cc6O)ccc54)CC3)NC(=O)CCNC(=O)[C@@H](CCC(=O)OC(C)(C)C)NC(=O)[C@@H](CCC(=O)OC(C)(C)C)NC(=O)[C@@H](CCC(=O)OC(C)(C)C)NC(=O)COCCOCCNC(=O)CC[C@H](C(=O)OC(C)(C)C)N3CCN(CC(=O)OC(C)(C)C)CCN(CC(=O)OC(C)(C)C)CCN(CC(=O)OC(C)(C)C)CC3)CC2)c1]

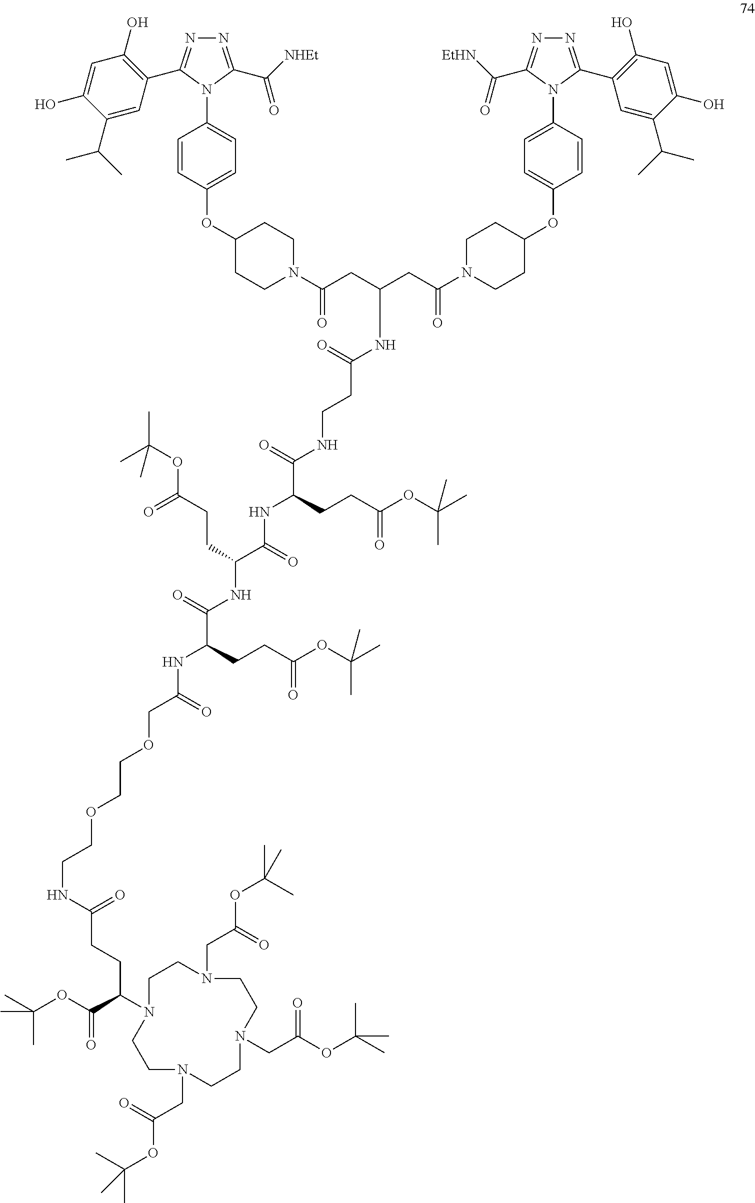

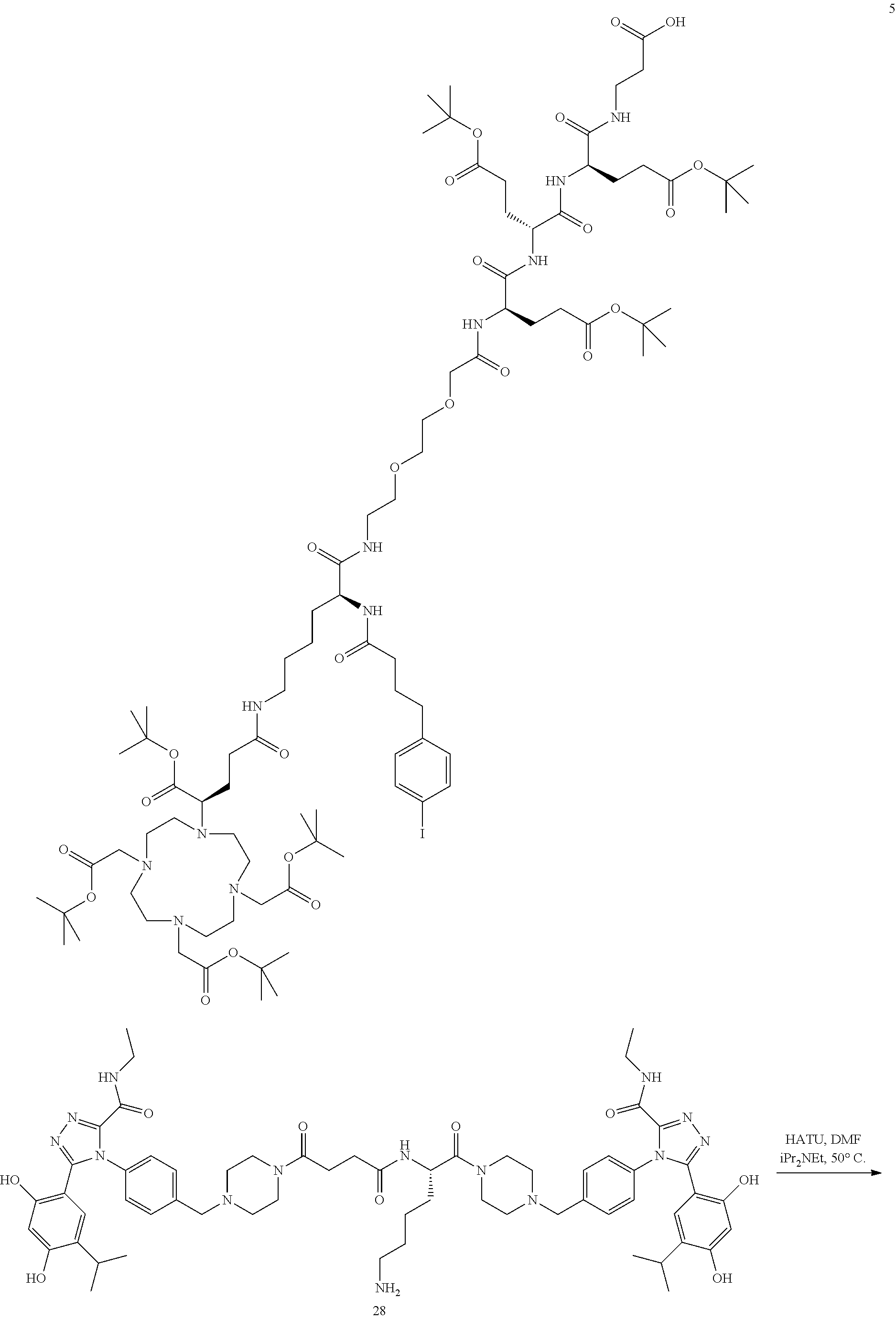 [SMILES: CC(C)(C)OC(=O)CC[C@@H](NC(=O)COCCOCCNC(=O)[C@H](CCCCNC(=O)CC[C@H](C(=O)OC(C)(C)C)N1CCN(CC(=O)OC(C)(C)C)CCN(CC(=O)OC(C)(C)C)CCN(CC(=O)OC(C)(C)C)CC1)NC(=O)CCCc1ccc(I)cc1)C(=O)N[C@H](CCC(=O)OC(C)(C)C)C(=O)N[C@H](CCC(=O)OC(C)(C)C)C(=O)NCCC(=O)O ; CCNC(=O)c1nnc(-c2cc(C(C)C)c(O)cc2O)n1-c1ccc(CN2CCN(C(=O)CCC(=O)N[C@@H](CCCCN)C(=O)N3CCN(Cc4ccc(-n5c(C(=O)NCC)nnc5-c5cc(C(C)C)c(O)cc5O)cc4)CC3)CC2)cc1]

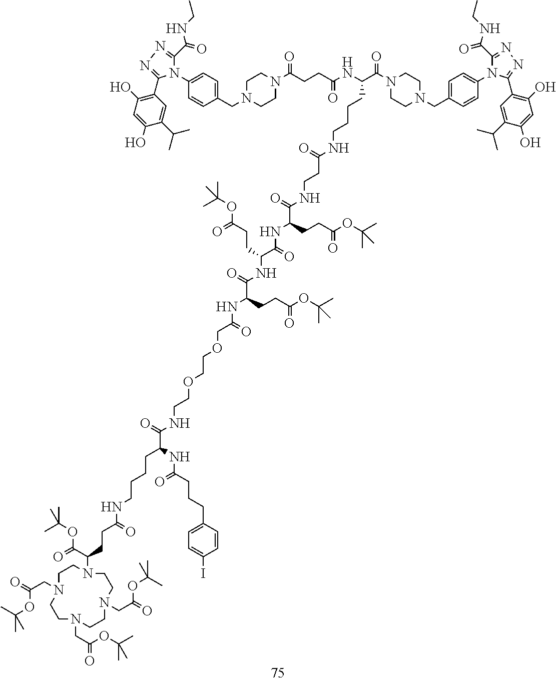 [SMILES: CCNC(=O)c1nnc(-c2cc(C(C)C)c(O)cc2O)n1-c1ccc(CN2CCN(C(=O)CCC(=O)N[C@@H](CCCCNC(=O)CCNC(=O)[C@@H](CCC(=O)OC(C)(C)C)NC(=O)[C@@H](CCC(=O)OC(C)(C)C)NC(=O)[C@@H](CCC(=O)OC(C)(C)C)NC(=O)COCCOCCNC(=O)[C@H](CCCCNC(=O)CC[C@H](C(=O)OC(C)(C)C)N3CCN(CC(=O)OC(C)(C)C)CCN(CC(=O)OC(C)(C)C)CCN(CC(=O)OC(C)(C)C)CC3)NC(=O)CCCc3ccc(I)cc3)C(=O)N3CCN(Cc4ccc(-n5c(C(=O)NCC)nnc5-c5cc(C(C)C)c(O)cc5O)cc4)CC3)CC2)cc1]

A vial was charged with 5 (17.7 mg, 9.45 μmol) and 28 trifluoroacetate salt (17.2 mg, 11.6 μmol), and a solution of HATU (4.6 mg, 12.3 μmop in DMF (2 mL) was added. Diisopropylethylamine (0.2 mL) was then added, and the reaction stirred at 50° C. for 1 h, the purified by preparative HPLC to give 75 (10.7 mg, 3.57 μma 37% yield). Compounds 76-87 were made in a similar manner.

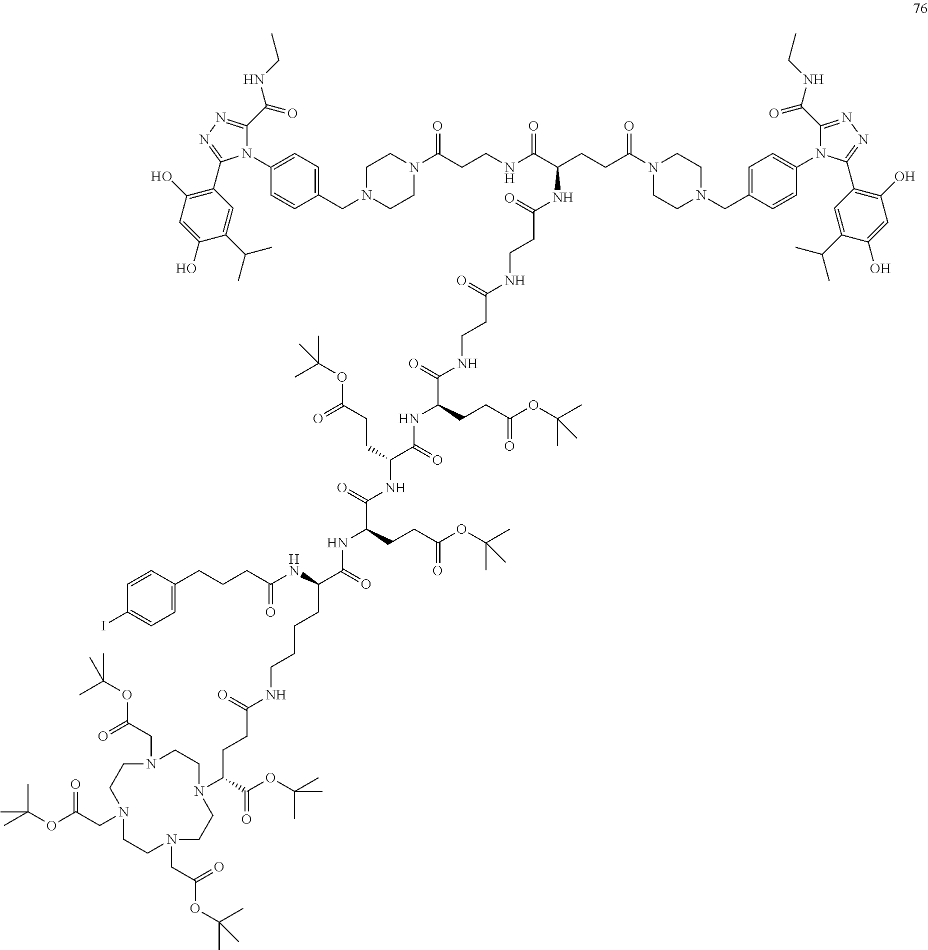 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)CCNC(=O)[C@@H](CCC(=O)OC(C)(C)C)NC(=O)[C@@H](CCC(=O)OC(C)(C)C)NC(=O)[C@@H](CCC(=O)OC(C)(C)C)NC(=O)[C@@H](CCCCNC(=O)CC[C@H](C(=O)OC(C)(C)C)N3CCN(CC(=O)OC(C)(C)C)CCN(CC(=O)OC(C)(C)C)CCN(CC(=O)OC(C)(C)C)CC3)NC(=O)CCCc3ccc(I)cc3)CC2)cc1]

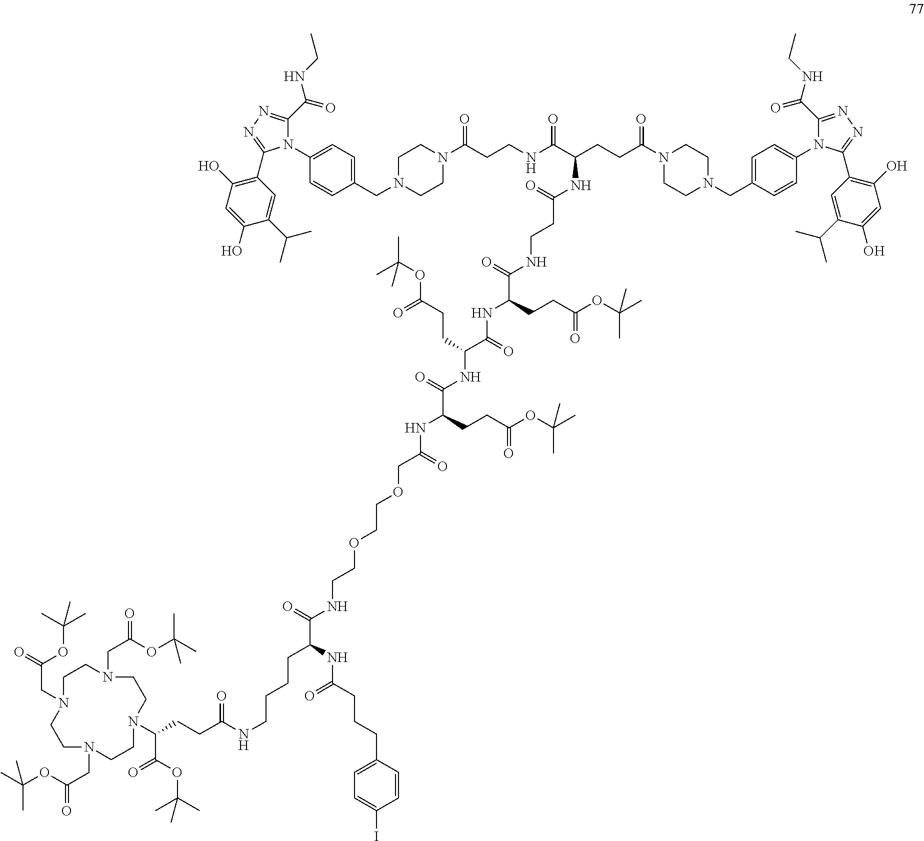 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)[C@@H](CCC(=O)OC(C)(C)C)NC(=O)[C@@H](CCC(=O)OC(C)(C)C)NC(=O)[C@@H](CCC(=O)OC(C)(C)C)NC(=O)COCCOCCNC(=O)[C@H](CCCCNC(=O)CC[C@H](C(=O)OC(C)(C)C)N3CCN(CC(=O)OC(C)(C)C)CCN(CC(=O)OC(C)(C)C)CCN(CC(=O)OC(C)(C)C)CC3)NC(=O)CCCc3ccc(I)cc3)CC2)cc1]

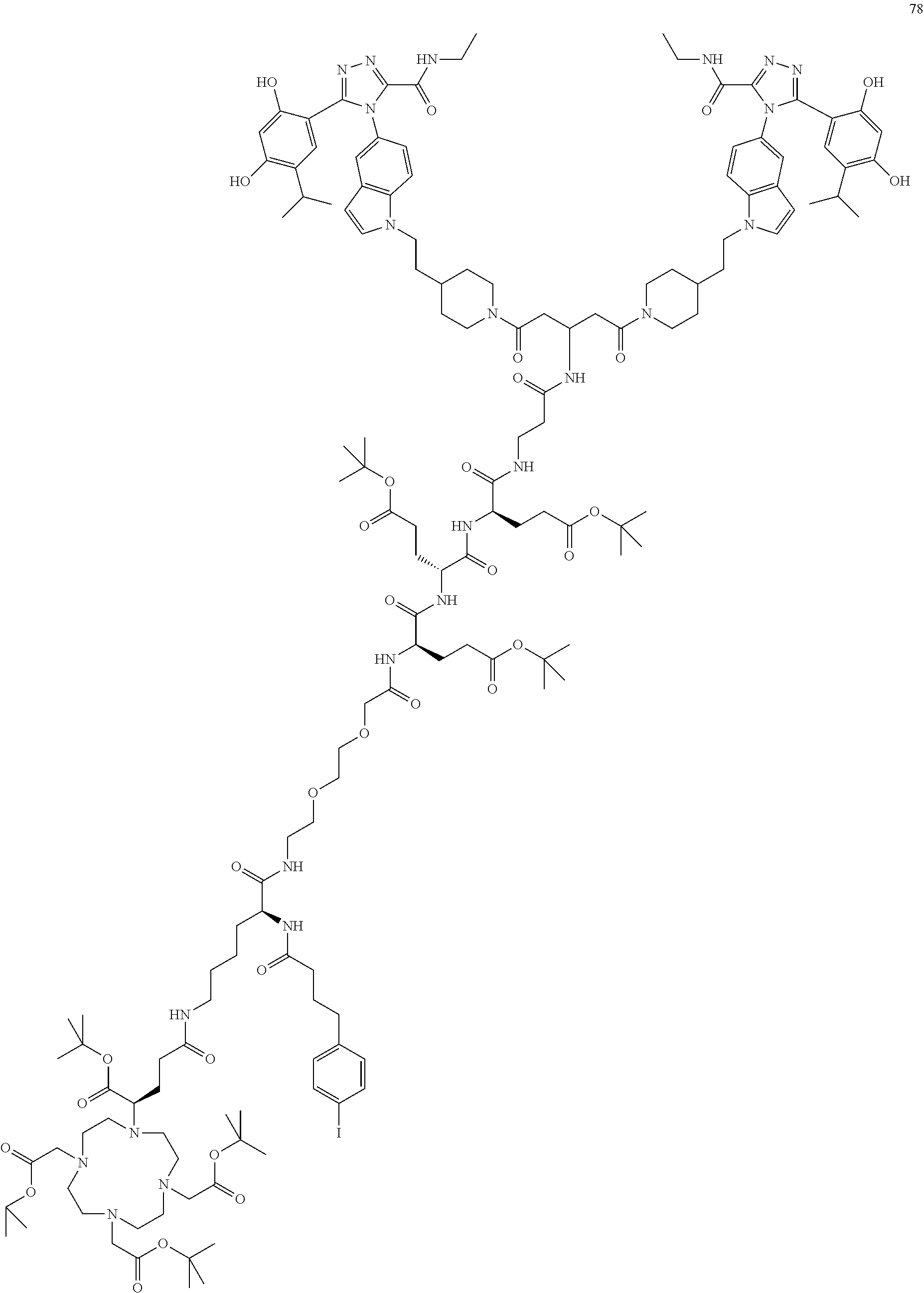 [SMILES: CCNC(=O)c1nnc(-c2cc(C(C)C)c(O)cc2O)n1-c1ccc2c(ccn2CCC2CCN(C(=O)CC(CC(=O)N3CCC(CCn4ccc5cc(-n6c(C(=O)NCC)nnc6-c6cc(C(C)C)c(O)cc6O)ccc54)CC3)NC(=O)CCNC(=O)[C@@H](CCC(=O)OC(C)(C)C)NC(=O)[C@@H](CCC(=O)OC(C)(C)C)NC(=O)[C@@H](CCC(=O)OC(C)(C)C)NC(=O)COCCOCCNC(=O)[C@H](CCCCNC(=O)CC[C@H](C(=O)OC(C)(C)C)N3CCN(CC(=O)OC(C)C)CCN(CC(=O)OC(C)(C)C)CCN(CC(=O)OC(C)(C)C)CC3)NC(=O)CCCc3ccc(I)cc3)CC2)c1]

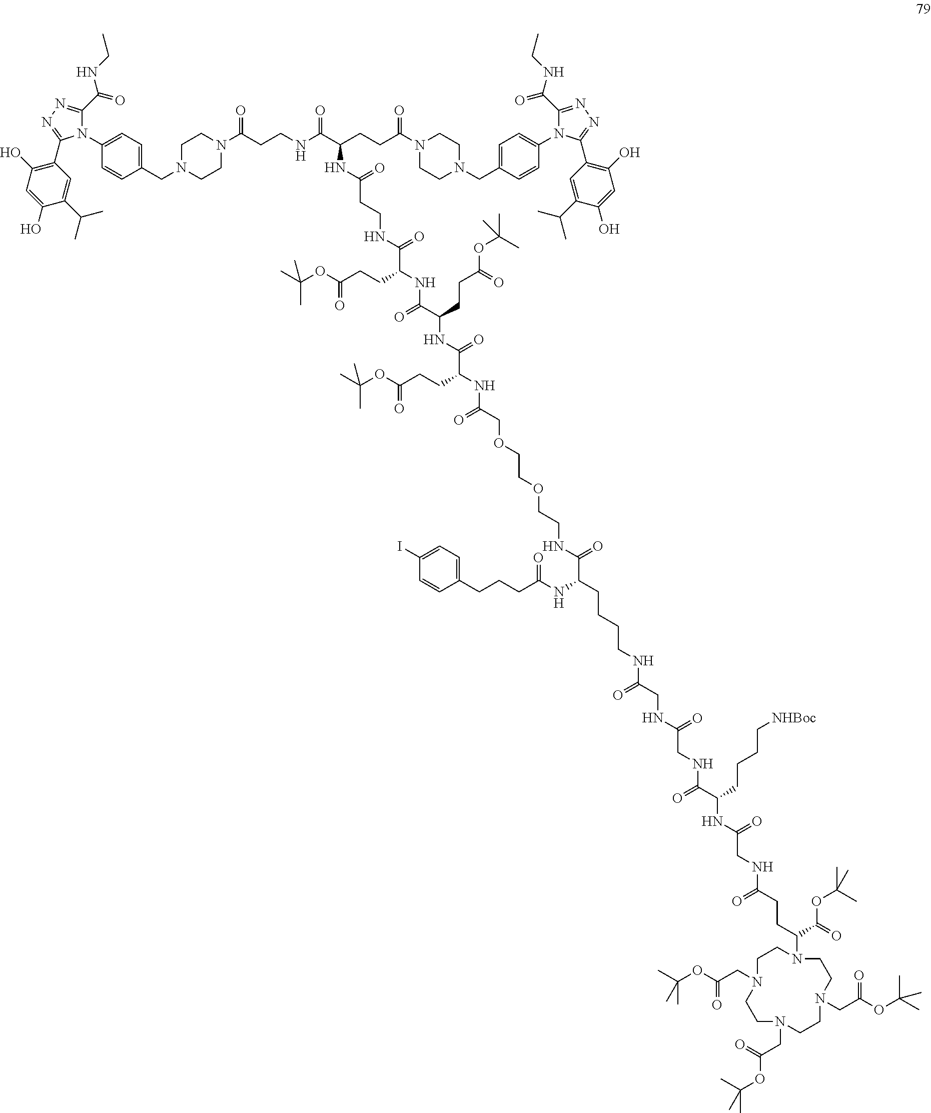

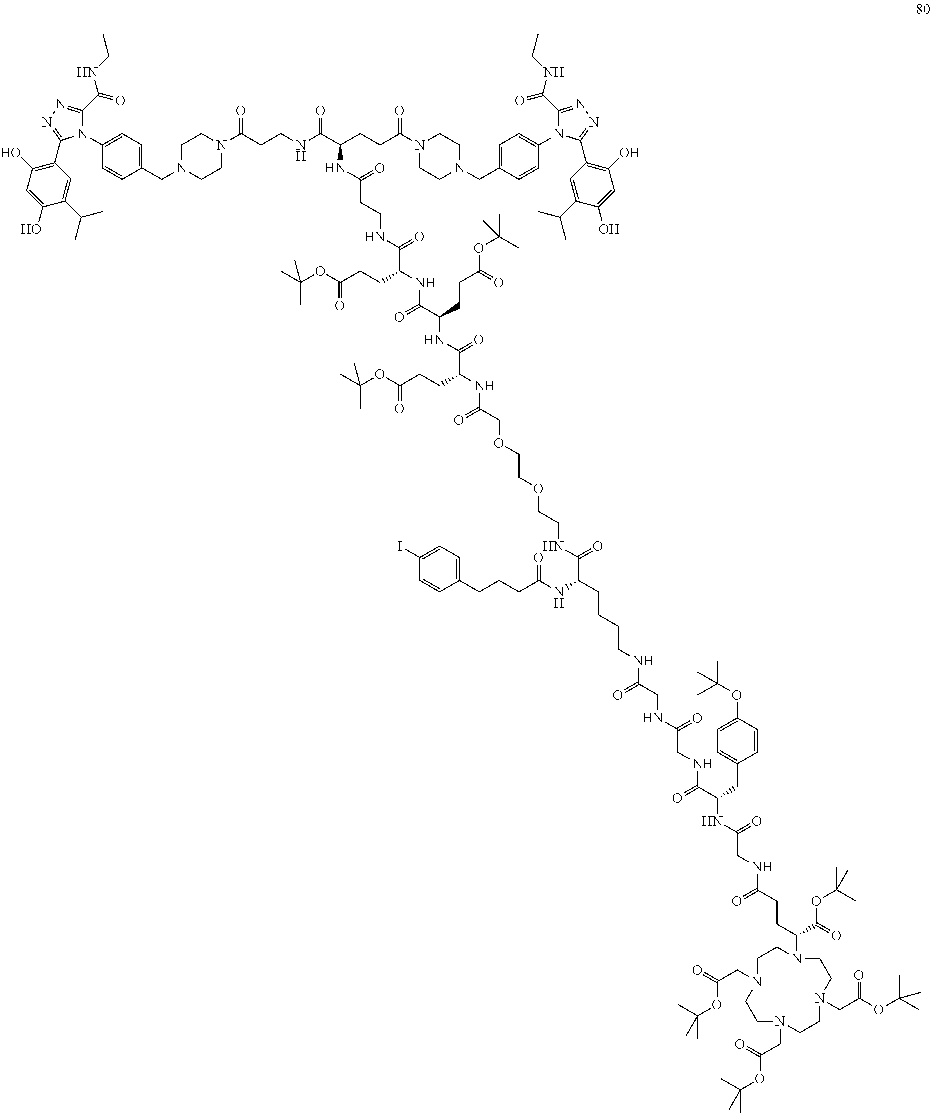 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)[C@@H](CCC(=O)OC(C)(C)C)NC(=O)[C@@H](CCC(=O)OC(C)(C)C)NC(=O)[C@@H](CCC(=O)OC(C)(C)C)NC(=O)COCCOCCNC(=O)[C@H](CCCCNC(=O)CNC(=O)CNC(=O)[C@H](Cc3ccc(OC(C)(C)C)cc3)NC(=O)CNC(=O)CC[C@H](C(=O)OC(C)(C)C)N3CCN(CC(=O)OC(C)(C)C)CCN(CC(=O)OC(C)(C)C)CCN(CC(=O)OC(C)(C)C)CC3)NC(=O)CCCc3ccc(I)cc3)CC2)cc1]

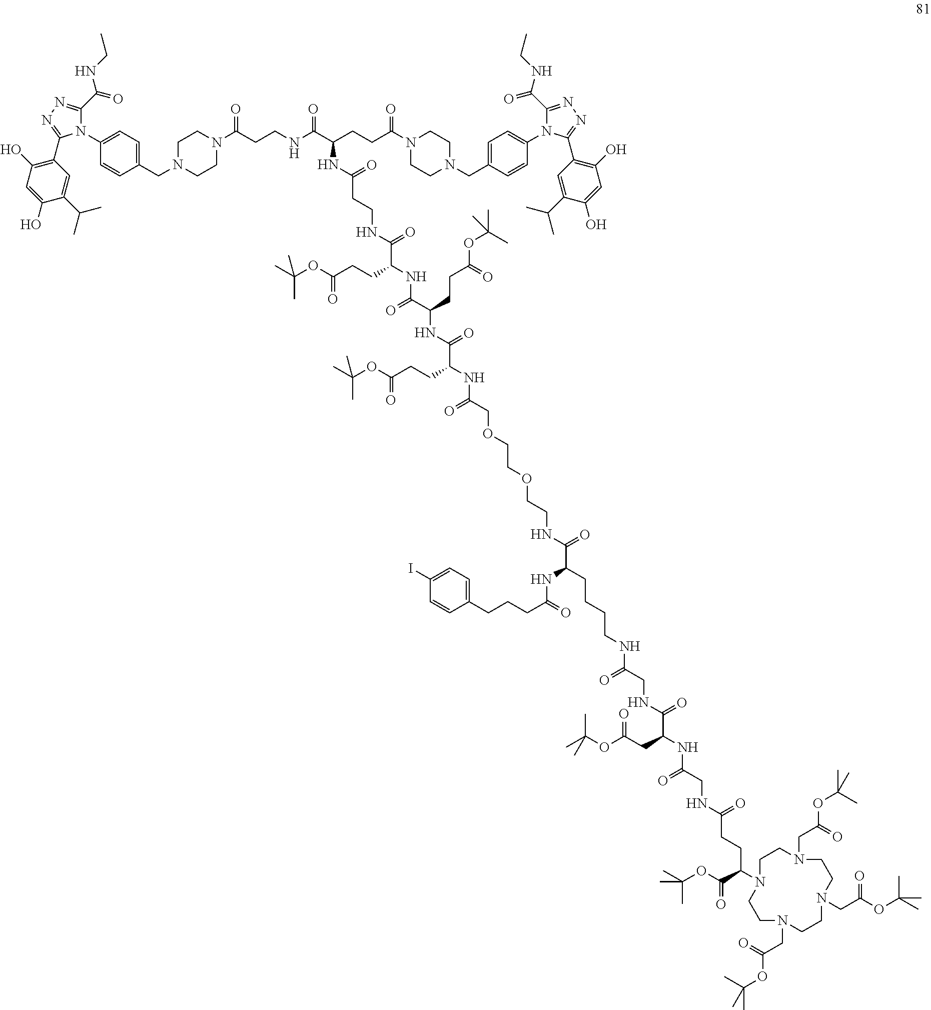 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)[C@@H](CCC(=O)OC(C)(C)C)NC(=O)[C@@H](CCC(=O)OC(C)(C)C)NC(=O)[C@@H](CCC(=O)OC(C)(C)C)NC(=O)COCCOCCNC(=O)[C@@H](CCCCNC(=O)CNC(=O)[C@H](CC(=O)OC(C)(C)C)NC(=O)CNC(=O)CC[C@H](C(=O)OC(C)(C)C)N3CCN(CC(=O)OC(C)(C)C)CCN(CC(=O)OC(C)(C)C)CCN(CC(=O)OC(C)(C)C)CC3)NC(=O)CCCc3ccc(I)cc3)CC2)cc1]

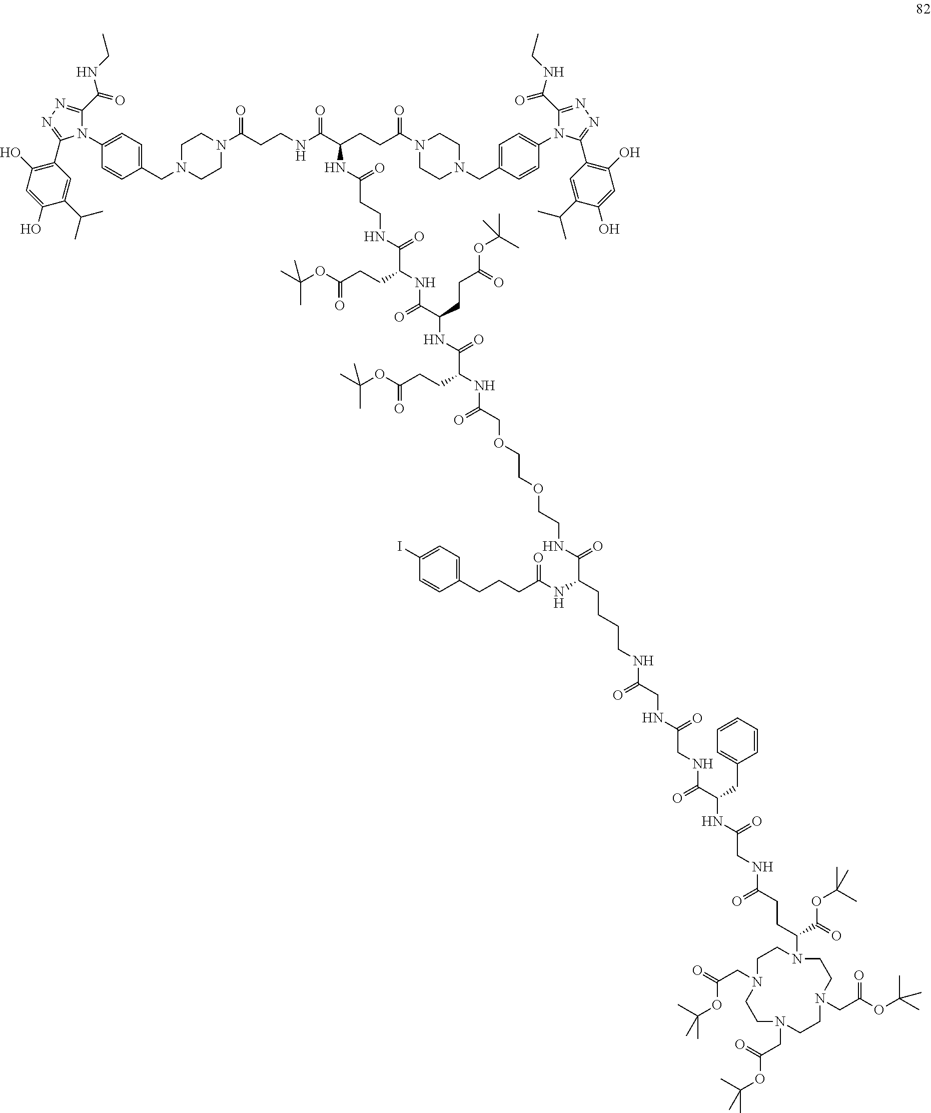 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)[C@@H](CCC(=O)OC(C)(C)C)NC(=O)[C@@H](CCC(=O)OC(C)(C)C)NC(=O)[C@@H](CCC(=O)OC(C)(C)C)NC(=O)COCCOCCNC(=O)[C@H](CCCCNC(=O)CNC(=O)CNC(=O)[C@H](Cc3ccccc3)NC(=O)CNC(=O)CC[C@H](C(=O)OC(C)(C)C)N3CCN(CC(=O)OC(C)(C)C)CCN(CC(=O)OC(C)(C)C)CCN(CC(=O)OC(C)(C)C)CC3)NC(=O)CCCc3ccc(I)cc3)CC2)cc1]

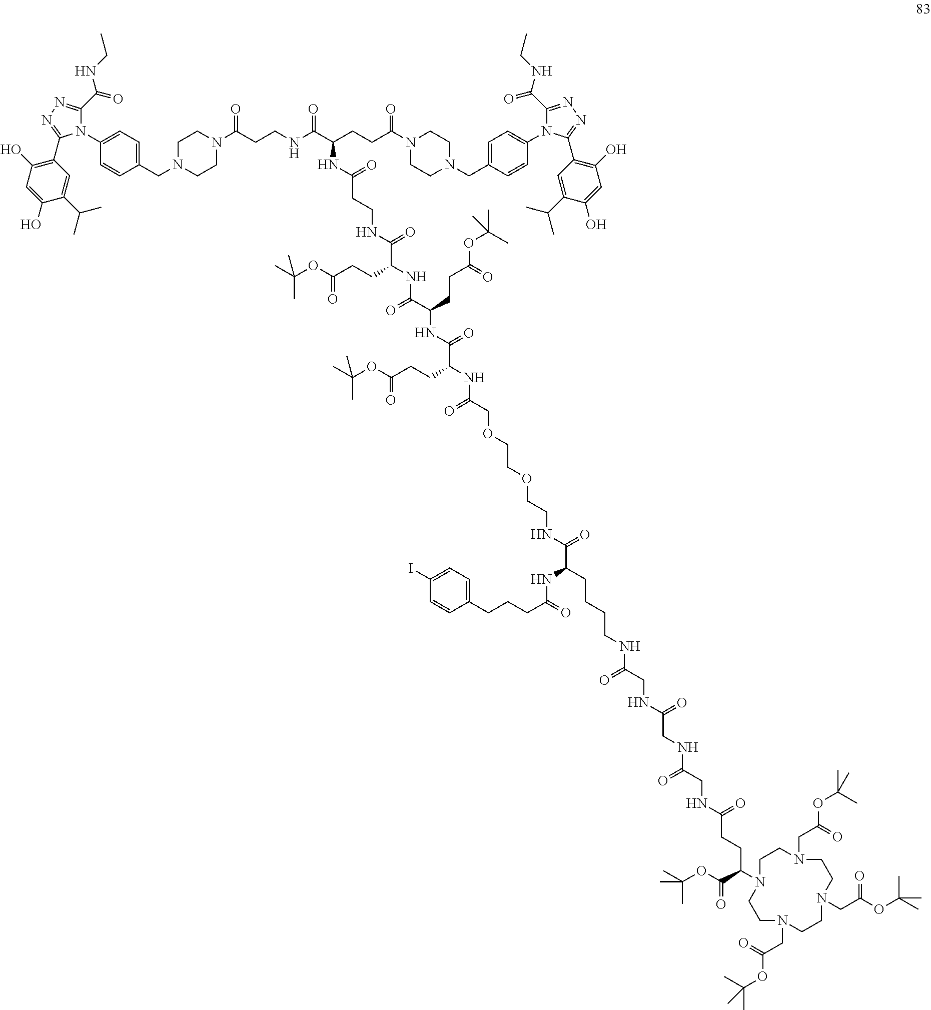 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)[C@@H](CCC(=O)OC(C)(C)C)NC(=O)[C@@H](CCC(=O)OC(C)(C)C)NC(=O)[C@@H](CCC(=O)OC(C)(C)C)NC(=O)COCCOCCNC(=O)[C@@H](CCCCNC(=O)CNC(=O)CNC(=O)CNC(=O)CC[C@H](C(=O)OC(C)(C)C)N3CCN(CC(=O)OC(C)(C)C)CCN(CC(=O)OC(C)(C)C)CCN(CC(=O)OC(C)(C)C)CC3)NC(=O)CCCc3ccc(I)cc3)CC2)cc1]

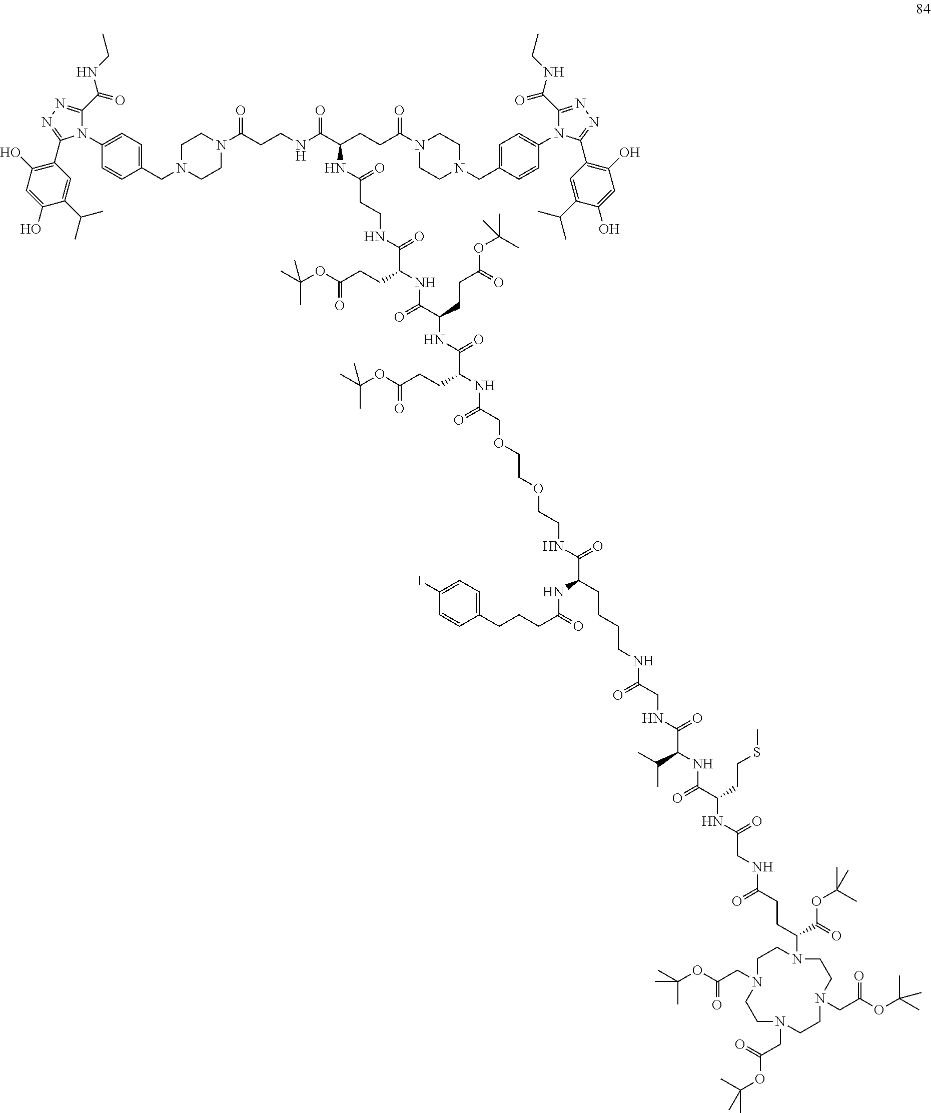 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)[C@@H](CCC(=O)OC(C)(C)C)NC(=O)[C@@H](CCC(=O)OC(C)(C)C)NC(=O)[C@@H](CCC(=O)OC(C)(C)C)NC(=O)COCCOCCNC(=O)[C@@H](CCCCNC(=O)CNC(=O)[C@@H](NC(=O)[C@H](CCSC)NC(=O)CNC(=O)CC[C@H](C(=O)OC(C)(C)C)N3CCN(CC(=O)OC(C)(C)C)CCN(CC(=O)OC(C)(C)C)CCN(CC(=O)OC(C)(C)C)CC3)C(C)C)NC(=O)CCCc3ccc(I)cc3)CC2)cc1]

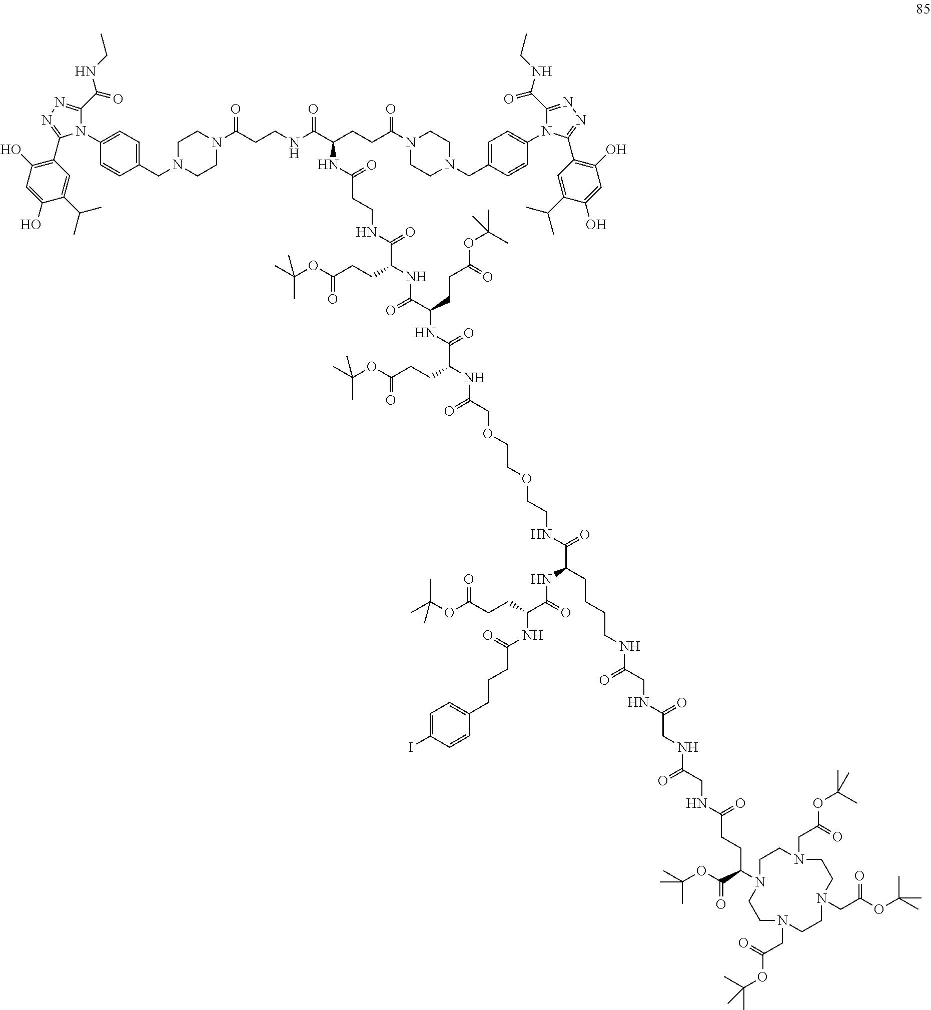 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)[C@@H](CCC(=O)OC(C)(C)C)NC(=O)[C@@H](CCC(=O)OC(C)(C)C)NC(=O)[C@@H](CCC(=O)OC(C)(C)C)NC(=O)COCCOCCNC(=O)[C@@H](CCCCNC(=O)CNC(=O)CNC(=O)CNC(=O)CC[C@H](C(=O)OC(C)(C)C)N3CCN(CC(=O)OC(C)(C)C)CCN(CC(=O)OC(C)(C)C)CCN(CC(=O)OC(C)(C)C)CC3)NC(=O)[C@@H](CCC(=O)OC(C)(C)C)NC(=O)CCCc3ccc(I)cc3)CC2)cc1]

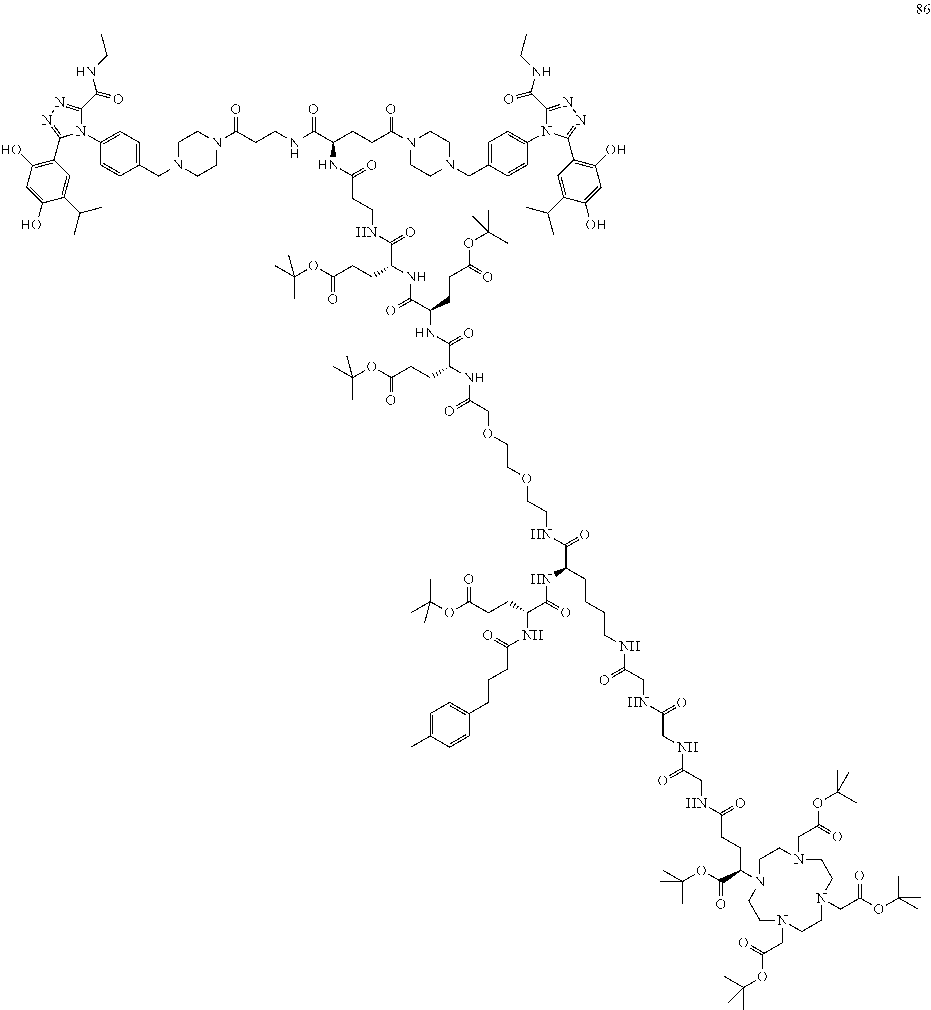 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)[C@@H](CCC(=O)OC(C)(C)C)NC(=O)[C@@H](CCC(=O)OC(C)(C)C)NC(=O)[C@@H](CCC(=O)OC(C)(C)C)NC(=O)COCCOCCNC(=O)[C@@H](CCCCNC(=O)CNC(=O)CNC(=O)CNC(=O)CC[C@H](C(=O)OC(C)(C)C)N3CCN(CC(=O)OC(C)(C)C)CCN(CC(=O)OC(C)(C)C)CCN(CC(=O)OC(C)(C)C)CC3)NC(=O)[C@@H](CCC(=O)OC(C)(C)C)NC(=O)CCCc3ccc(C)cc3)CC2)cc1]

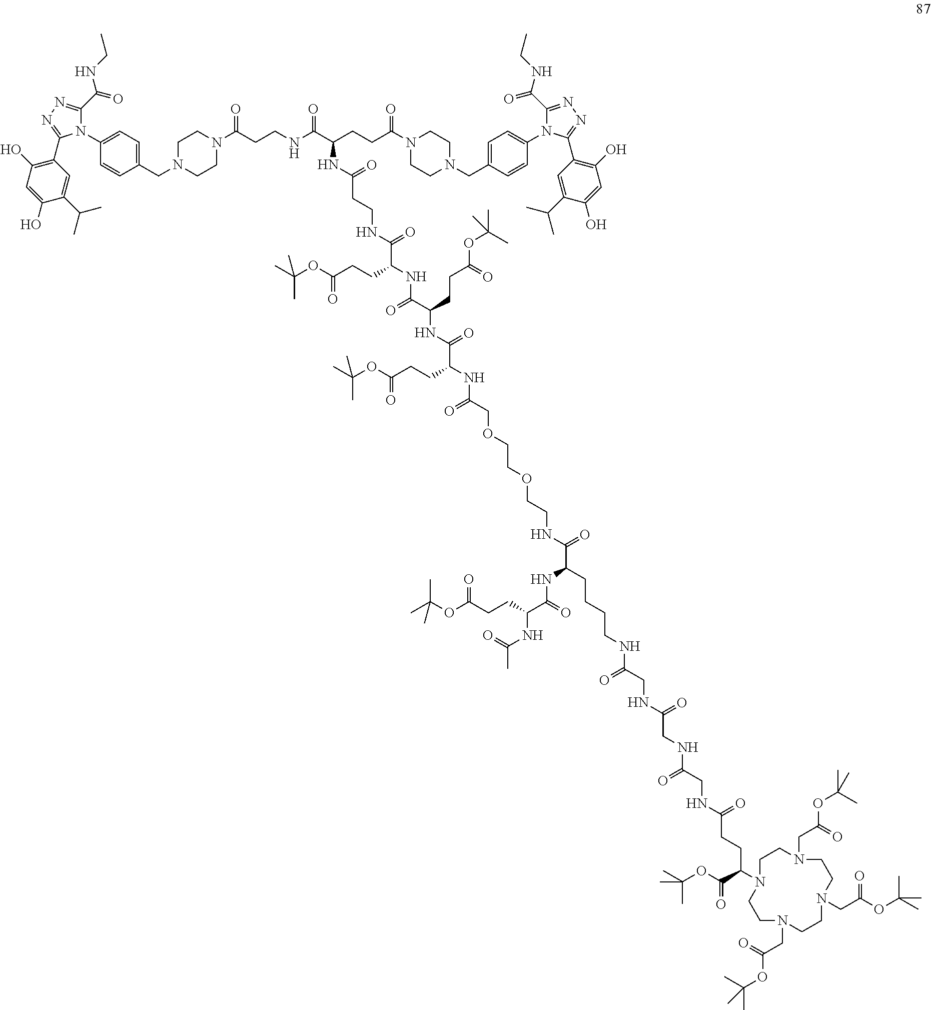 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)[C@@H](CCC(=O)OC(C)(C)C)NC(=O)[C@@H](CCC(=O)OC(C)(C)C)NC(=O)[C@@H](CCC(=O)OC(C)(C)C)NC(=O)COCCOCCNC(=O)[C@@H](CCCCNC(=O)CNC(=O)CNC(=O)CNC(=O)CC[C@H](C(=O)OC(C)(C)C)N3CCN(CC(=O)OC(C)(C)C)CCN(CC(=O)OC(C)(C)C)CCN(CC(=O)OC(C)(C)C)CC3)NC(=O)[C@@H](CCC(=O)OC(C)(C)C)NC(C)=O)CC2)cc1]

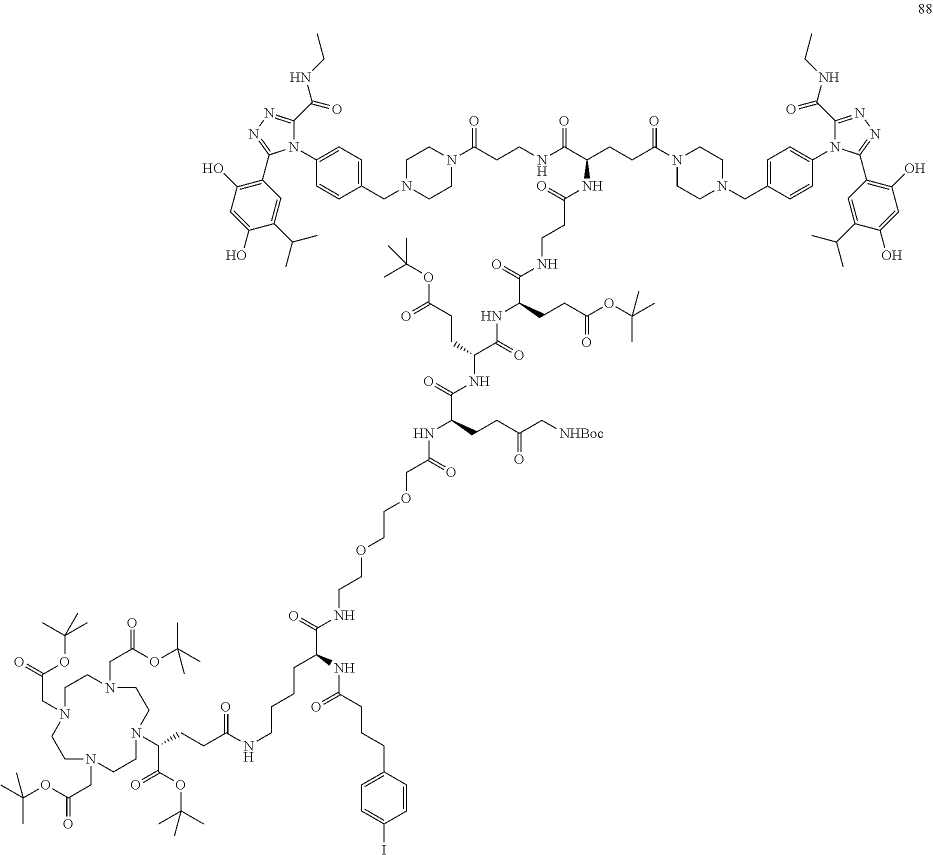 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)[C@@H](CCC(=O)OC(C)(C)C)NC(=O)[C@@H](CCC(=O)OC(C)(C)C)NC(=O)[C@@H](CCC(=O)CNC(=O)OC(C)(C)C)NC(=O)COCCOCCNC(=O)[C@H](CCCCNC(=O)CC[C@H](C(=O)OC(C)(C)C)N3CCN(CC(=O)OC(C)(C)C)CCN(CC(=O)OC(C)(C)C)CCN(CC(=O)OC(C)(C)C)CC3)NC(=O)CCCc3ccc(I)cc3)CC2)cc1]

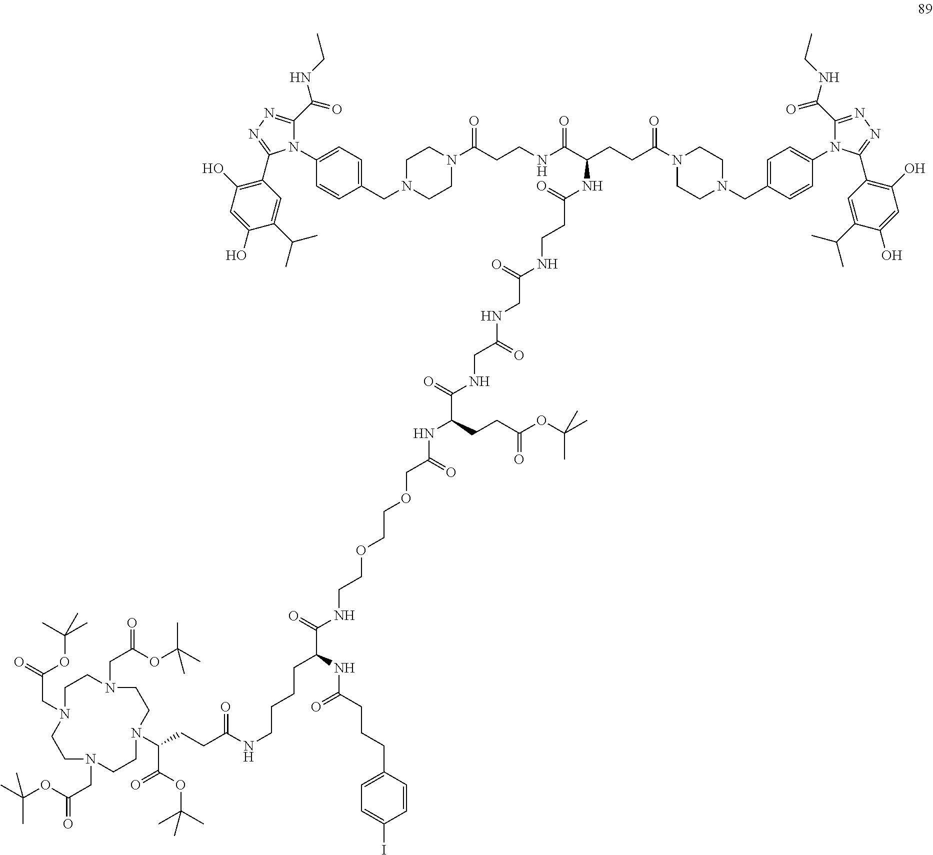 [SMILES: CCNC(=O)c1nnc(-c2cc(C(C)C)c(O)cc2O)n1-c1ccc(CN2CCN(C(=O)CCNC(=O)[C@@H](CCC(=O)N3CCN(Cc4ccc(-n5c(C(=O)NCC)nnc5-c5cc(C(C)C)c(O)cc5O)cc4)CC3)NC(=O)CCNC(=O)CNC(=O)CNC(=O)[C@@H](CCC(=O)OC(C)(C)C)NC(=O)COCCOCCNC(=O)[C@H](CCCCNC(=O)CC[C@H](C(=O)OC(C)(C)C)N3CCN(CC(=O)OC(C)(C)C)CCN(CC(=O)OC(C)(C)C)CCN(CC(=O)OC(C)(C)C)CC3)NC(=O)CCCc3ccc(I)cc3)CC2)cc1]

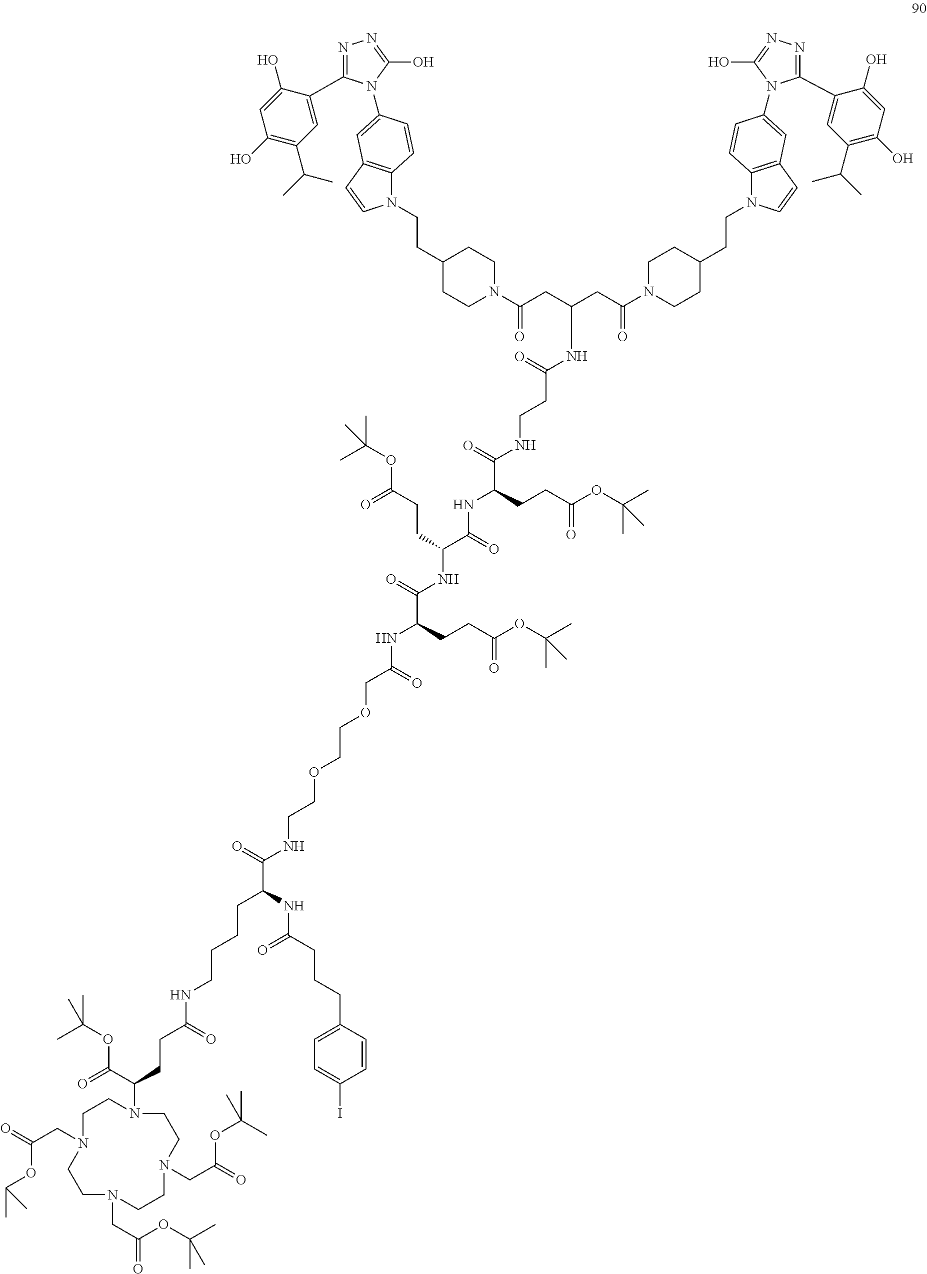 [SMILES: CC(C)OC(=O)CN1CCN(CC(=O)OC(C)(C)C)CCN(CC(=O)OC(C)(C)C)CCN([C@H](CCC(=O)NCCCC[C@H](NC(=O)CCCc2ccc(I)cc2)C(=O)NCCOCCOCC(=O)N[C@H](CCC(=O)OC(C)(C)C)C(=O)N[C@H](CCC(=O)OC(C)(C)C)C(=O)N[C@H](CCC(=O)OC(C)(C)C)C(=O)NCCC(=O)NC(CC(=O)N2CCC(CCn3ccc4cc(-n5c(O)nnc5-c5cc(C(C)C)c(O)cc5O)ccc43)CC2)CC(=O)N2CCC(CCn3ccc4cc(-n5c(O)nnc5-c5cc(C(C)C)c(O)cc5O)ccc43)CC2)C(=O)OC(C)(C)C)CC1]

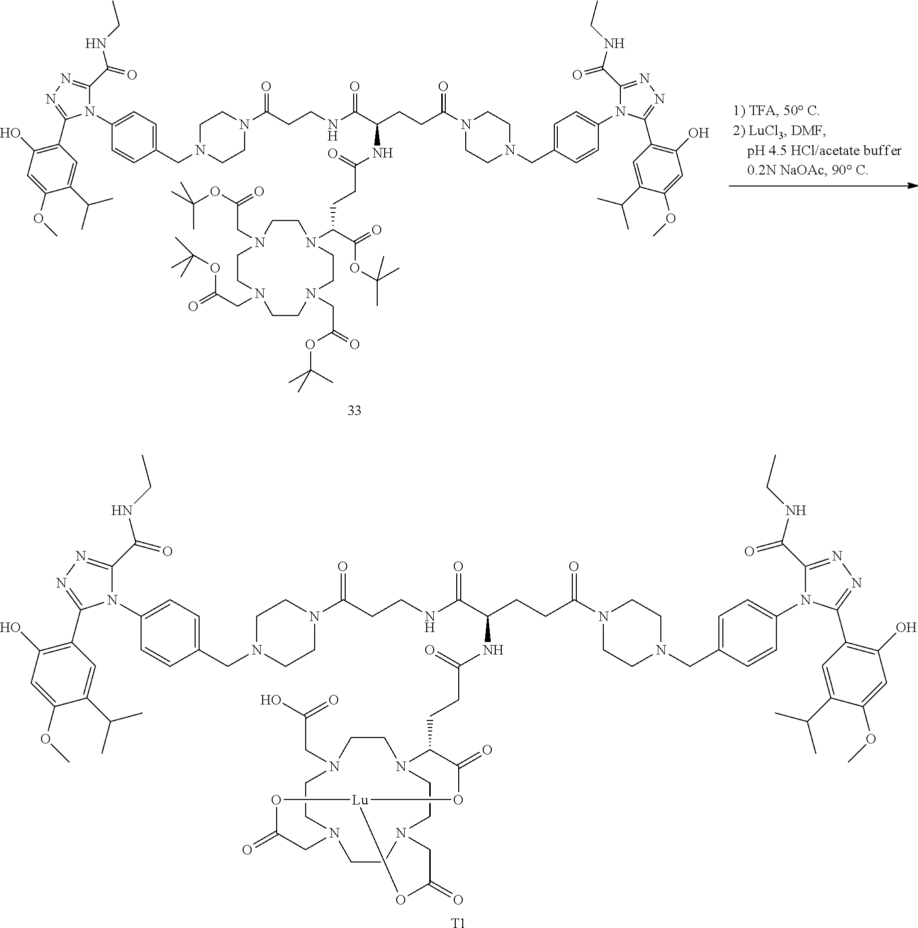

Typical procedure for labeling with cold lutetium:

33 (22.0 mg, 12.1 μmol) was dissolved in trifluoroacetic acid, and the solution heated to 50° C. for 1 h. Toluene (5 mL) was added, and all solvents removed under vacuum. To the remaining residue was added a solution of lutetium(III) chloride (6.25 mg, 22.2 μmol) in pH 4.5 HCl/acetate buffer (1 mL). 0.2N sodium acetate (1 mL) was added, and the solution heated to 90° C. for 20 min. The solution was then cooled to room temperature and purified by preparative HPLC to provide T-2743 (17.3 mg, 9.78 μmol, 80% yield).

The following table lists the starting materials used in the generic lutetium conjugation procedure above, final products, and major ion found in the mass spectrum:

| Starting compound | Product | ESI m/z found: |
|---|---|---|
| 33 | T1 | 885.4 [(M + 2)/2] |
| 34 | T2 | 835.8 [(M + 2)/2] |
| 54 | T3 | 1337.4 [(M + 2)/2] |
| 68 | T4 | 1172.9 [(M + 2)/2] |
| 69 | T5 | 1137.8 [(M + 2)/2] |
| 35 | T6 | 872.3 [(M + 2)/2] |
| 59 | T7 | 1075.9 [(M + 2)/2] |
| 70 | T8 | 1303.9 [(M + 2)/2] |
| 71 | T9 | 1294.4 [(M + 2)/2] |
| 36 | T10 | 887.9 [(M + 2)/2] |
| 58 | T11 | 1050.4 [(M + 2)/2] |
| 55 | T12 | 1374.4 [(M + 2)/2] |
| 56 | T13 | 1366.4 [(M + 2)/2] |
| 57 | T14 | 1366.4 [(M + 2)/2] |
| 50 | T15 | 1475.4 [(M + 2)/2] |
| 75 | T16 | 1387.4 [(M + 2)/2] |
| 90 | T17 | 1344.9 [(M + 2)/2] |
| 37 | T18 | 1664.6 (M + 1) |
| 38 | T19 | 887.8 [(M + 2)/2] |
| 39 | T20 | 835.9 [(M + 2)/2] |
| 40 | T21 | 836.8 [(M + 2)/2] |
| 79 | T22 | 1523.0 [(M + 2)/2] |
| 80 | T23 | 1540.5 [(M + 2)/2] |
| 82 | T24 | 1532.1 [(M + 2)/2] |
| 41 | T25 | 857.3 [(M + 2)/2] |
| 42 | T26 | 864.4 [(M + 2)/2] |
| 73 | T27 | 1189.5 [(M + 2)/2] |
| 72 | T28 | 1123.8 [(M + 2)/2] |
| 74 | T29 | 1138.4 [(M + 2)/2] |
| 43 | T30 | 842.8 [(M + 2)/2] |
| 44 | T31 | 885.8 [(M + 2)/2] |
| 77 | T32 | 1372.9 [(M + 2)/2] |
| 76 | T33 | 1300.8 [(M + 2)/2] |
| 83 | T34 | 1458.6 [(M + 2)/2] |
| 84 | T35 | 1545.5 [(M + 2)/2] |
| 85 | T36 | 1523.4 [(M + 2)/2] |
| 81 | T37 | 1487.9 [(M + 2)/2] |
| 86 | T38 | 1467.4 [(M = 2)/2] |
| 87 | T39 | 1408.1 [(M + 2)/2] |
| 45 | T40 | 1159.4 [(M + 2)/2] |
| 88 | T41 | 1372.2 [(M + 2)/2] |
| 89 | T42 | 1301.0 [(M + 2)/2] |
| 46 | T43 | 816.3 [(M + 2)/2] |

### Example 2: Biodistribution Studies

Lutetium accumulation was measured in tumor, plasma and healthy tissues of NCI-H460 tumor-bearing mice (lung cancer). NCI-H460 tumor bearing mice were dosed with 0.5 mg/kg of lutetium conjugates. At the time points indicated, mice were sacrificed and tumor, liver, kidneys and plasma were taken. All tissues were analyzed for lutetium content by ICP-MS, and lutetium uptake in % ID/g determined by the following equation:

% ID/g=((tissue lutetium in ppb)/175)*(molecular weight of conjugate))/(0.5*(mouse weight in grams)*10)

Biodistribution at 24 h and 72 h is given in the table below.

| | % ID/g uptake | | | | | | | |
|---|---|---|---|---|---|---|---|---|
| Com- | H460 tumor | | Kidney | | Liver | | Plasma | |
| pound | 24 h | 72 h | 24 h | 72 h | 24 h | 72 h | 24 h | 72 h |
| T3 | 7.13 | 2.48 | 17.00 | 6.08 | 2.32 | 0.01 | 0.56 | 0.01 |
| T4 | 3.06 | 2.19 | 4.86 | 2.28 | 0.59 | 0.62 | 0.21 | 0.09 |
| T5 | 1.74 | 0.00 | 3.14 | 1.08 | 0.68 | 0.23 | 0.16 | 0.01 |
| T6 | 7.00 | 4.52 | 6.77 | 2.57 | 10.70 | 8.90 | 1.65 | 1.89 |
| T7 | 7.54 | 4.05 | 24.70 | 13.10 | 1.88 | 1.01 | 4.71 | 0.42 |
| T8 | 1.00 | 0.44 | 3.24 | 0.84 | 0.30 | 0.07 | 0.17 | 0.00 |
| T9 | 2.43 | 0.93 | 8.15 | 2.78 | 0.62 | 0.25 | 26.50 | 1.53 |
| T10 | 3.91 | 3.18 | 14.70 | 7.74 | 7.28 | 4.34 | 2.29 | 0.37 |
| T11 | 6.37 | 1.74 | 14.90 | 5.21 | 1.03 | 0.46 | 0.51 | 0.02 |
| T12 | 9.01 | 5.87 | 34.00 | 18.60 | 3.73 | 2.03 | 5.08 | 0.41 |
| T15 | 2.16 | 0.70 | 12.50 | 4.37 | 1.78 | 1.16 | 2.79 | 0.14 |

Biodistribution at 24 h and Tumor/Kidney ratio (T/K), Tumor/Liver ratio (T/L), Tumor/Plasma ratio (T/P) are given in the table below.

| | | Tumor | Kidney | Liver | | | | |
|---|---|---|---|---|---|---|---|---|
| | | uptake, | uptake, | uptake, | Plasma, | | | |
| | | % ID/g, | % ID/g | % ID/g | % ID/g | | | |
| Compound | Ligand spacer | 24 h | 24 h | 24 h | 24 h | T/K | T/L | T/P |
| CMP48 | H-Pipz-AlaAsp(Apa-D- | 4.64 | 1.50 | 1.99 | 0.95 | 3.08 | 2.33 | 4.87 |
| | Ser3-Lu- | | | | | | | |
| | DOTAGA)Pipz-H | | | | | | | |
| CMP44 | H-Pipz-AlaAsp(Apa-L- | 4.39 | 1.34 | 1.88 | 1.78 | 3.28 | 2.34 | 2.47 |
| | Ser3-Lu- | | | | | | | |
| | DOTAGA)Pipz-H | | | | | | | |
| CMP42 | H(OH)—O-Pipd-PEG12- | 0.57 | 0.26 | 0.06 | 0.02 | 2.23 | 9.89 | 26.76 |
| | Lu-DOTA | | | | | | | |
| CMP41 | H—O-Pipd-PEG12-Lu- | 4.39 | 0.86 | 0.69 | 0.52 | 5.10 | 6.34 | 8.37 |
| | DOTA | | | | | | | |
| CMP40 | H(OH)—O-Pipd-PEG4- | 0.71 | 0.14 | 0.01 | 0.02 | 5.08 | 62.28 | 29.23 |
| | Lu-DOTA | | | | | | | |
| CMP39 | H—O-Pipd-PEG4-Lu- | 1.74 | 0.48 | 0.47 | 0.41 | 3.63 | 3.67 | 4.25 |
| | DOTA | | | | | | | |
| CMP36 | H—O-Pipd- | 3.32 | 3.36 | 8.16 | 0.45 | 0.99 | 0.41 | 7.34 |
| | ApaGlu(PEG-12-Lu- | | | | | | | |
| | DOTA)Pipd-O—H | | | | | | | |
| CMP35 | H—O-Pipd- | 8.776 | 11.87 | 17.67 | 2.212 | 0.74 | 0.50 | 3.97 |
| | ApaGlu(PEG4-Lu- | | | | | | | |
| | DOTA)Pipd-O—H | | | | | | | |
| CMP34 | H--Pipd-PEG12-Lu- | 4.43 | 1.12 | 1.45 | 0.80 | 3.96 | 3.06 | 5.54 |
| | DOTA | | | | | | | |
| CMP33 | H-Pipd-PEG12-Lu- | 3.32 | 1.62 | 0.91 | 0.65 | 2.05 | 3.64 | 5.11 |
| | DOTA | | | | | | | |
| CMP32 | H-Azd-PEG12-Lu- | 2.52 | 1.17 | 0.48 | 0.37 | 2.15 | 5.26 | 6.81 |
| | DOTA | | | | | | | |
| CMP31 | H--Pipd-PEG4-Lu- | 4.20 | 0.84 | 1.83 | 0.81 | 5.00 | 2.30 | 5.19 |
| | DOTA | | | | | | | |
| CMP30 | H-Pipd-PEG4-Lu-DOTA | 1.95 | 0.76 | 3.16 | 0.71 | 2.57 | 0.62 | 2.75 |
| CMP29 | H-Azd-PEG4-Lu- | 2.45 | 1.12 | 1.10 | 0.60 | 2.19 | 2.23 | 4.08 |
| | DOTA | | | | | | | |
| CMP28 | H--Pipd-AlaAsp(Lu- | 8.01 | 8.90 | 10.10 | 7.11 | 0.90 | 0.79 | 1.13 |
| | DOTAGA)Pipd--H | | | | | | | |
| CMP27 | H--Pipd-AlaAsp(Lu- | 7.50 | 6.66 | 14.98 | 4.89 | 1.13 | 0.50 | 1.53 |
| | DOTA)Pipd--H | | | | | | | |
| CMP24 | H--Pipd-SuccLys(Lu- | 12.39 | 4.81 | 11.53 | 13.96 | 2.58 | 1.07 | 0.89 |
| | DOTA)-Pipd--H | | | | | | | |
| CMP51 | H--Pipz-Apa-RGlu3- | 7.54 | 10.37 | 1.35 | 3.01 | 0.7 | 5.6 | 2.5 |
| | Spacer-Trz-Albu- | | | | | | | |
| | Spacer-Lu-DOTAGA | | | | | | | |
| CMP8 | H--Pipz-HpaApd-(Lu- | 5.86 | 2.91 | 3.50 | 1.46 | 2.01 | 1.67 | 4.01 |
| | DOTAGA)-Hpa-Pipz-- | | | | | | | |
| | H | | | | | | | |
| CMP6 | H—O-Pipd-PEG4-Albu- | 3.40 | 0.62 | 0.68 | 1.72 | 5.48 | 4.98 | 1.98 |
| | Lu-DOTA | | | | | | | |
| CMP5 | H—O-Pipd-PEG12- | 12.05 | 3.95 | 3.80 | 8.48 | 3.05 | 3.17 | 1.42 |
| | Albu-Lu-DOTA | | | | | | | |
| CMP4 | H--Pipd-PEG4-Albu- | 8.21 | 3.24 | 2.31 | 9.48 | 2.53 | 3.55 | 0.87 |
| | Lu-DOTA | | | | | | | |
| CMP3 | H--Pipd-PEG12-Albu- | 17.36 | 5.13 | 7.26 | 20.57 | 3.38 | 2.39 | 0.84 |
| | Lu-DOTA | | | | | | | |
| CMP2 | H—O-Pipd-PEG4-Lu- | 0.927 | 0.438 | 0.469 | 0.369 | 2.12 | 1.98 | 2.51 |
| | DOTAGA | | | | | | | |
| CMP1 | H-Pipz-PEG4-Lu- | 1.167 | 0.91 | 0.607 | 0.152 | 1.28 | 1.92 | 7.68 |
| | DOTA | | | | | | | |
| CMP52 | H-Pipz-PEG4-Lu- | 1.541 | 1.37 | 0.524 | 0.404 | 1.12 | 2.94 | 3.81 |
| | DOTAGA | | | | | | | |

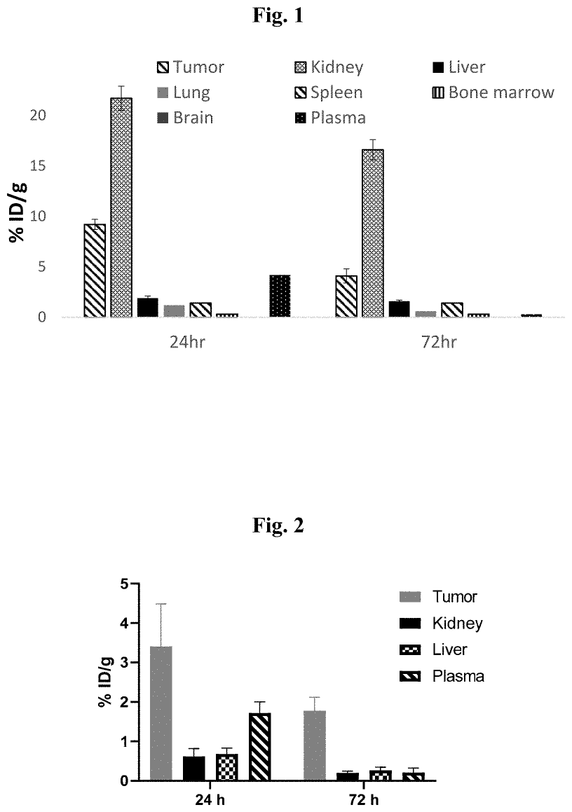

In another study with the NCI-H460 tumor model, the distribution of the radioactive (hot) analog of CMP51 (177Lu) and the distribution of the non-radioactive (cold) analog of CMP51 (175Lu) at 24 hours and 72 hours were tested and compared. The radioactive analog showed the same level of tumor uptake and retention as the cold analog. As shown in FIG. 1, the kidney levels of the hot CMP51 were higher than the tumor levels. High tumor/tissue ratios were observed for the other tissues.

In yet another study with the NCI-H460 tumor model, the distribution of the cold analog of CMP6 was measured at 24 hours and 72 hours. As shown in FIG. 2, CMP6 showed significantly reduced kidney uptake and retention. Liver uptake was also significantly reduced. High tumor to liver and tumor to kidney ratios were observed. These radios were enhanced at 72 hours.

### Example 3: In Vitro HSP90 Binding Studies Using the Conjugates

HSP90 binding was determined by a competitive fluorescence polarization assay with purified N-terminal HSP90α. A series of dilutions of the test compounds were prepared with 10% DMSO in assay buffer and 10 μl of the dilution was added to a 100 μl reaction so that the final concentration of DMSO is 1% in all reactions. The reactions were conducted at room temperature for 3 hours in a 100 μl mixture containing assay buffer, 5 nM FITC Labeled Geldanamycin, 350 ng of N-terminal HSP90α, and the test compound. Fluorescence intensity was measured at an excitation of 485 nm and an emission of 530 nm using a Tecan Infinite M1000 microplate reader. Fluorescence intensity is converted to fluorescence polarization using the Tecan Magellan6 software. The fluorescence polarization data were analyzed using the computer software, Graphpad Prism. The fluorescence polarization (FPt) in absence of the compound in each data set was defined as 100% activity. In the absence of protein and the compound, the value of fluorescent polarization (FPb) in each data set was defined as 0% activity. The percent activity in the presence of the compound was calculated according to the following equation: % activity=(FP−FPb)/(FPt−FPb)×100%, where FP=the fluorescence polarization in the presence of the compound. HSP90 binding data are below.

| Compound | HSP90 Kd (nM) |
|---|---|
| T4 | 0.45 |
| T6 | 0.45 |
| T8 | 0.48 |
| T9 | 0.47 |
| T10 | 0.77 |
| T11 | 0.70 |
| T15 | 0.43 |

### Example 4: Determining the Permeability of Conjugates

In order to test the ability of the conjugates to enter cells, an artificial membrane permeability assay (“PAMPA”) is used. PAMPAs are useful tool for predicting in vivo drug permeability for drugs that enter cells by passive transport mechanisms. LC/MS is used in conjunction with PAMPA assays to determine the ability of the conjugates to permeate cells.

Pre-coated PAMPA plates are warmed to room temperature for at least 30 minutes prior to adding assay components.

Stock solutions are prepared with the conjugates to be tested. In order to make a working solution, either 50 μL of 100 μM Stock in DMSO+950 μL of PBS or 50 μL of 200 μM stock is added to 96 deep well plate, resulting in a 5 μM final concentration or a 10 μM final concentration, respectively. 3004 of the working solution containing each conjugate to be tested is added to the appropriate well of a donor PAMPA plate. 200 μL of PBS is added into the corresponding wells of an acceptor PAMPA plates.

The acceptor plate is lowered onto the donor plate and allowed to incubate for five hours. After five hours, a 50 μL aliquot is removed from each well of each plate and added into a new 96 deep-well plate.

100 μL of methanol containing a predetermined internal standard control compound is added to each aliquot and analyzed by LC/MS. The permeability of each conjugate is calculated.

### Example 5: Synthesis of the Conjugates CMP1-CMP50

The conjugates of the invention may be prepared using any convenient methodology. In a rational approach, the conjugates are constructed from their individual components, targeting moiety, in some cases a linker, and active agent moiety or precursors thereof. In some embodiments, a linker or a precursor thereof is attached to a targeting moiety or a precursor thereof followed by coupling with an active agent moiety or a precursor thereof to furnish the conjugate. In some embodiments, a linker or a precursor thereof is attached to an active agent moiety or a precursor thereof followed by coupling with a targeting moiety or a precursor thereof to furnish the conjugate. The components can be covalently bonded to one another through functional groups, as is known in the art, where such functional groups may be present on the components or introduced onto the components using one or more steps, e.g., oxidation reactions, reduction reactions, cleavage reactions and the like. Functional groups that may be used in covalently bonding the components together to produce the pharmaceutical conjugate include: hydroxy, sulfhydryl, amino, and the like. The particular portion of the different components that are modified to provide for covalent linkage will be chosen so as not to substantially adversely interfere with that components desired binding activity, e.g., for the active agent moiety, a region that does not affect the target binding activity will be modified, such that a sufficient amount of the desired drug activity is preserved. Where necessary and/or desired, certain moieties on the components may be protected using blocking groups, as is known in the art, see, e.g., Green & Wuts, Protective Groups in Organic Synthesis (John Wiley & Sons) (1991).

Alternatively, the conjugate can be produced using known combinatorial methods to produce large libraries of potential conjugates which may then be screened for identification of a bifunctional, molecule with the pharmacokinetic profile. Alternatively, the conjugates may be produced using medicinal chemistry and known structure-activity relationships for the targeting moiety and the active agent moiety. In particular, this approach will provide insight as to where to join the two moieties to the linker. Conjugates CMP1-CMP50 can also be synthesized as shown in Example 1. The targeting moieties, linkers, and the active agent moieties of conjugates CMP1-CMP50 can also be synthesized as shown in Example 1.

The PEG spacers of the present disclosure can be commercially available or can be synthesized by one skilled in the art using routine organic chemistry. In a rational approach, the PEG spacers can be constructed from polyethylene glycol (PEG). The PEG spacers can be constructed from (PEG)ₙ, wherein n is an integer between 1 to 20. (PEG)ₙ can be covalently bonded to functional groups, as is known in the art, where such functional groups may be present on the (PEG)ₙ or introduced onto the (PEG)ₙ using one or more steps. When necessary and/or desired, certain moieties on the (PEG)ₙ may be protected using blocking groups, as is known in the art, see, e.g., Green & Wuts, Protective Groups in Organic Synthesis (John Wiley & Sons) (1991). The PEG linkers of the present disclosure can be homobifunctional or heterobifunctional. For example, in some embodiments, the PEG linkers can have carboxylic acid group on one end and amino group on the other end. The PEG linkers can be synthesized using discrete, monodisperse or polydisperse PEG reagents. The peptides/peptide linkers of the present disclosure can be synthesized according to the methods as shown in Example 1.

CMP1-CMP50 can be synthesized similarly according to the synthetic methods of the conjugates shown in Example 1. CMP2 is synthesized by combining the targeting moiety, TM10 with 100, followed by coupling with (R)-tert-Bu4-DOTAGA and labeling with cold lutetium as shown below. CMP1, CMP22, CMP 29-CMP34 and CMP39-CMP42 are made in a similar manner.

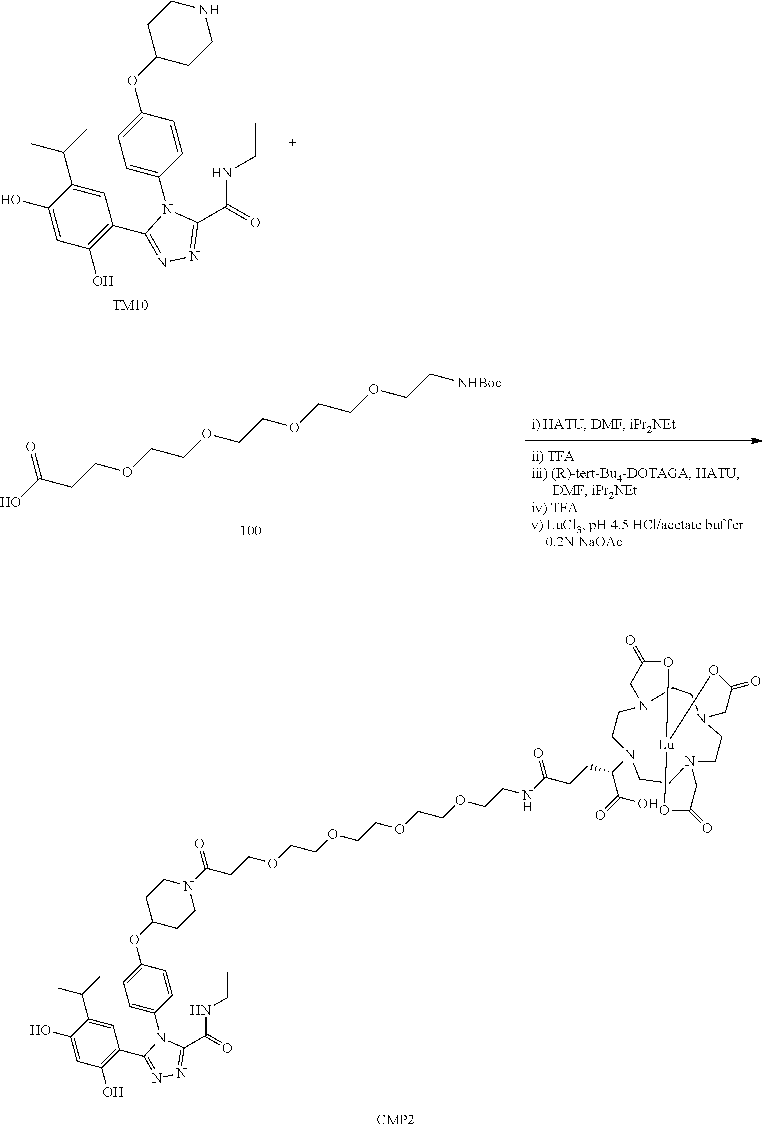

CMP3 is synthesized by combining the linker with the appropriate DOTA reagent to afford 101, followed by coupling with the targeting moiety TMb, and labeling with cold lutetium as shown below. CMP4-CMP6 and CMP23 are made in a similar manner.

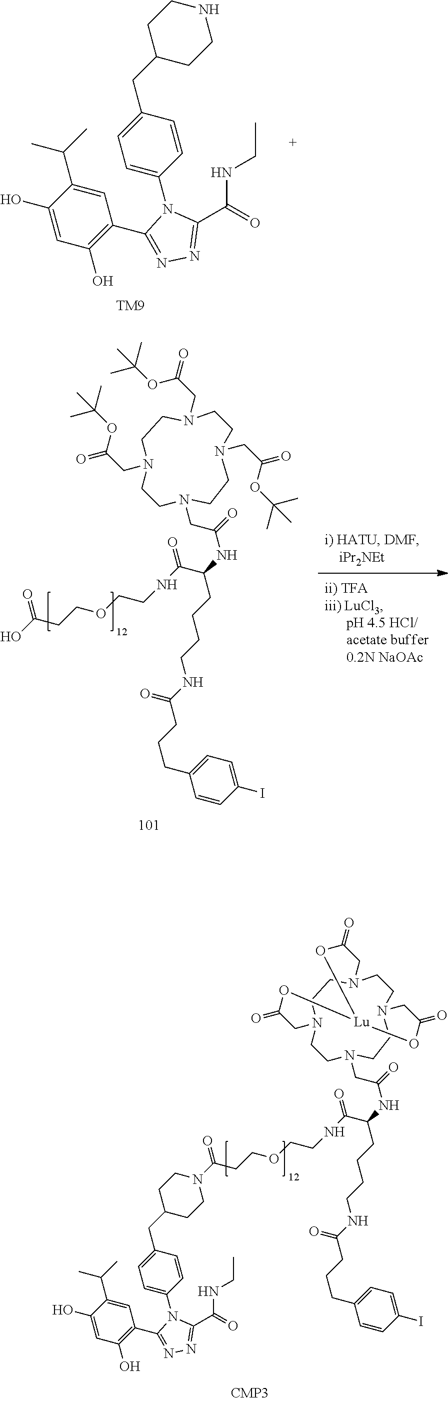

CMP10 is synthesized by reacting the targeting moiety, TM3 with 102, followed by coupling with (R)-tert-Bu4-DOTAGA and labeling with cold lutetium as shown below. CMP7-CMP9, CMP11, CMP12, CMP17-CMP21 and CMP24-CMP28 are made in a similar manner.

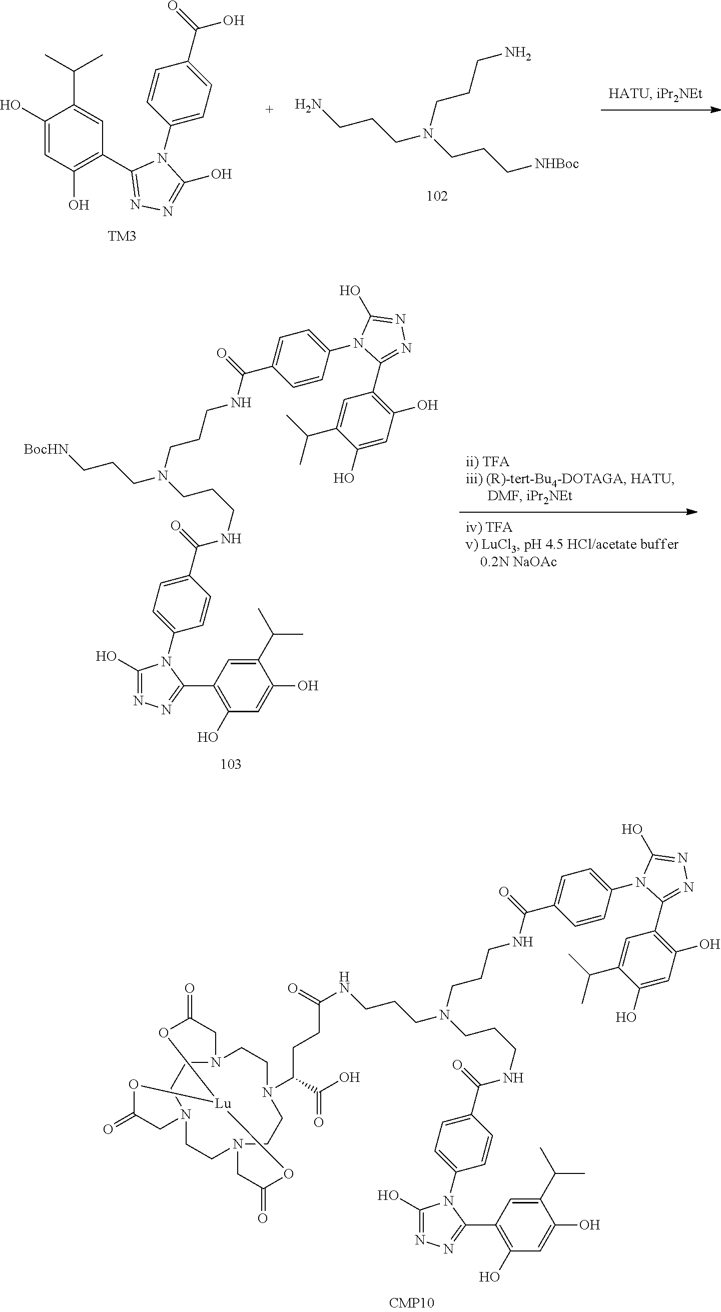

CMP13 is synthesized by coupling the targeting moiety, TM2 with 104 to afford 105. 105 is then coupled with peptide 106, synthesized according to methods demonstrated in Example 1, followed by coupling with (R)-tert-Bu4-DOTAGA and labeling with cold lutetium as shown below. CMP14-CMP16, CMP35-CMP38 and CMP43-CMP50 are made in a similar manner.

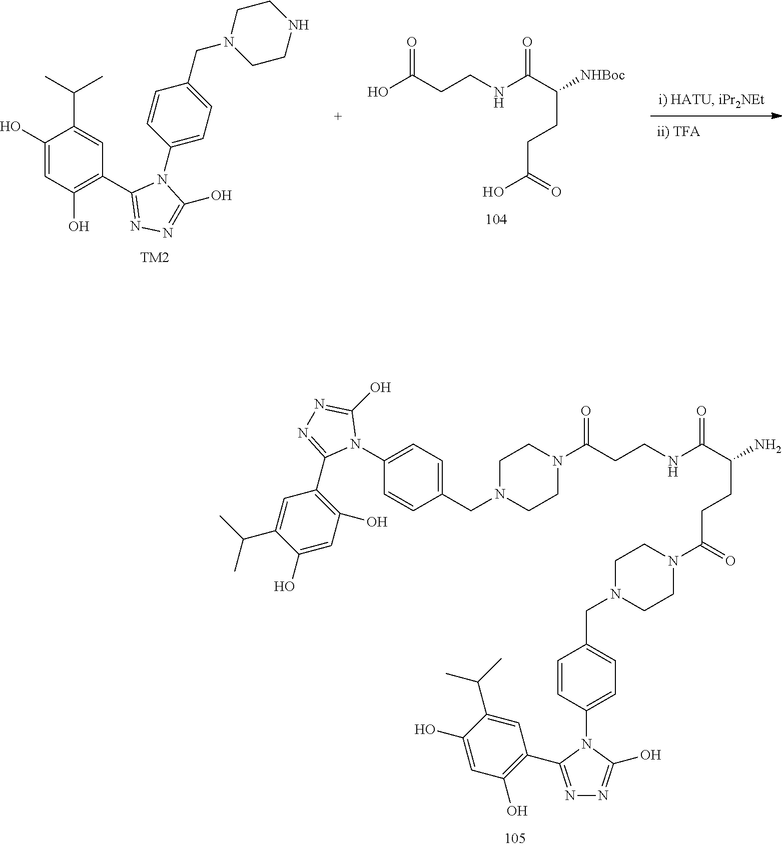

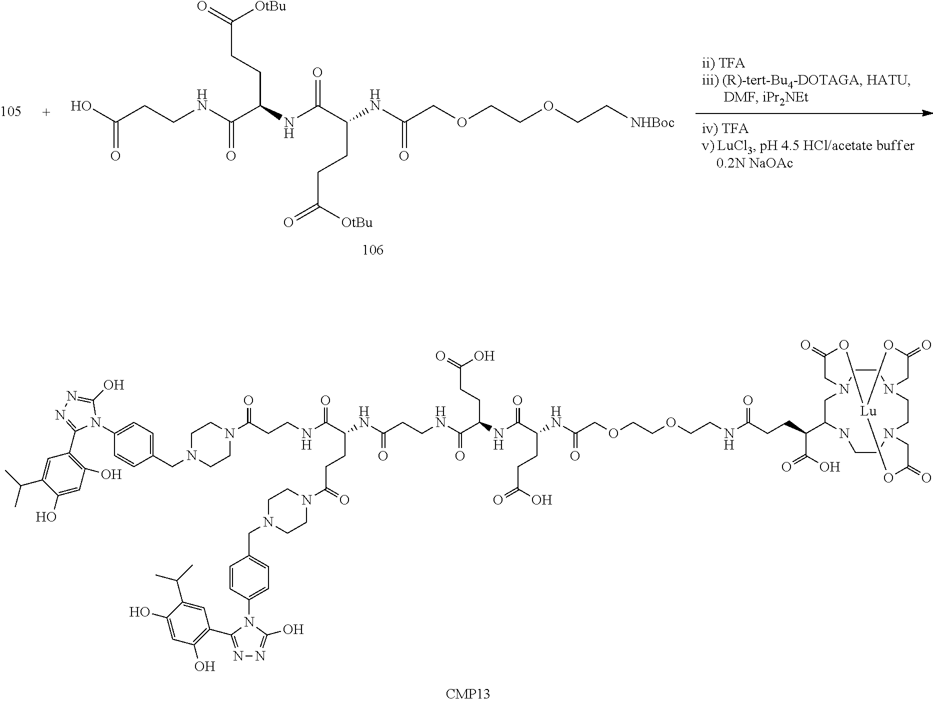

1. A conjugate comprising an active agent coupled, via a linker, to at least one targeting moiety (TM), wherein the TM binds to HSP90, wherein the active agent comprises a radioactive agent or a chelating agent for a radioactive agent, and wherein the conjugate the conjugate comprises a structure of Formula X10:

[symbol]

2. The conjugate of claim 1, wherein the linker comprises a spacer, wherein the spacer comprises at least one amino acid or analog(s) thereof.

3. The conjugate of claim 2, wherein the spacer comprises 2 amino acids or analogs thereof, 3 amino acids or analogs thereof, 4 amino acids or analogs thereof, or 5 amino acids or analogs thereof.

4. The conjugate of claim 2, wherein the conjugate comprises a structure of Formula A10:

[symbol]

5. The conjugate of claim 2, wherein the TM is selected from TM1, TM2, TM3, TM4, TM5, TM8, TM9, TM10, TM11, TM12, TM13, TM14, TM6, TM7, or TM15.

6. The conjugate of claim 2, wherein the conjugate is T1, T2, T4, T5, T6, T8, T9, T10, T27, T28, T29, T39, CMP13, CMP14, CMP15, CMP16, CMP23, CMP37, CMP38, CMP43, CMP44, CMP45, CMP46, CMP47, CMP48, CMP49, CMP50, T1′, T2′, T4′, T5′, T6′, T8′, T9′, T10′, T27′, T28′, T29′, T39′, CMP13′, CMP14′, CMP15′, CMP16′, CMP23′, CMP37′, CMP38′, CMP43′, CMP44′, CMP45′, CMP46′, CMP47′, CMP48′, CMP49′, CMP50′, or a radioactive analog or pharmaceutically acceptable salt thereof.

7. The conjugate of claim 1, wherein the linker comprises a spacer, wherein the spacer comprises polyethylene glycol (PEG).

8. The conjugate of claim 7, wherein the spacer is (PEG)4 or (PEG)12.

9. The conjugate of claim 7, wherein the conjugate comprises a structure of

[symbol]

10. The conjugate of claim 7, wherein the TM is selected from TM1, TM2, TM3, TM4, TM5, TM8, TM9, TM10, TM11, TM12, TM13, TM14, TM6, TM7, or TM15.

11. The conjugate of claim 7, wherein the conjugate is CMP1, CMP2, CMP29, CMP30, CMP31, CMP32, CMP33, CMP34, CMP35, CMP36, CMP39, CMP40, CMP41, CMP42, CMP52, CMP1′, CMP2′, CMP29′, CMP30′, CMP31′, CMP32′, CMP33′, CMP34′, CMP35′, CMP36′, CMP39′, CMP40′, CMP41′, CMP42′, CMP52′, or a radioactive analog or pharmaceutically acceptable salt thereof.

12. The conjugate of claim 1, wherein the conjugate comprises at least one pharmacokinetic modulating unit (PMU).

13. The conjugate of claim 12, wherein the PMU binds to albumin.

14. The conjugate of claim 13, wherein the PMU comprises

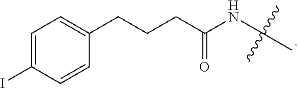 [SMILES: *NC(=O)CCCc1ccc(I)cc1]

15. The conjugate of claim 12, wherein the conjugate comprises a structure of

[symbol]

16. The conjugate of claim 12, wherein the TM is selected from TM1, TM2, TM3, TM4, TM5, TM8, TM9, TM10, TM11, TM12, TM13, TM14, TM6, TM7, or TM15.

17. The conjugate of claim 12, wherein the conjugate is CMP3, CMP4, CMP5, CMP6, T3, T7, T11, T12, T13, T14, T15, T16, T17, T22, T23, T32, T24, T33, T34, T35, T36, T37, T38, T41, T42, CMP3′, CMP4′, CMP5′, CMP6′, T3′, T7′, T11′, T12′, T13′, T14′, T15′, T16′, T17′, T22′, T23′, T32′, T24′, T33′, T34′, T35′, T36′, T37′, T38′, T41′, T42′, or a radioactive analog or pharmaceutically acceptable salt thereof.

18. The conjugate of claim 1, wherein the TM is selected from TM1, TM2, TM3, TM4, TM5, TM8, TM9, TM10, TM11, TM12, TM13, TM14, TM6, TM7, or TM15.

19. The conjugate of claim 1, wherein the conjugate is CMP7, CMP8, CMP9, CMP10, CMP 11, CMP12, CMP17, CMP18, CMP19, CMP20, CMP21, CMP22, CMP24, CMP25, CMP26, CMP27, CMP28, T18, T19, T20, T21, T25, T26, T30, T31, T43, T40, CMP7′, CMP8′, CMP9′, CMP10′, CMP11′, CMP12′, CMP17′, CMP18′, CMP19′, CMP20′, CMP21′, CMP22′, CMP24′, CMP25′, CMP26′, CMP27′, CMP28′, T18′, T19′, T20′, T21′, T25′, T26′, T30′, T31′, T43′, T40′, or a radioactive analog or pharmaceutically acceptable salt thereof.

20. A pharmaceutical composition comprising the conjugate of claim 1 and at least one pharmaceutically acceptable excipient.

21. A method of reducing cell proliferation comprising administering a therapeutically effective amount of at least one conjugate of claim 1 to the cell.

22. The method of claim 21, wherein the cell is a cancer cell.

23. The method of claim 22, wherein the cancer cell is a small-cell lung cancer cell, a non-small-cell lung cancer cell, a sarcoma cell, a pancreatic cancer cell, a breast cancer cell, or a colon cancer cell.

24. A method of treating cancer, comprising administering the pharmaceutical composition of claim 20.

25. The method of claim 24, wherein the cancer is small-cell lung cancer, non-small-cell lung cancer, sarcoma, pancreatic cancer, breast cancer, or colon cancer.
